(19) United States
(12) Patent Application Publication
(10) Pub. No.: US 2011/0263687 A1
(43) Pub. Date: Oct. 27, 2011

(54) RNA MOLECULES AND USES THEREOF

(75) Inventors: John Stanley Mattick, St. Lucia (AU); Ryan J. Taft, St. Lucia (AU); Piero Carninci, Wako-shi (JP)

(73) Assignees: RIKEN, Wako-shi (JP); THE UNIVERSITY OF QUEENSLAND, St. Lucia (AU)

(21) Appl. No.: 12/936,836

(22) PCT Filed: Apr. 7, 2009
(86) PCT No.: PCT/AU2009/000423

§ 371 Date: Mar. 8, 2011

Related U.S. Application Data
(60) Provisional application No. 61/043,012, filed on Apr. 7, 2008.

(51) Int. Cl. A61K 31/7088; C12N 5/10; C12Q 1/68; C40B 40/06; C07H 21/02; A61P 35/00; A61P 35/02; A61P 25/28; A61P 25/16; A61P 3/10; A61P 19/02; A61P 29/00; A61P 9/10; A61P 9/00; A61P 35/04; C12N 15/85

(52) U.S. Cl. 514/44 R; 435/320.1; 435/325; 435/6.1; 506/16; 536/24.1

(57) ABSTRACT

The present invention relates to substantially single-stranded isolated RNA molecules comprising 18 to 19 contiguous nucleotides that corresponds to a non-protein-coding genomic DNA sequence located between −60 and +120 nucleotides from a transcription start site in a mammalian genome. Specifically, the isolated RNA molecules have a high GC content (>60%), are nuclear specific, and may be associated with aberrant gene regulation and/or transcription in various mammalian diseases and conditions. The isolated RNA molecules, modified RNA molecules and fragments thereof may be particularly useful for the diagnosis, prognosis and treatment of diseases such as Crohn's disease, Alzheimer's disease, Parkinson's disease, rheumatoid arthritis, myocardial infarction, diabetes, congenital developmental disorders, coronary heart disease and cancer such as breast cancer, lymphoma, leukemia, aggressive metastatic brain cancers, colorectal cancer, gastric cancer, ovarian cancer and pituitary tumors.

### FIELD OF THE INVENTION

THIS INVENTION relates to molecular biology and particularly RNA molecules. More particularly, this invention relates to non-protein-coding, small RNA molecules associated with gene regulatory activity.

### BACKGROUND OF THE INVENTION

Small regulatory RNAs, are known to be present in all kingdoms of life and involved in many, if not most, fundamental cellular processes (Chu and Rana, 2007; Mattick and Makunin, 2005). For example, the best-studied class of small RNA, microRNAs (miRNAs), are vital regulators of gene expression in eukaryotes (Pillai et al., 2007; Vasudevan et al., 2007) and their mis-regulation is associated with multiple disease states (Rooij and Olson, 2007; Zhang et al., 2007).

Promoter associated small RNAs (PASRs) were identified in a recent comprehensive microarray-based study of the mammalian transcriptome (Kapranov et al., 2007). Due to the limitations of the arrays, however, little is known about the characteristics of these RNAs. Northern blot analyses of selected sequences revealed a range of RNAs larger than 22 nucleotides.

### SUMMARY OF THE INVENTION

Despite the observations that miRNAs are prevalent in mammals, it has remained unclear whether there are as yet unidentified classes of small non-coding RNAs involved in regulating gene transcription and developmental pathways in mammalian and other genomes.

In one broad form, the invention relates to a small RNA molecule that comprises a nucleotide sequence that corresponds to a genomic DNA sequence associated with gene regulation.

In a first aspect, the invention provides a substantially single-stranded isolated RNA molecule that comprises a nucleotide sequence comprising no more than 25 contiguous nucleotides that corresponds to a non-protein-coding genomic DNA sequence associated with gene regulation.

In one preferred form, said isolated RNA molecule comprises 14-22 contiguous nucleotides.

In another preferred form, said isolated RNA molecule comprises 18 or 19 contiguous nucleotides.

Typically, although not exclusively, the isolated RNA molecule is located in, or obtainable from, a cell nucleus.

Preferably, the non-protein-coding genomic DNA sequence associated with gene regulation is located between −200 and +300 nucleotides from a transcription start site (TSS) in a genome.

In a particular form, the nucleotide sequence of the isolated RNA molecule corresponds to a genomic DNA sequence located between −60 and +120 nucleotides from a transcription start site in a genome.

Preferably, the genome is of a eukaryote.

More preferably, the genome is of a metazoan.

Even more preferably, the genome is a vertebrate or mammalian genome.

Advantageously, the genome is of a human.

In certain embodiments, the nucleotide sequence of the isolated RNA molecule is GC enriched.

This aspect of the invention also provides a modified, isolated RNA molecule, a fragment of an isolated RNA molecule and/or an RNA or DNA molecule at least partly complementary to said isolated RNA molecule.

In a second aspect, the invention provides a genetic construct which comprises or encodes one or a plurality of:
(i) an isolated RNA molecule according to the first aspect;
(ii) a fragment of the isolated RNA molecule according to the first aspect;
(iii) a modified RNA molecule according to the first aspect; and/or
(iv) an at least partly complementary RNA or DNA molecule according to the first aspect.

In one particular embodiment, the genetic construct is an expression construct comprising a DNA sequence complementary to one or a plurality of the isolated RNA molecules of the first aspect operably linked or connected to one or more regulatory nucleotide sequences.

In a third aspect, the invention provides a method of identifying the isolated RNA molecule of the first aspect, said method including the step of isolating one or more of said isolated RNA molecules from a nucleic acid sample obtained from an organism.

In a fourth aspect, the invention provides a method of identifying the isolated RNA molecule of the first aspect, said method including the step of identifying a DNA sequence in a genome of an organism which is complementary to the nucleotide sequence of said one or more of said isolated RNA molecules.

In a fifth aspect, the invention provides a computer-readable storage medium or device encoded with data corresponding to one or more of:
(i) an isolated RNA molecule according to the first aspect;
(ii) a fragment of the isolated RNA molecule according to the first aspect;
(iii) a modified RNA molecule according to the first aspect; and/or
(iv) an at least partly complementary RNA or DNA molecule according to the first aspect;

In a sixth aspect, the invention provides a method of identifying a regulatory region in a genome, said method including the step of identifying an isolated RNA molecule according to the first aspect to thereby identify said regulatory region.

In one particular embodiment, said regulatory region is a transcriptionally active location and/or region of the genome.

In another particular embodiment, said regulatory region comprises a regulatory element such as an enhancer.

In yet another particular embodiment, said regulatory region is a non-transcribed region.

In a seventh aspect, the invention provides a method of determining whether a mammal has, or is predisposed to, a disease or condition associated with one or more regulatory regions of a genome, said method including the step of determining whether said mammal comprises one or more isolated RNA molecules according to the first aspect, wherein the or each nucleotide sequence of said one or more isolated RNA molecules corresponds to a genomic DNA sequence associated with said disease or condition.

In one particular embodiment, said regulatory region is a transcriptionally active location and/or region.

Preferably, the mammal is a human.

In an eighth aspect, the invention provides a nucleic acid array comprising a plurality of isolated RNA molecules according to the first aspect, immobilized, affixed or otherwise mounted to a substrate.

In a ninth aspect, the invention provides an antibody which binds:
(i) an isolated RNA molecule according to the first aspect;
(ii) a fragment of the isolated RNA molecule according to the first aspect;
(iii) a modified RNA molecule according to the first aspect; and/or
(iv) an at least partly complementary RNA or DNA molecule according to the first aspect.

In a tenth aspect, the invention provides a kit comprising one or more isolated RNA molecules according to the first aspect, or one or more isolated nucleic acids respectively complementary thereto, and/or an antibody according to the ninth aspect, and one or more detection reagents.

In an eleventh aspect, the invention provides a method of treating a disease or condition in a mammal, said method including the step of administering to the mammal a therapeutic agent selected from the group consisting of:
(i) an isolated RNA molecule according to the first aspect;
(ii) a fragment of the isolated RNA molecule according to the first aspect;
(iii) a modified RNA molecule according to the first aspect;
(iv) an at least partly complementary RNA or DNA molecule according to the first aspect; and/or
(v) an antibody according to the ninth aspect;
   to thereby treat said disease or condition.

In one non-limiting embodiment, said disease or condition is associated with aberrant regulatory activity of one or more genes.

In another non-limiting embodiment, said disease or condition is associated with aberrant transcriptional activity of one or more genes.

Preferably, the mammal is a human.

In a twelfth aspect the invention provides a pharmaceutical composition comprising a therapeutic agent selected from the group consisting of:
(i) an isolated RNA molecule according to the first aspect;
(ii) a fragment of the isolated RNA molecule according to the first aspect;
(iii) a modified RNA molecule according to the first aspect
(iv) an at least partly complementary RNA or DNA molecule according to the first aspect; and
(v) an antibody according to the ninth aspect
   and a pharmaceutically acceptable carrier, diluent or excipient.

In one embodiment, the pharmaceutical composition is for treating a disease or condition, such as but not limited to a disease or condition associated with aberrant regulatory activity of one or more genes.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

FIG. 1. List of human tiRNA sequences (SEQ ID NOs: 1-16913). The specific tiRNAs are listed 5′ to 3′ end (left to right). The sequences are listed in DNA format and thus the DNA base T (Thymine) corresponds to the RNA base U (Uracil).

FIG. 2. Representative tiRNA sequences from three metazoan species. (A) Mouse (SEQ ID NOs 16914-17013); (B) chicken (SEQ ID NOs: 17014-17113); and (C) Drosophila tiRNAs (SEQ ID NOs 17114-17213) were identified in NCBI Geo libraries GSE10364 (Tam et al., 2008), GSE10686 (Glazov et al., 2008), and GSE7448 (Ruby et al., 2007). The specific tiRNAs are listed 5′ to 3′ end (left to right). The sequences are listed in DNA format and thus the DNA base T (Thymine) corresponds to the RNA base U (Uracil).

FIG. 3. Example tiRNA loci. In A and B regions of RNA PolII and Sp1 bindings and a CpGs are depicted as dark bars as annotated. (A) A UCSC screen shot displaying a cluster of tiRNAs and active promoters at the 5′ end of CITED4, which, congruent with the THP-1 monocytic leukemia model, is known to be involved in oligodendroglia) cancers (Tews et al., 2007). (B) Chicken tiRNAs mapped to the human genome, and human tiRNAs are conserved at EIF4G2. (C) Drosophila tiRNAs at the TSS of Adh.

FIG. 4. Distribution and size characteristics of tiRNAs. (a, b, c) Genome-wide distribution of small RNA 5′ ends with respect to TSSs. Black lines indicate the transcription start site, and black arrows depict the direction of transcription. Colored bars represent windows of 10 nt, and those above the x axis depict small RNAs with the same strand orientation as TSSs. Bars below the x axis (negative values) indicate small RNAs antisense to TSSs. The abbreviation ‘k’ indicates thousands. (a) THP-1 small RNA density with respect to all deepCAGE-defined TSSs (blue) or Refgene TSSs (red). Human tiRNAs are found at 1,665 human Refgene TSSs. A detailed depiction of the relationship between sense-strand deepCAGE tags and small RNAs downstream of the TSS is shown in FIG. 8. The abundance of deepCAGE tags antisense to the TSS is shown as a black line below the x axis, and correlates well with the density of small RNAs antisense and upstream of the TSS. Eighteen percent of all TSSs that have sense-strand tiRNAs also have antisense tiRNAs upstream. (b) Chicken small RNA density with respect to Refgene-annotated TSSs from libraries made from embryos collected at day 5 (brown), 7 (orange) and 9 (yellow), which intersected with 320, 507 and 231 Refgene TSS, respectively. Forty-seven percent of Refgene TSSs with sense-strand tiRNAs have antisense tiRNAs upstream. (c) Drosophila small RNAs from Chung et al. (depicted) and Ruby et al. (FIG. 5) are dominantly downstream of the TSS. TiRNAs are present at 9,423 and 2,876 Refgene TSS, respectively. Twenty-nine percent of Drosophila Refgene TSSs with sense-strand tiRNAs also have antisense tiRNAs upstream. (d, e, f) Size distribution of small RNAs that map to the same strand and −60 to +120 relative to the TSS, or on the opposite strand within 400 nt upstream of the TSS. The range of small RNA sizes varies between species owing to different sequencing technology constraints and library preparation techniques. In human (d), chicken (e) and Drosophila (f and FIG. 5), sense and antisense tiRNAs show the same overall size distributions and are dominantly and independently 18 nt. Antisense tiRNAs represent approximately one-third of the small RNAs depicted in each graph. Drosophila shows a minor peak of 21-nt RNAs, which are almost exclusively antisense and upstream of the TSS and may be endogenous siRNAs.

FIG. 5. Drosophila tiRNAs size and position characteristics. Small RNAs were obtained from Ruby et al. (a) The black line indicates the transcription start site, and the black arrow depicts the direction of transcription. Gray bars represent windows of 10 nt, and those above the x axis depict small RNAs with the same strand orientation as the TSS. Bars below the x axis (negative values) indicate small RNAs antisense to the TSS. Small RNAs are dominantly upstream and in the same orientation as the TSS. (b) Small RNAs that map to the same strand and are found in the region −60 to +120 relative to the TSS, or on the opposite strand within 400 nt upstream of the TSS, are dominantly 18 nt.

FIG. 6. Expression of genes with and without tiRNAs. (a) The relationship between gene expression and the occurrence of tiRNAs in human was investigated by comparing the relative expression of all Refgenes with tiRNAs at any time point (1,318 tiRNAs, 947 genes) with Refgenes that do not have tiRNAs at any time point (3,368 genes). Human Refgenes with tiRNAs (gray) at any time point are more highly expressed at each time point than Refgenes without tiRNAs throughout the PMA time series (white). (b) The relationship between tiRNA and gene expression in Drosophila was queried across three emybronic time points. Gene expression data was obtained from Arbeitman et al., and small RNAs from Chung et al. (2008). We found 801, 593, and 647 genes with 2,440, 1,302 and 2,011 tiRNAs in 0-1 h, 2-6 h and 6-10 h embryos, respectively. Drosophila genes with tiRNAs (gray) are more highly expressed than those without tiRNAs (white). *P<0.01, **P<0.001, ***P<0.0001.

FIG. 7. ChIP-chip enrichment of promoters with tiRNAs. The proportion of deepCAGE-defined promoters without tiRNAs (black), deepCAGE promoters with tiRNAs that are not found at canonical protein coding genes (white), and deepCAGE promoters at Refgene TSSs with tiRNAs (gray) associated with regions of the genome showing H3K9 aceylation or PU.1, RNA Pol II, or Sp1 binding is shown. The total number of deepCAGE promoters in each class is indicated above each bar.

FIG. 8. The genome-wide distribution of THP-1 small RNA 5′ends (black bars) and deepCAGE abundance (gray line) relative to transcription start sites (black bar and arrow, indicating the direction of transcription) shows an ˜20 nt offset between peak densities, indicating that tiRNAs are not truncated 5′ capped transcripts.

FIG. 9. Distribution of THP-1 small RNAs at 1 nt resolution with respect the most highly expressed deepCAGE tag from active promoters identified as either broad with peak (PB) or single peak (SP). The black bar and arrow indicate transcription start and the direction of transcription, respectively.

FIG. 10. Size distribution of unannotated THP-1 small RNAs in the most 3′ decile of annotated Refgenes. 3′ end associated small RNAs and tiRNAs are significantly different sizes (P<10⁻⁴; one tailed T-test).

FIG. 11. Size distribution of small RNA tags from CE5, CE7, and CE9.

FIG. 12. Size distribution of chicken small RNAs from the most 3′ decile of Refgenes. 3′ end small RNAs and tiRNAs are significantly different in size (P<10⁻⁴; one tailed T-test).

FIG. 13. Density distribution of THP-1 small RNAs 5′ ends at 0 h time at (A) 10 nt and (B) 1 nt density resolution. The black bar and arrow indicate transcription start and the direction of transcription, respectively. (C) 0 h tiRNA size distribution.

FIG. 14. Illumina expression analysis of Refgenes at time point 0 h with active promoters in comparison to those with active promoters and tiRNAs.

FIG. 15. Enrichment of all 0 h time point deepCAGE tag defined promoters, those with tiRNAs, and those at Refgene TSSs with tiRNAs for H3K9-aceylation or PU.1, RNA Polymerase II, and Sp1 binding.

FIG. 16. tiRNAs (vertical dashes) are associated with ETS1, the only gene known to be significantly associated with monocytic leukemia progression, consistent with the THP-1 cell model.

FIG. 17. Size and abundance of small RNAs that map −60-120 to a Refgene TSS. Nuclear small RNAs (black) show characteristics typical of tiRNAs. Cytosolic small RNAs (grey) are very weakly expressed proximal to Refgene TSSs and are dominantly 21 nt.

FIG. 18. tiRNA chromatin mark enrichments.

FIG. 19. Unannotated 18 nt small RNAs are enriched at specific chromatin marks. All unannotated small RNAs (black), which are dominantly 18 nt, and the subset of unannotated small RNAs (also dominantly 18 nt) that do not map within a UCSC KnownGene annotation (grey) are over-represented at active chromatin markers (left half of the graph) and under-represented at “silencing” chromatin markers (right third of the graph).

### DETAILED DESCRIPTION OF THE PREFERRED EMBODIMENT

To investigate transcription start site-associated small RNAs in detail, the present inventors analyzed the relationship between transcriptional start sites and small RNAs present in deep sequencing libraries from human cells, mouse, chicken, and Drosophila.

The present invention arises from the surprising discovery of a novel class of “transcription initiation” RNA molecules (tiRNAs) that may be associated with gene regulation. In a particular embodiment, tiRNA may comprise a nucleotide sequence corresponding to a region of a genome located at or near a transcriptional start site (TSS), for example, within between −200 and +300 nucleotides of a TSS, or within −60 to +120 nucleotides of a TSS.

These small RNA molecules exhibit different characteristics to the small non-coding RNA molecules (miRNA) previously identified. The present invention is based on the inventors' identification of tiRNA molecules, the manipulation of these tiRNAs and the use of tiRNAs to characterize their role and function in cells. The invention also concerns methods and compositions for identifying tiRNAs, arrays comprising tiRNAs (tiRNA array) and use of tiRNAs for diagnostic, therapeutic and prognostic applications in mammals, particularly humans.

For the purposes of this invention, by “isolated” is meant present in an environment removed from a natural state or otherwise subjected to human manipulation. Isolated material may be substantially or essentially free from components that normally accompany it in its natural state, or may be manipulated so as to be in an artificial state together with components that normally accompany it in its natural state. The term “isolated” also encompasses terms such as “enriched”, “purified”, “synthetic” and/or “recombinant”.

The term “nucleic acid” as used herein designates single- or double-stranded mRNA, RNA, cRNA, RNAi and DNA inclusive of cDNA and genomic DNA. Nucleic acids may comprise naturally-occurring nucleotides or synthetic, modified or derivatized bases (e.g. inosine, methyinosine, pseudouridine, methylcytosine etc). Nucleic acids may also comprise chemical moieties coupled thereto to them. Examples of chemical moieties include, but are not limited to, locked nucleic acids (LNAs), peptide nucleic acids (PNAs), cholesterol, 2′O-methyl, Morpholino, and fluorophores such as HEX, FAM, Fluorescein and FITC.

According to a first aspect, the invention provides a substantially-single stranded, isolated RNA molecule (referred to herein as a “transcription initiation RNA” or “tiRNA”) comprising no more than 25 contiguous nucleotides that corresponds to a non-protein-coding genomic DNA sequence associated with gene regulation.

Preferably, the tiRNA molecule comprises 14-22 contiguous nucleotides.

Typically, the tiRNA molecule comprises 18 or 19 contiguous nucleotides.

Preferably, said non-protein-coding genomic DNA sequence is located between −200 and +300 nucleotides from a transcription start site in a genome.

More preferably, the nucleotide sequence of the tiRNA molecule corresponds to a genomic DNA sequence located between −60 and +120 nucleotides from a transcription start site in a genome.

Typically, the 5′ end of a tiRNA molecule corresponds to a genomic DNA sequence located between −50 and +70 nucleotides from a transcription start site in a genome.

In this context, “corresponding to” and “corresponds to” means that the tiRNA molecule has a nucleotide sequence of, or a sequence complementary to, a genomic DNA nucleotide sequence. It will be appreciated that this definition should take into account that RNA uses a U instead of a T, as found in DNA.

Typically, the tiRNA does not encode a peptide or a protein encoded by a genome. Accordingly, the tiRNA comprises a nucleotide sequence that is referred to herein as “non-coding”.

While in one embodiment said tiRNA molecule has a nucleotide sequence transcribed from the corresponding DNA sequence, it will be appreciated that said tiRNA molecule may be chemically-synthesized de novo, rather than transcribed from a DNA sequence.

Chemical synthesis of RNA is well known in the art. Non-limiting examples include RNA synthesis using TOM amidite chemistry, 2-cyanoethoxymethyl (CEM), a 2′-hydroxyl protecting groups and fast oligonucleotide deprotecting groups.

As hereinbefore described, the nucleotide sequence of a tiRNA molecule is typically GC rich. By this is meant, that the percent GC content of the nucleotide sequence is substantially greater than the average GC content of the genome from which the tiRNA is derived. This GC contect also differs from that of miRNAs.

On average, the GC content of tiRNAs is greater than 50%, greater than about 55%, greater than about 60%, greater than about 65%, or greater than about 70% compared to about 50% for miRNAs.

It will be appreciated that this comparison is organism dependent hence the actual GC content will vary for tiRNAs of each different organism.

For example, in humans the average GC content of tiRNAs is about 72% whereas the average GC content of tiRNAs in chicken is about 65%.

It will also be appreciated that tiRNAs typically, although not necessarily, comprise a nucleotide sequence that is located within at least one CpG island.

It will further be appreciated that tiRNAs typically, although not necessarily, comprise a nucleotide sequence that comprises at least one CpG dinucleotide.

As evident from the foregoing, a tiRNA may be transcribable from a regulatory region of a genome.

In one embodiment, said regulatory region is associated with the transcription of a gene or locus encoding a protein, a regulatory RNA or other transcriptionally primed region.

In one particular embodiment, said regulatory region is a transcriptionally active region.

In many cases, but not exclusively, a tiRNA transcribable from a regulatory region of a genome may be associated with an RNA polymerase II promoter and/or an Sp1 transcription factor binding site.

It will further be appreciated that a tiRNA and the regulatory region (e.g. a TSS) with which it is associated, typically, although not necessarily, maps to a Refgene promoter or promoter region.

It will also be appreciated that Refgene promoters or promoter regions associated with tiRNAs typically, although not necessarily, exhibit no Gene Ontology term enrichment.

In some particular embodiments, the tiRNAs may be located at a TSS associated with a non-protein-coding gene or a weakly expressed non-canonical gene.

It will also be appreciated that the tiRNAs may, in some embodiments, be located at a TSS of a regulatory element that regulates the transcription of a gene at a distal location.

Typically, the regulatory element is an enhancer although without limitation thereto.

Accordingly, interference of a tiRNA at a regulatory element, such as an enhancer, may influence the transcription and/or expression of a gene that is located distally (e.g. up to thousands of bases away) to said tiRNA.

In certain embodiments, a tiRNA may be located at a region of a genome with (i) PolII binding and/or (ii) a high density of chromatin marks.

In one particular embodiment, the isolated tiRNA molecule of the invention is associated with one or more chromatin marks.

By “chromatin mark” is meant a specific signature that is indicative of a genomic region with increased gene regulatory activity.

Typically, although not exclusively, genes associated with a high density of the isolated tiRNA molecules show enrichment for chromatin marks such as H2AK5ac, H2AK9ac, H2AZ, H2BK120ac, H2BK12ac, H2BK20ac, H2BK5ac, H3K18ac, H3K23ac, H3K27ac, H3K36ac, H3K36me1, H3K4ac, H3K4me3, H3K79me2, H3K79me3, H3K9ac, H4K12ac, H4K16ac, H4K20me1, H4K5ac, H4K8ac, H4K91ac.

In some cases, genes associated with a high density of tiRNA molecules may also be associated with PolII binding and H2AZ histones.

It will therefore be appreciated that the isolated tiRNA molecules may be directly involved in the regulation of chromatin modification, activation and/or repression of gene expression.

For example, some nuclear-specific isolated tiRNA molecules may be enriched at genomic regions comprising “activating” chromatin marks such as H3K9ac, H3K4me3, and H3K120ac and may be under-represented or absent at regions with “silencing” chromatin marks.

Typically, an isolated tiRNA molecule that is over-represented at an active chromatin mark is involved in gene regulation by facilitating changes to chromatin structure.

Typically, although not exclusively, tiRNA molecules do not form secondary structures, such as stem and loop structures. Accordingly, tiRNA molecules are substantially free of internal base-pairing. In this context, by “substantially free” is meant fewer than 3, 2 or 1 internal base pairs.

Therefore, in one particularly preferred embodiment, the invention provides a substantially single-stranded isolated tiRNA molecule, wherein said isolated tiRNA molecule comprises a nucleotide sequence that:
(i) consists of 18 or 19 contiguous nucleotides that corresponds to a non-protein-coding genomic DNA sequence located between −60 and +120 nucleotides from a transcription start site (TSS) in a mammalian genome;
(ii) comprises a 5′ end that corresponds to a genomic DNA sequence located between −50 and +70 nucleotides from a TSS in a mammalian genome;
(iii) comprises a GC content greater than 60%;
(iv) is located within at least one CpG island
(v) comprises at least one CpG dinucleotide;
(vi) is transcribable from a regulatory region of a genome located at or near a TSS associated with an RNA polymerase II promoter and/or an Sp1 transcription factor binding site; and
(vii) is substantially free of internal base-pairing.

Preferably, the genome is a human genome.

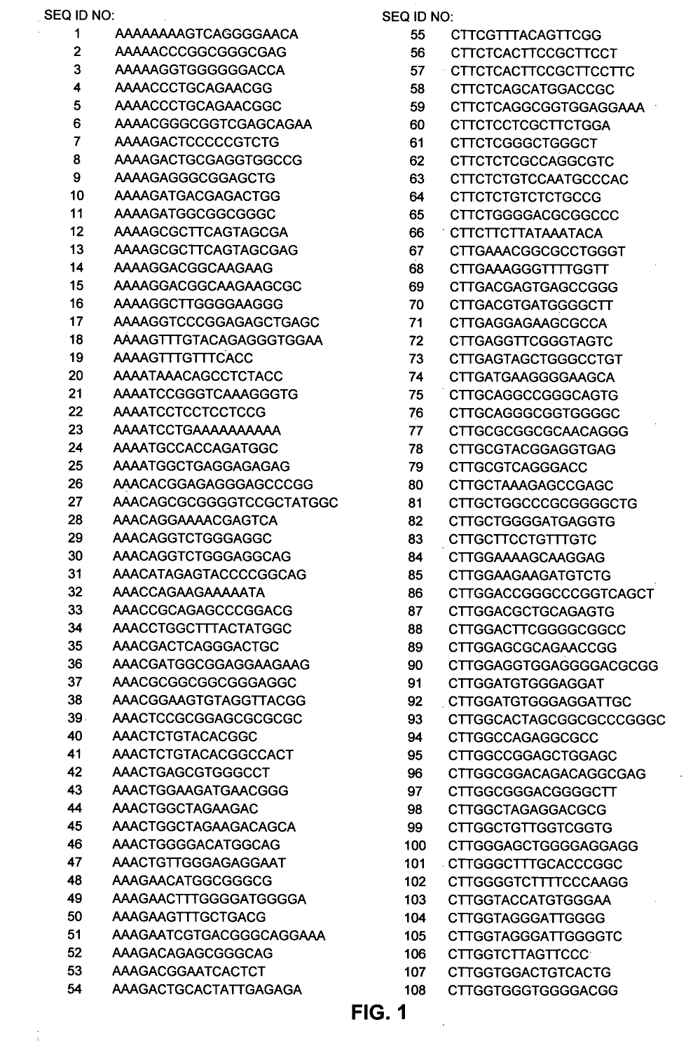

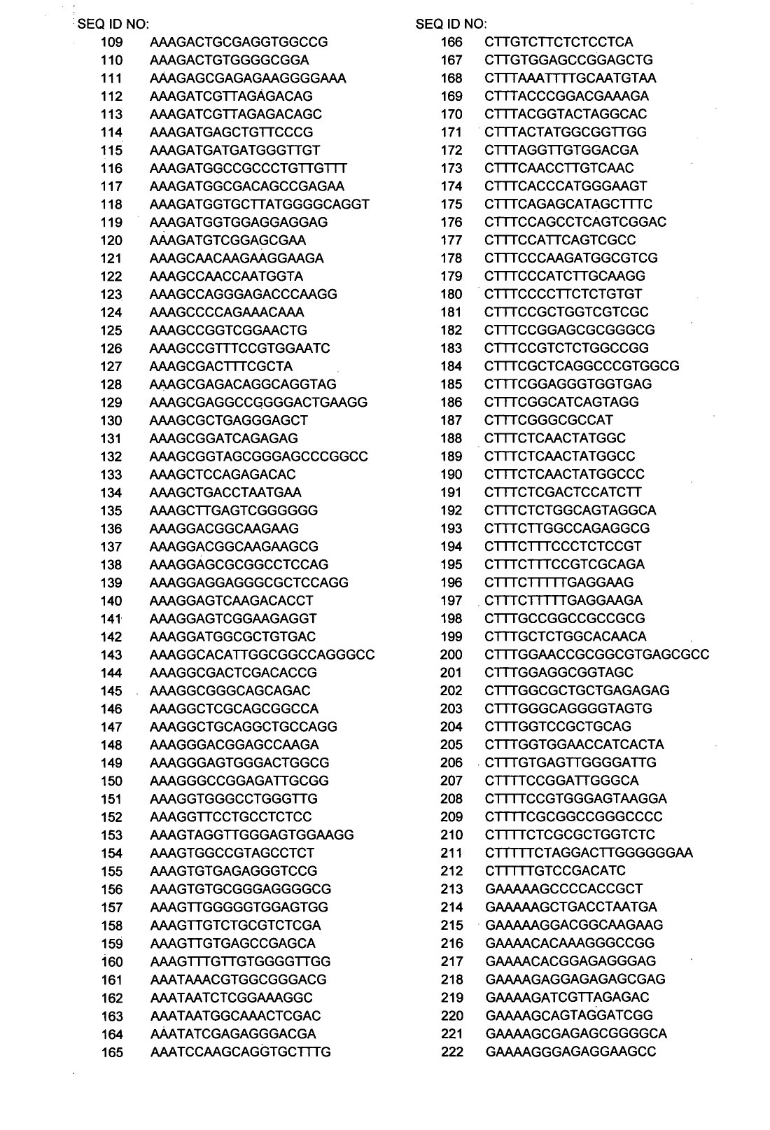

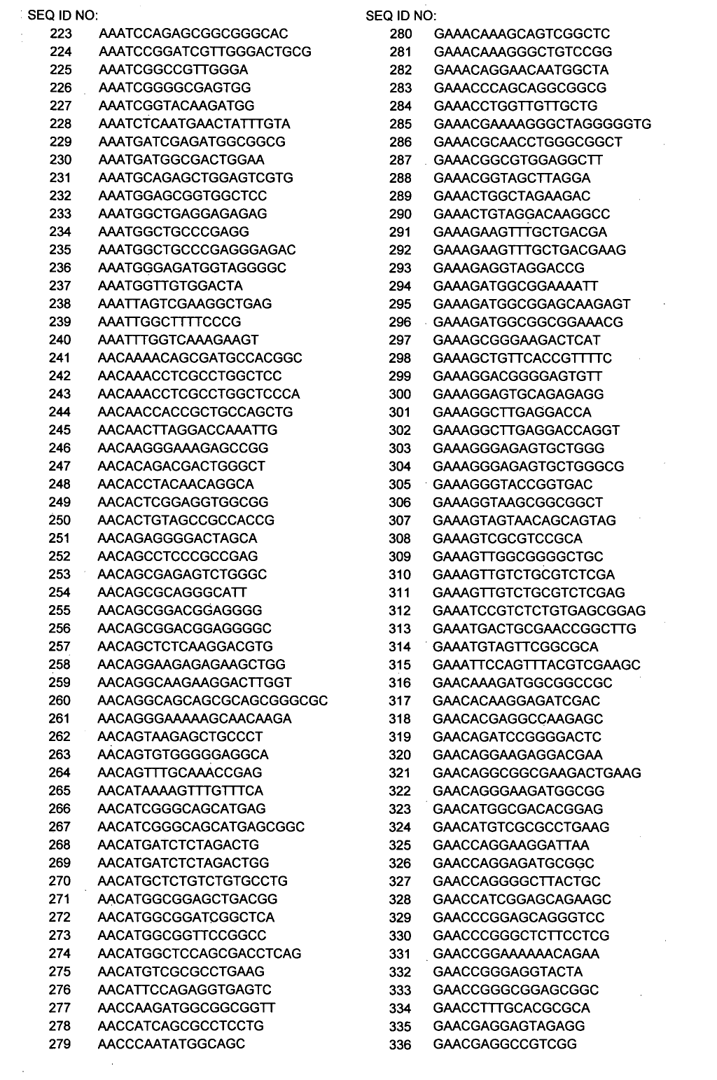

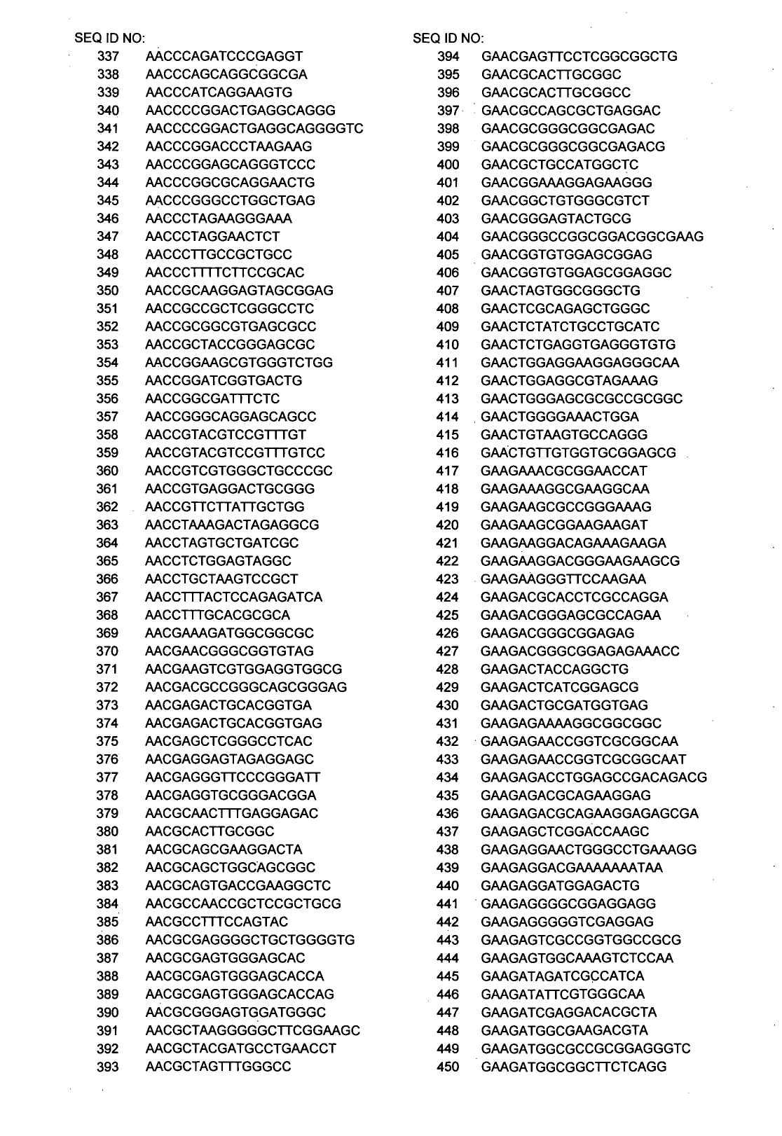

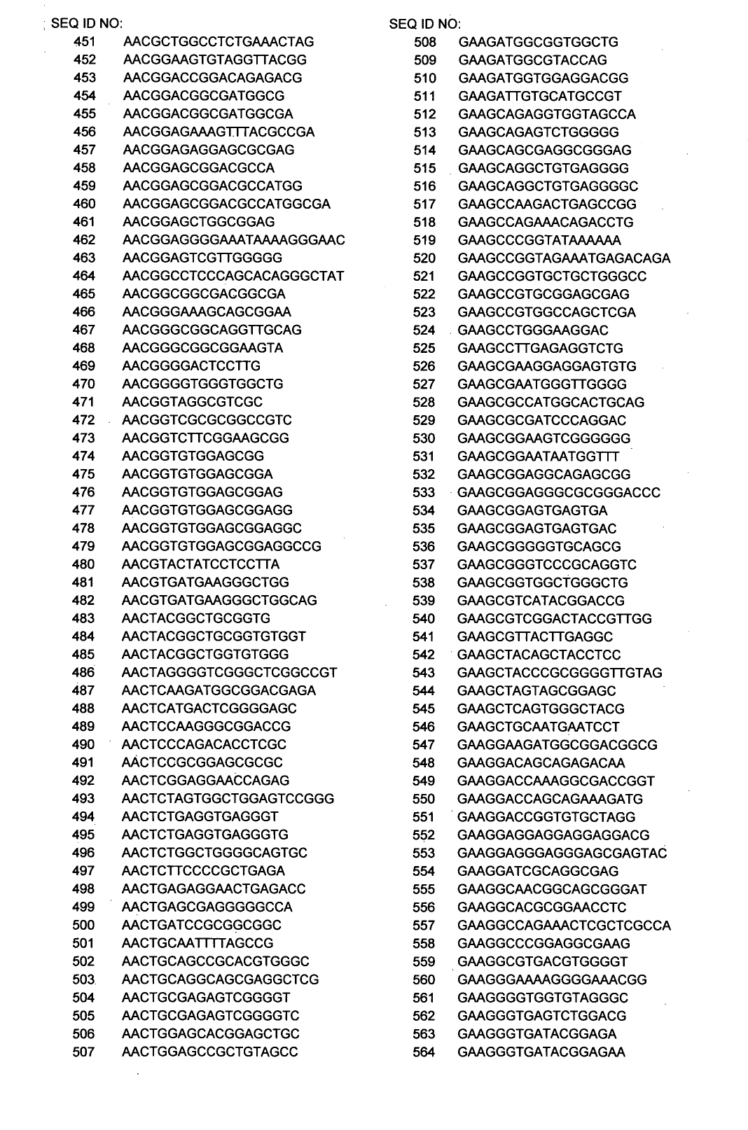

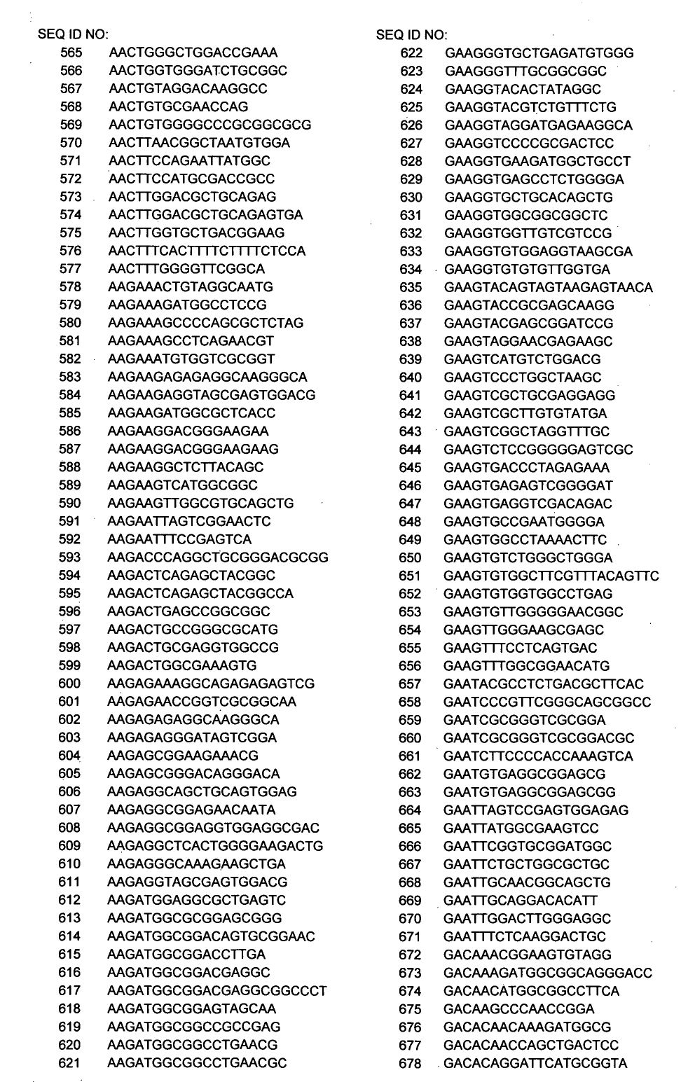

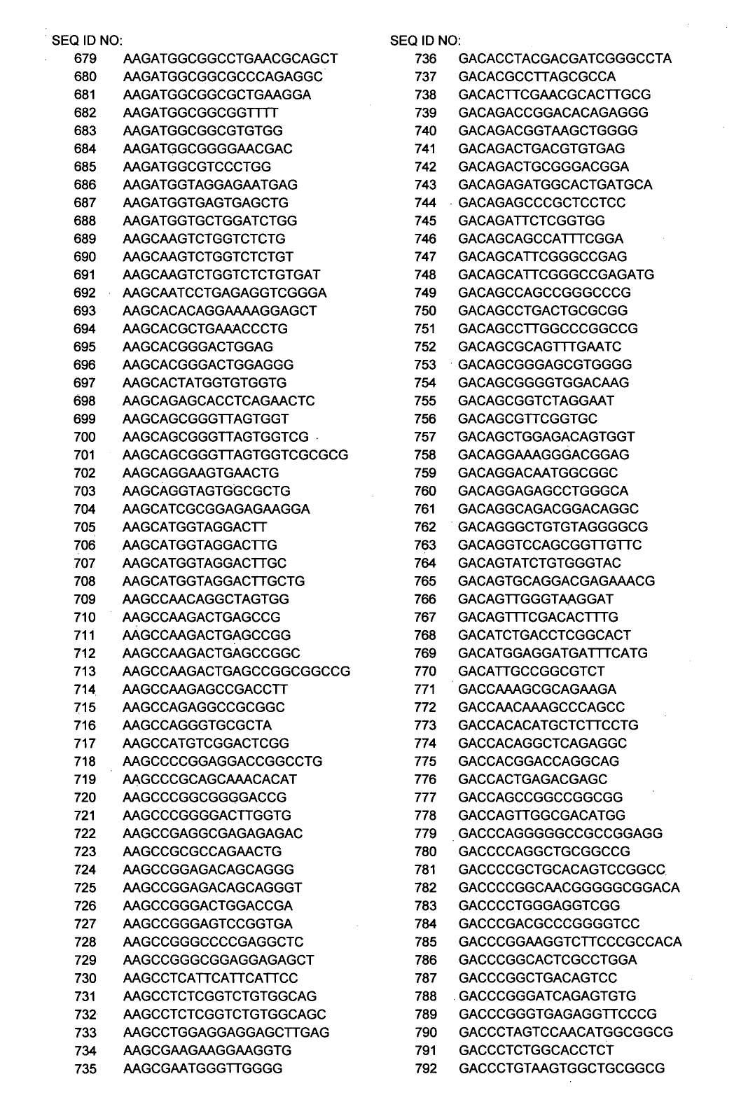

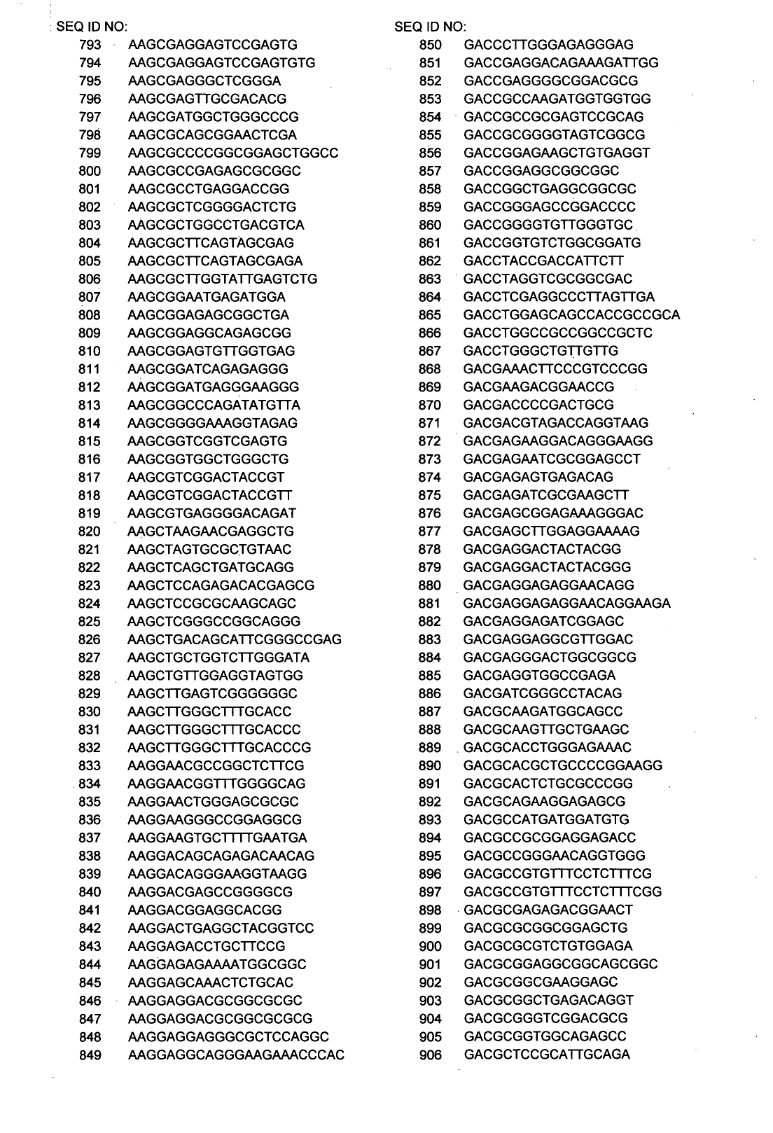

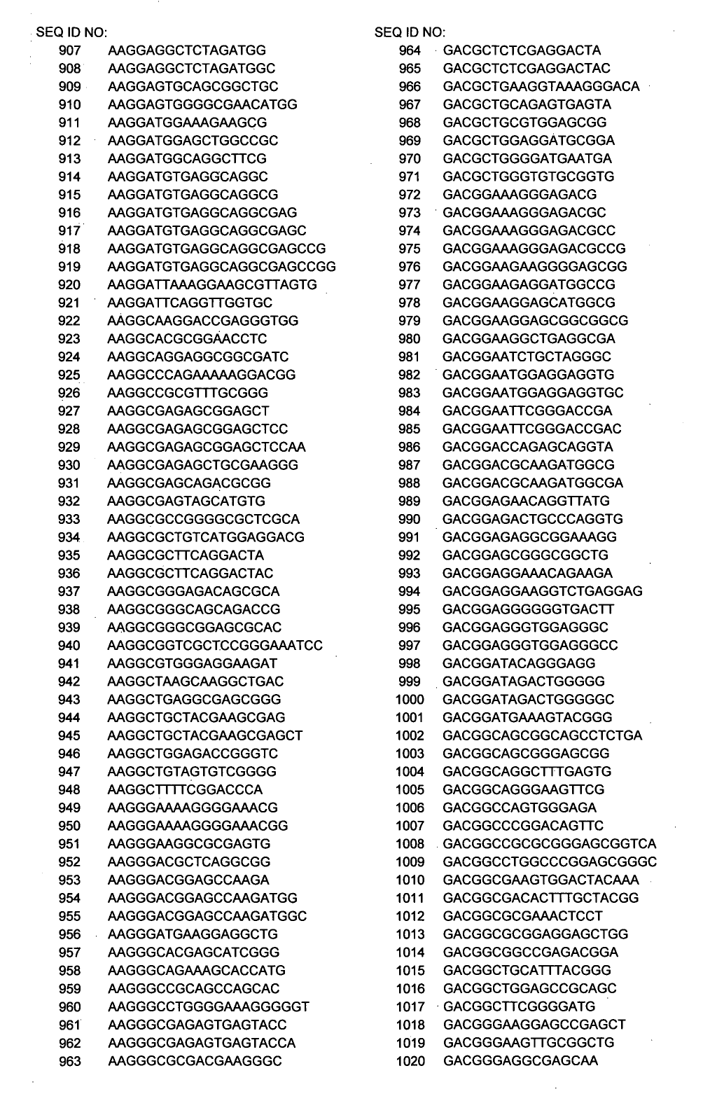

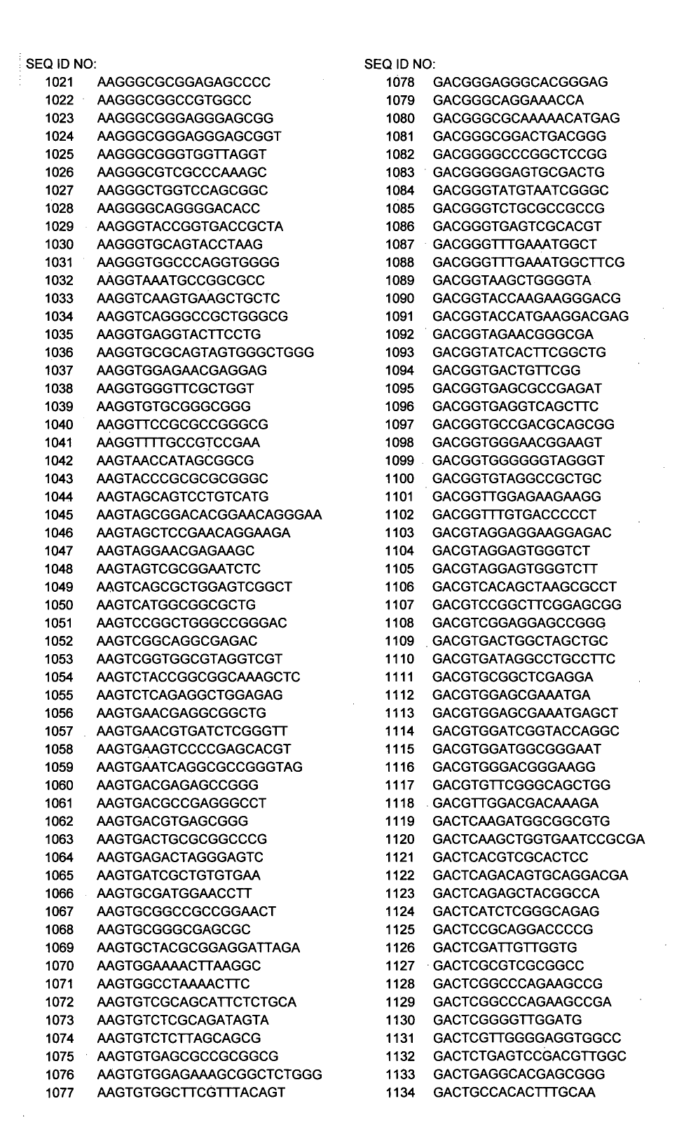

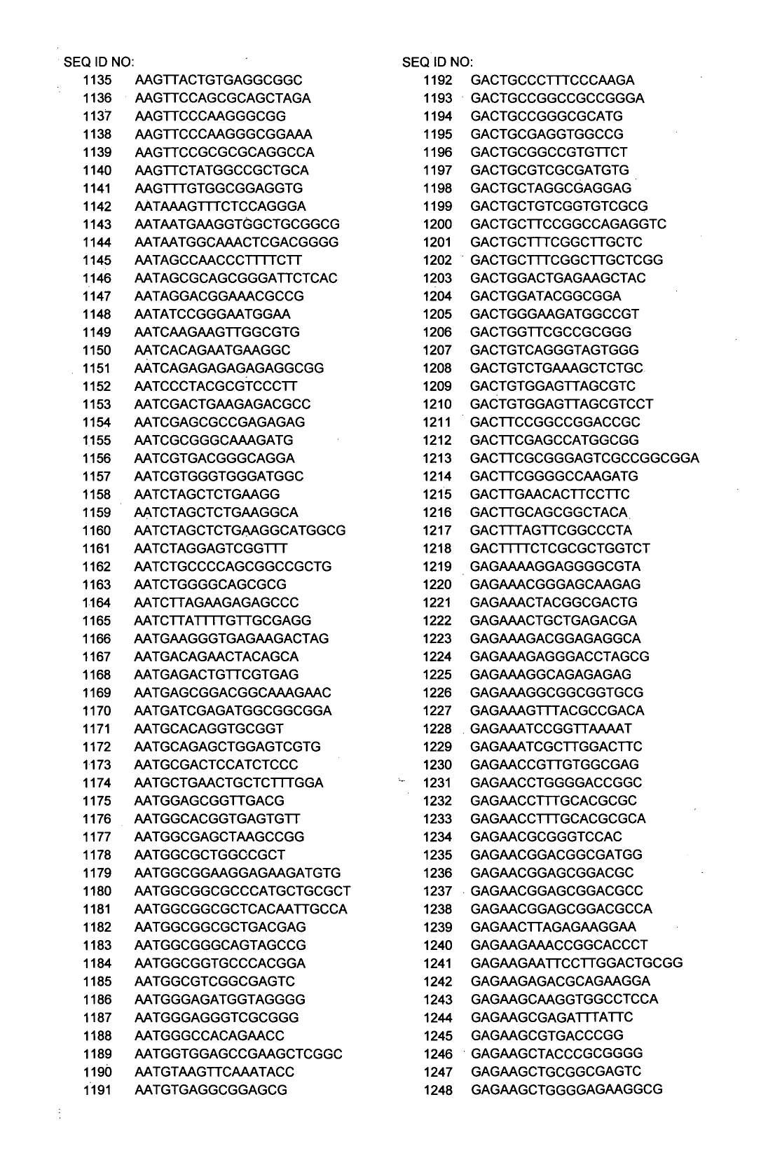

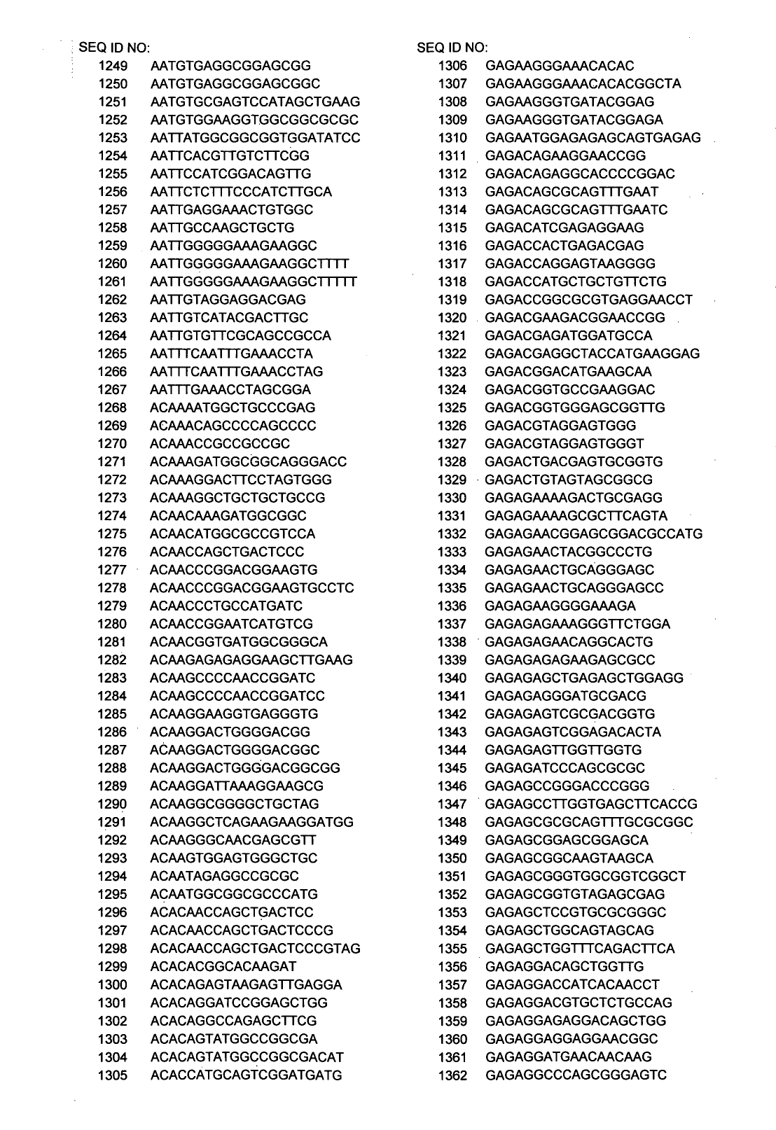

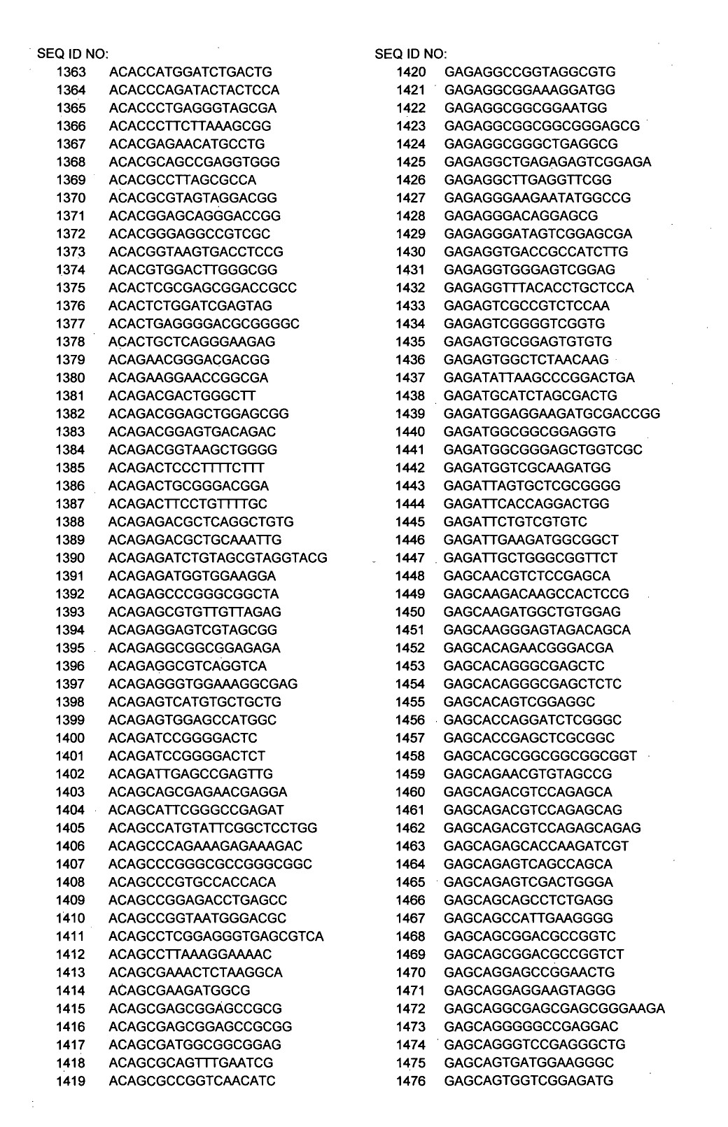

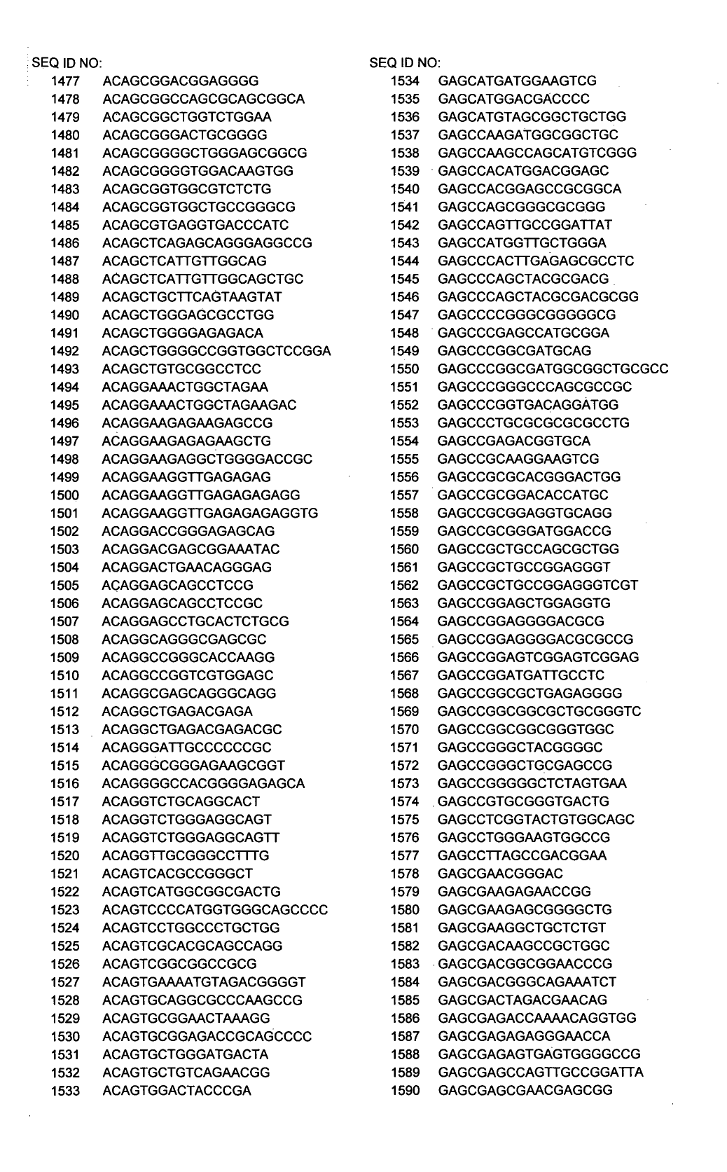

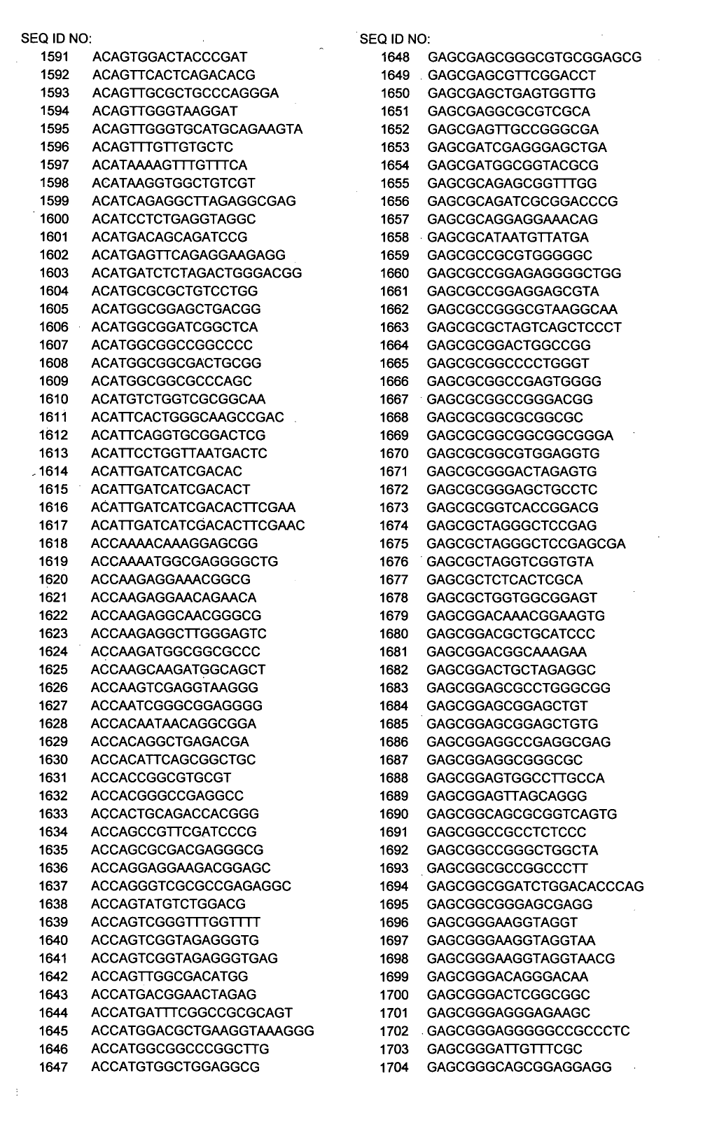

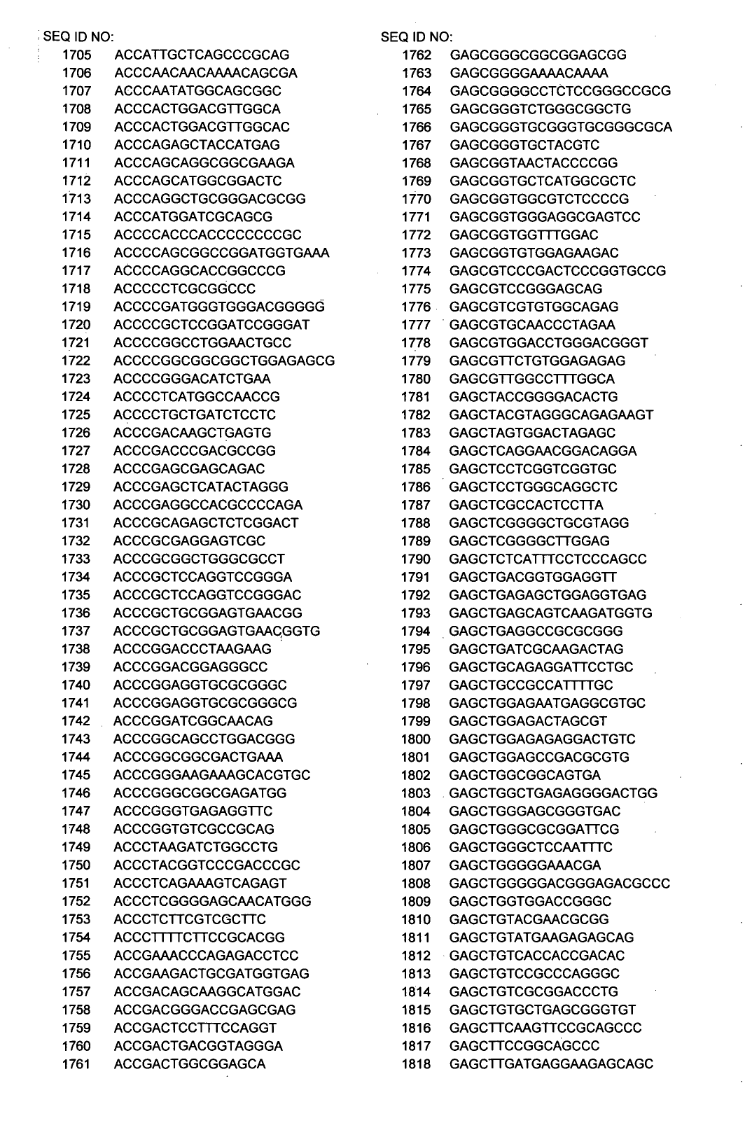

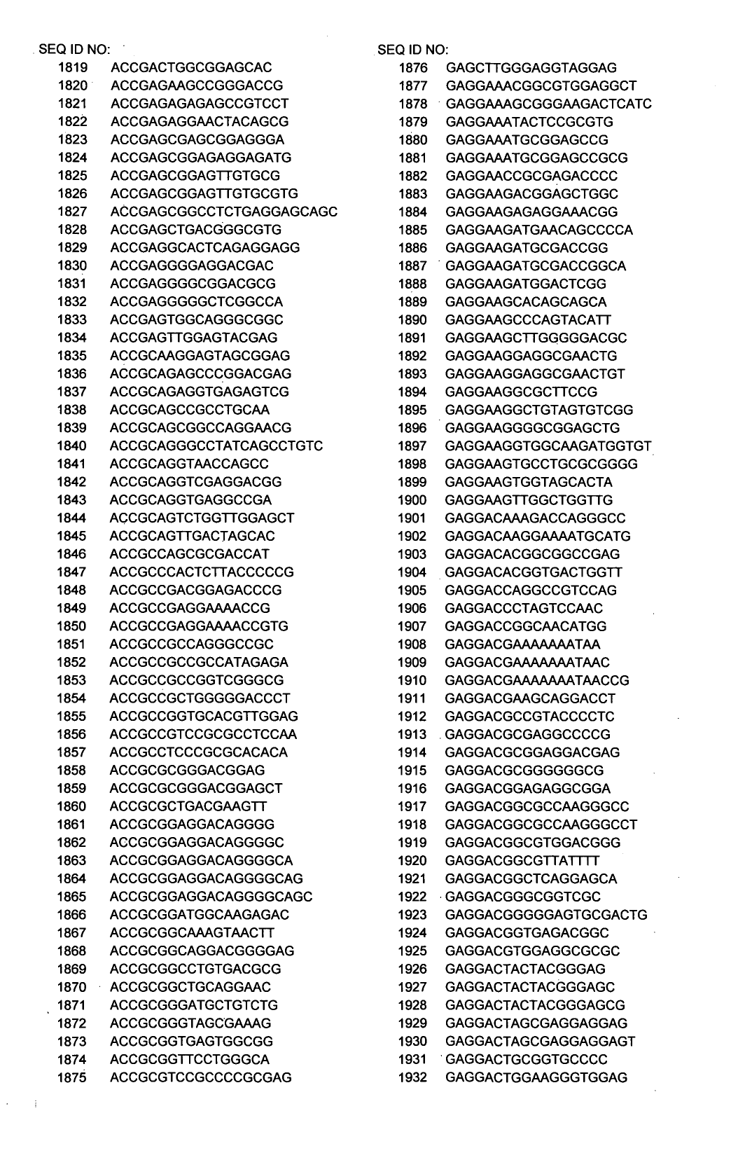

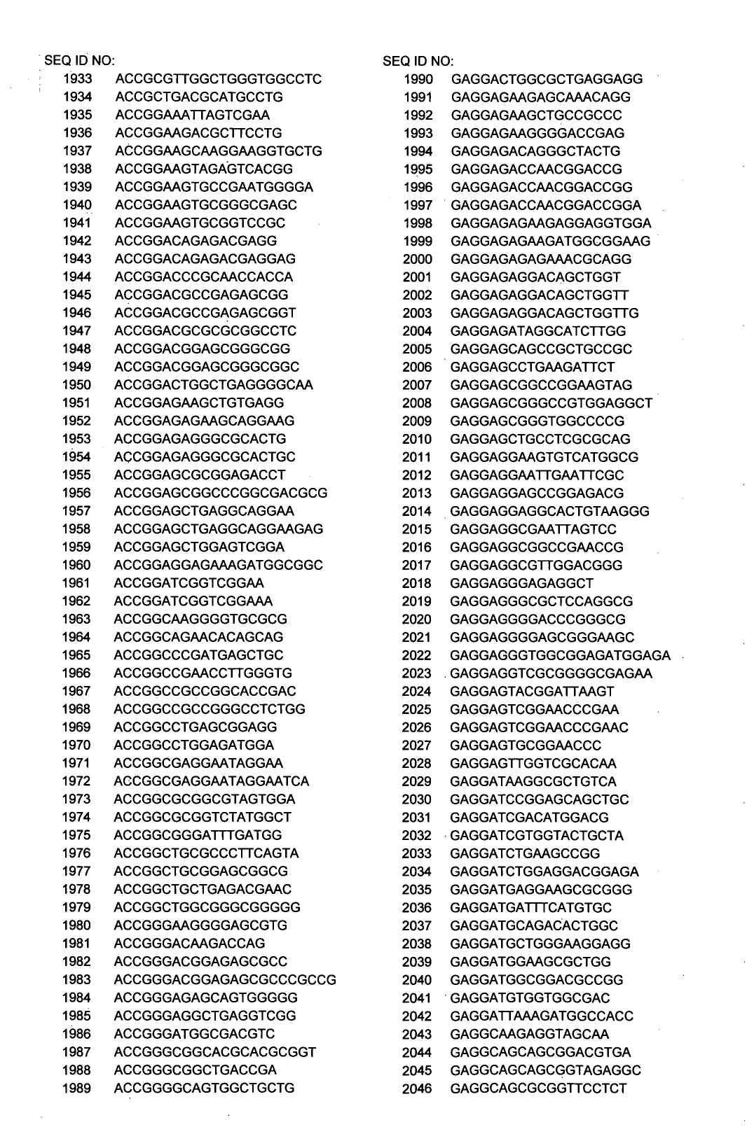

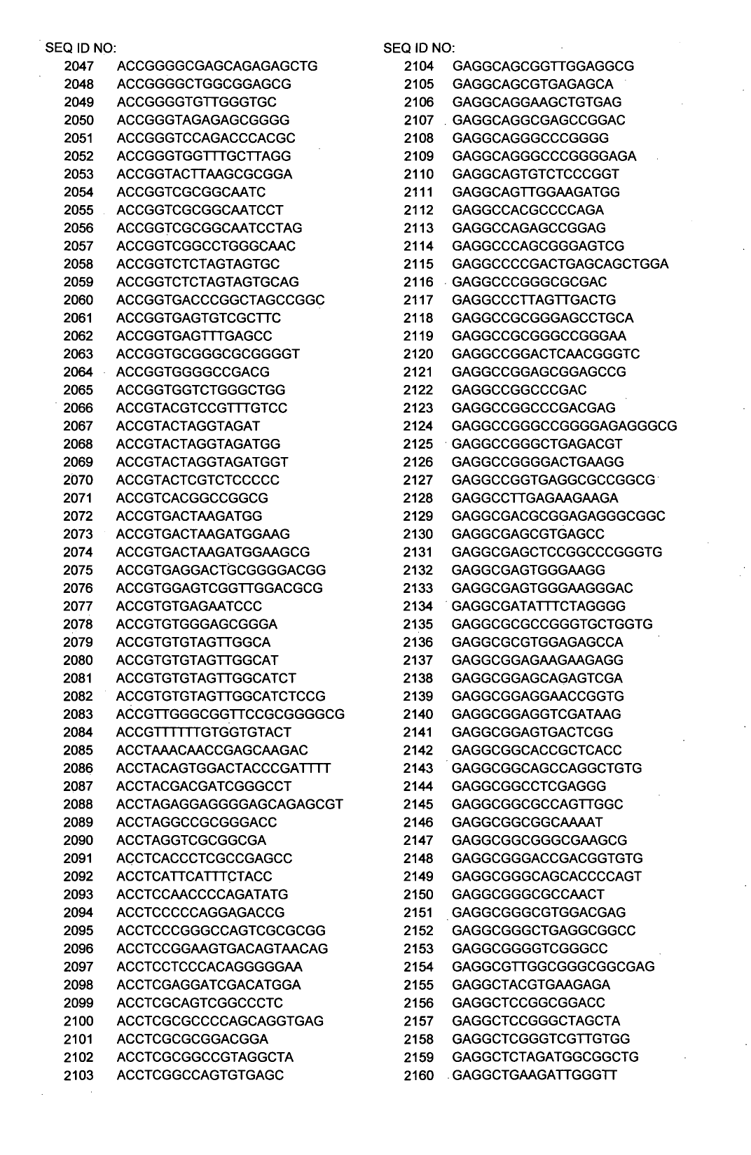

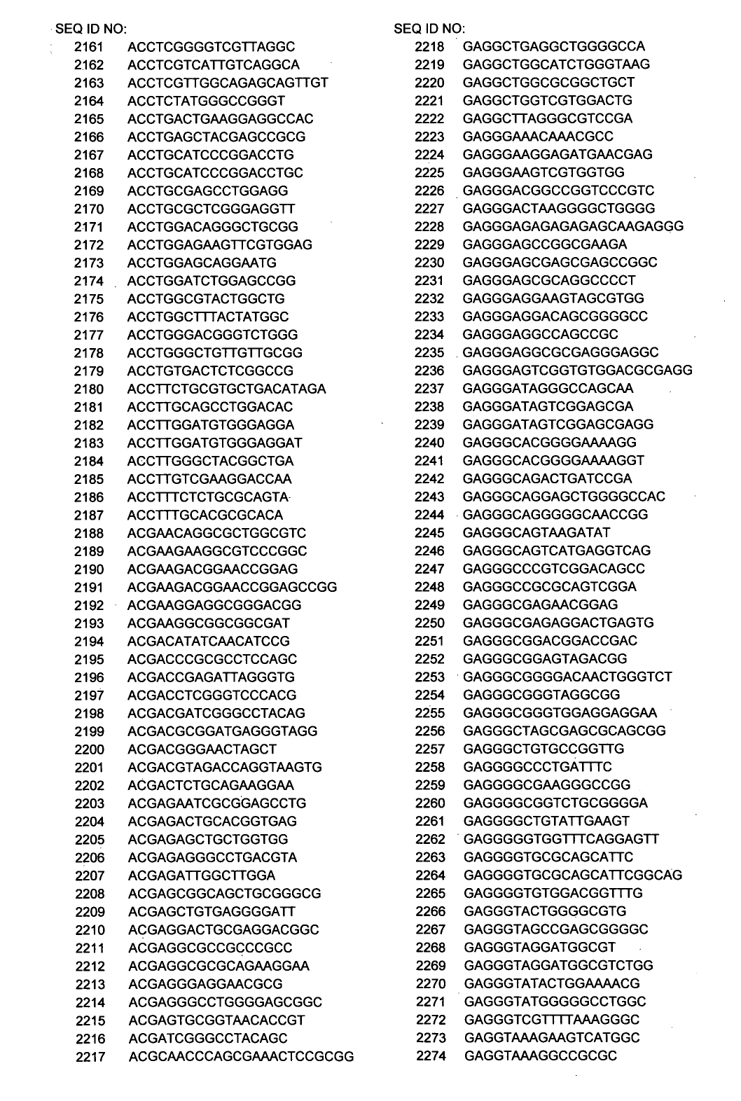

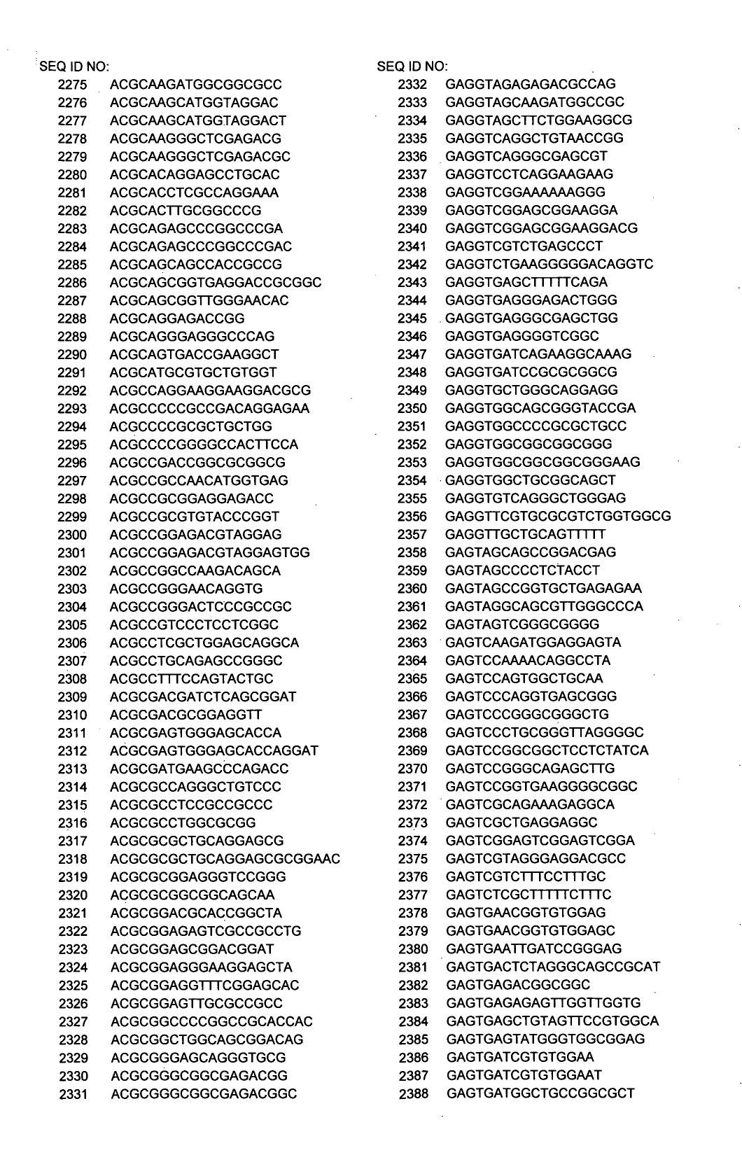

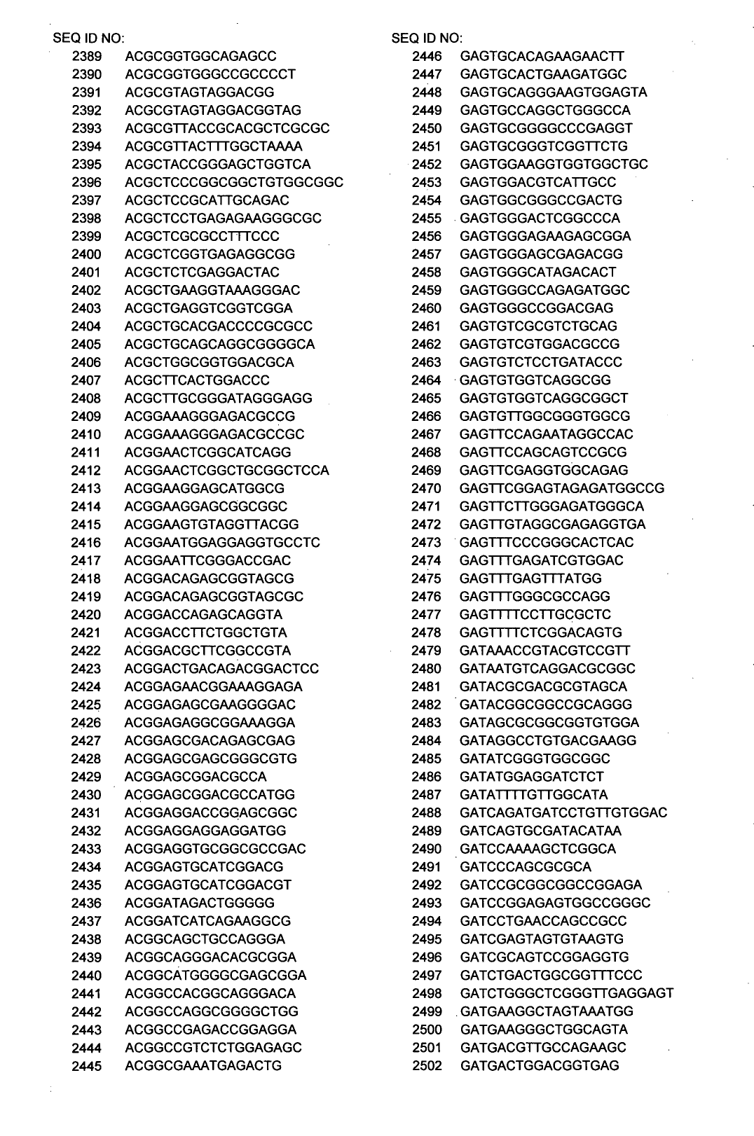

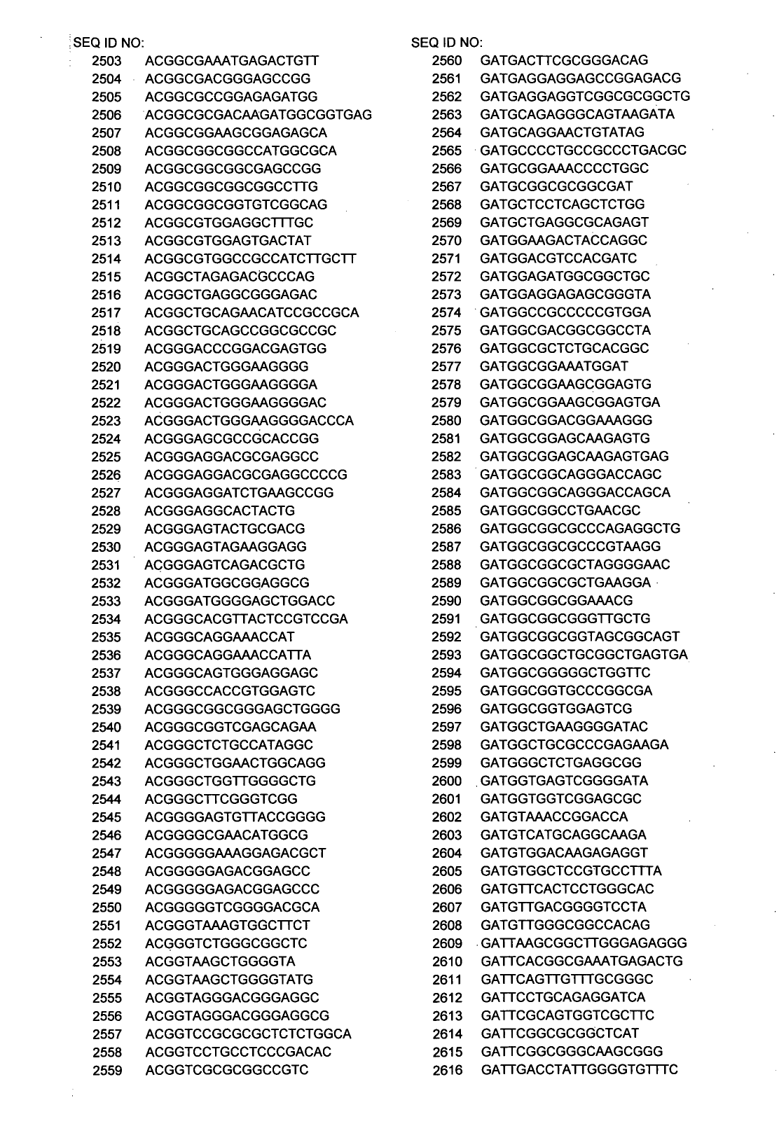

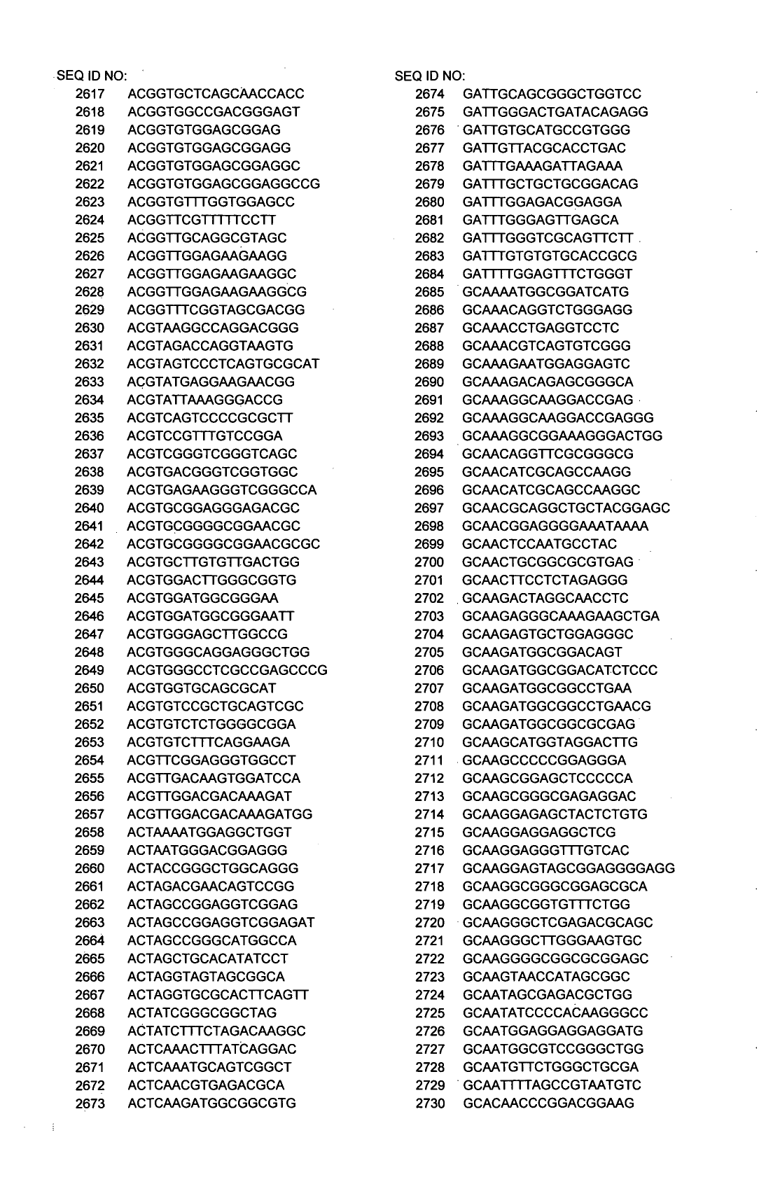

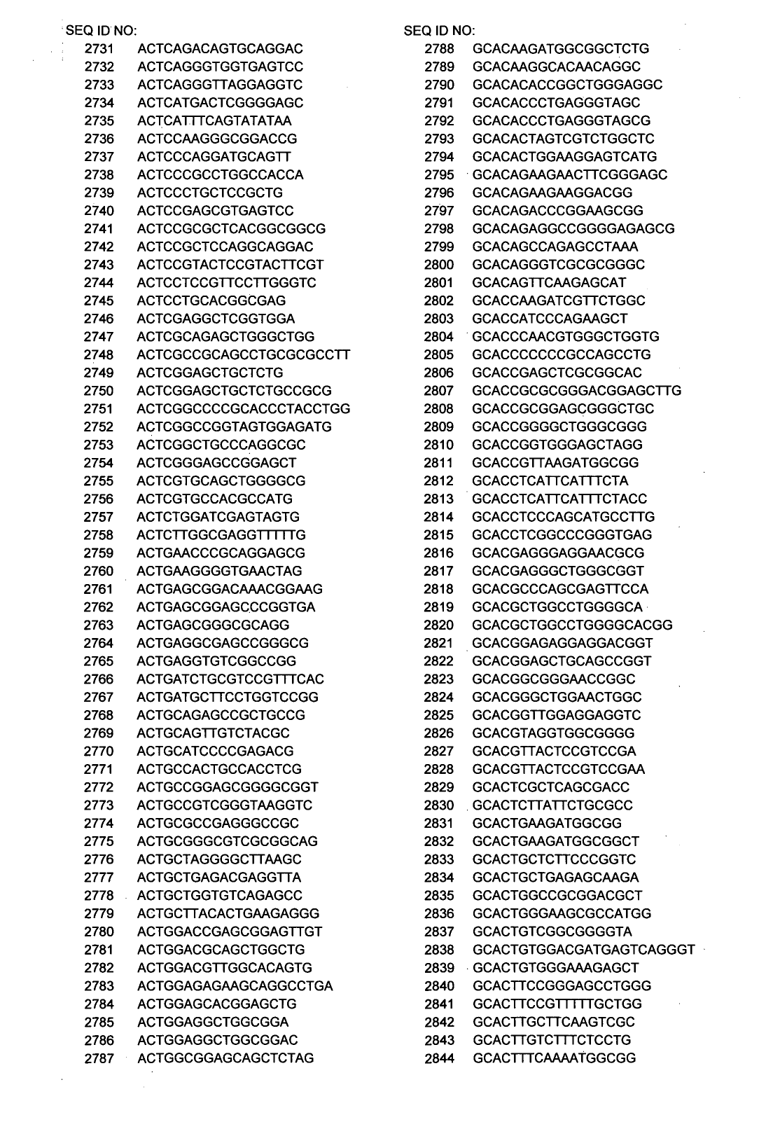

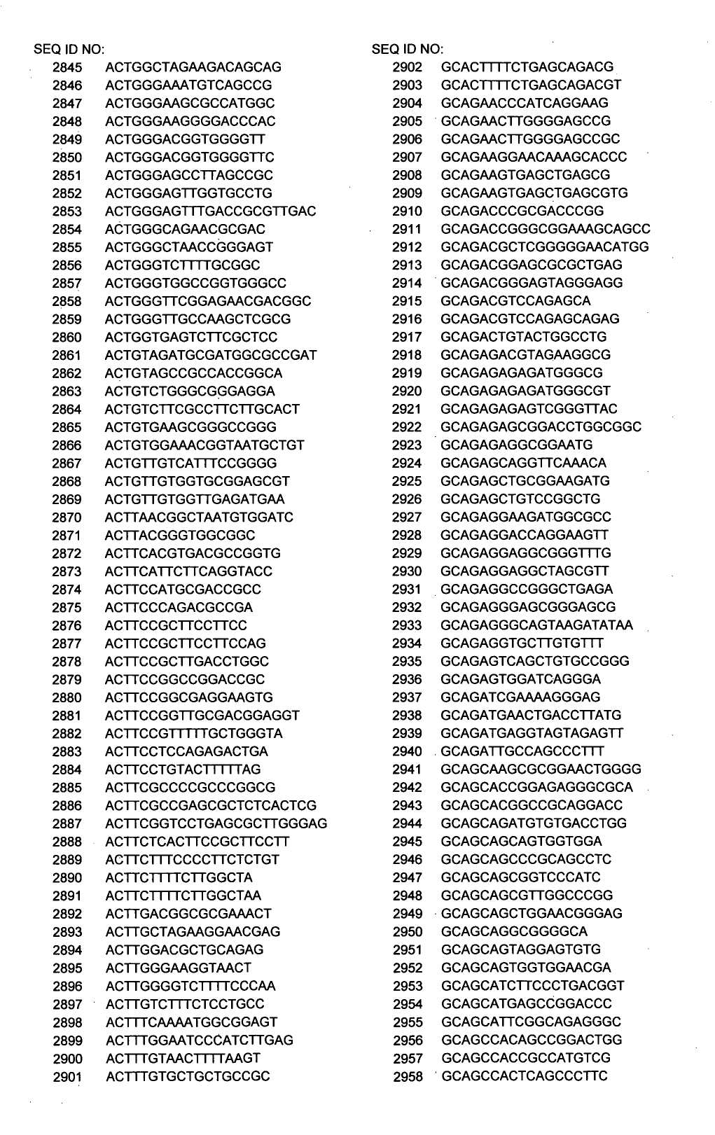

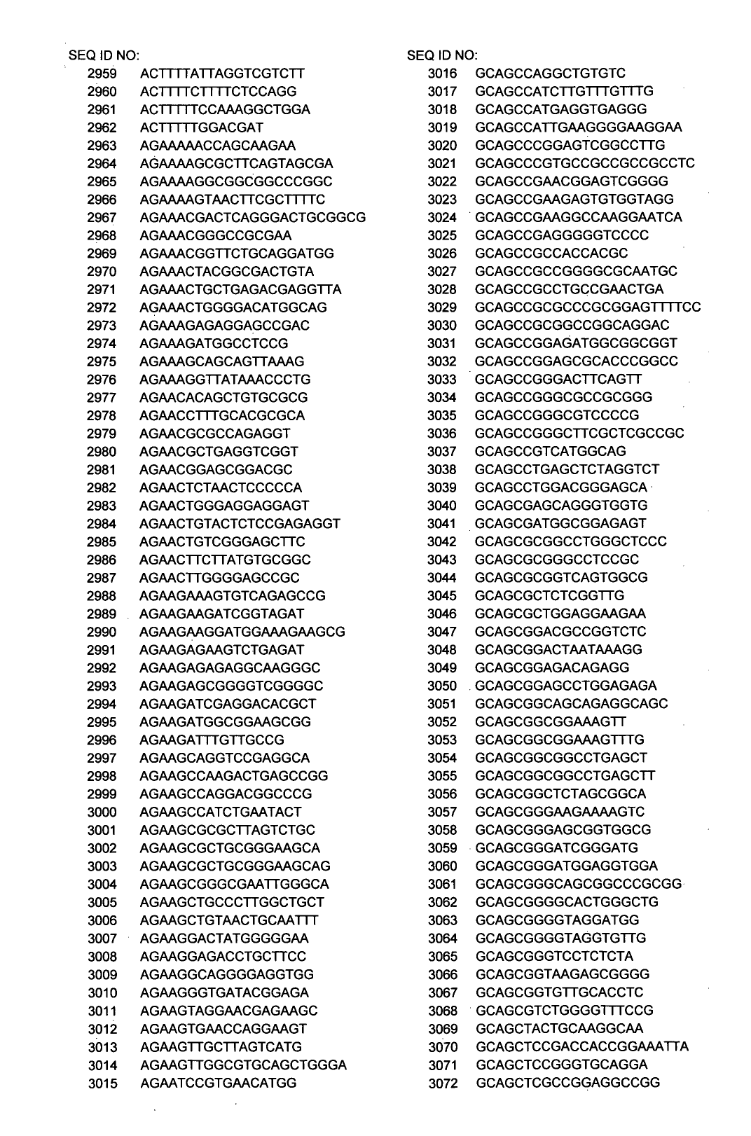

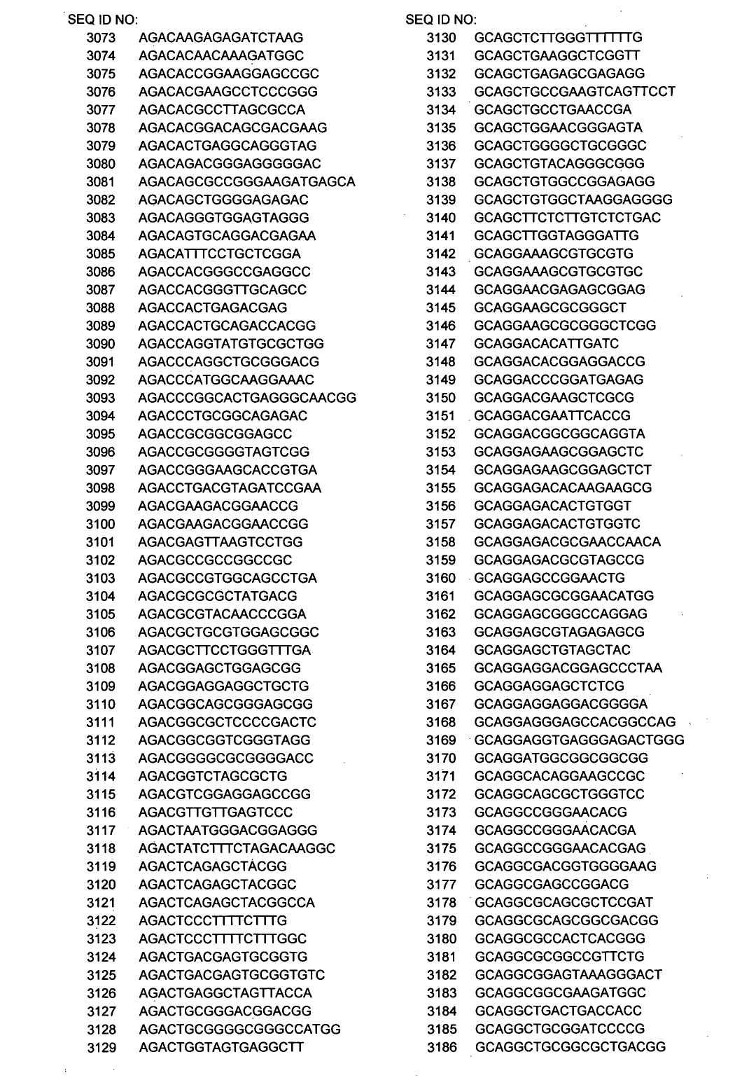

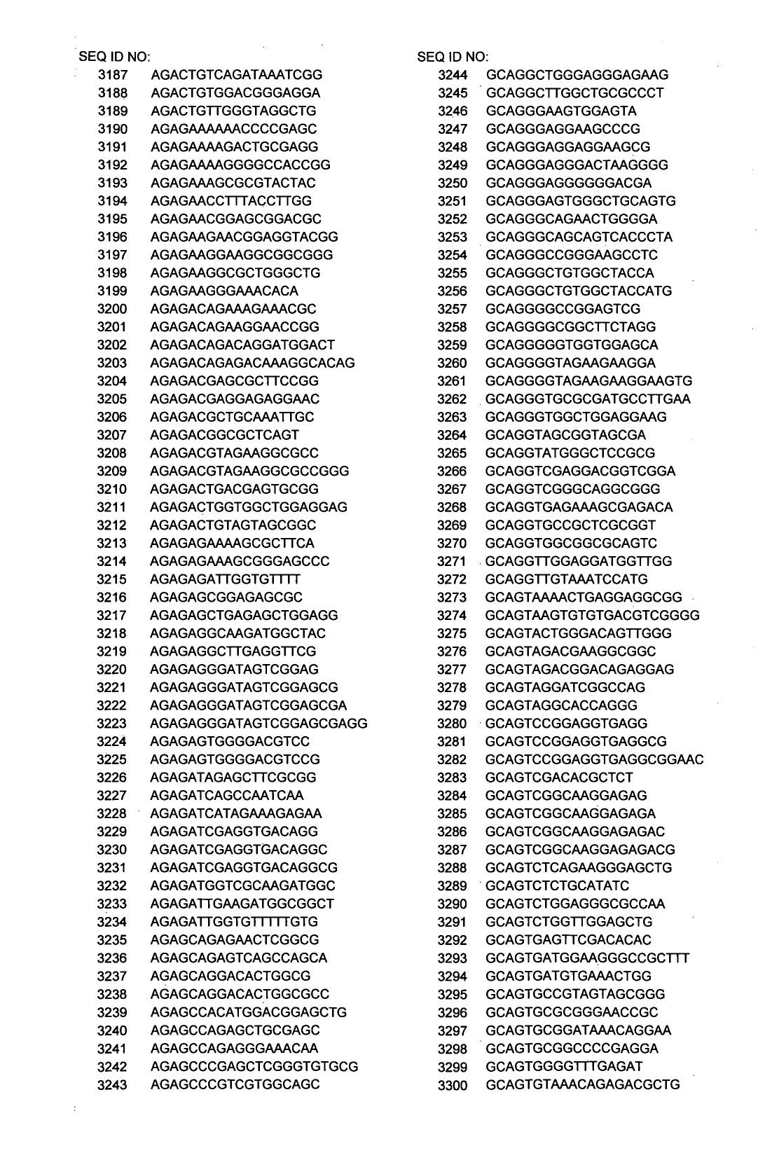

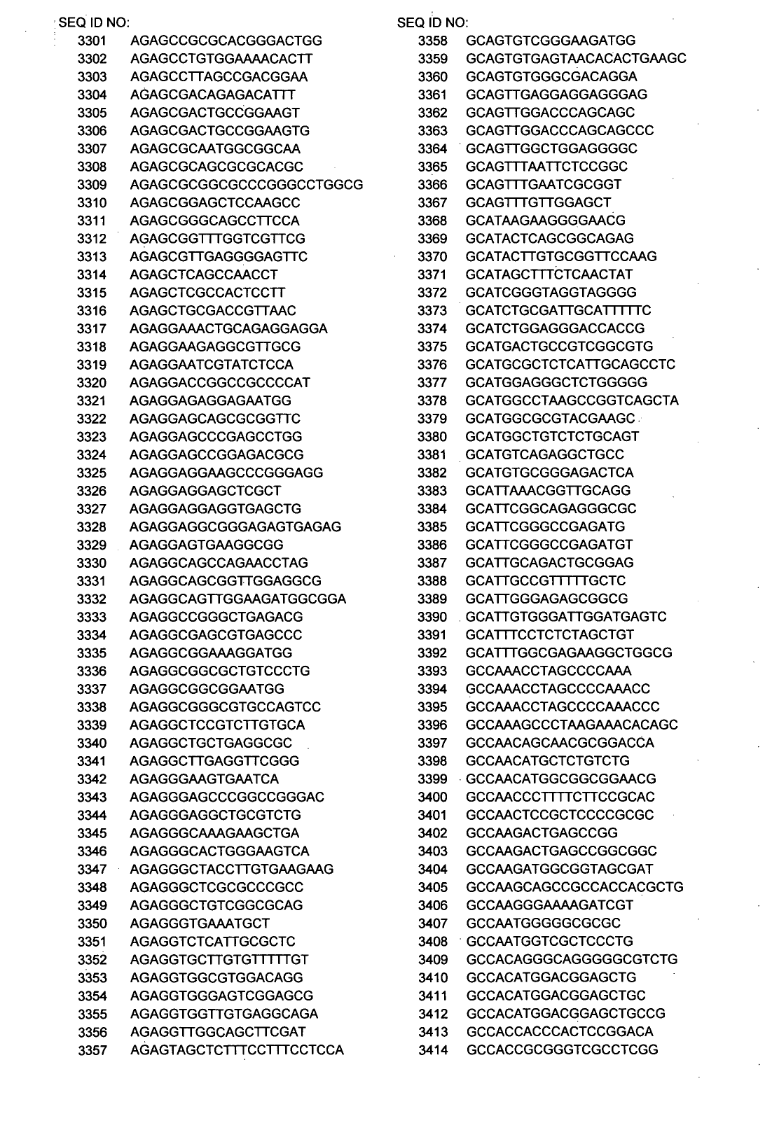

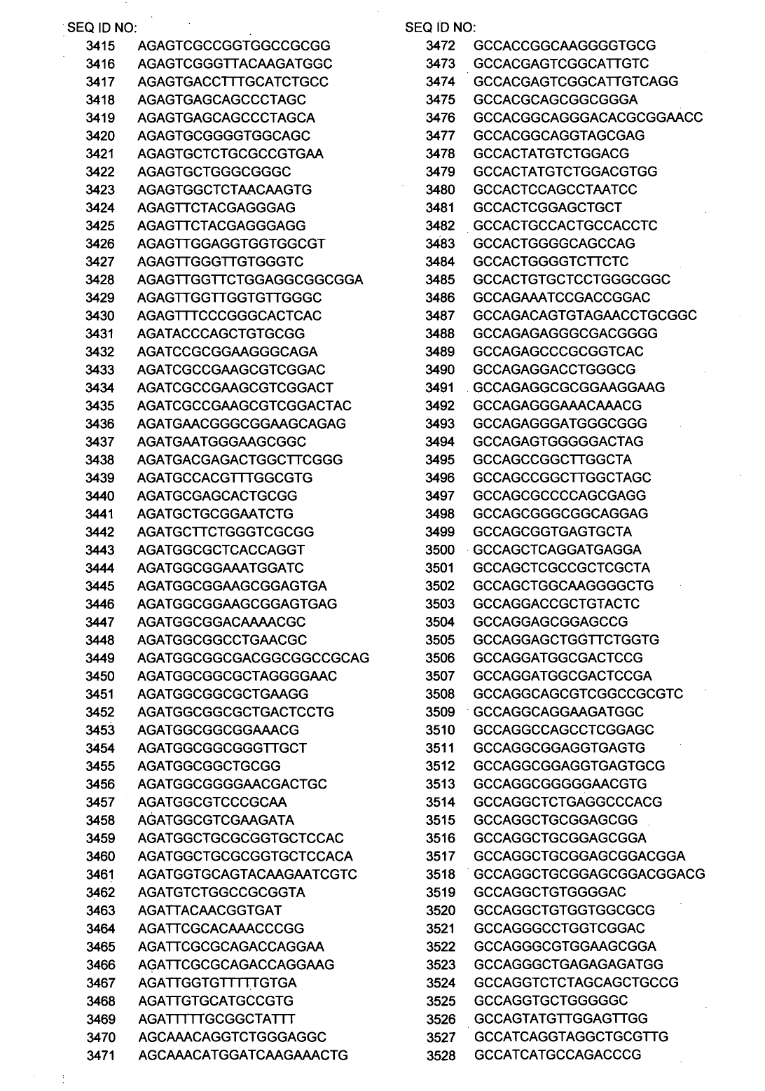

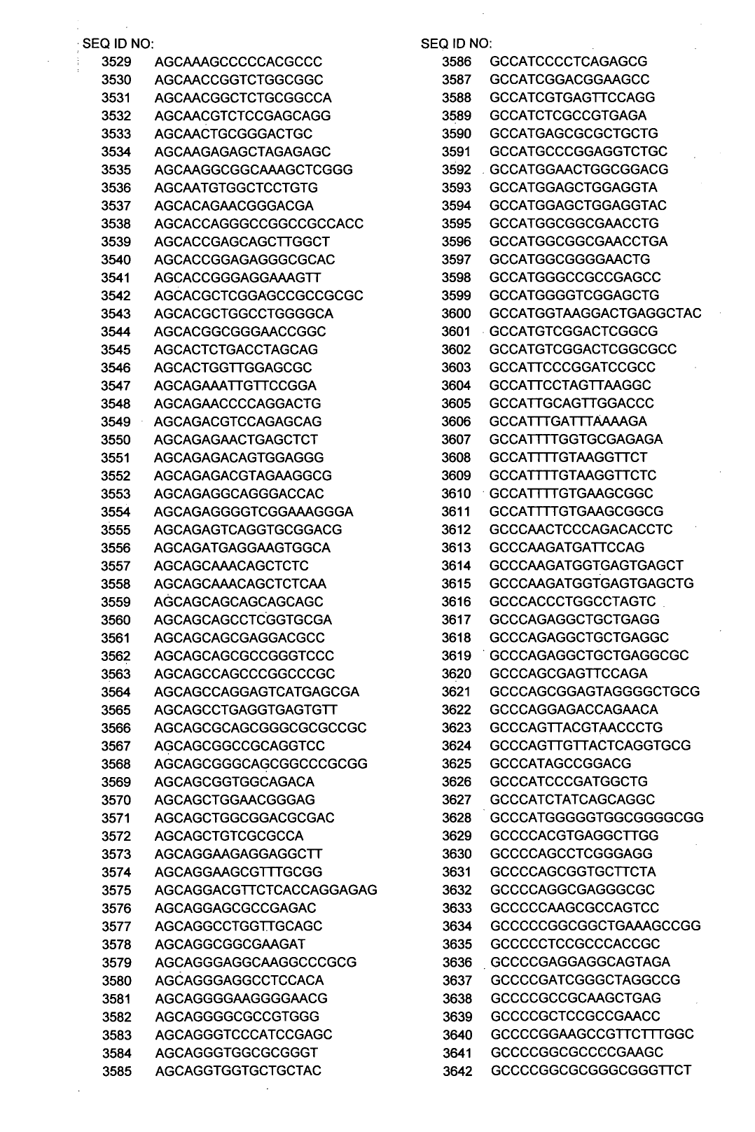

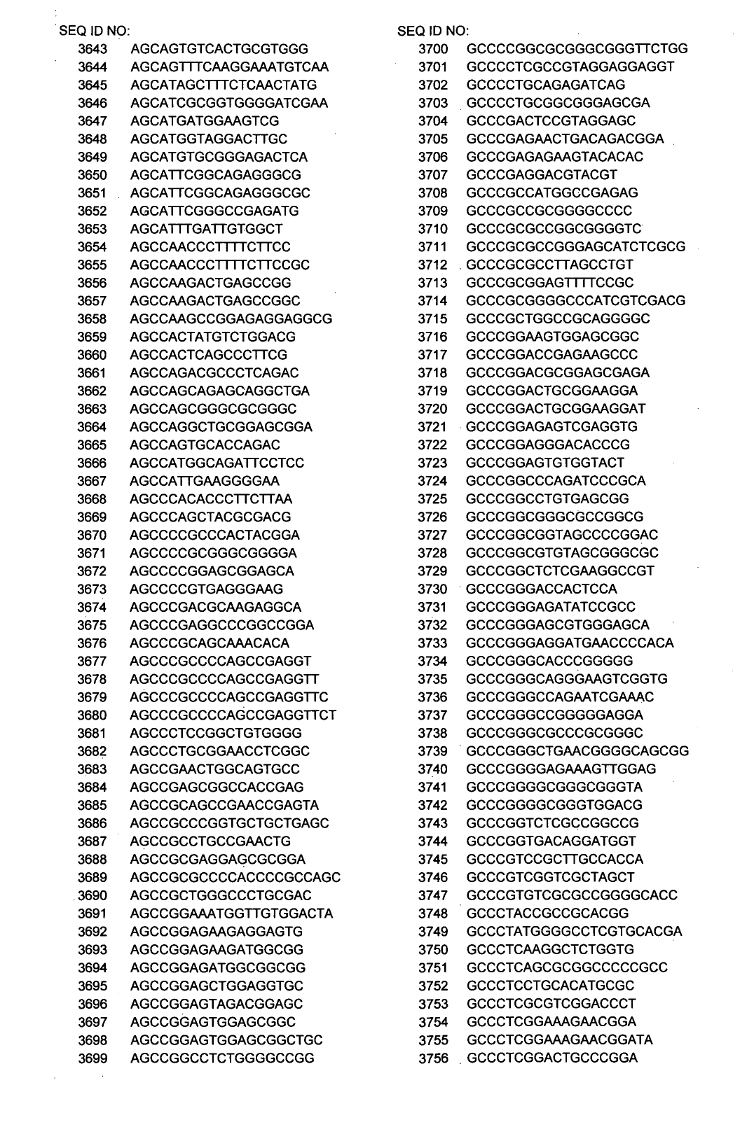

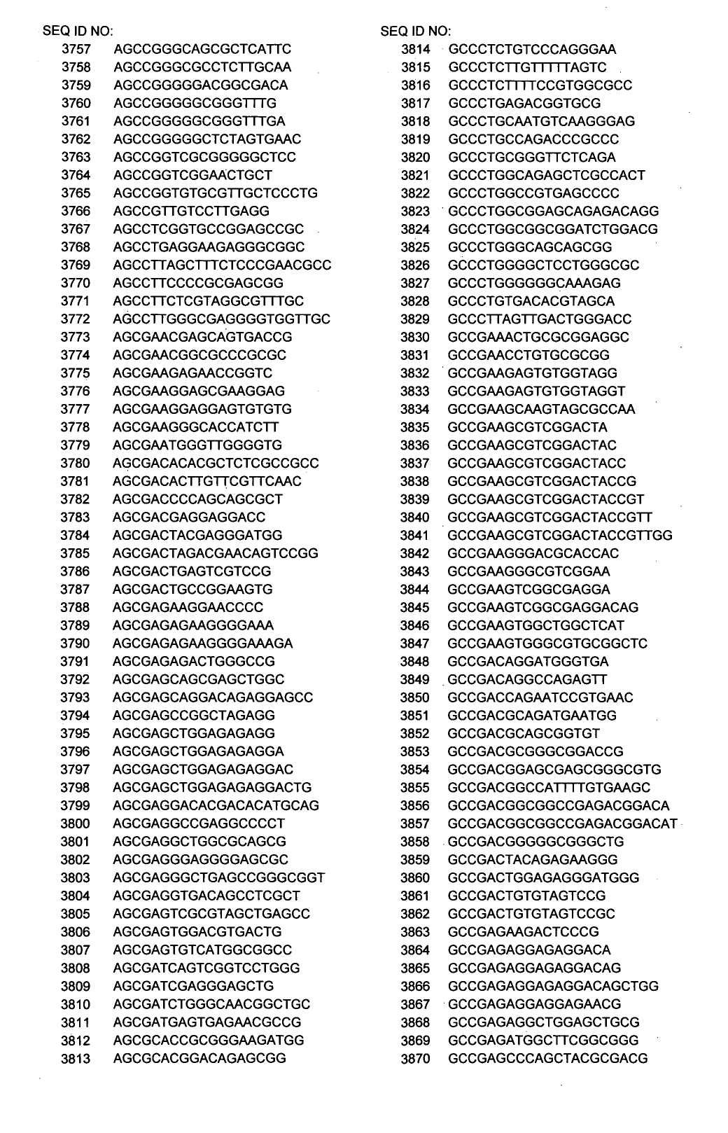

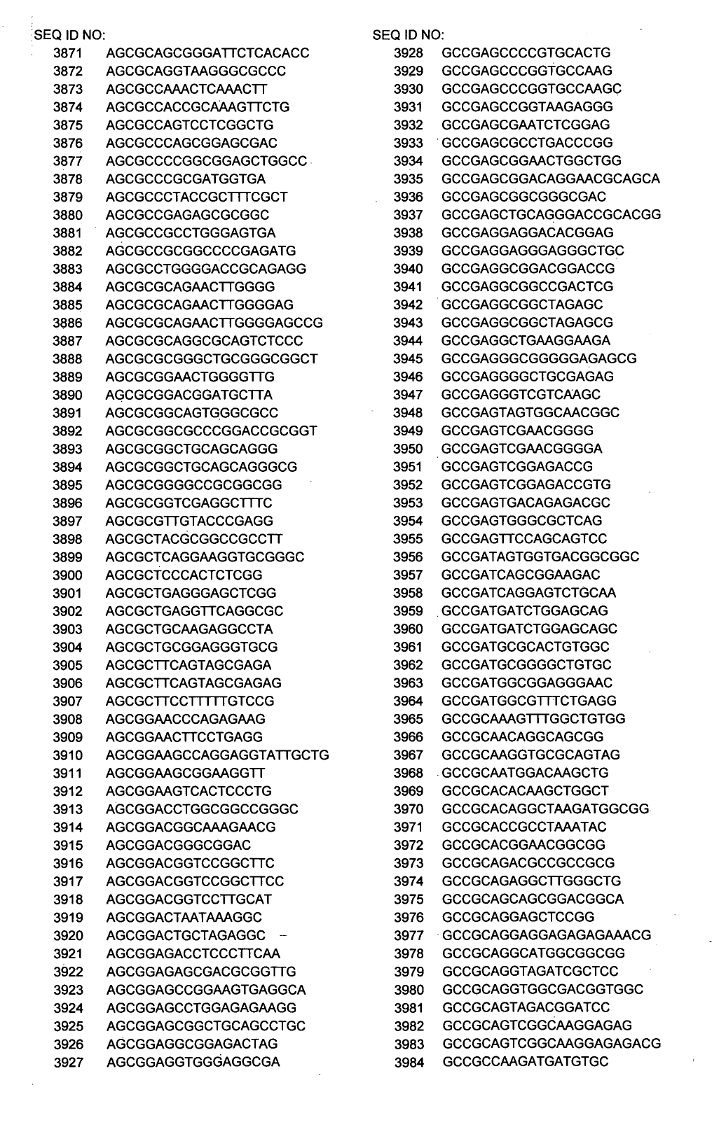

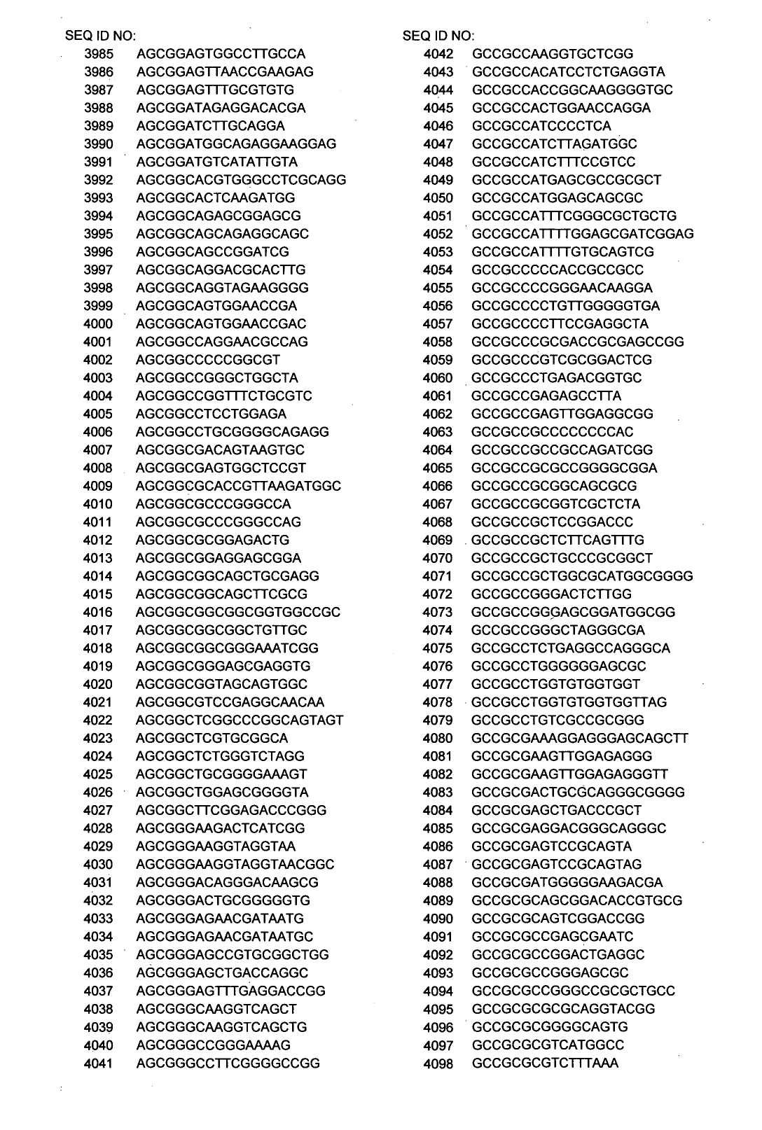

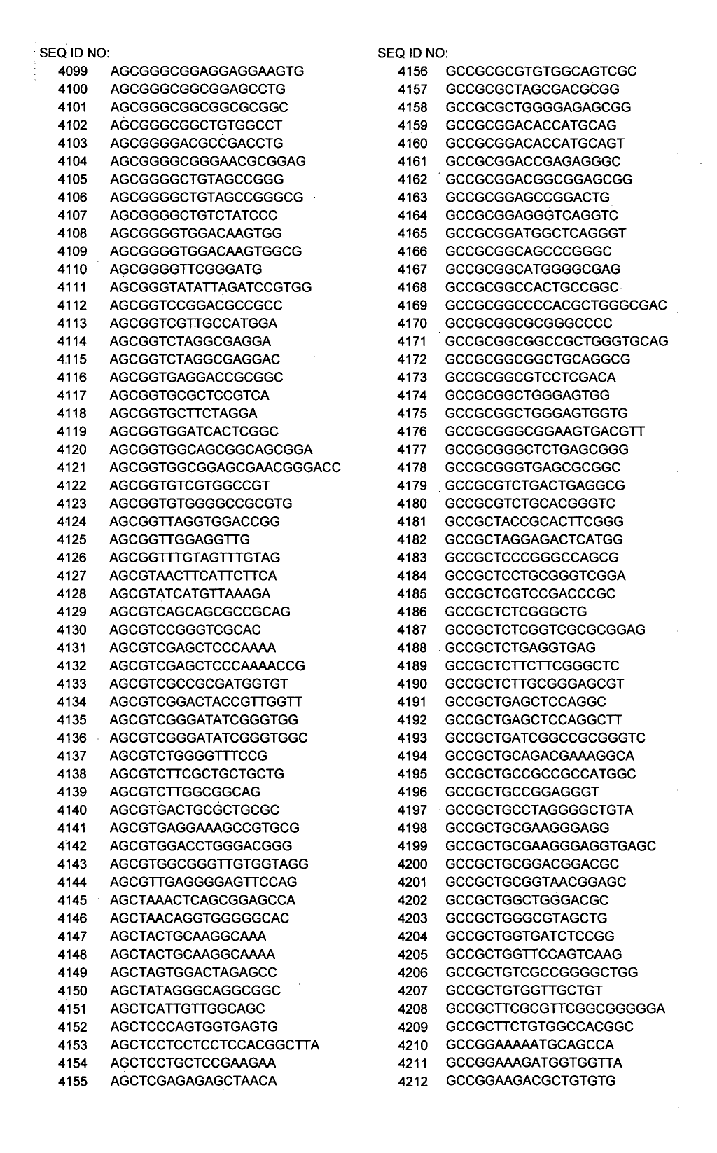

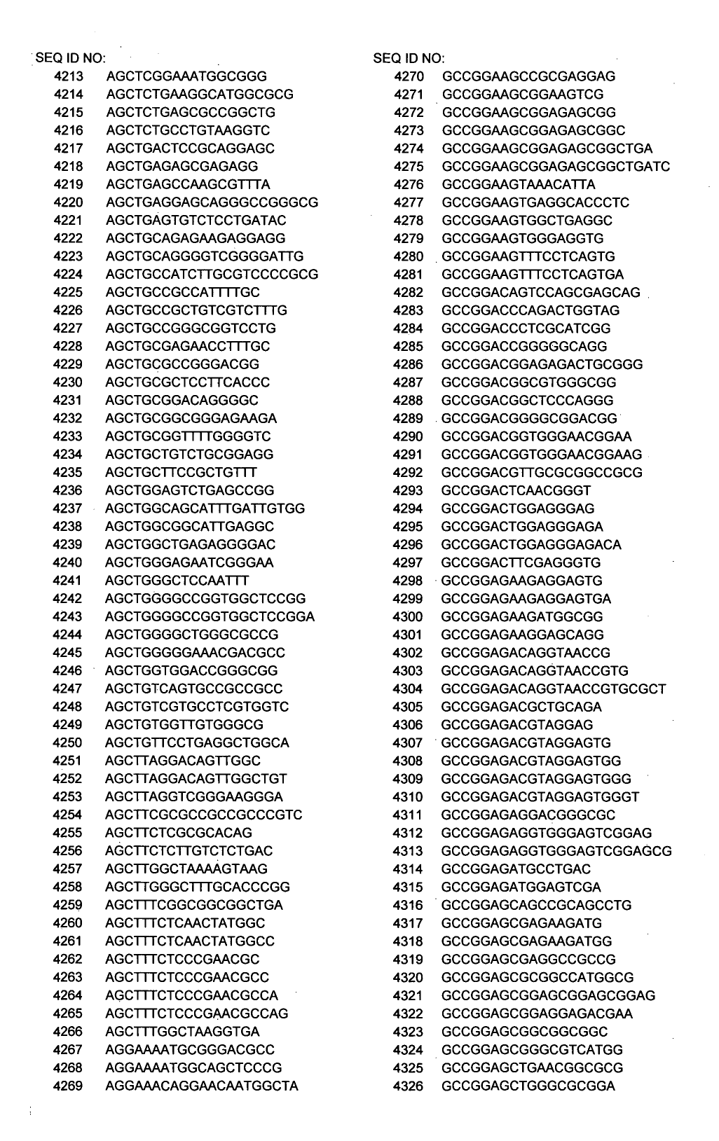

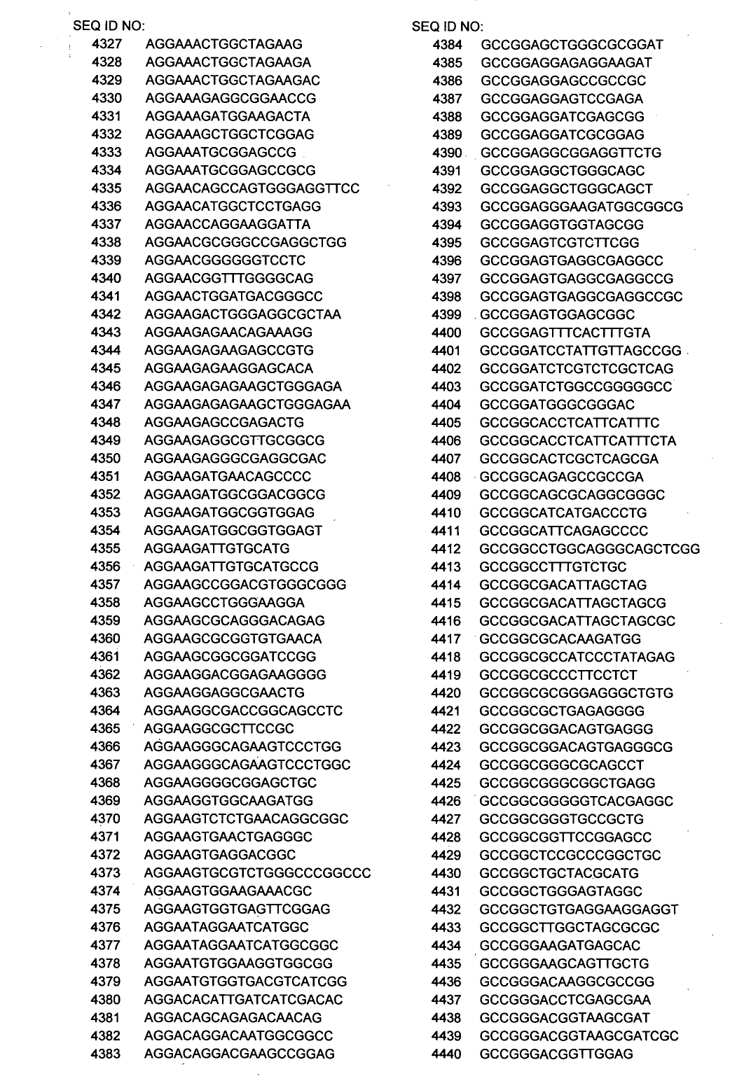

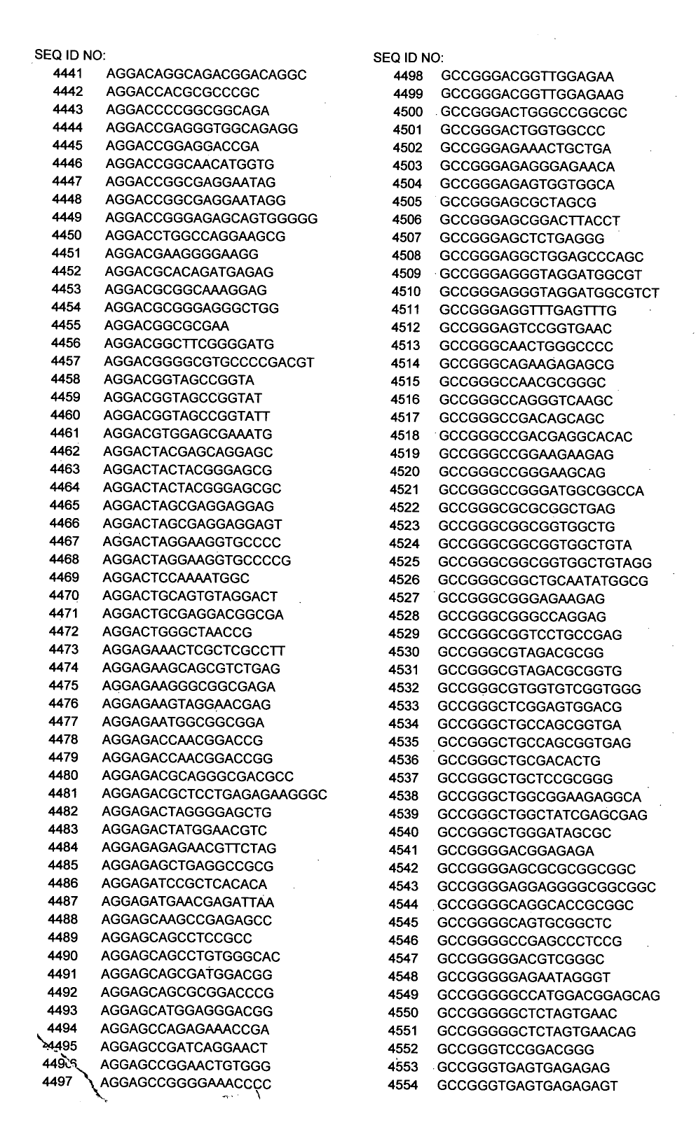

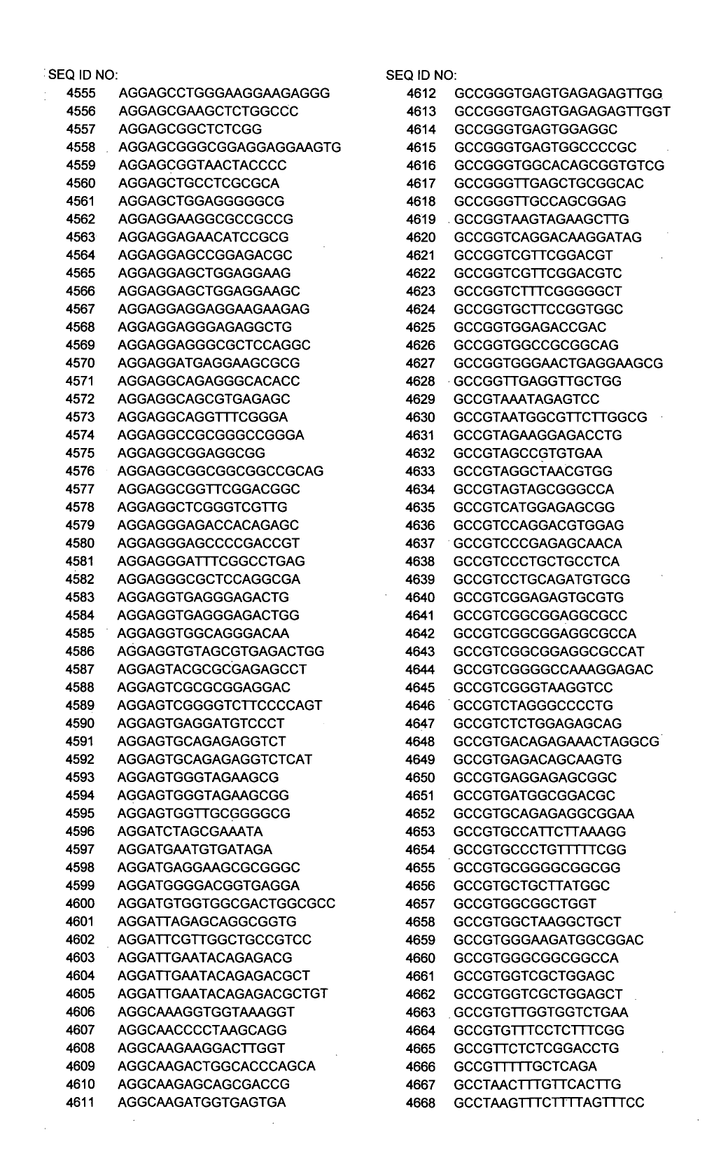

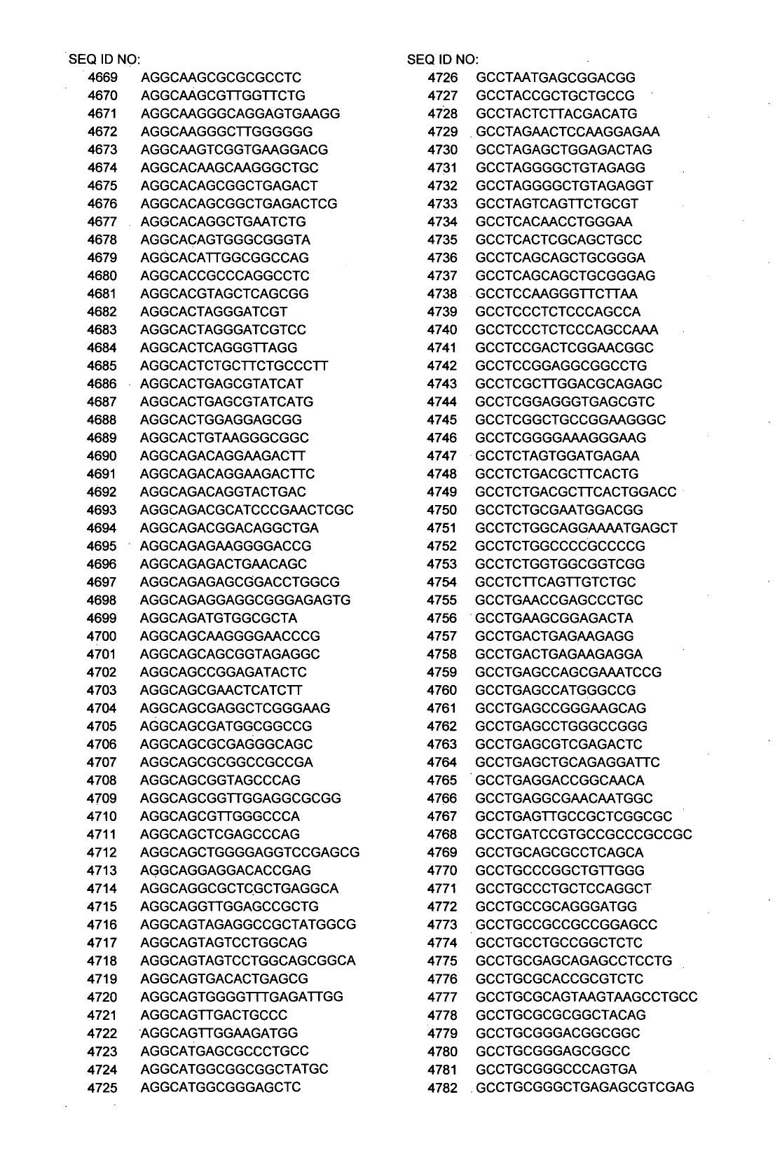

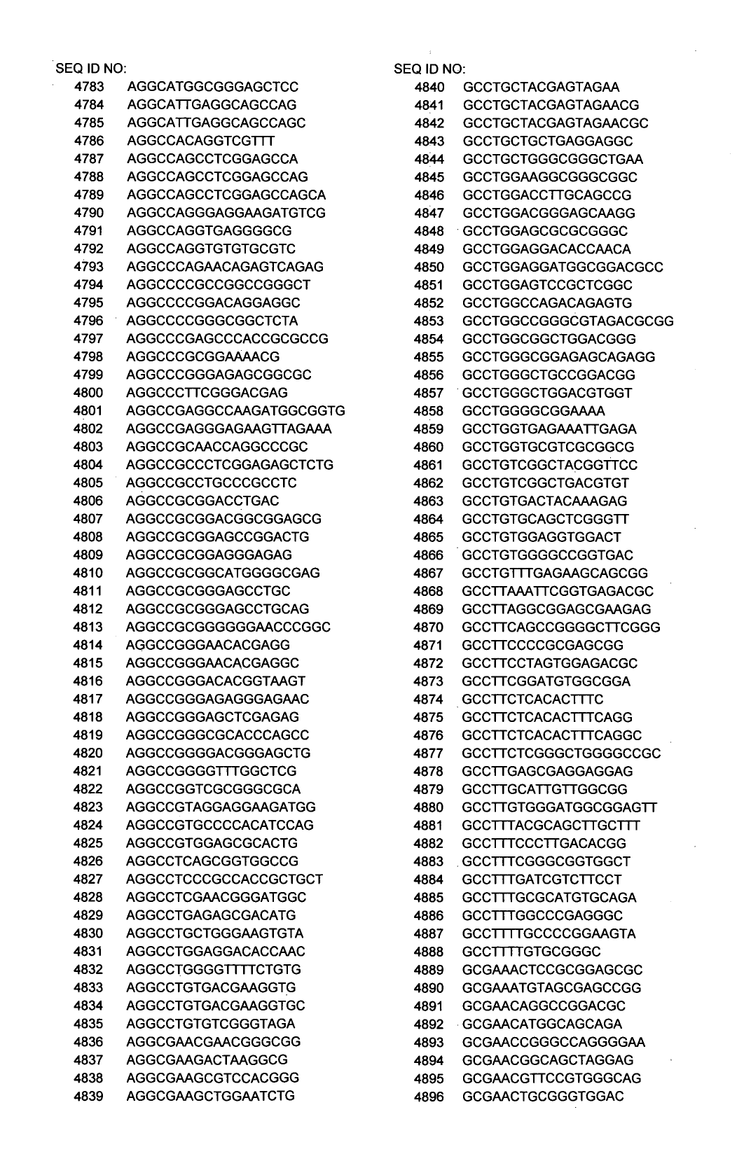

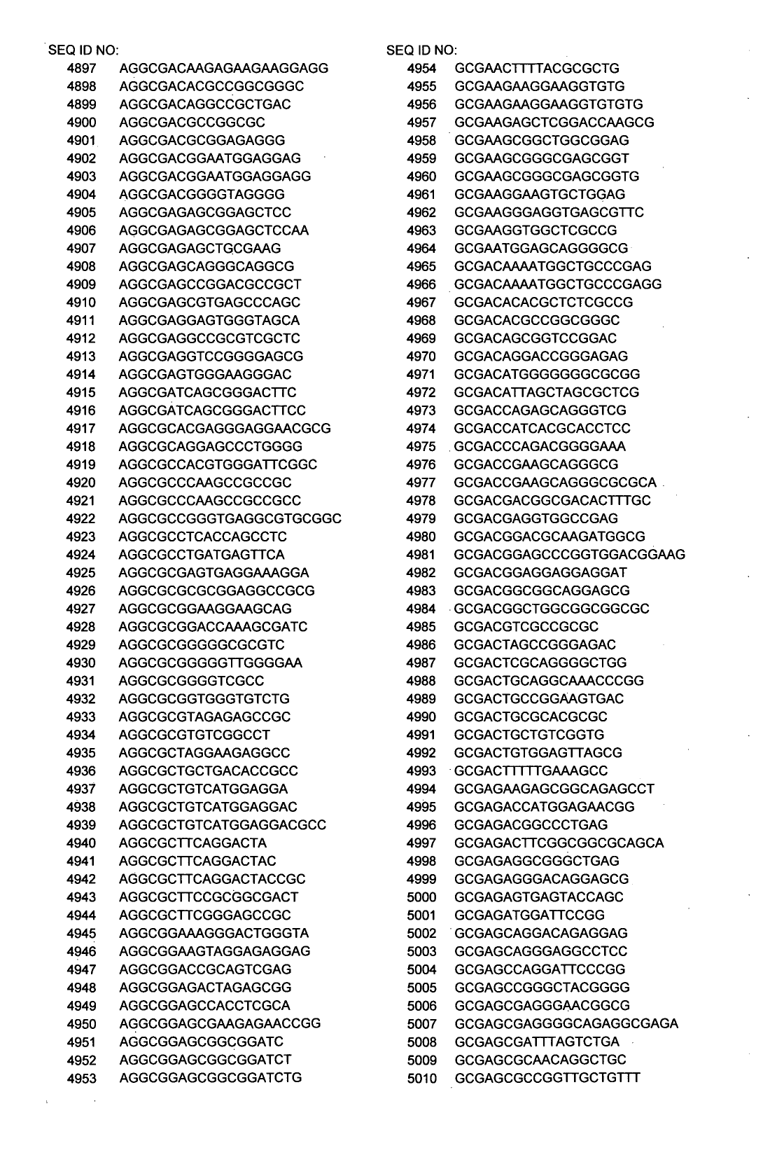

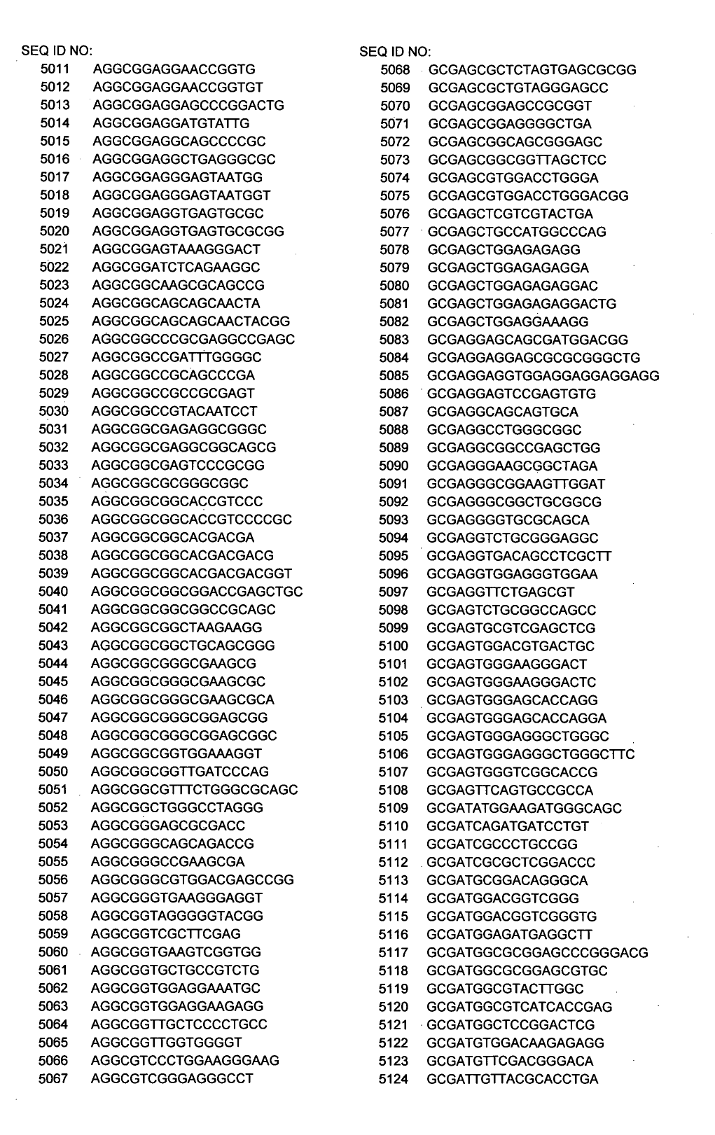

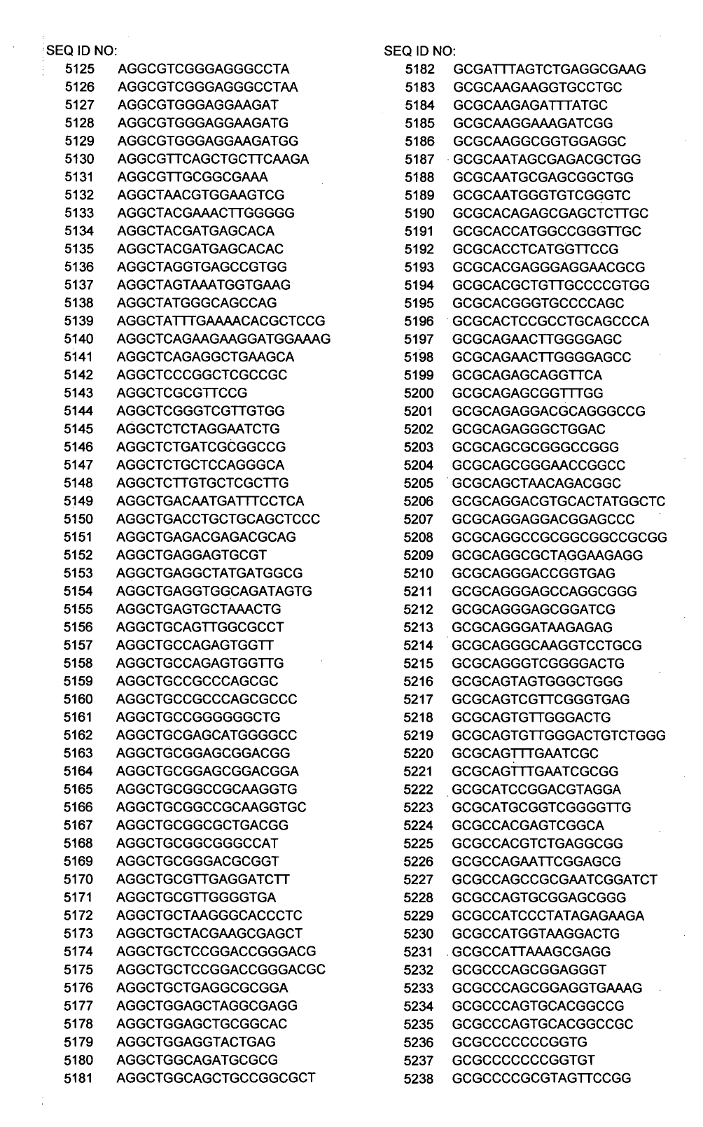

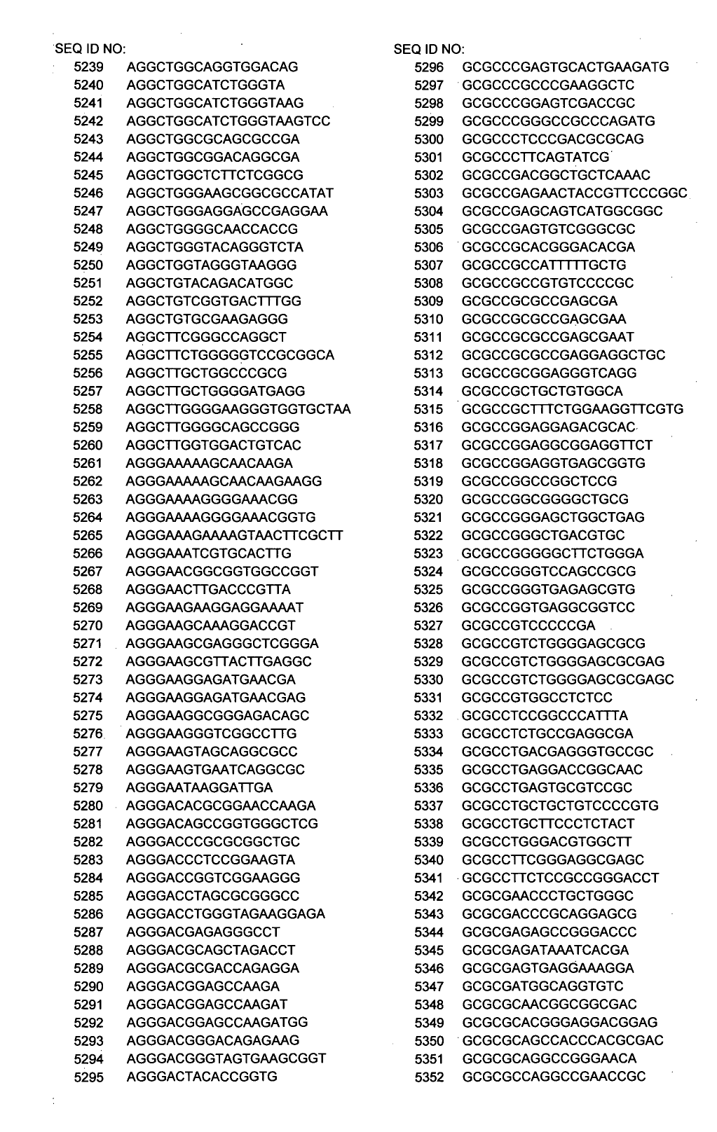

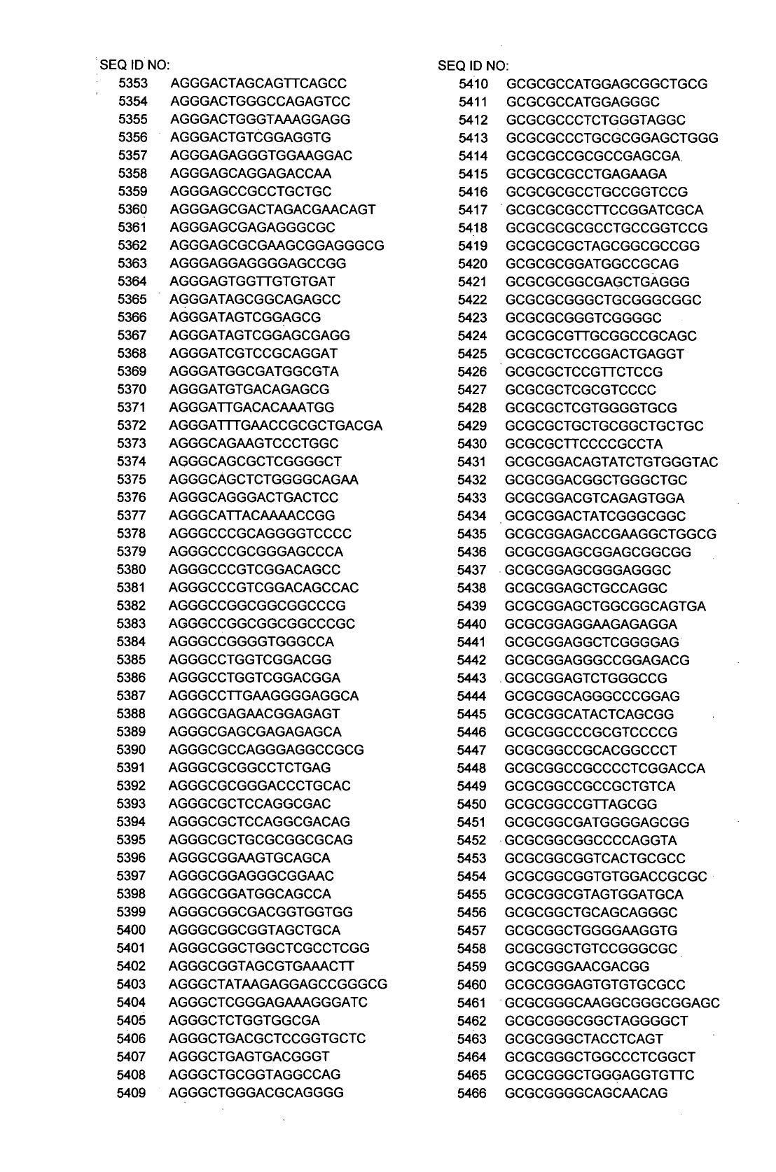

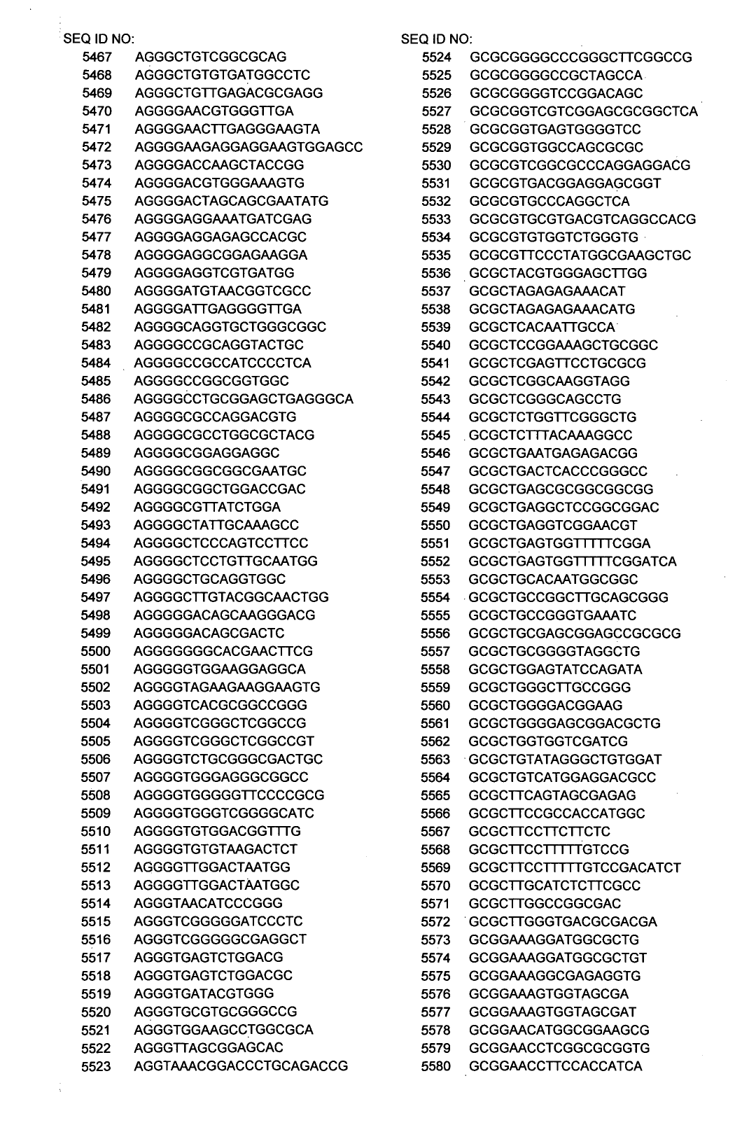

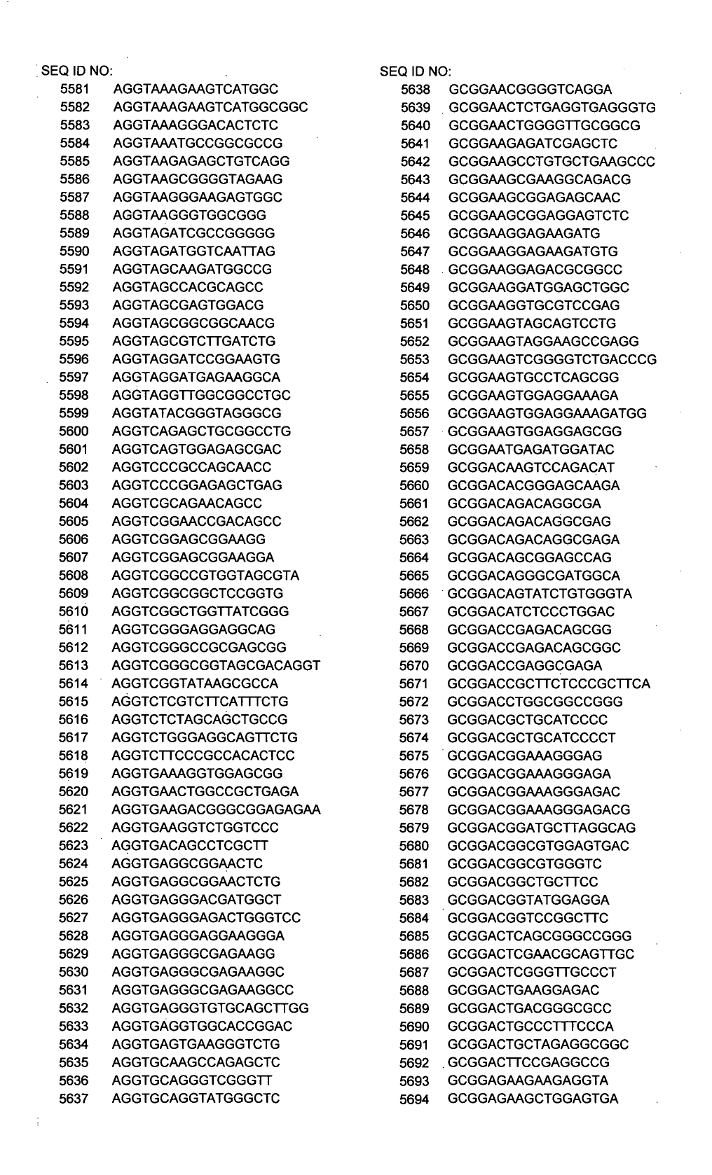

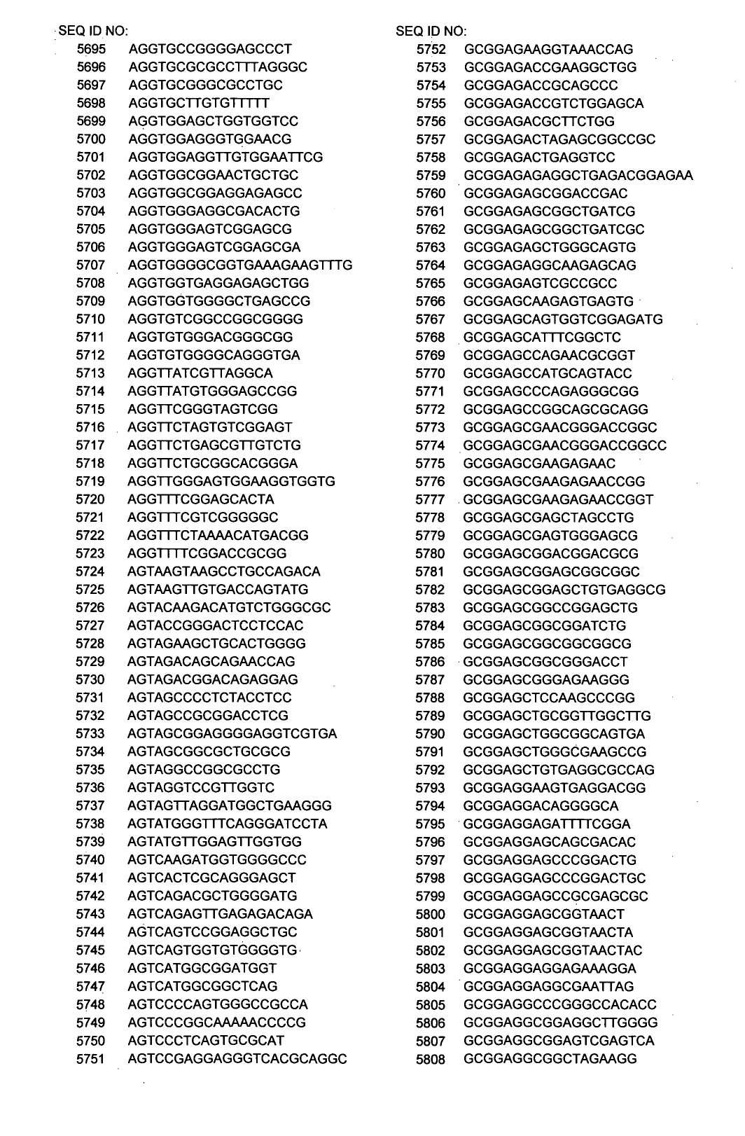

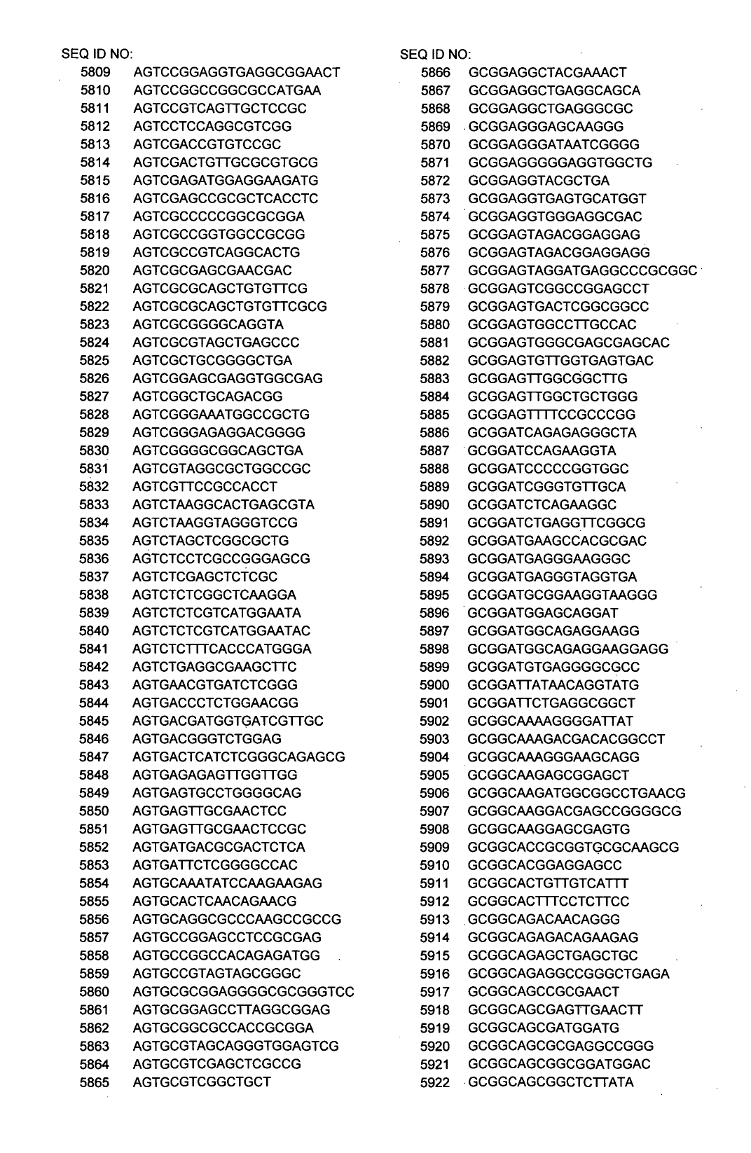

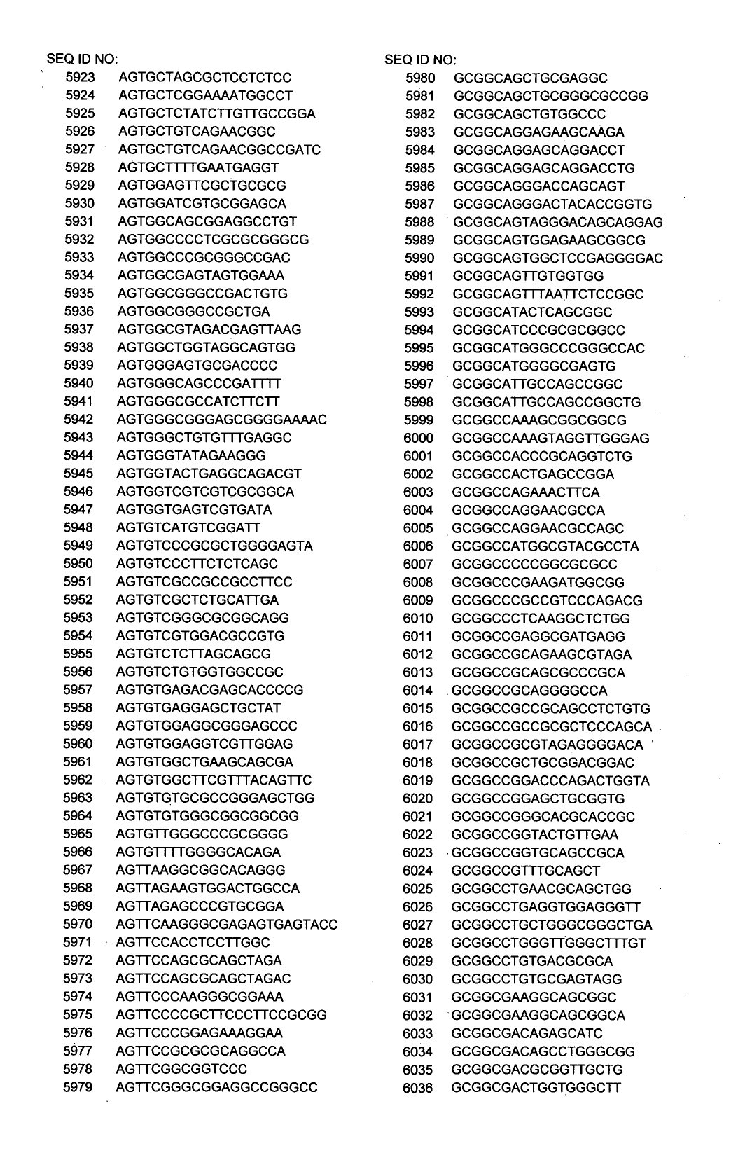

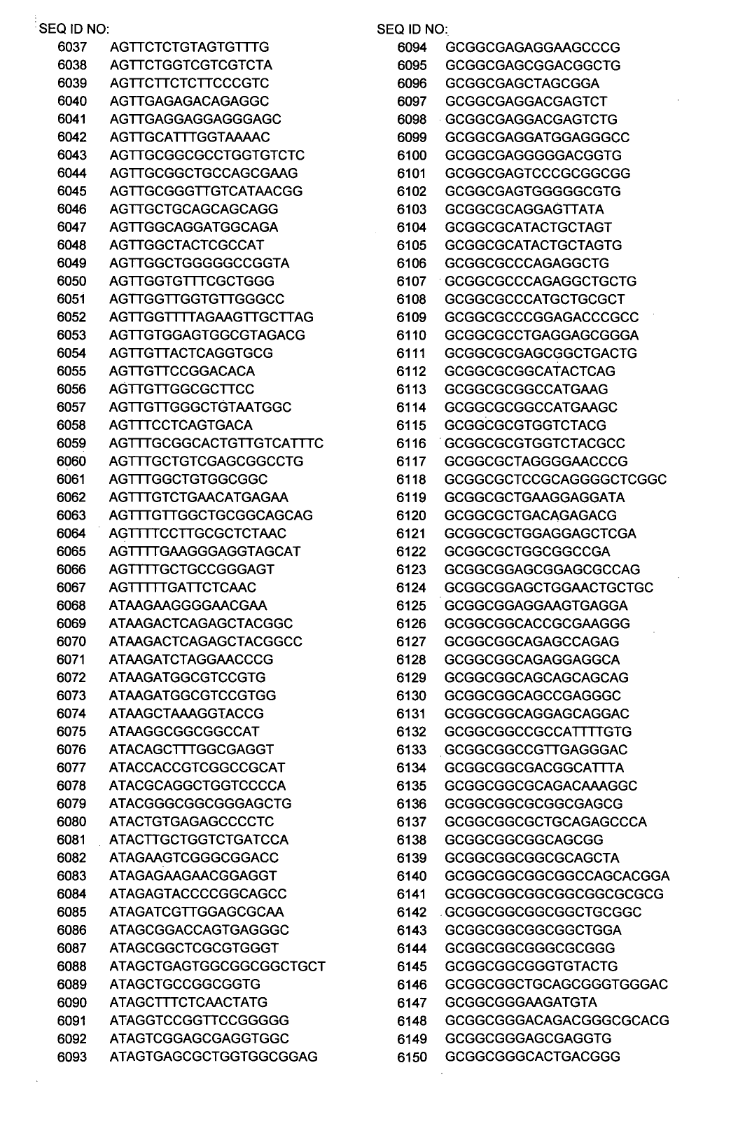

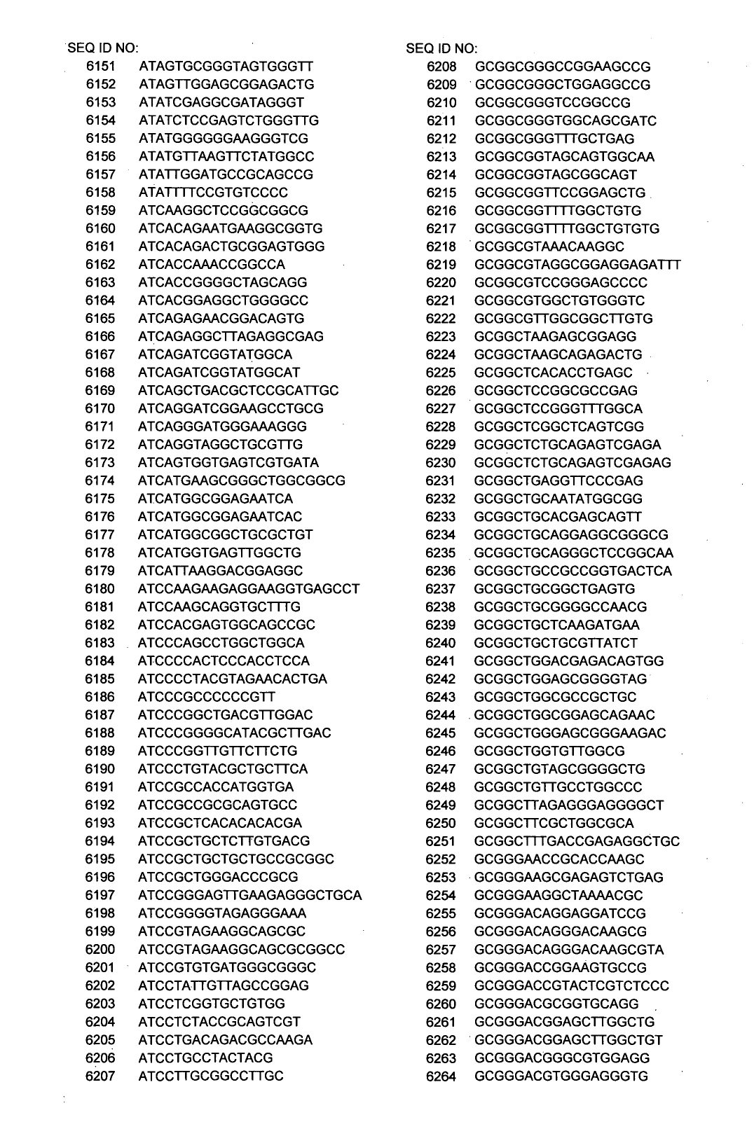

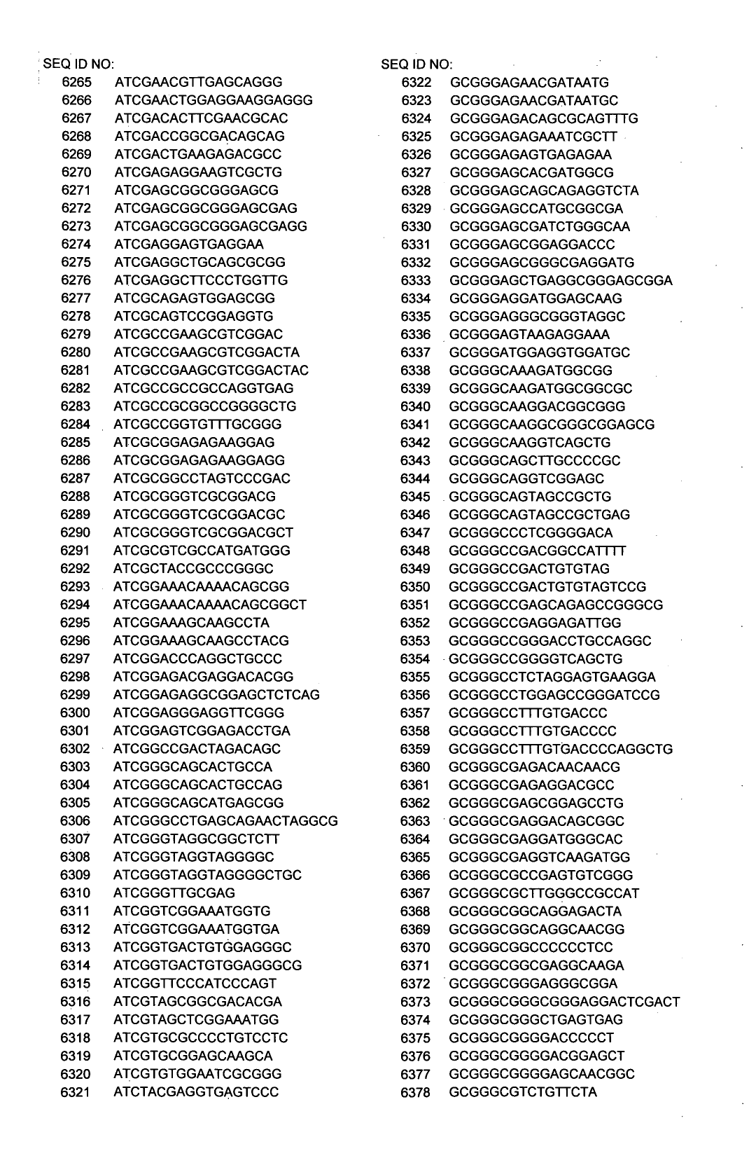

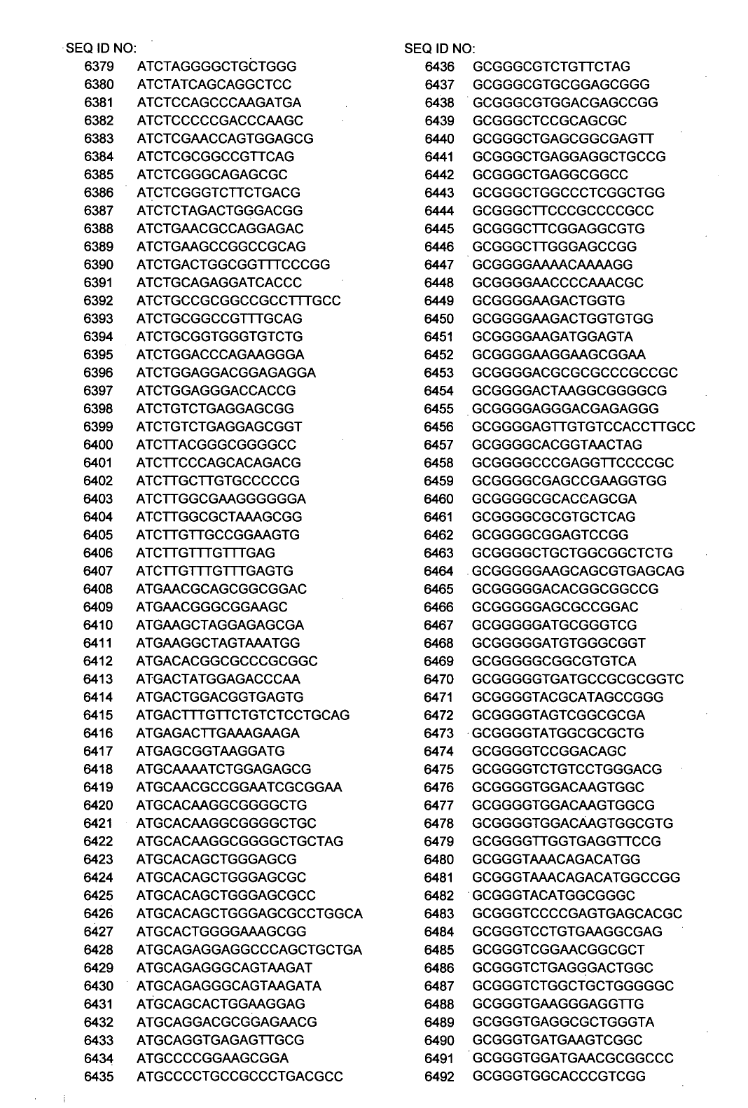

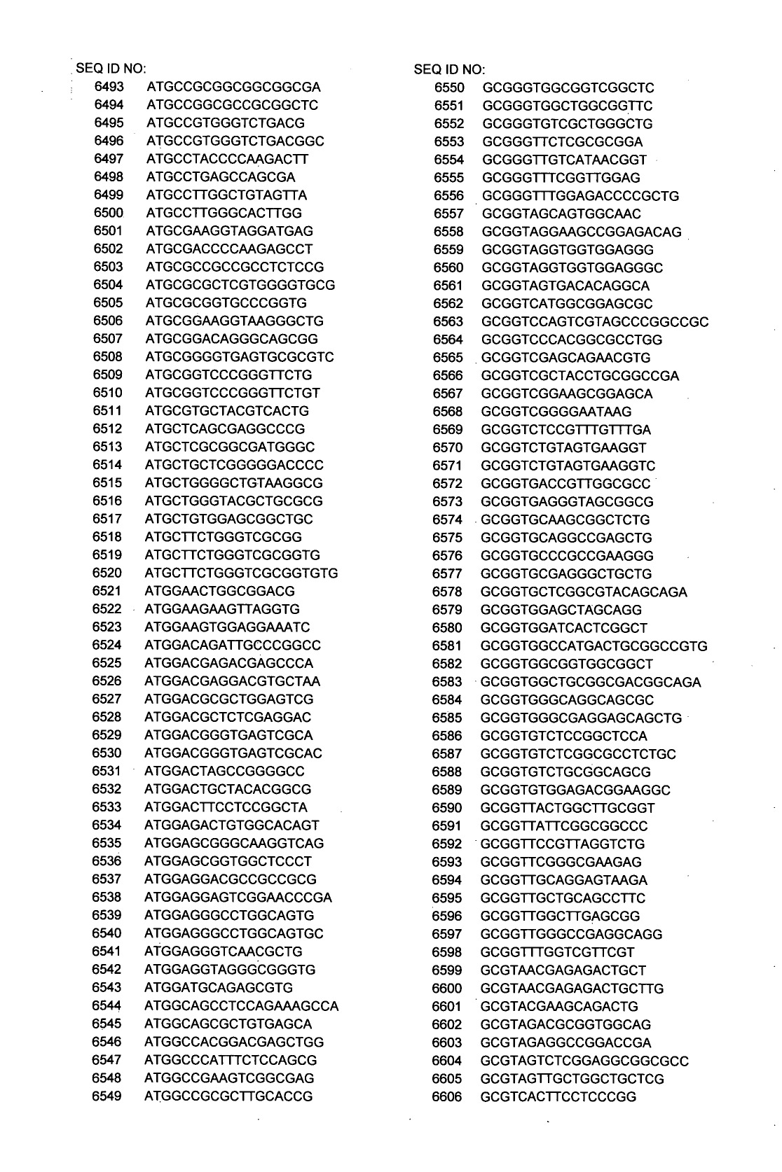

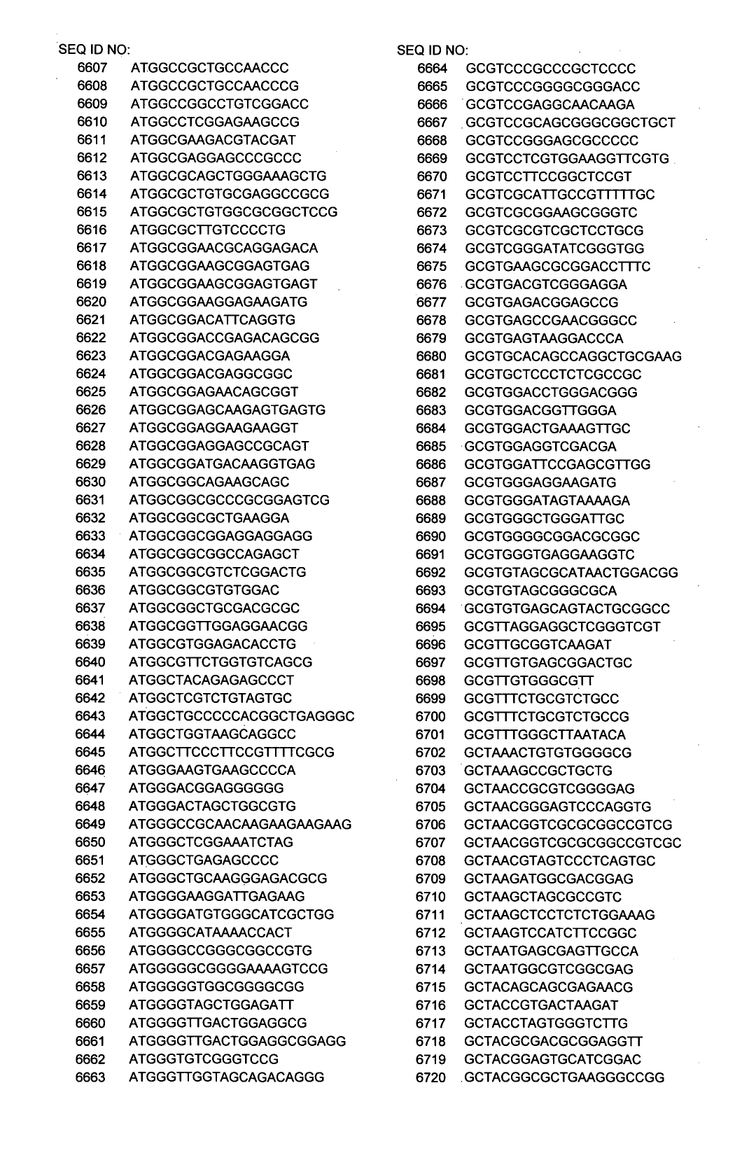

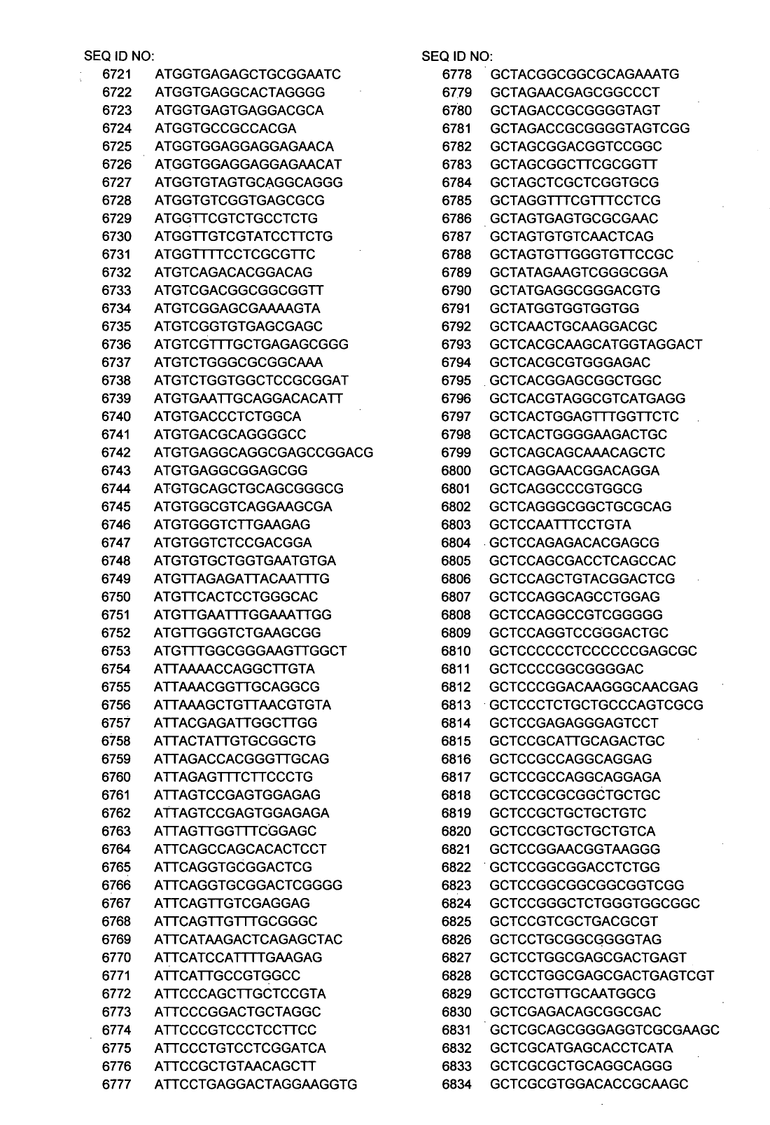

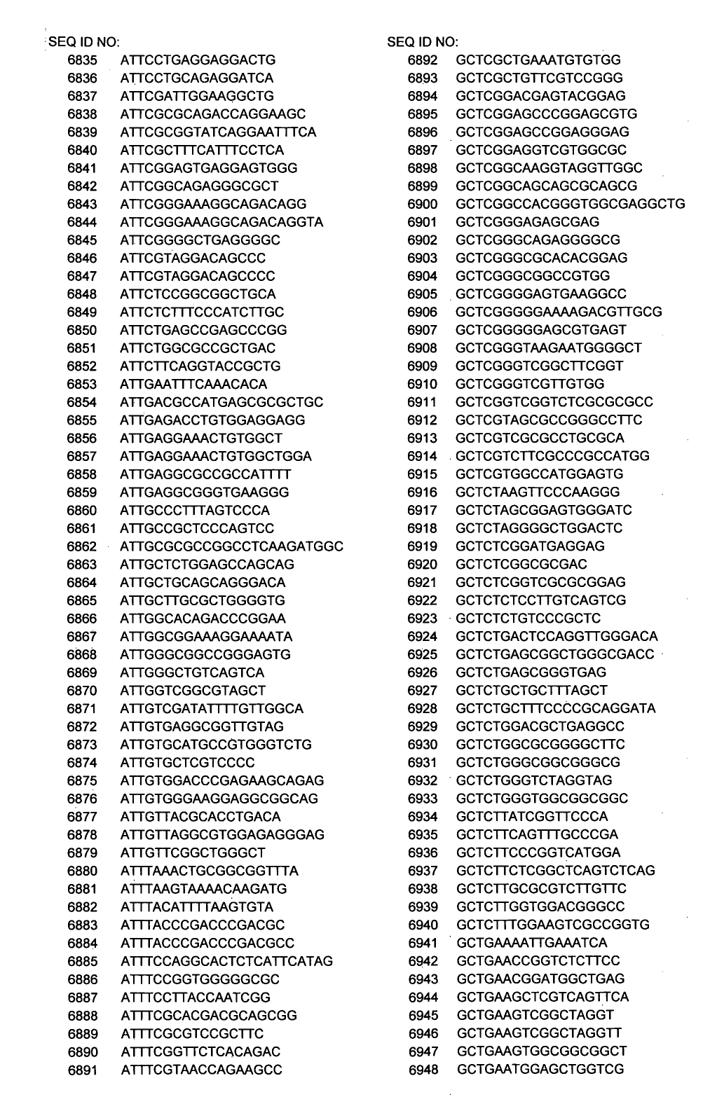

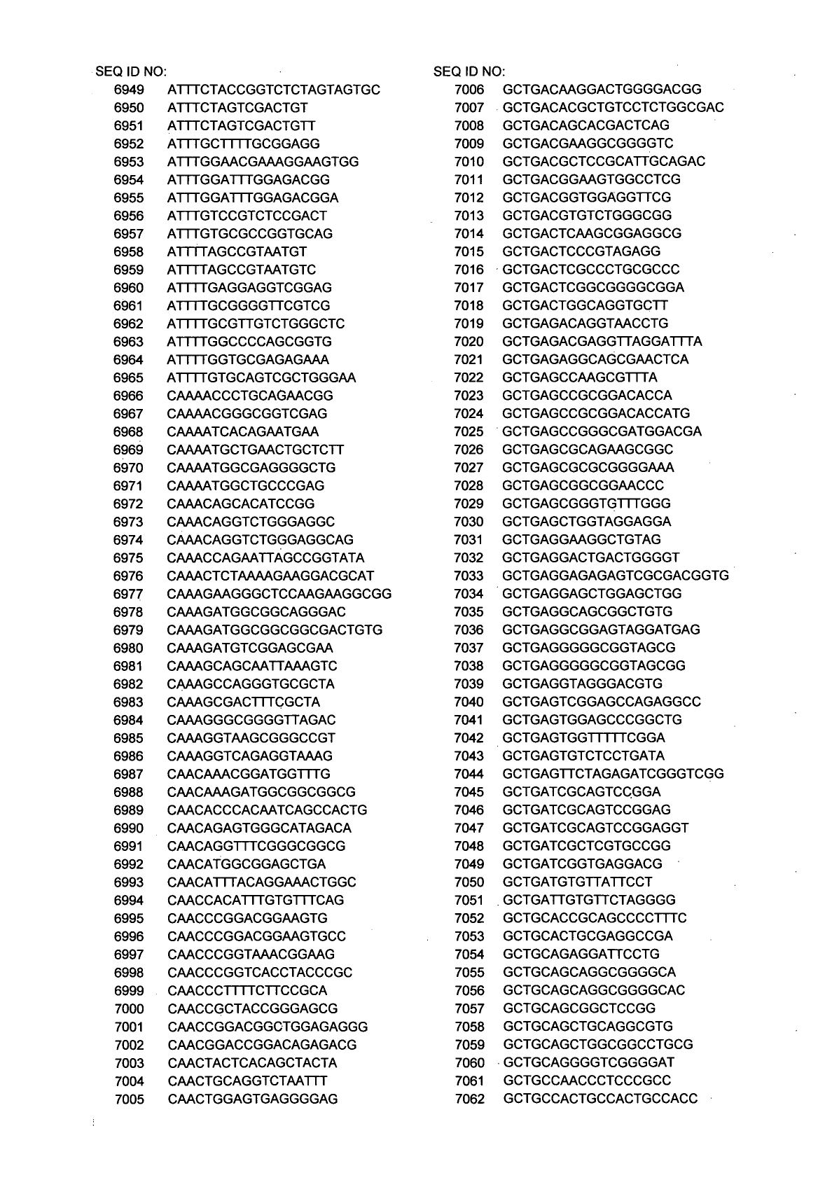

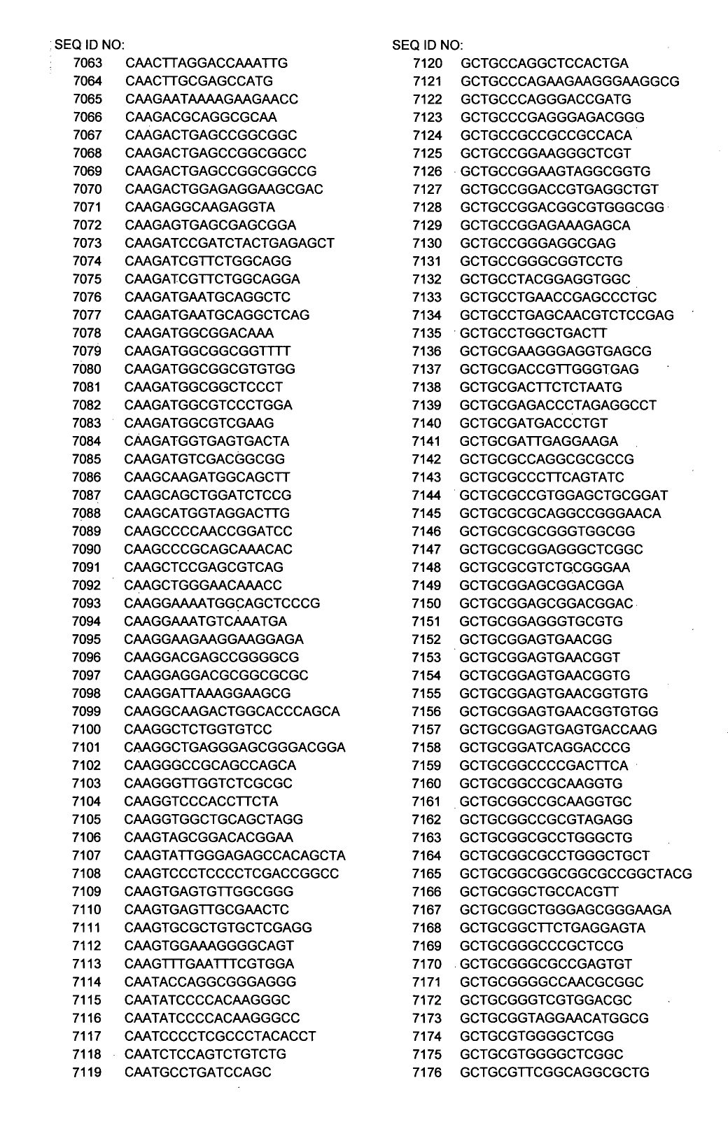

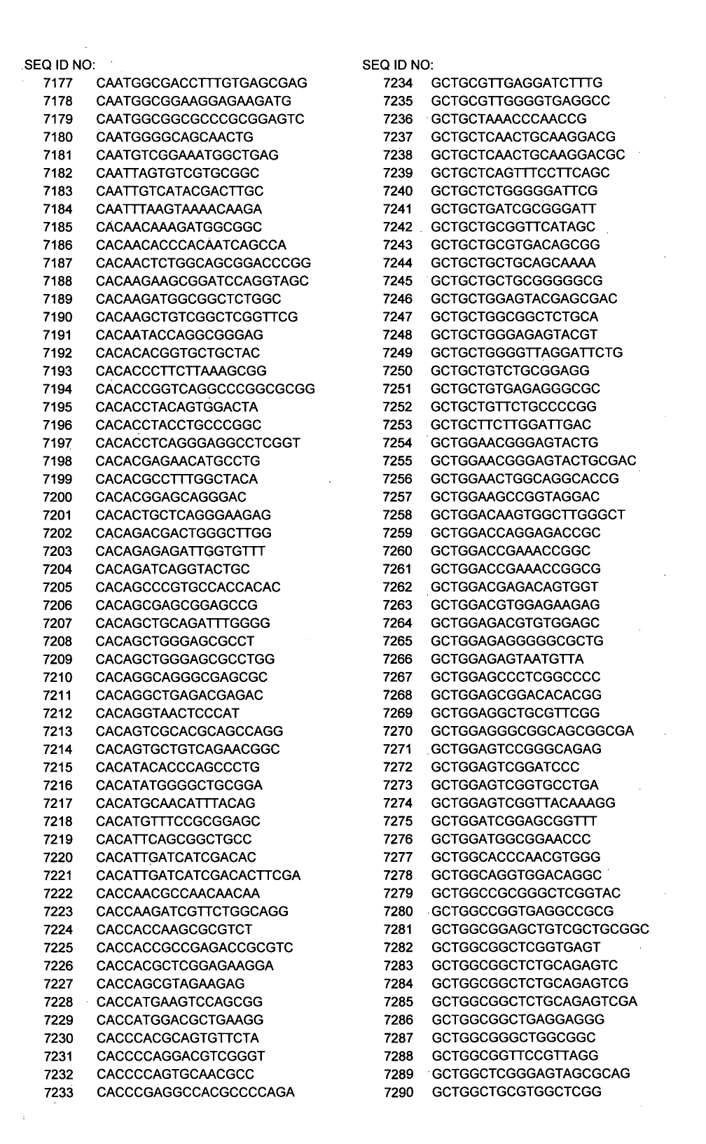

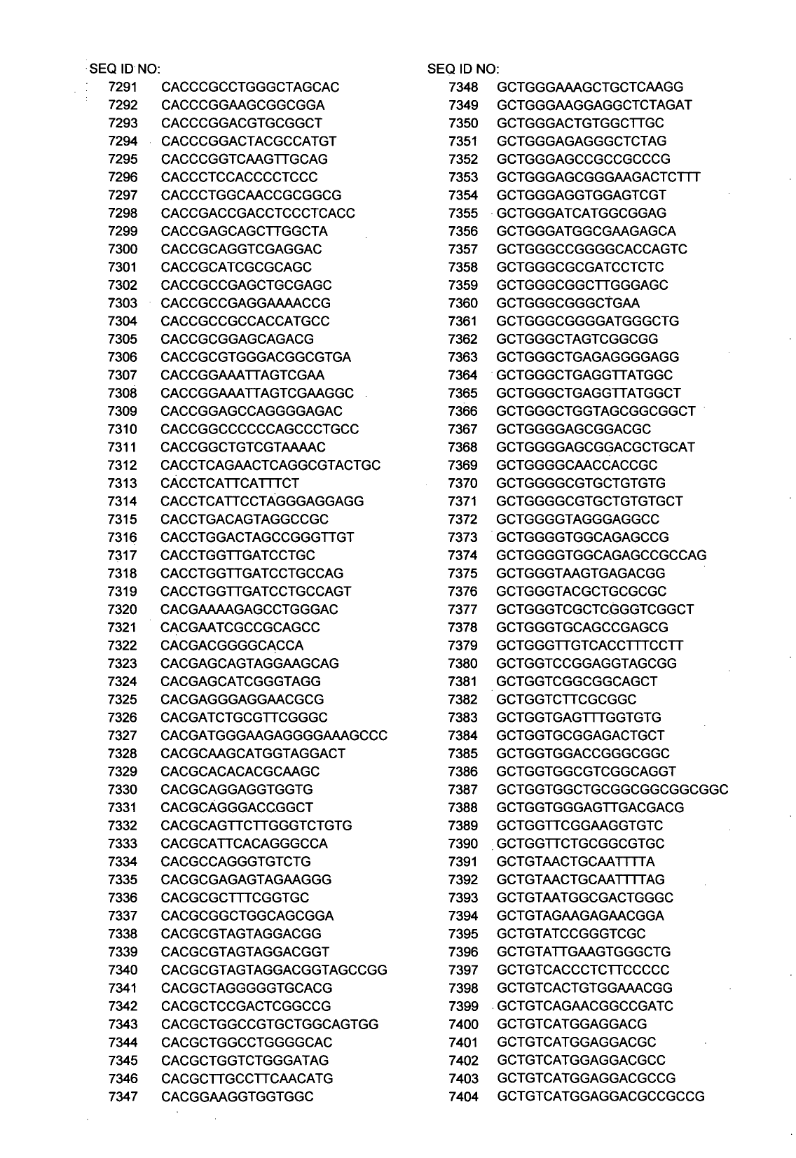

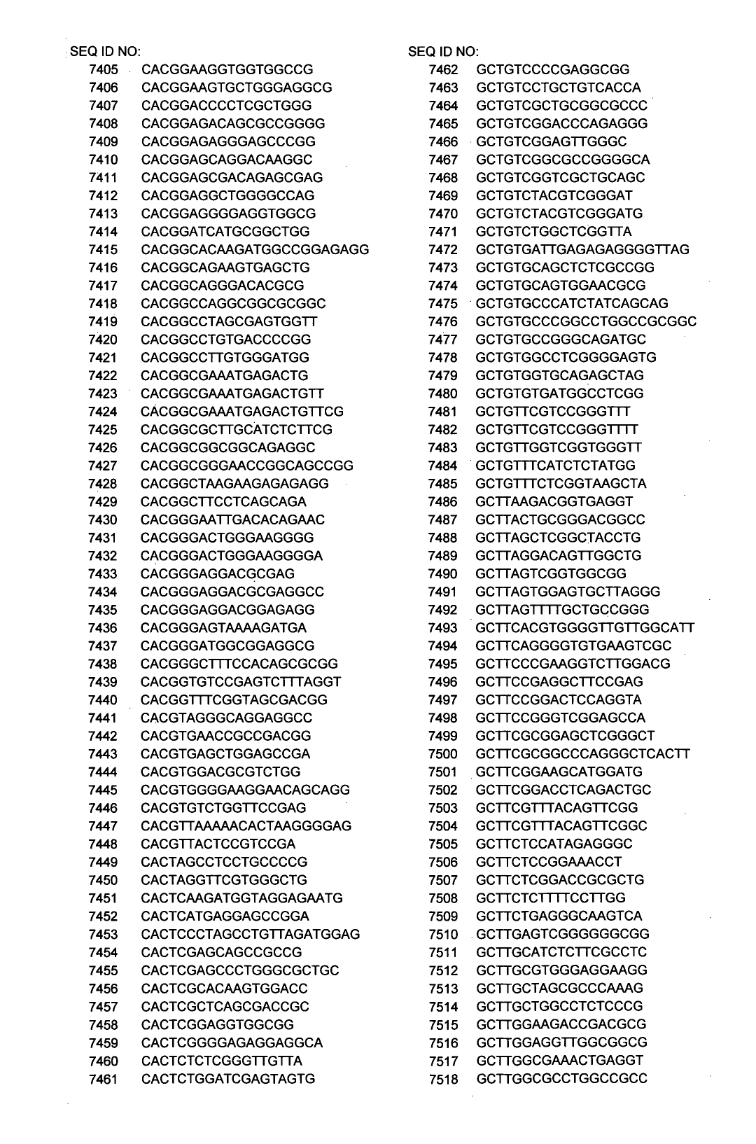

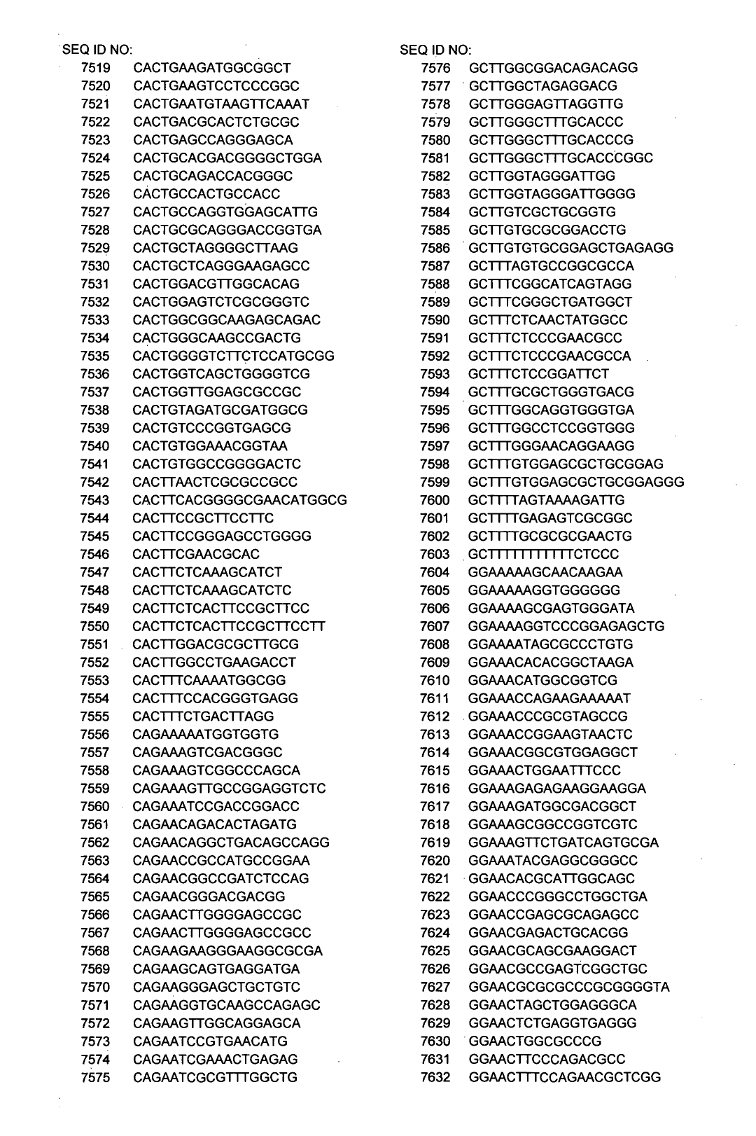

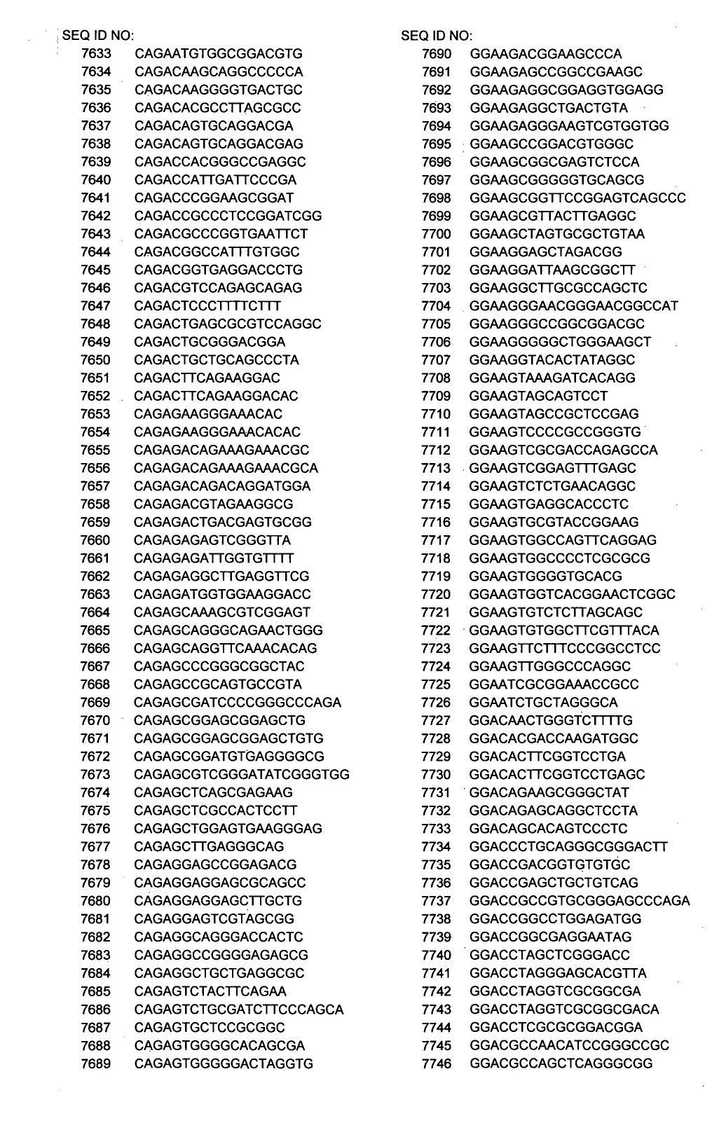

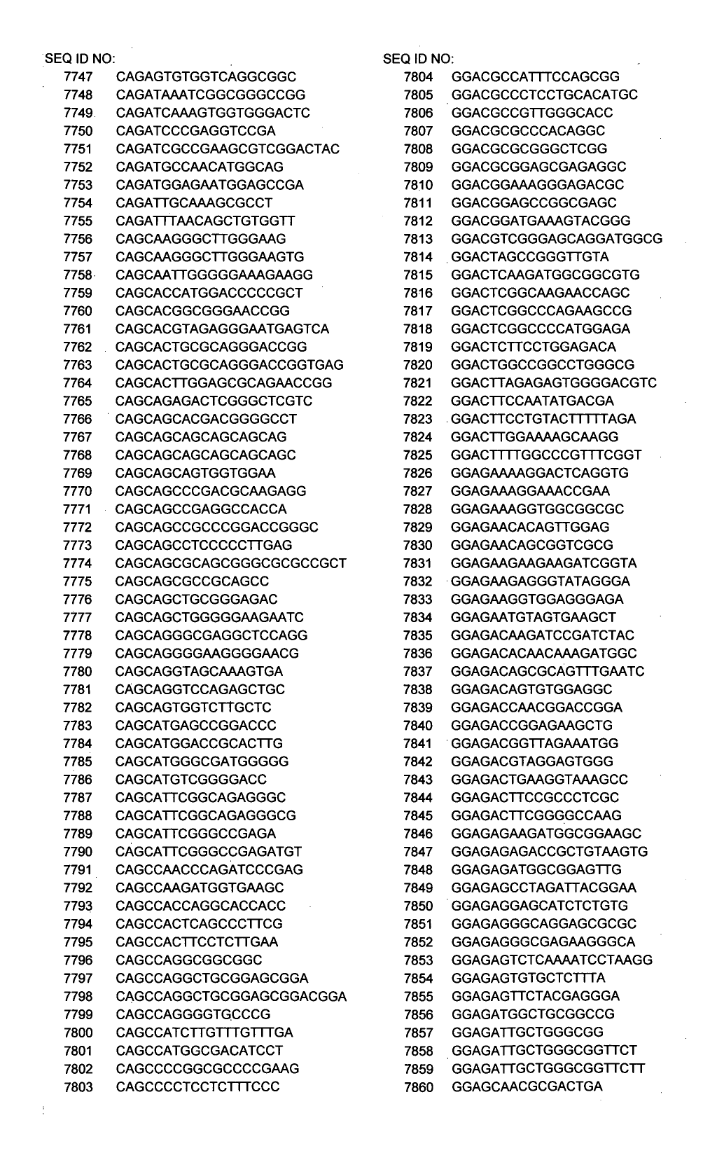

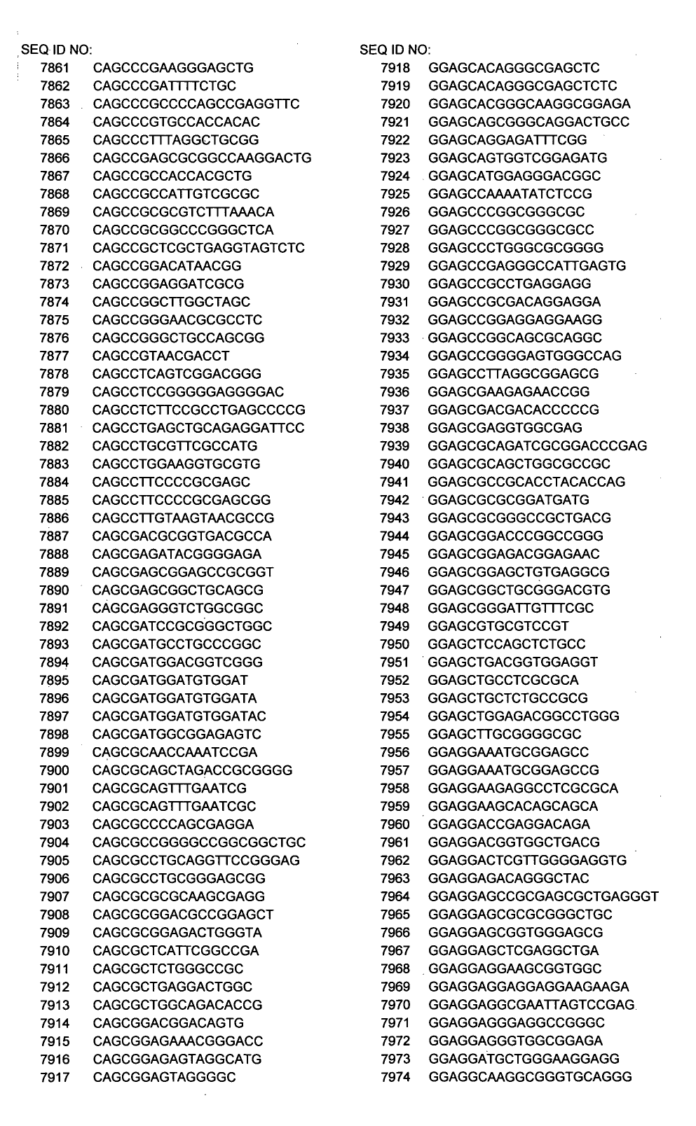

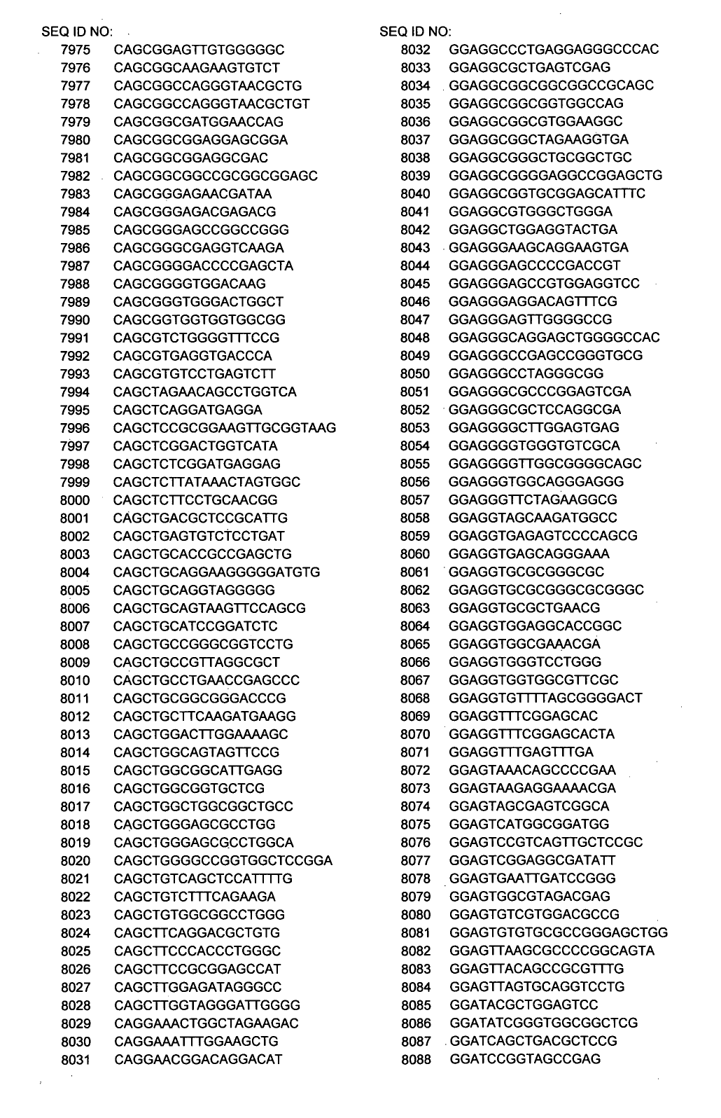

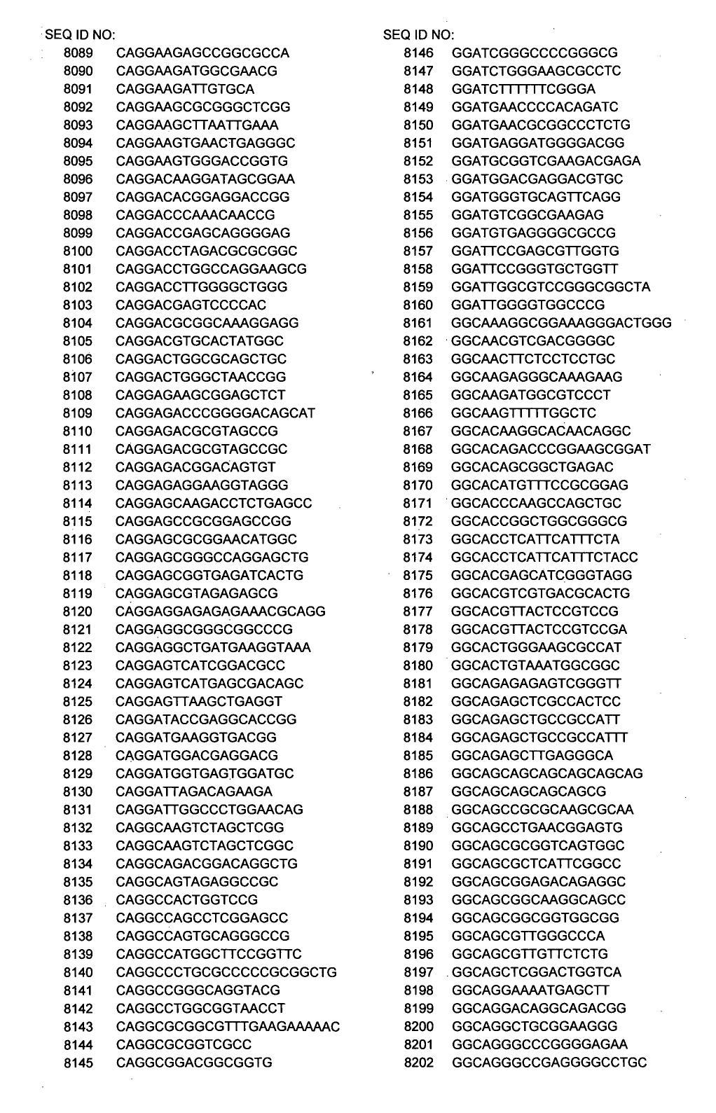

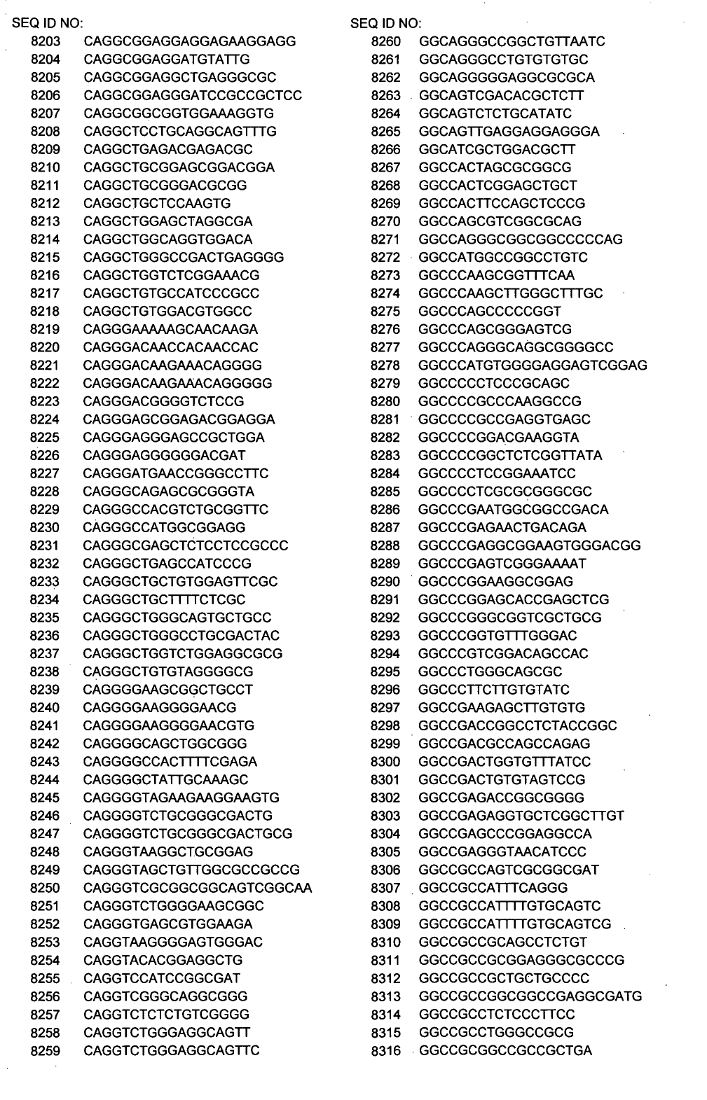

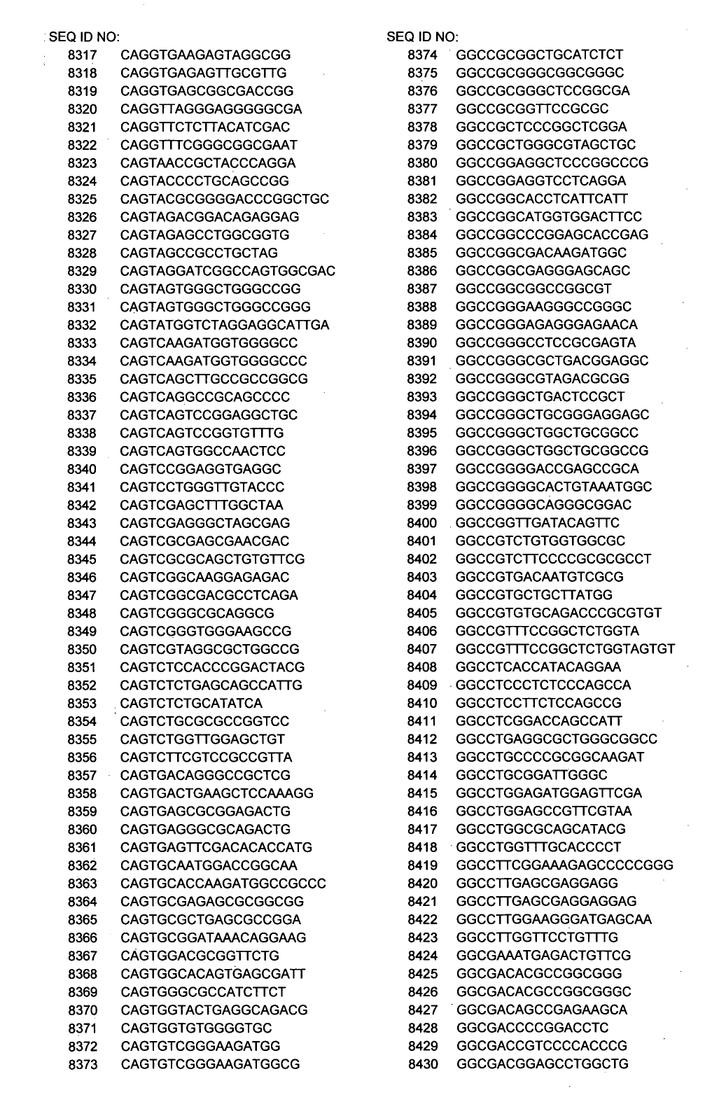

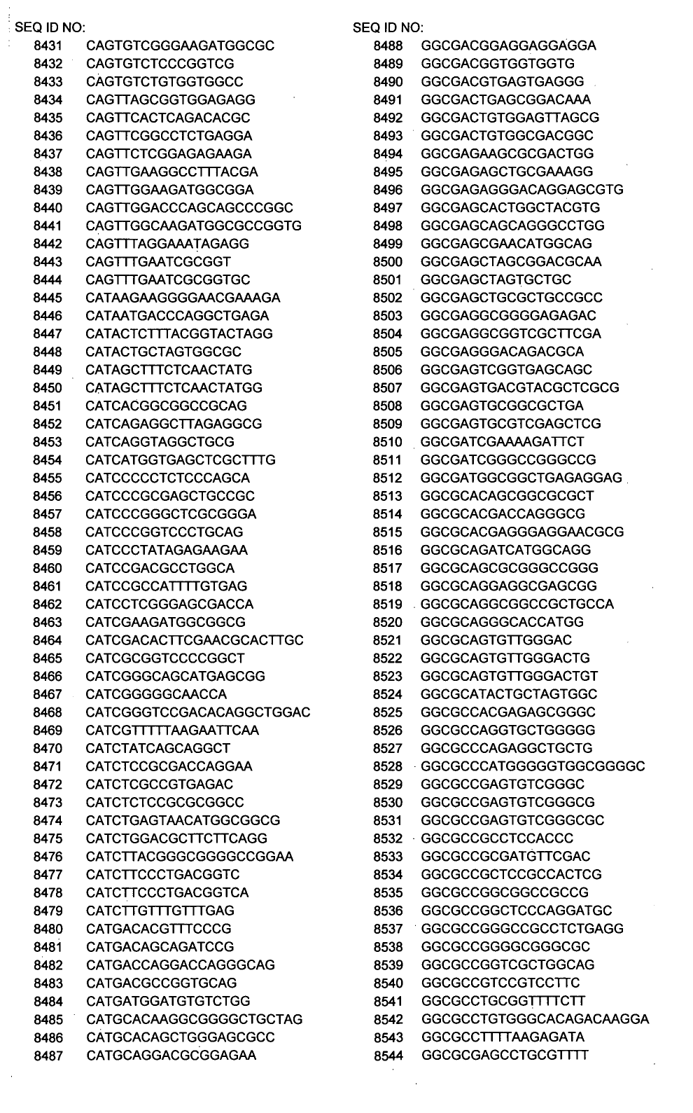

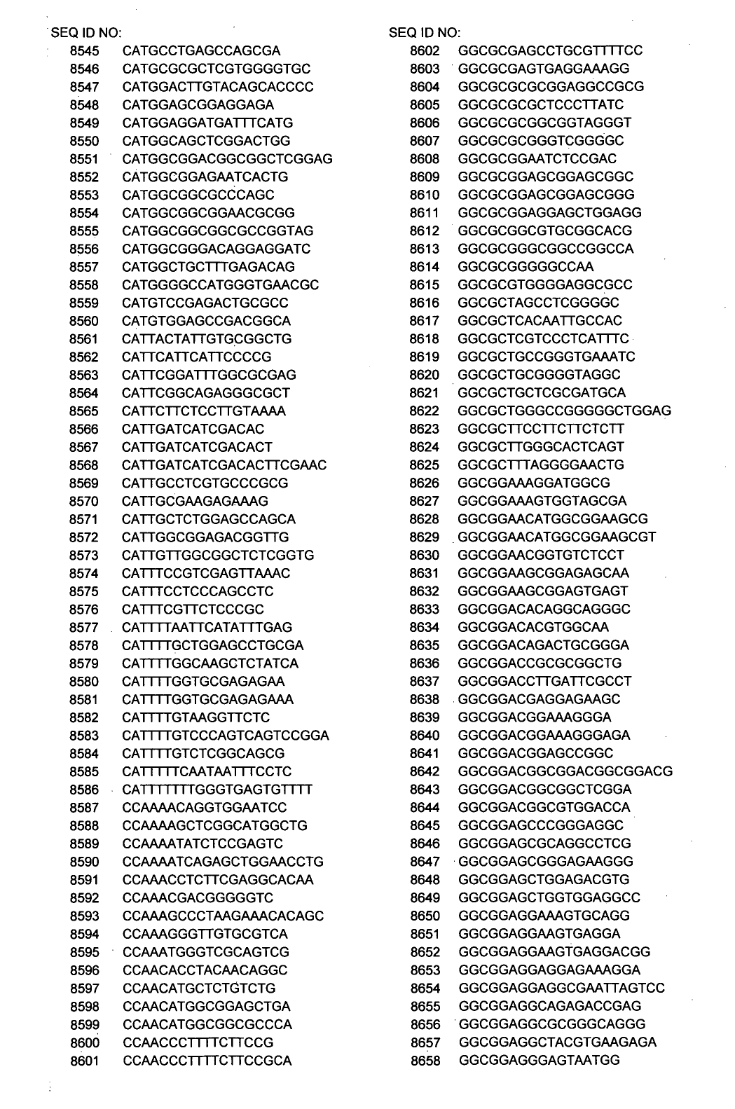

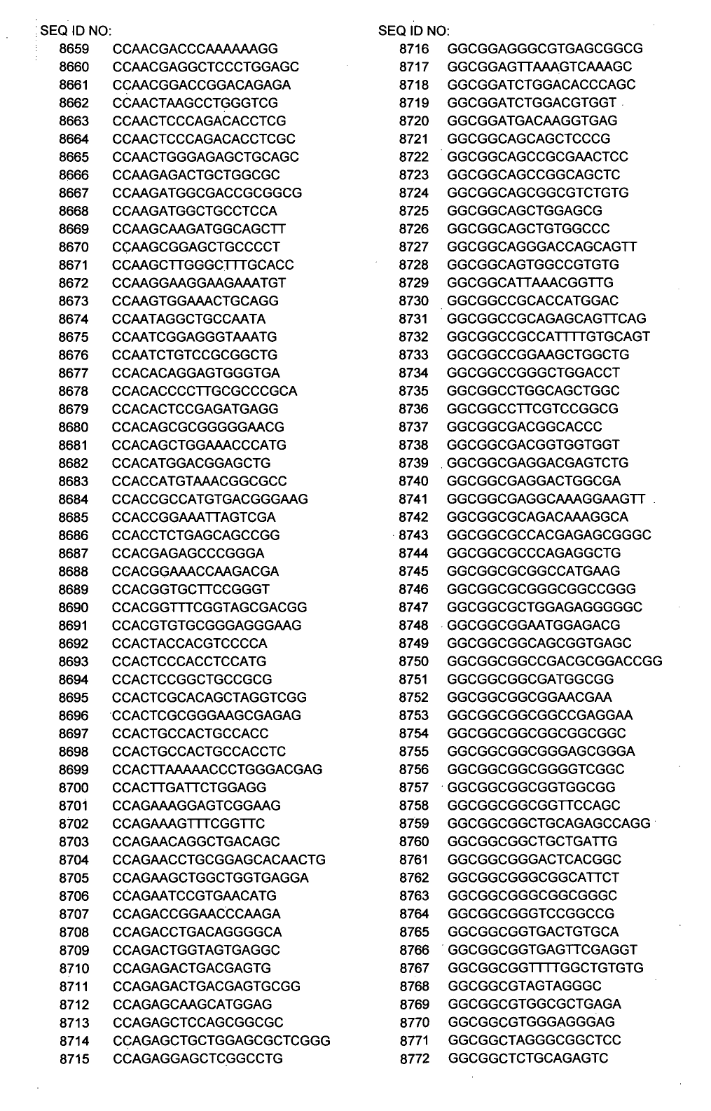

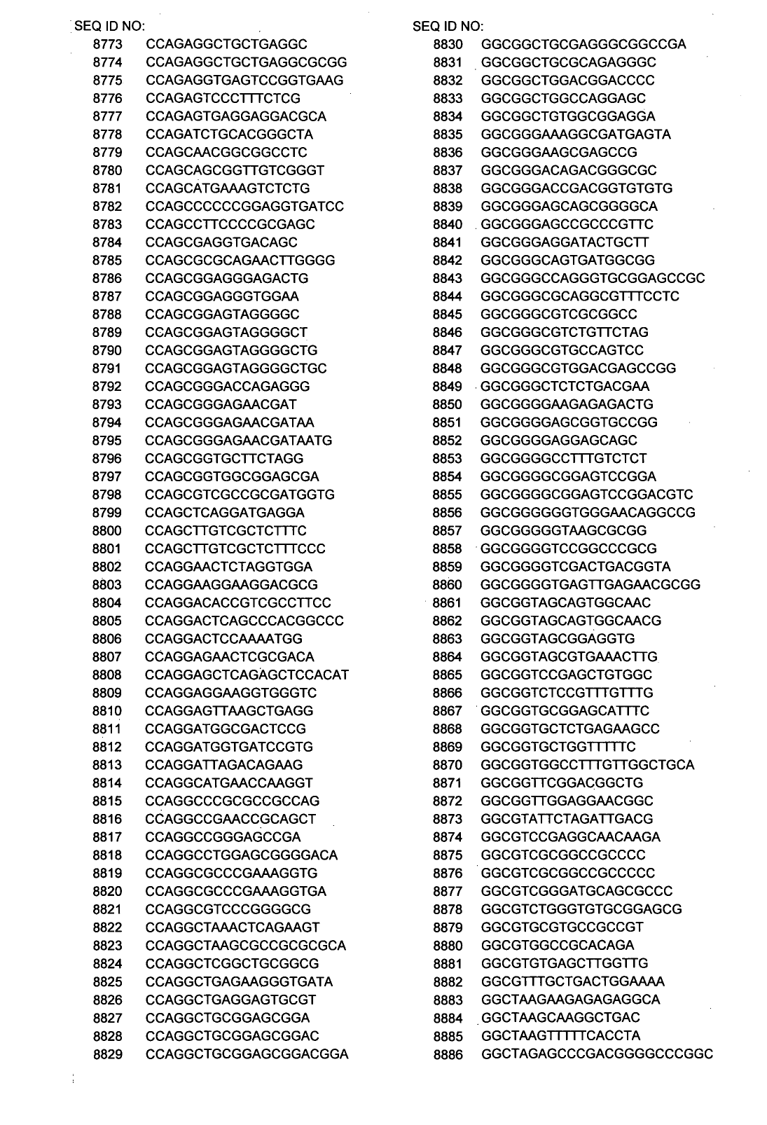

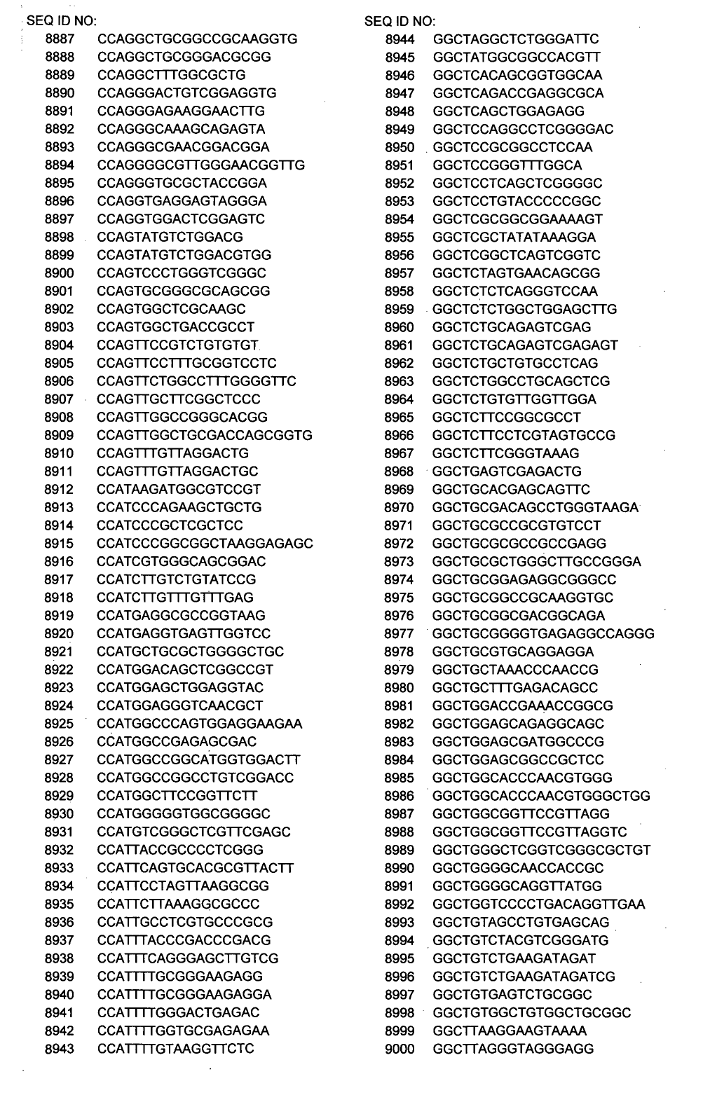

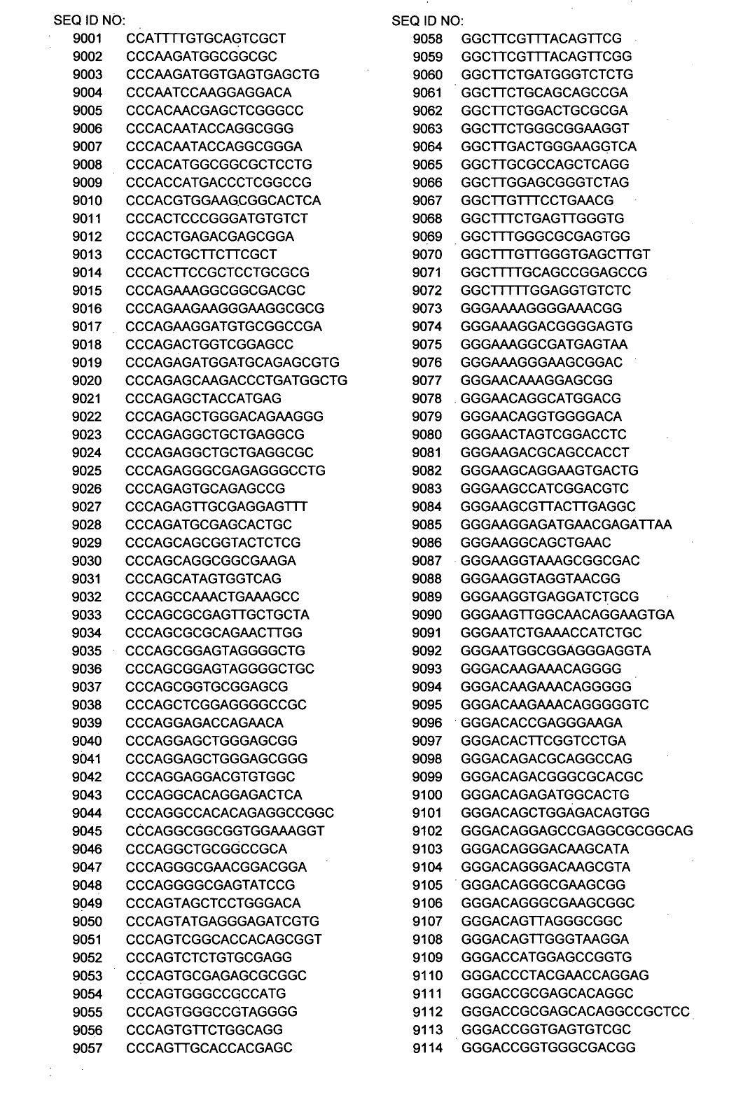

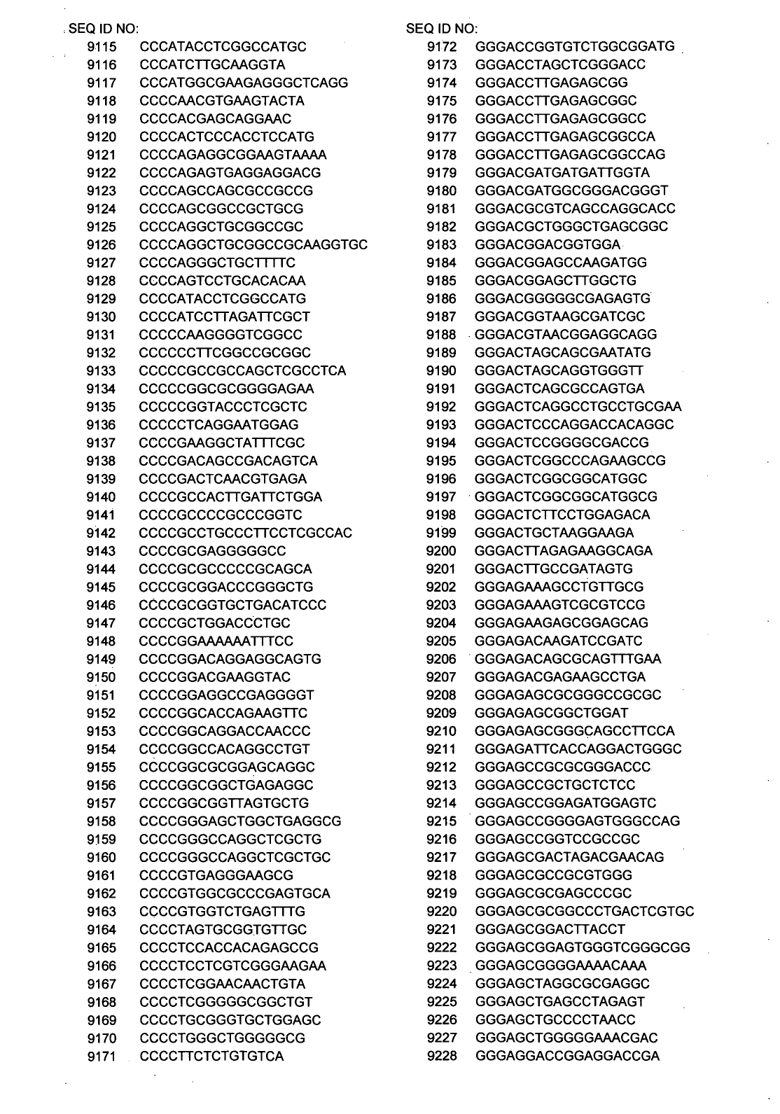

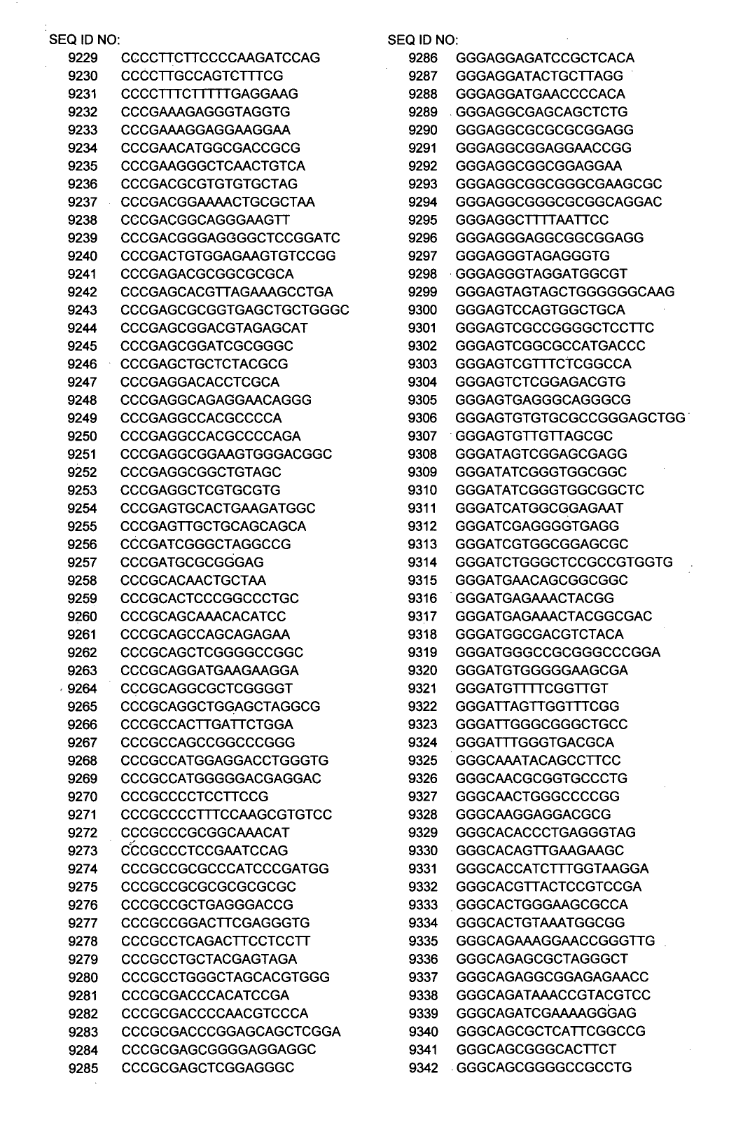

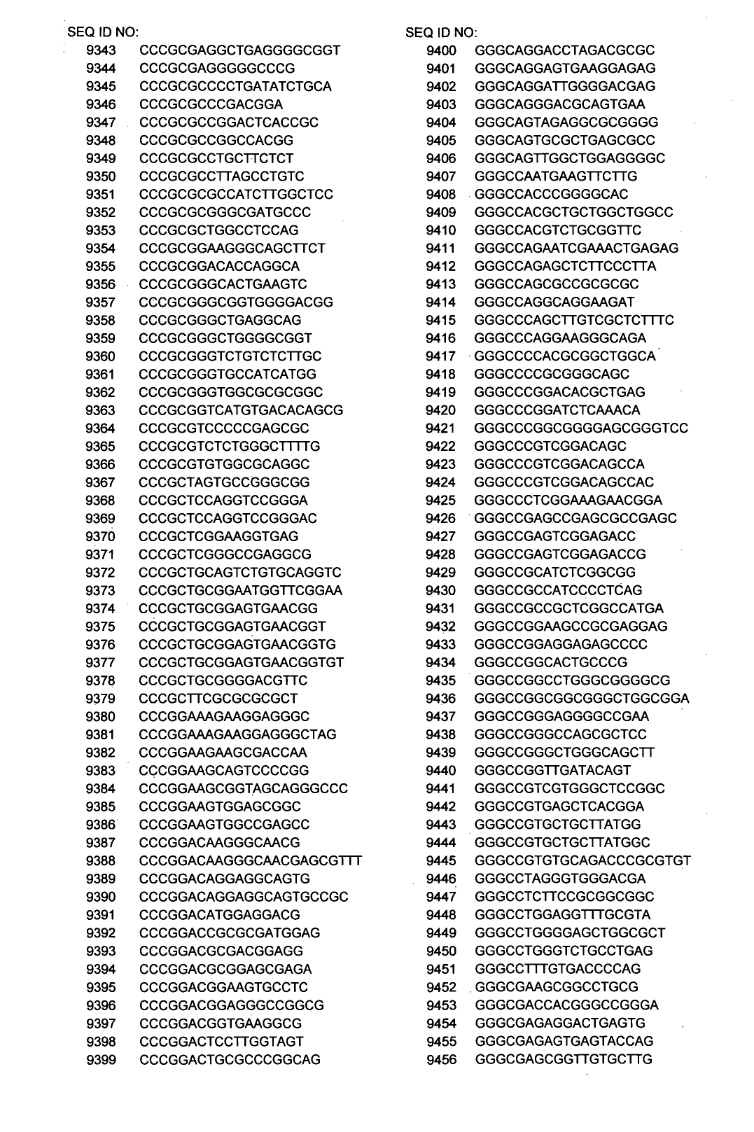

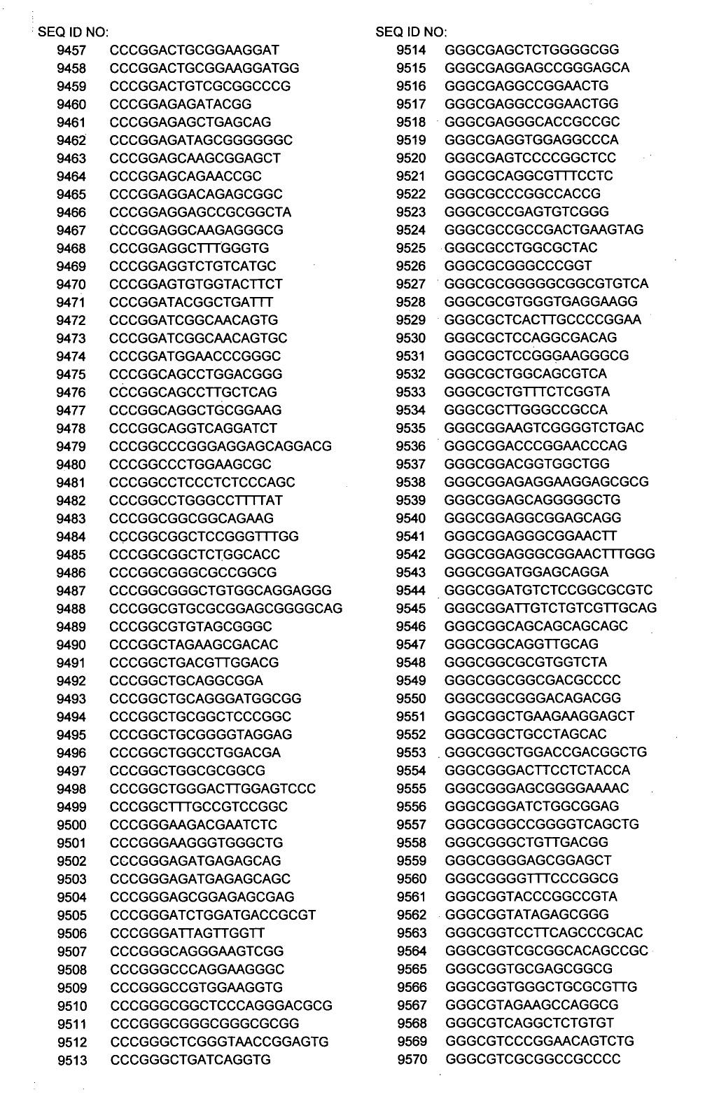

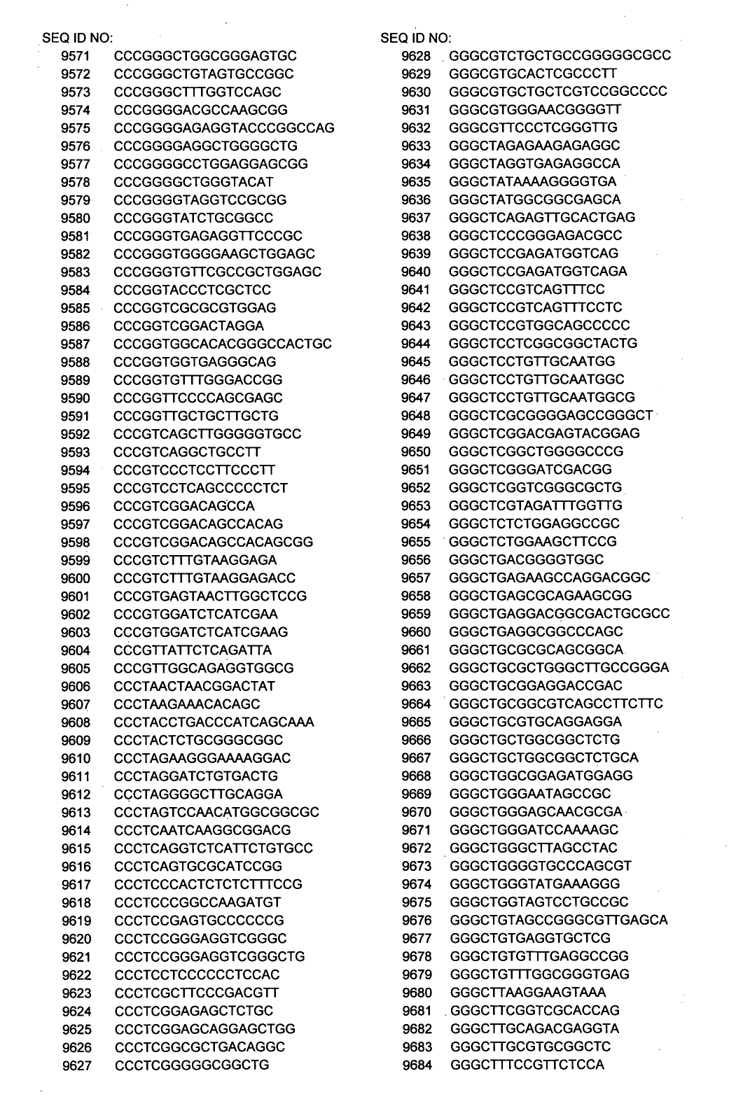

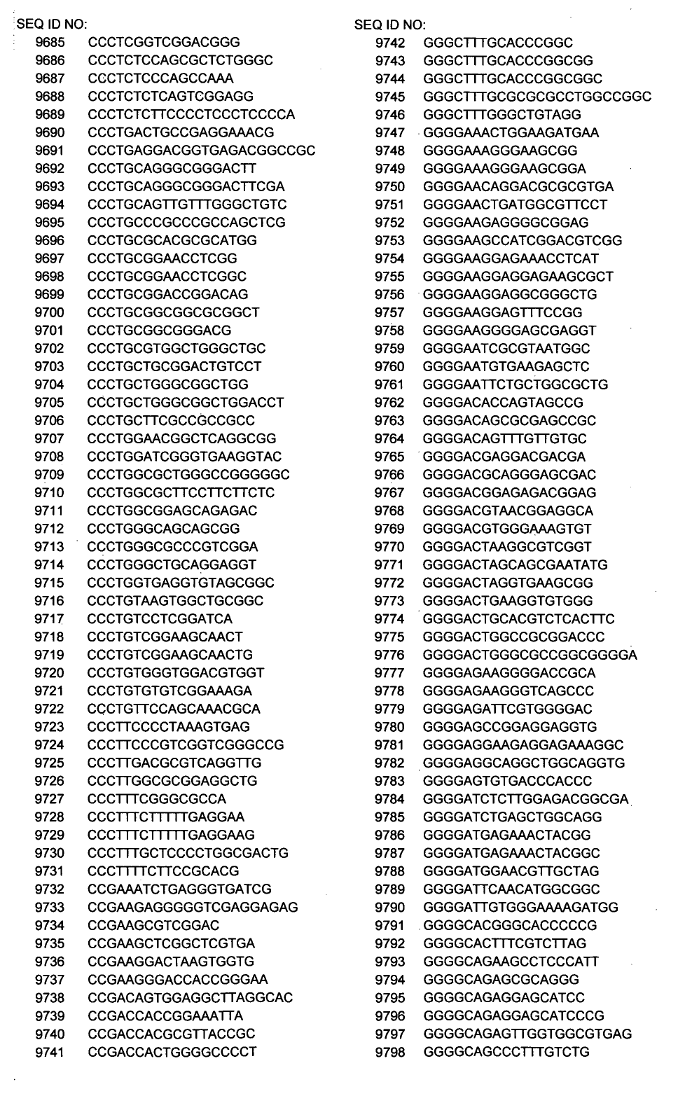

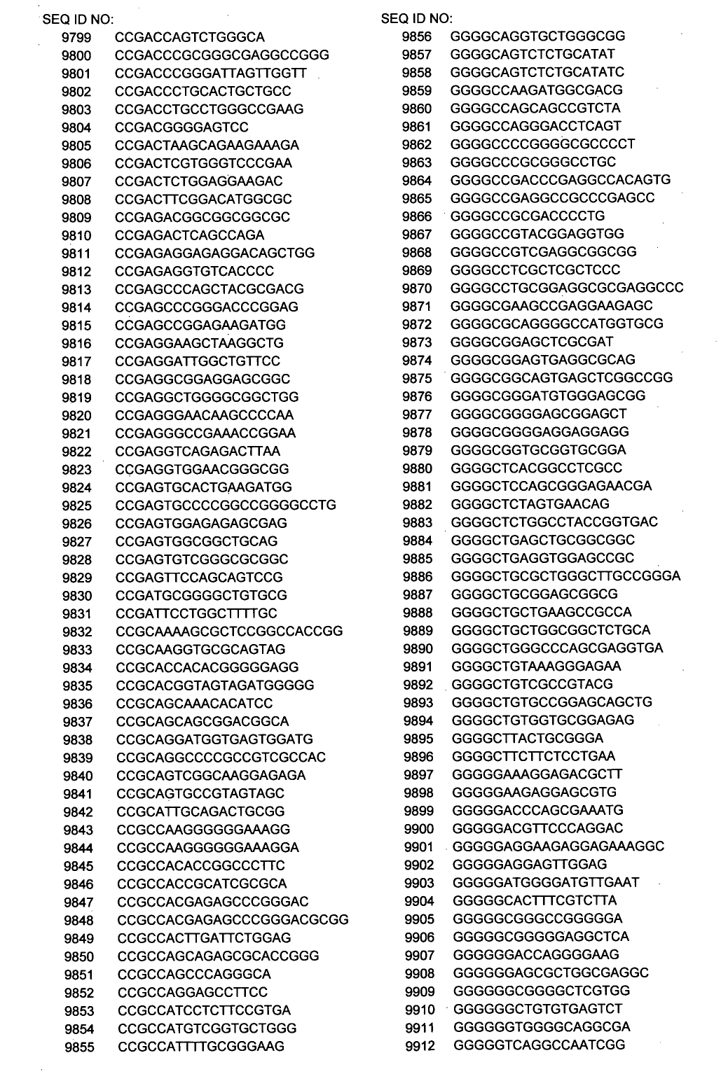

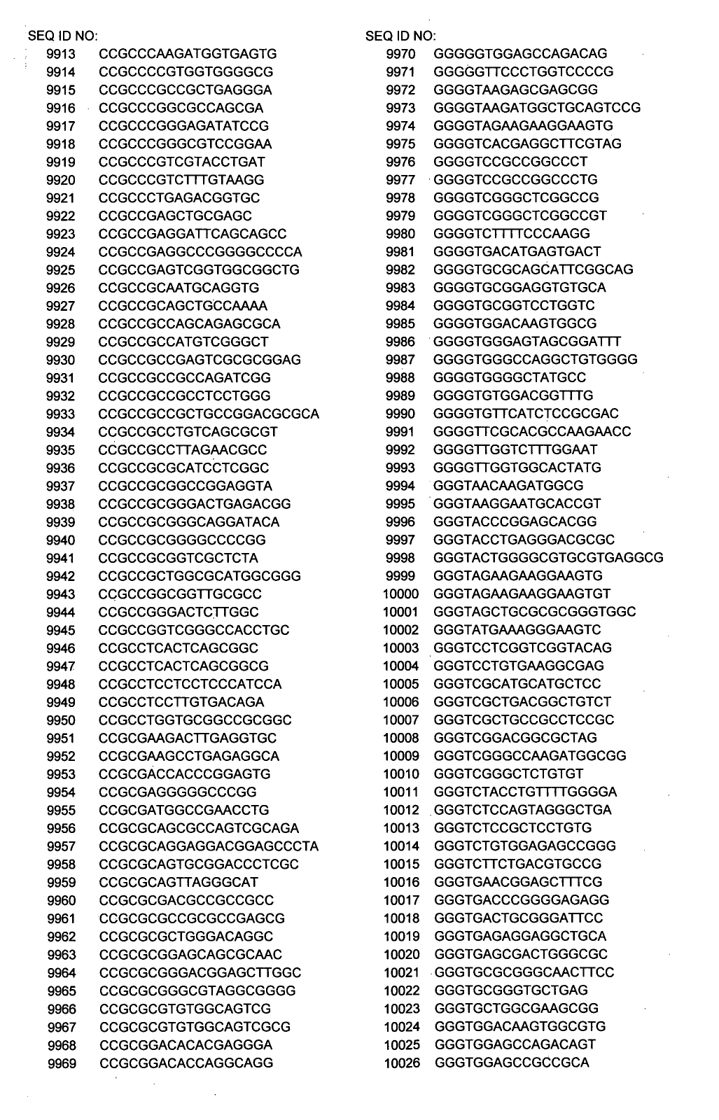

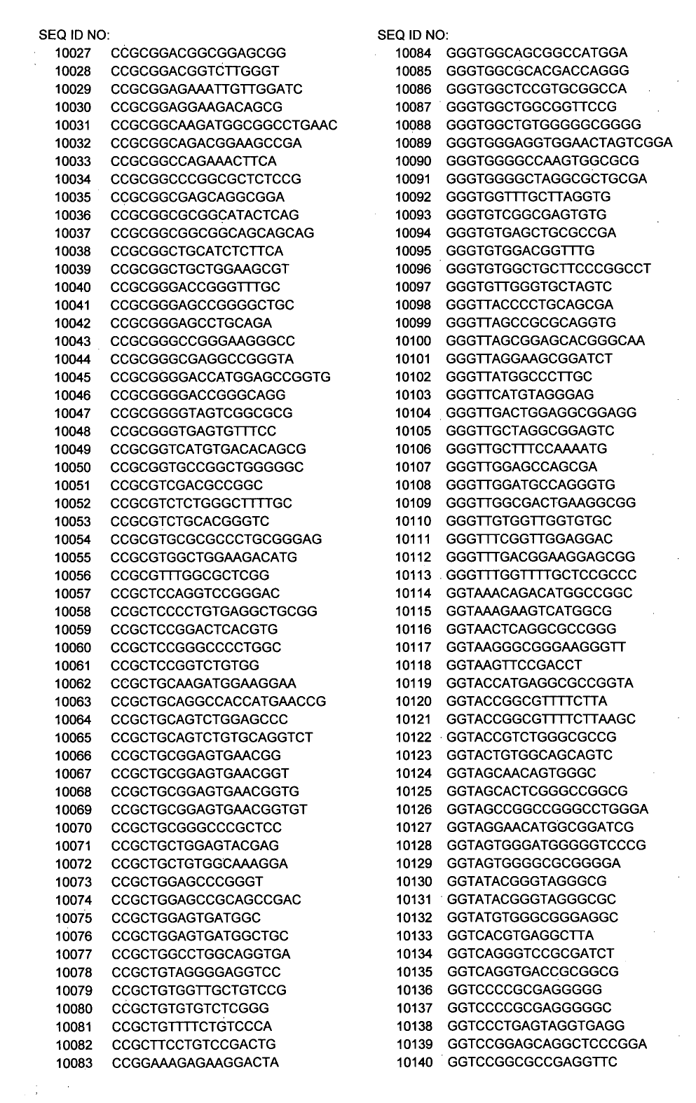

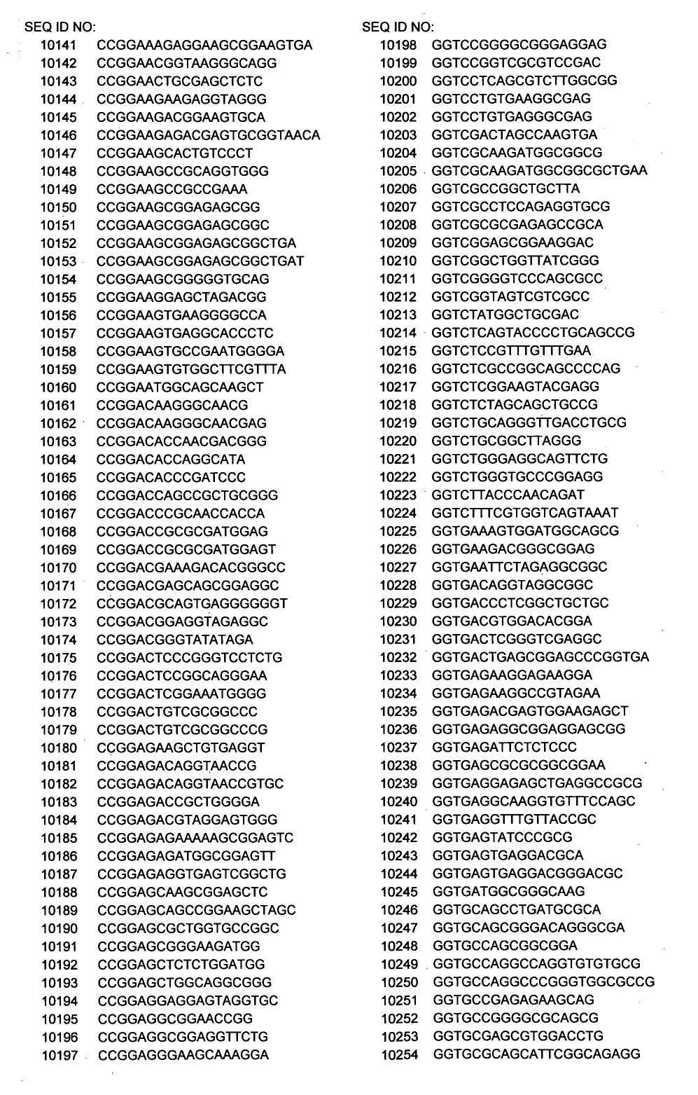

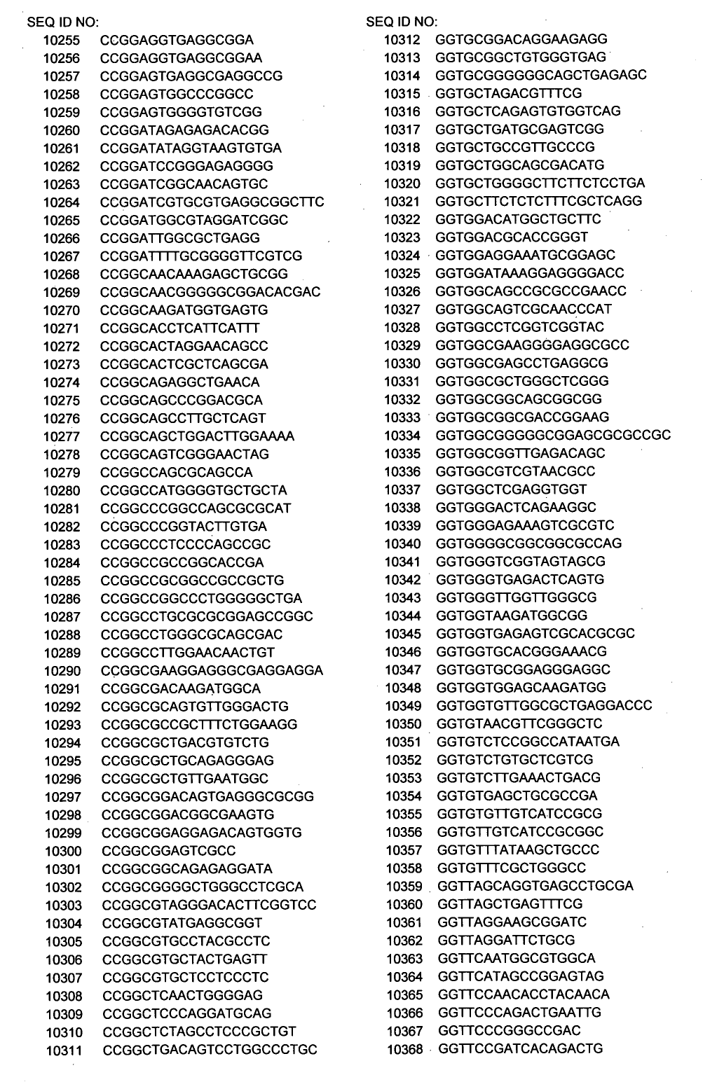

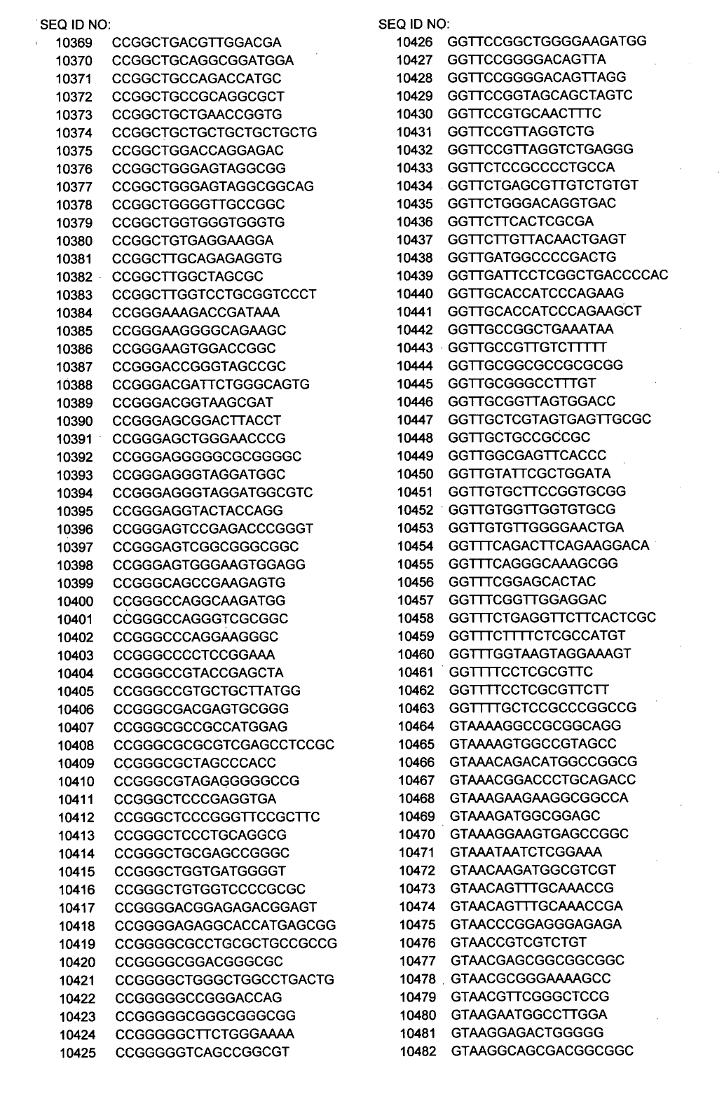

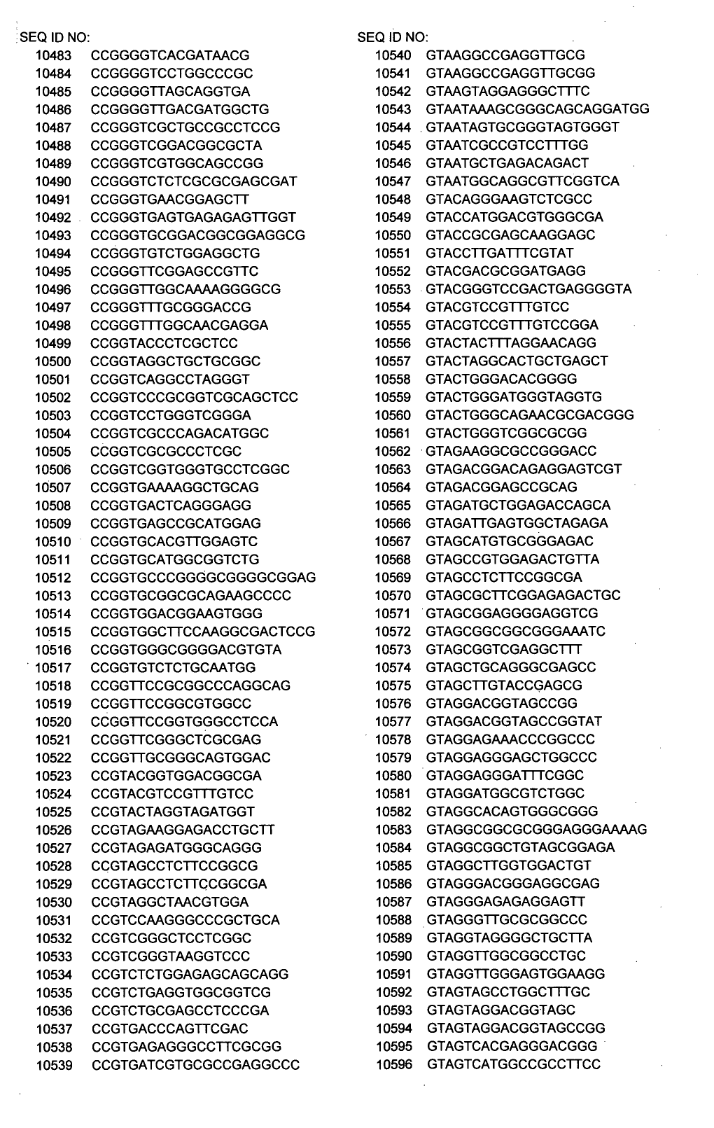

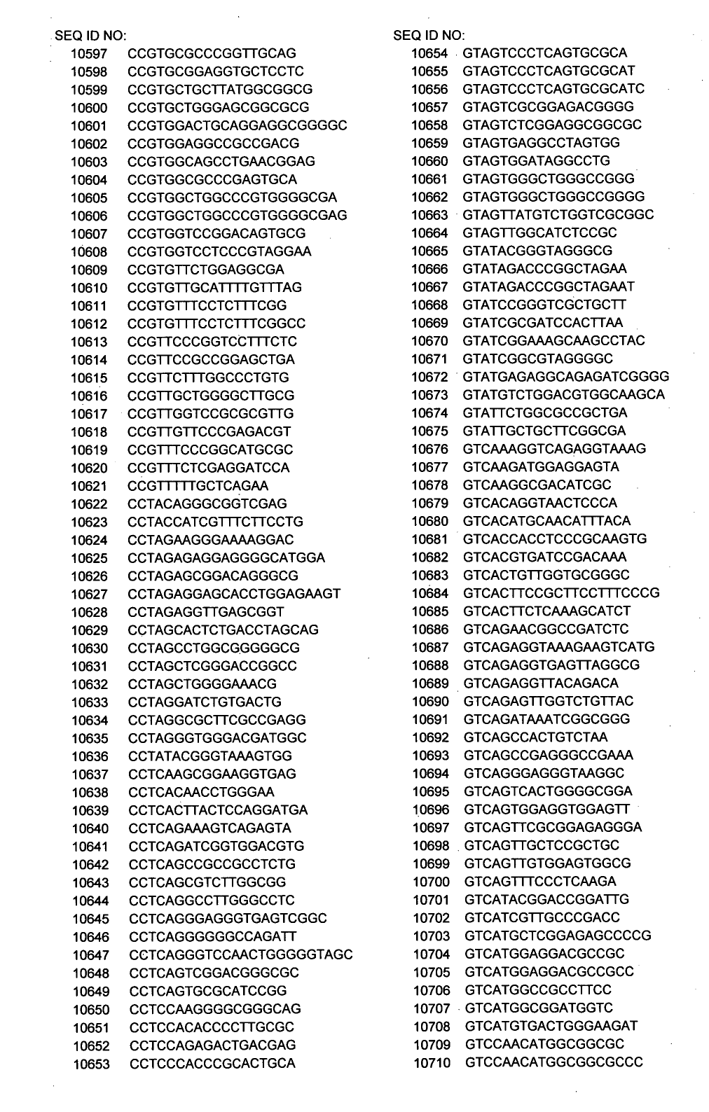

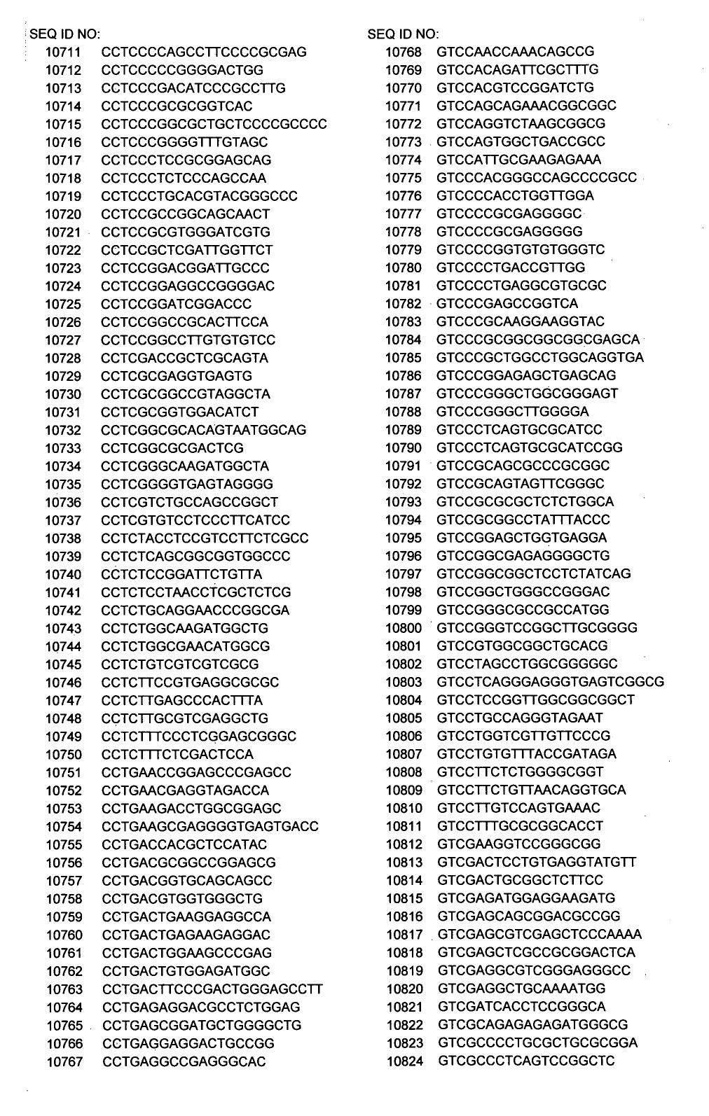

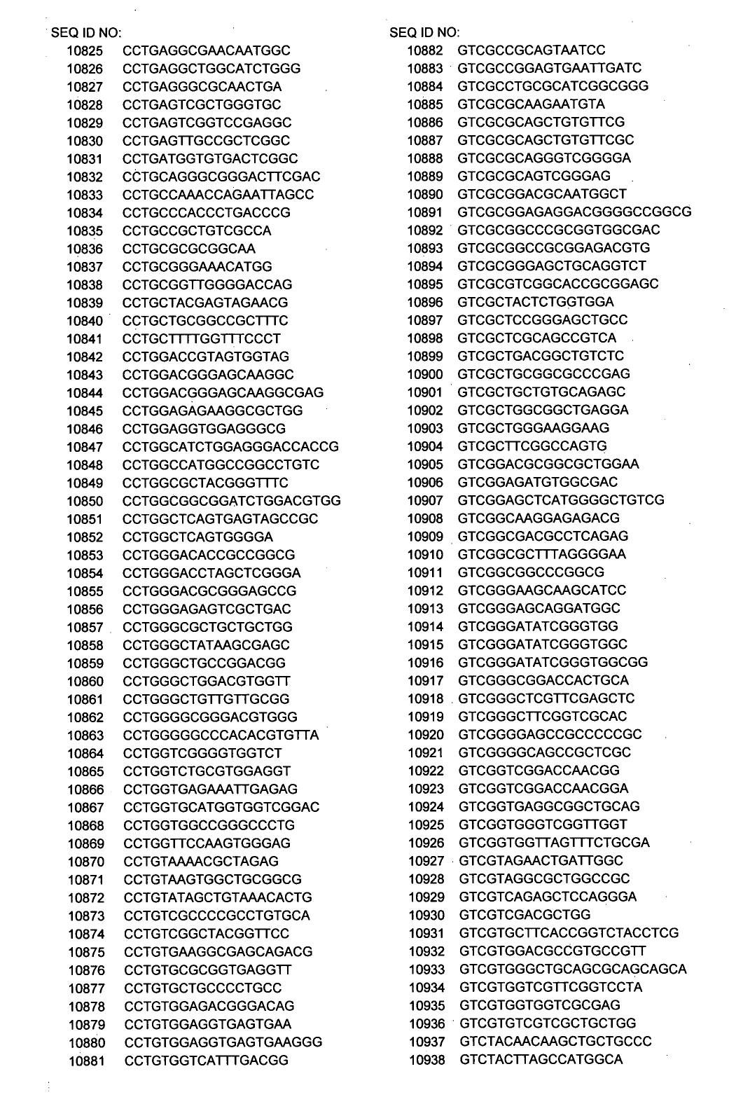

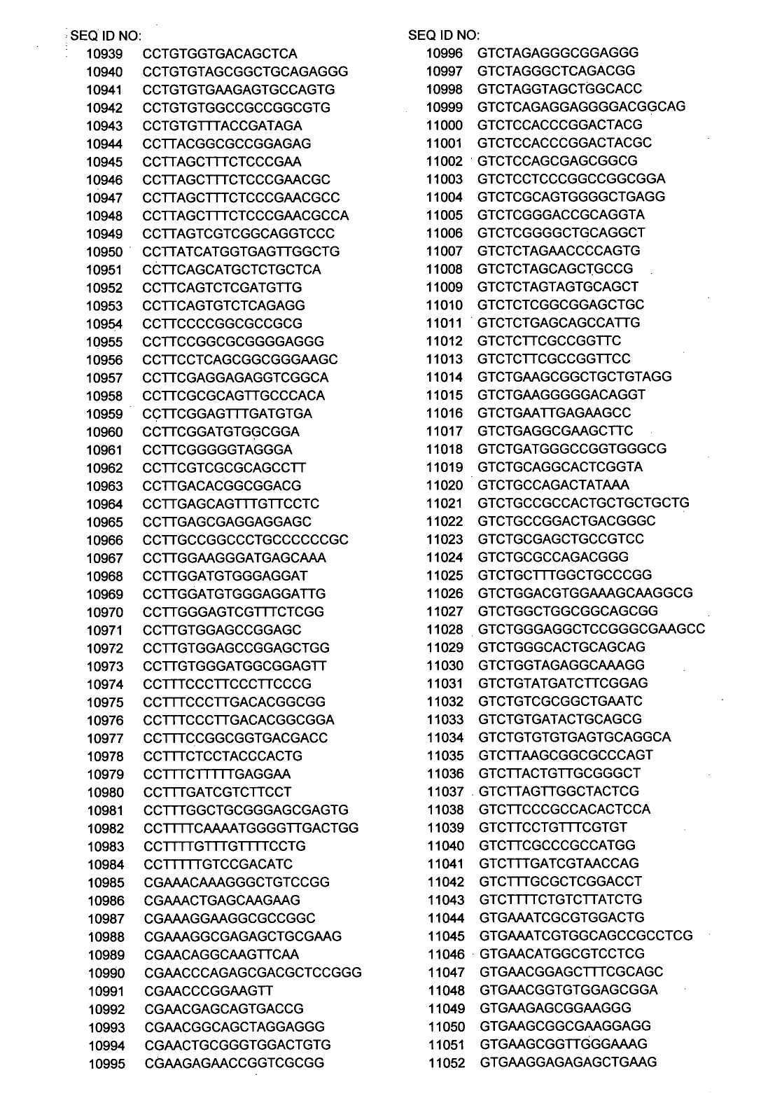

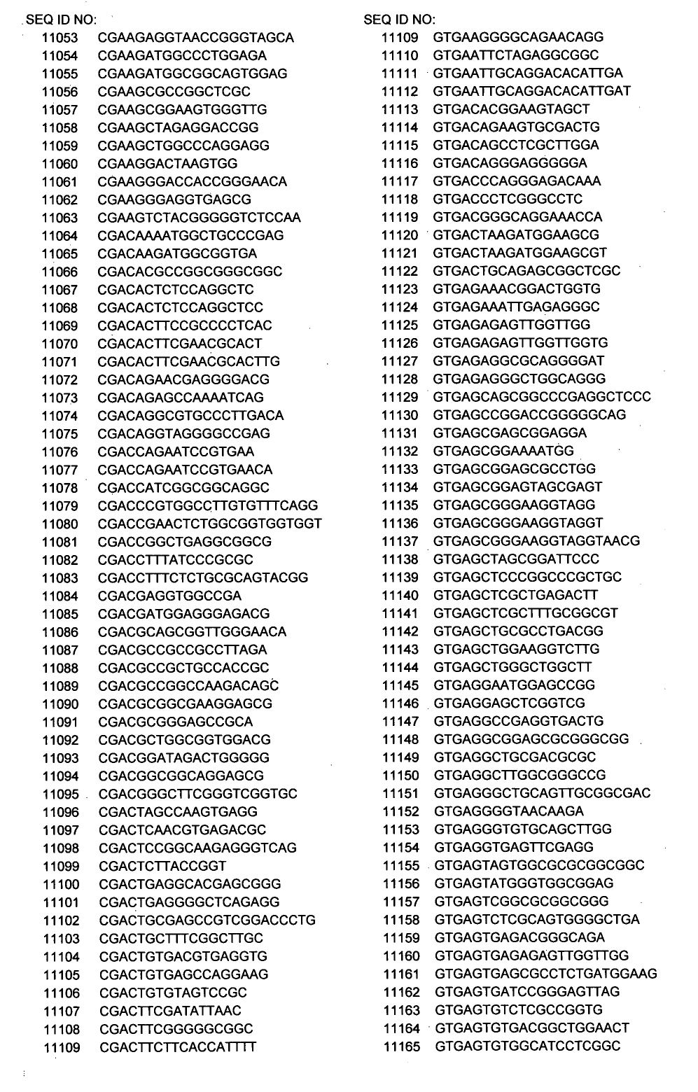

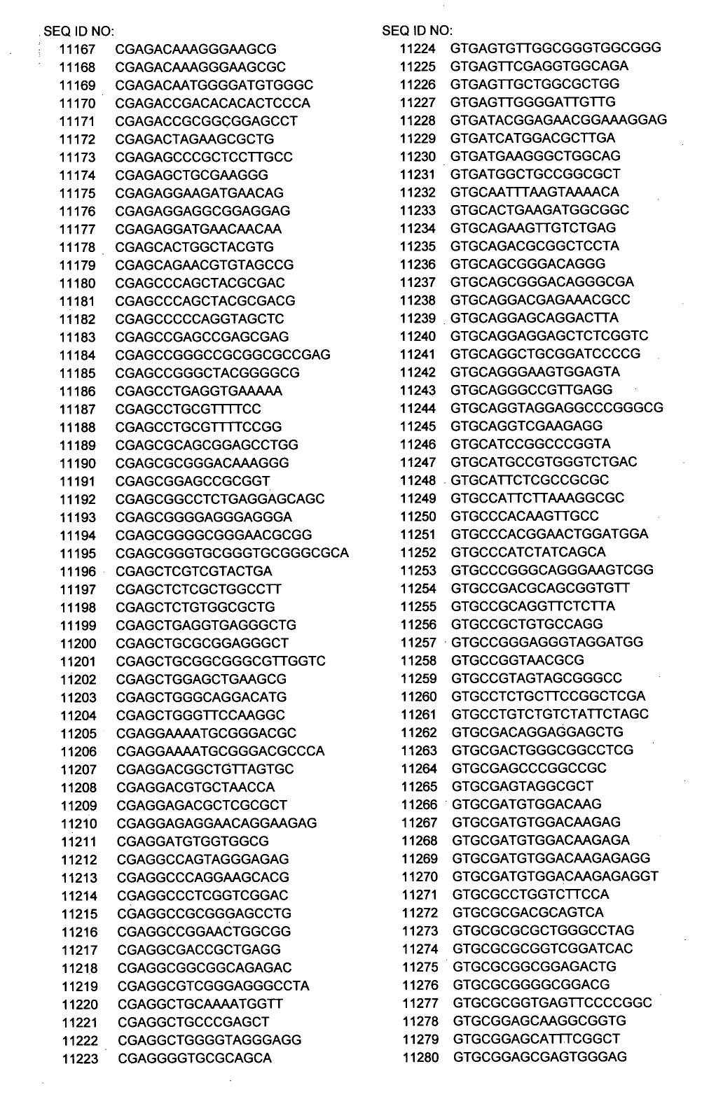

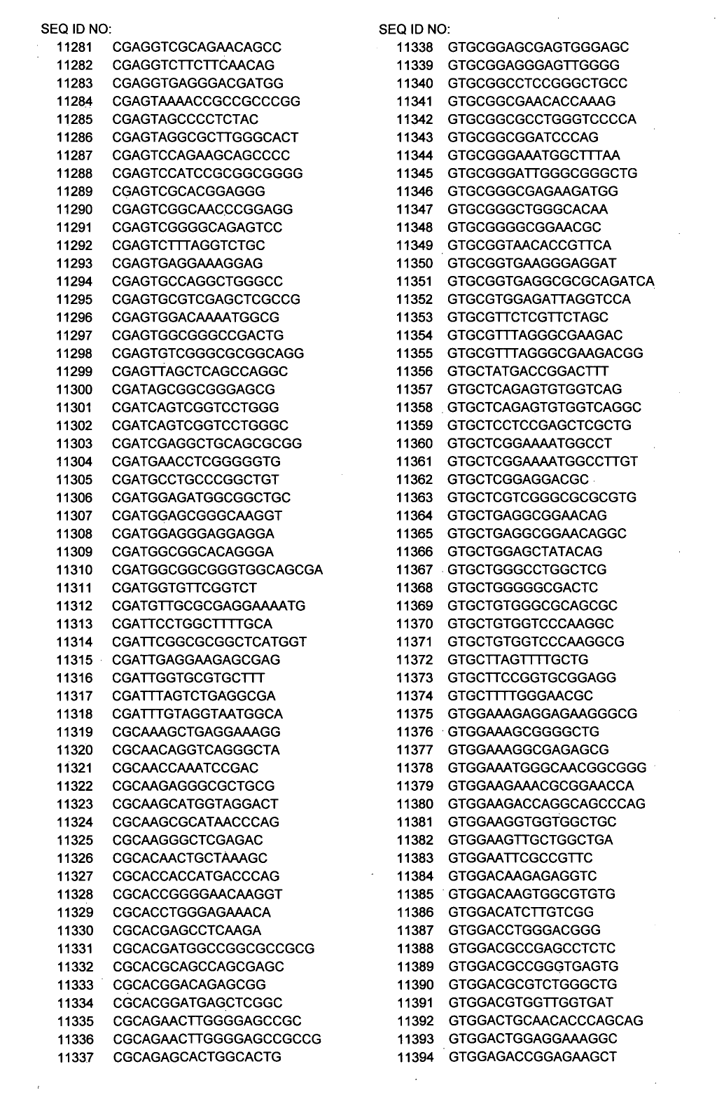

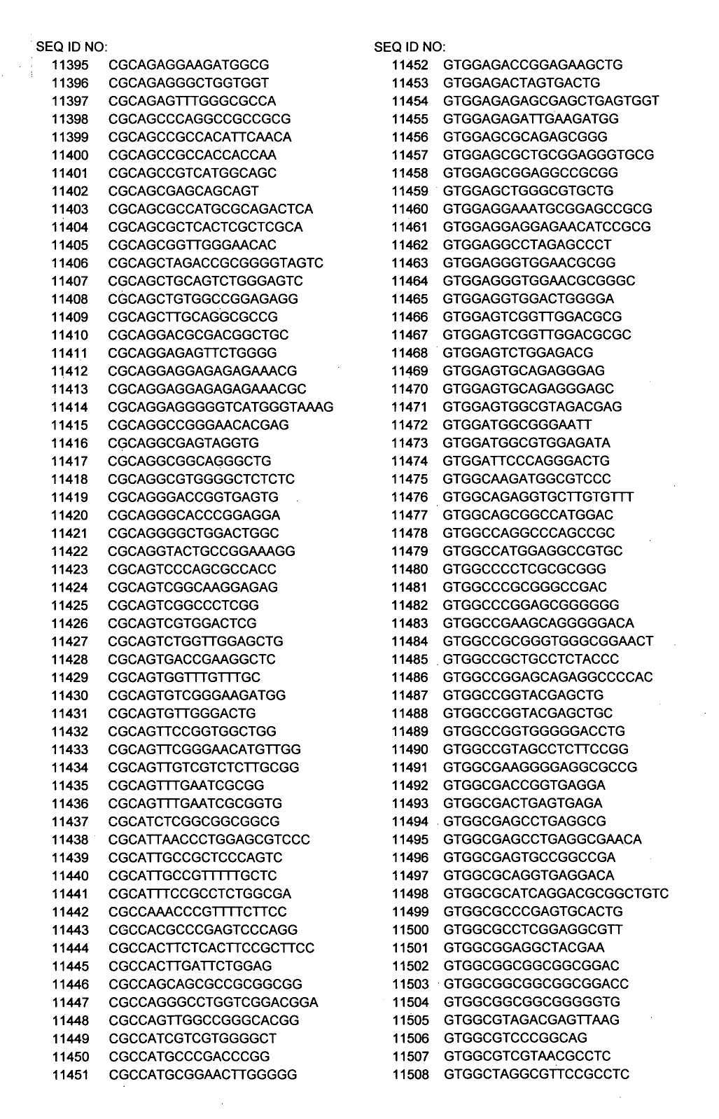

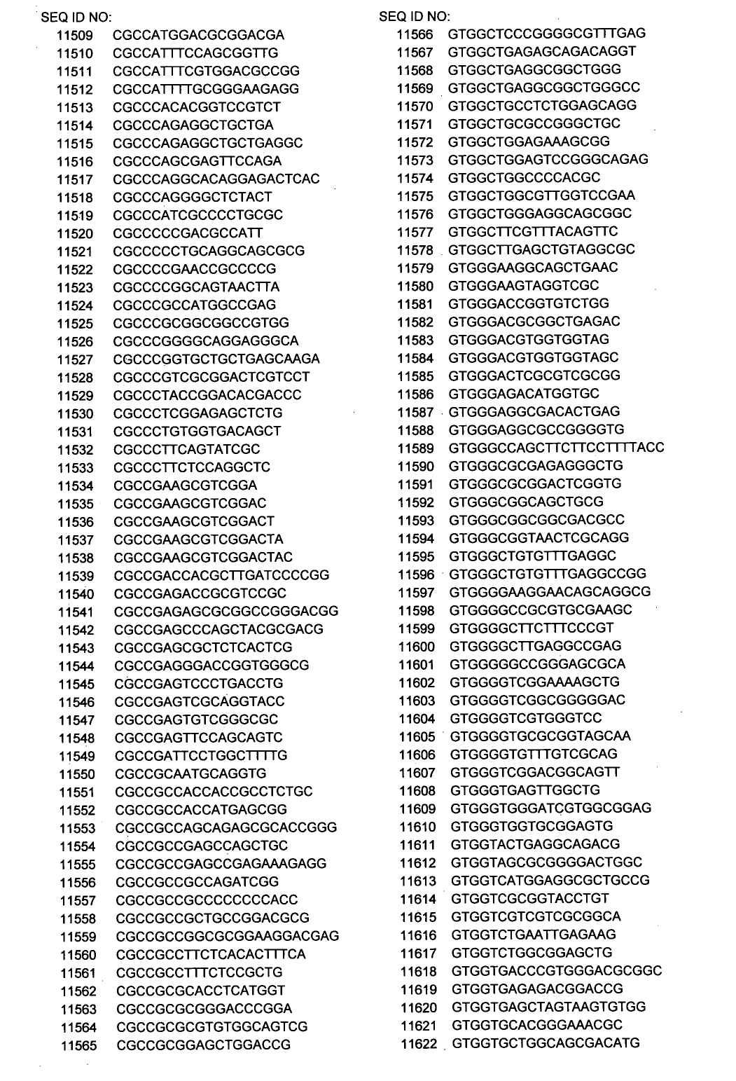

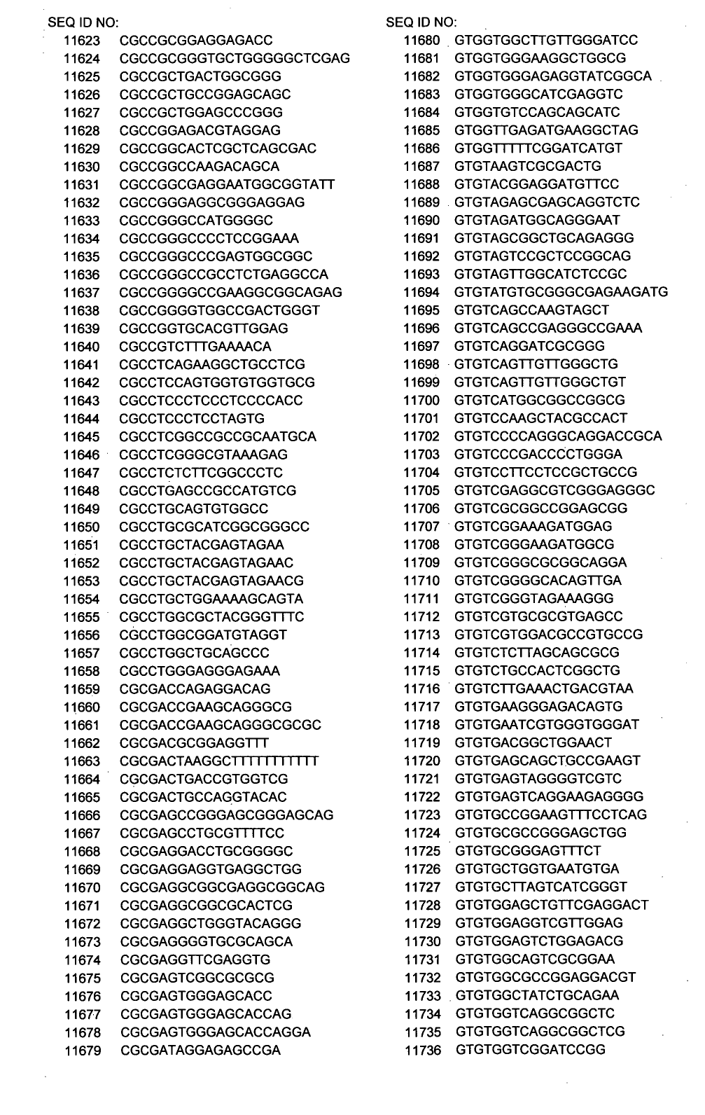

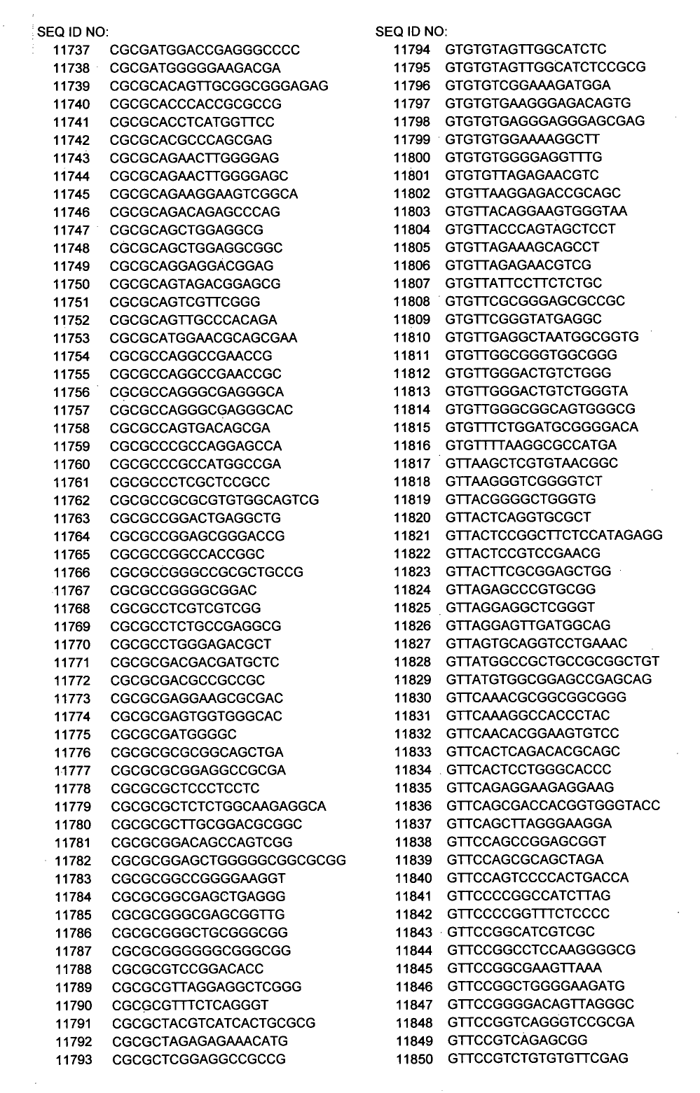

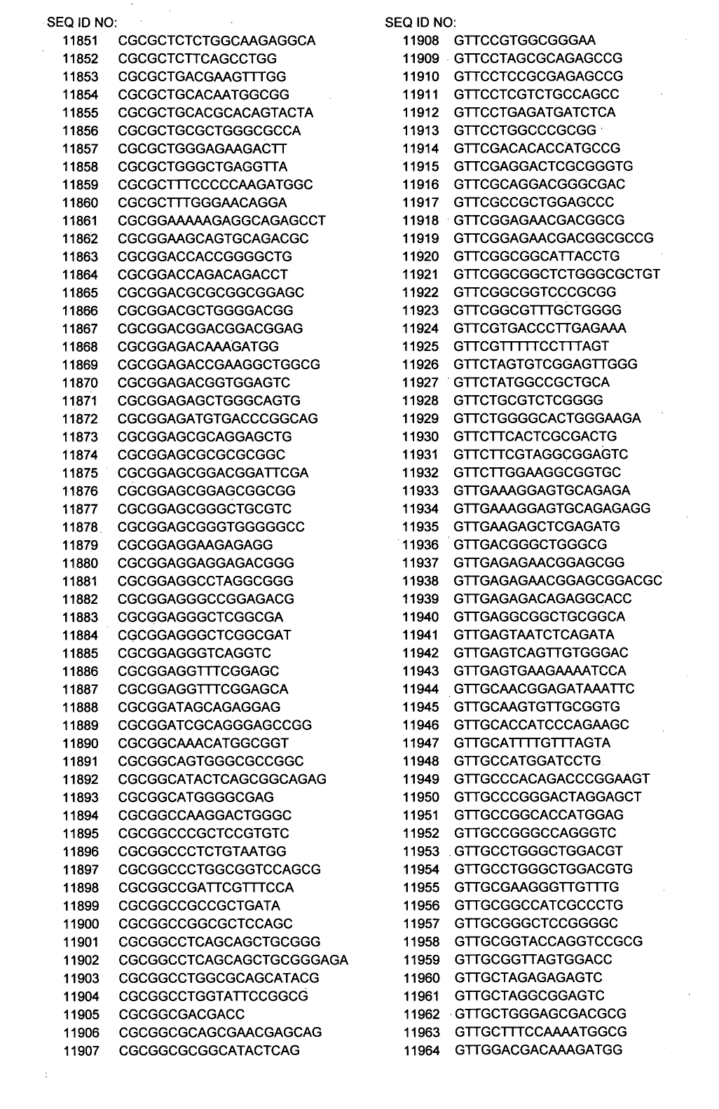

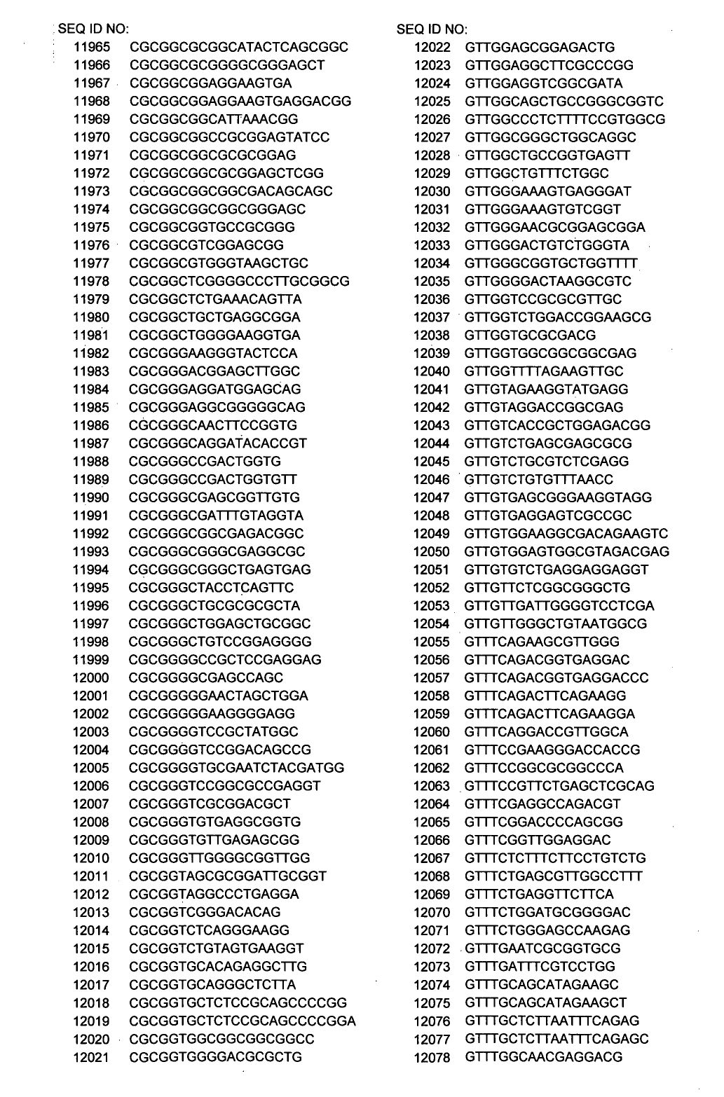

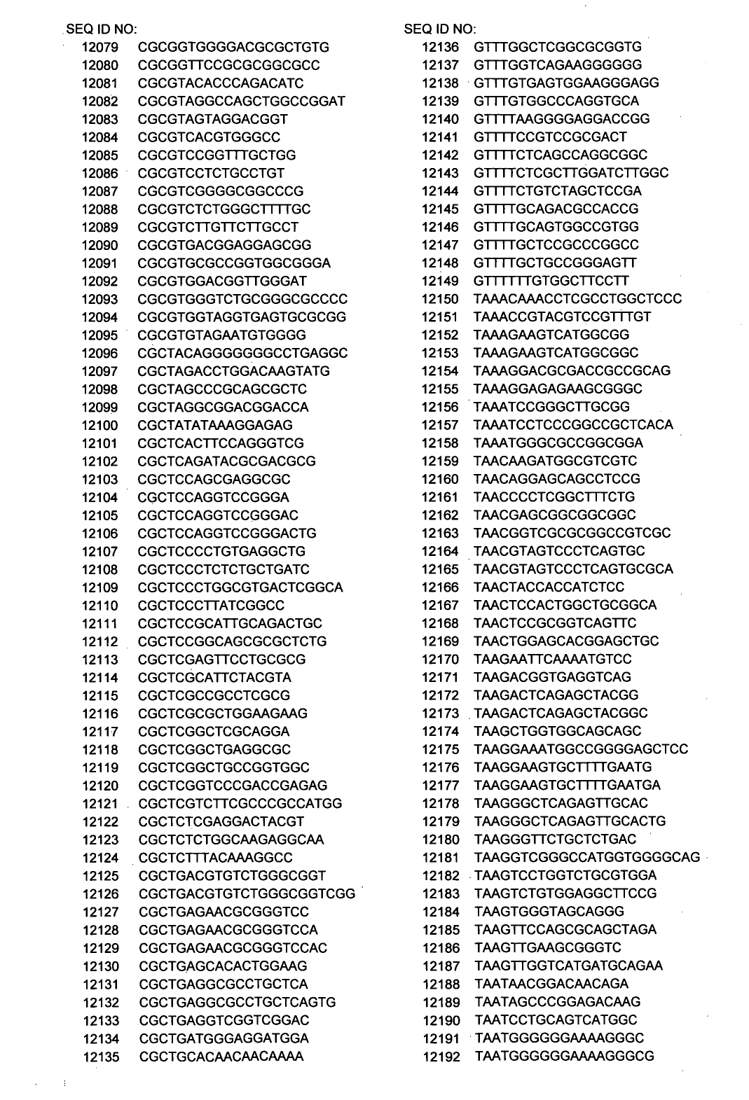

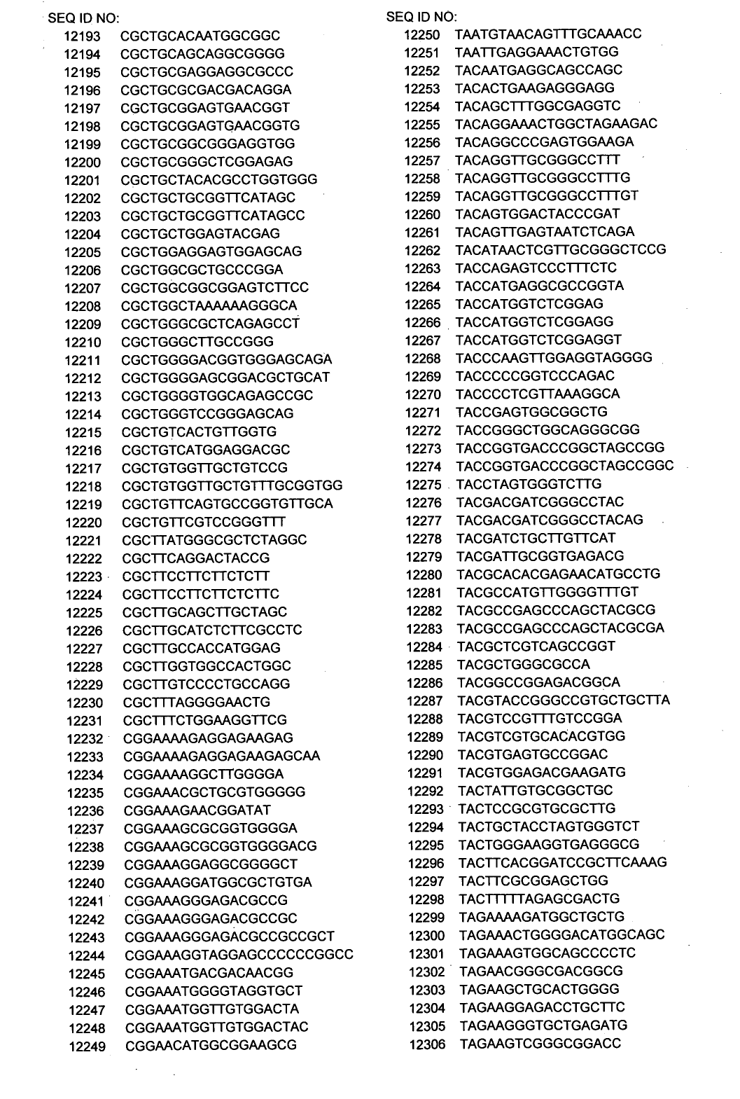

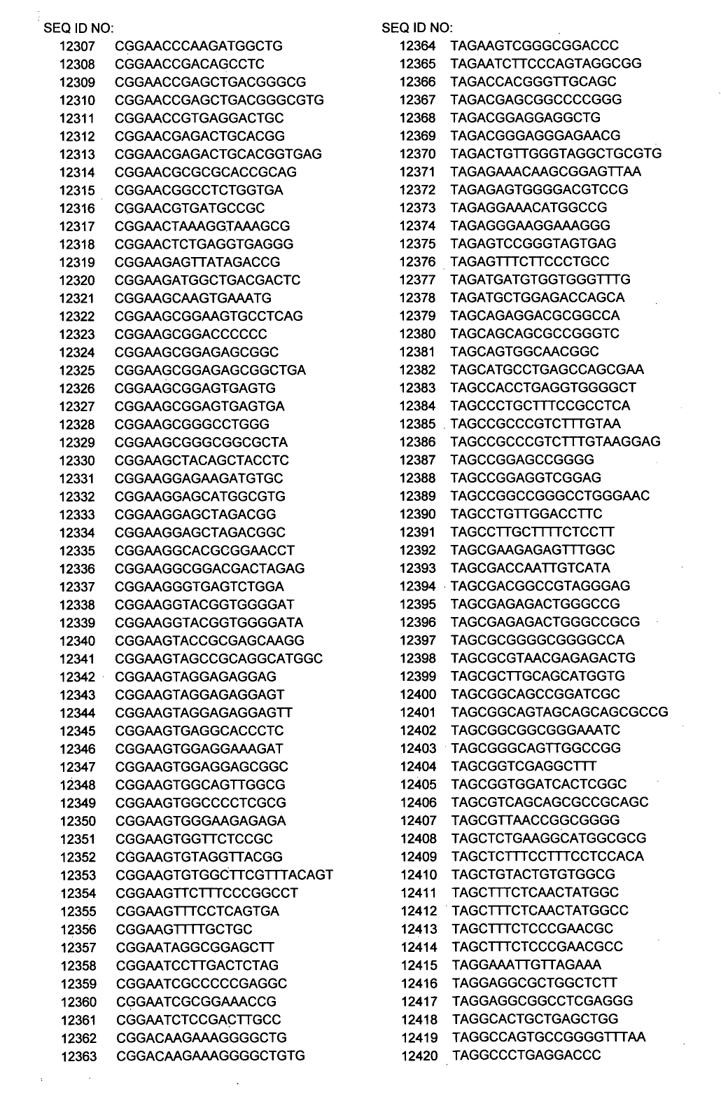

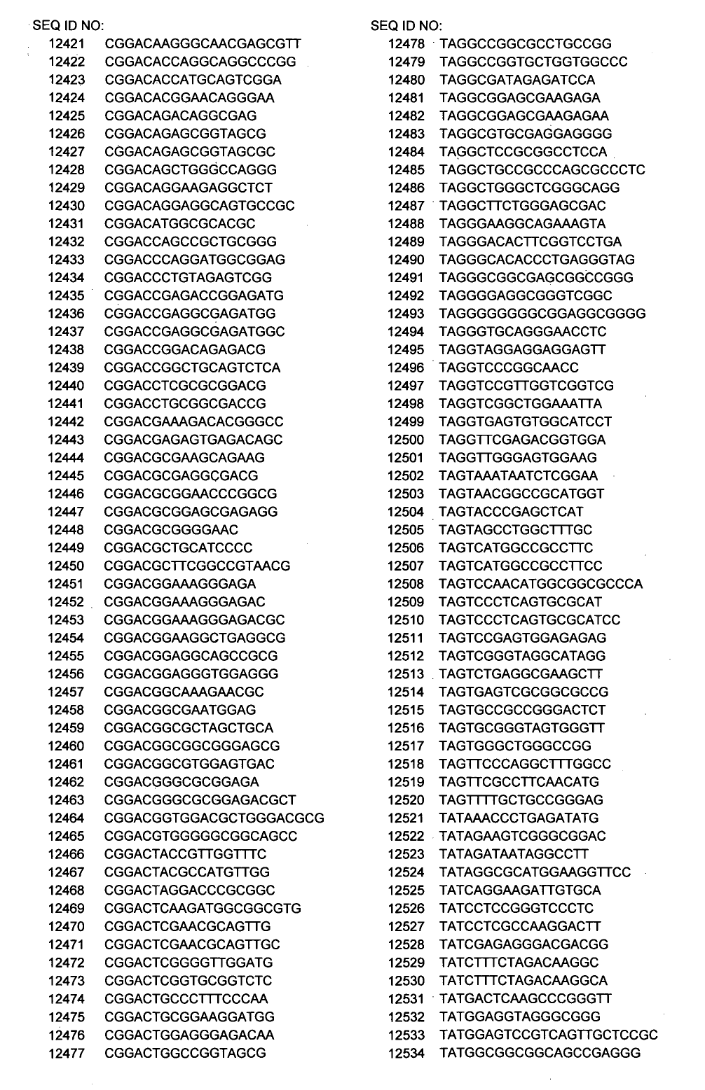

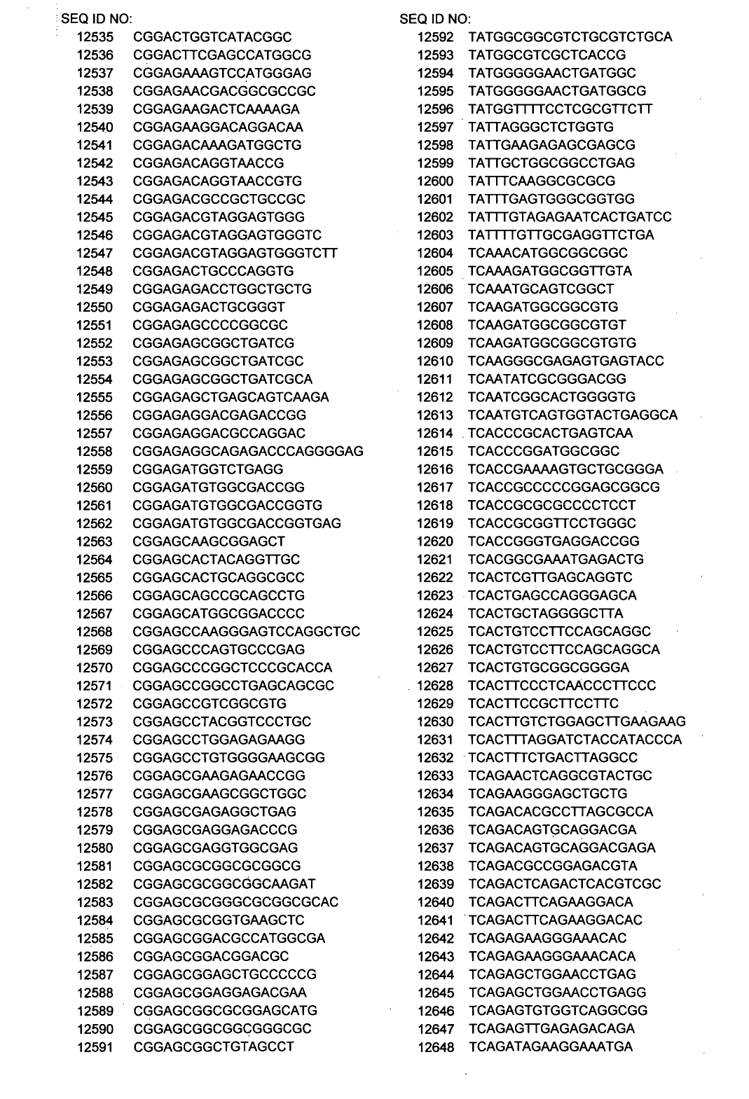

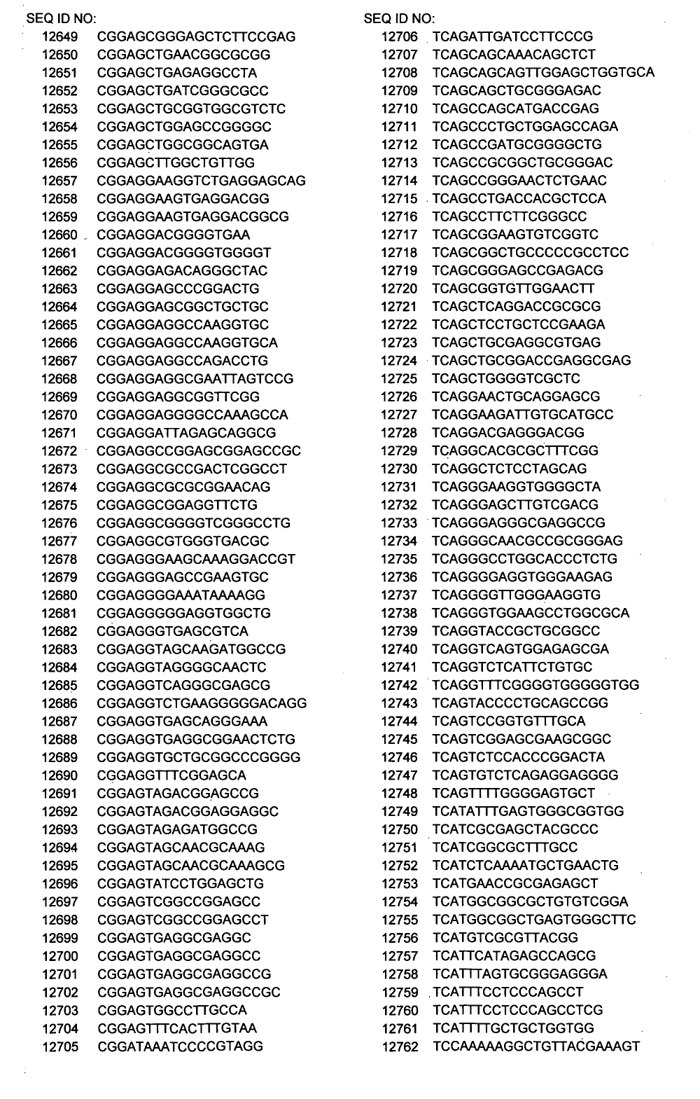

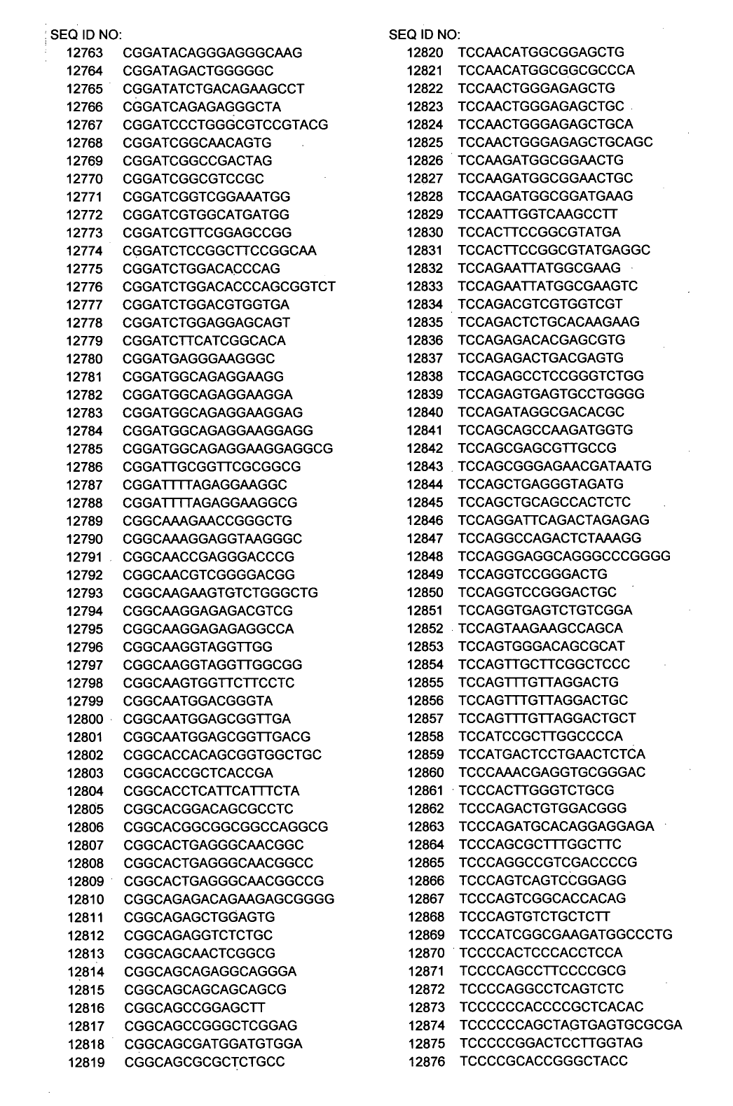

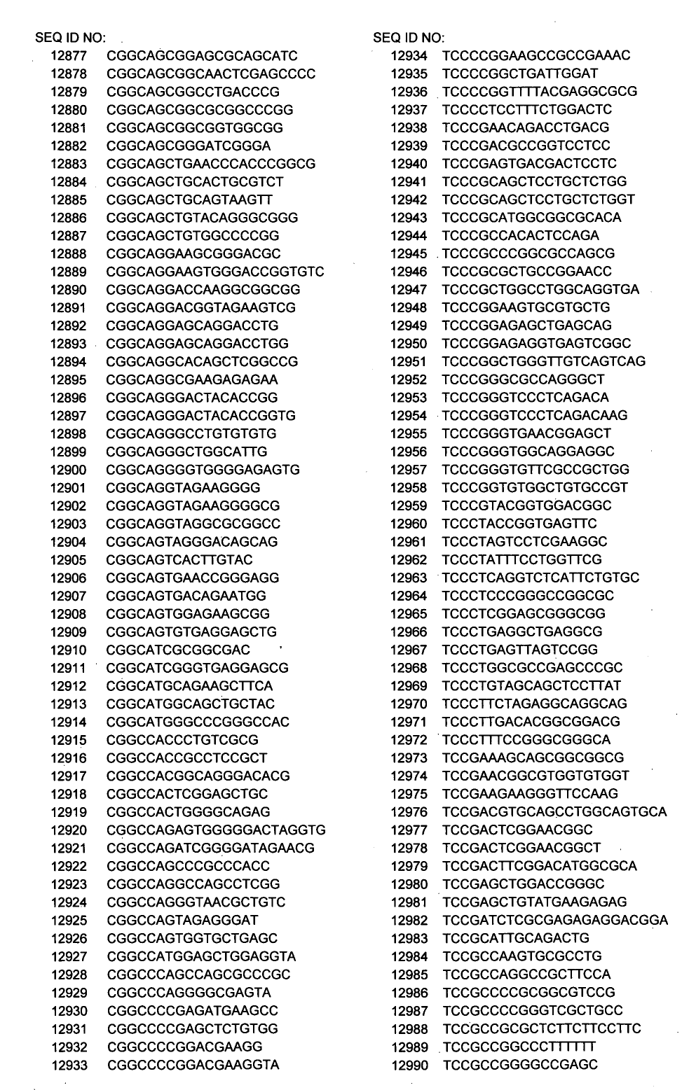

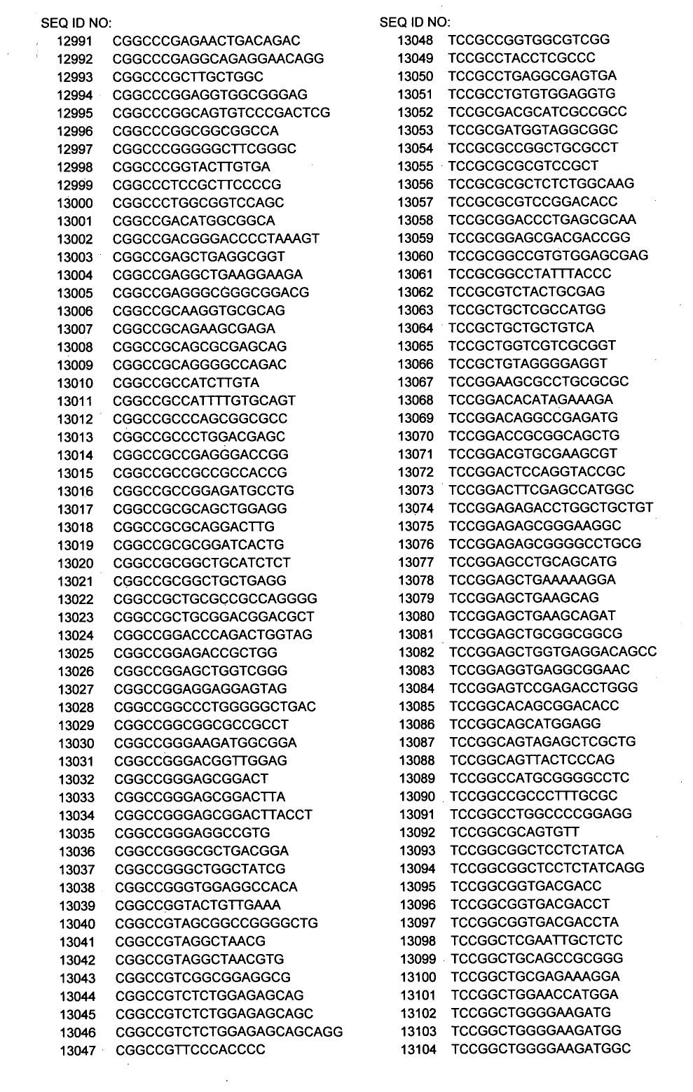

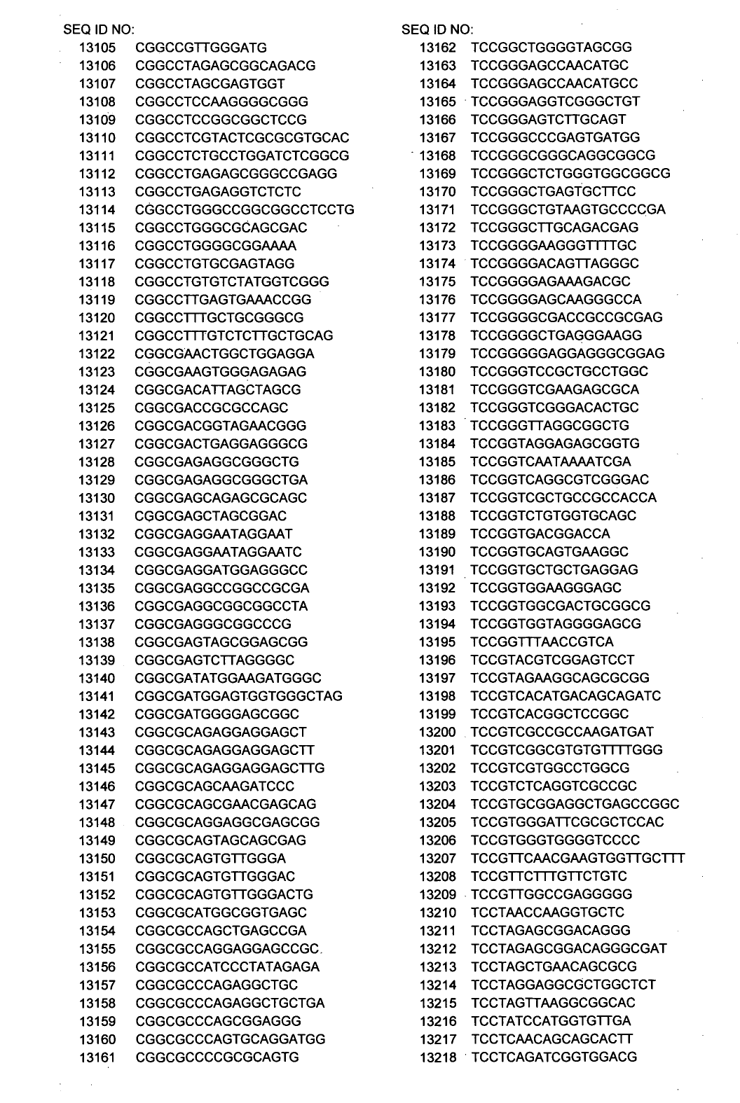

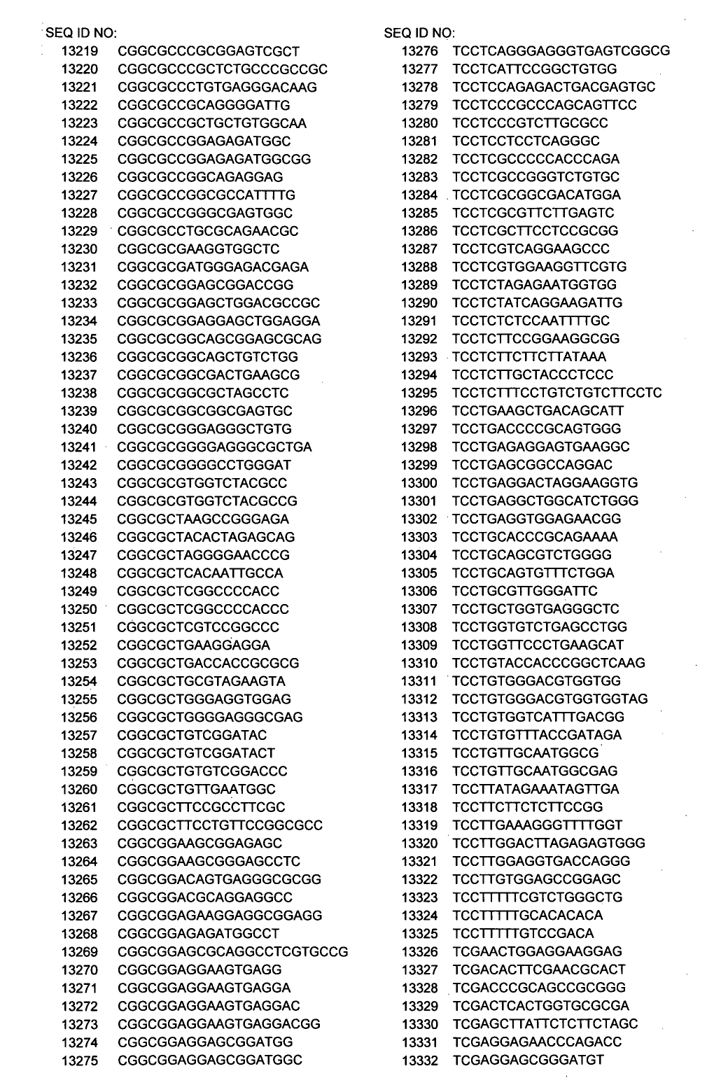

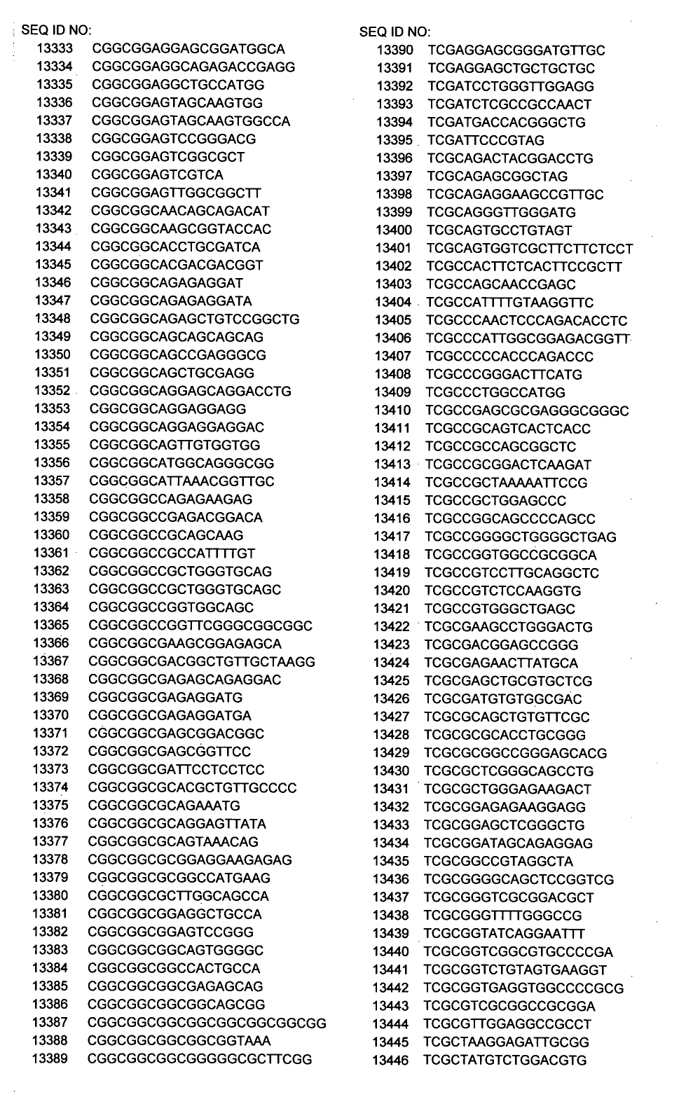

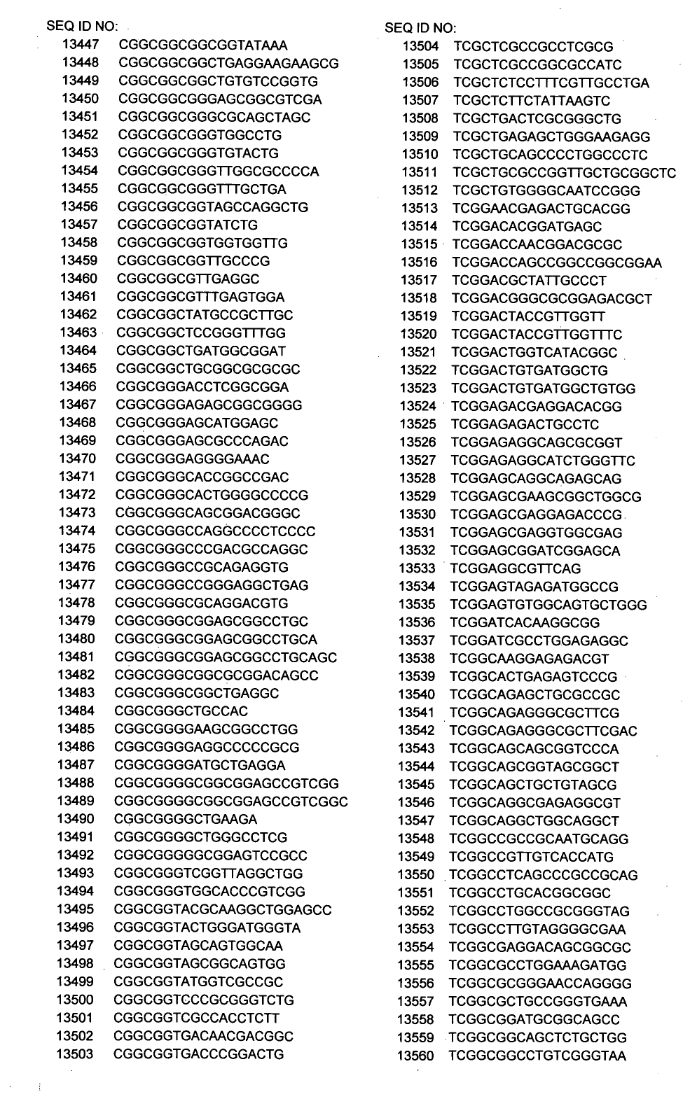

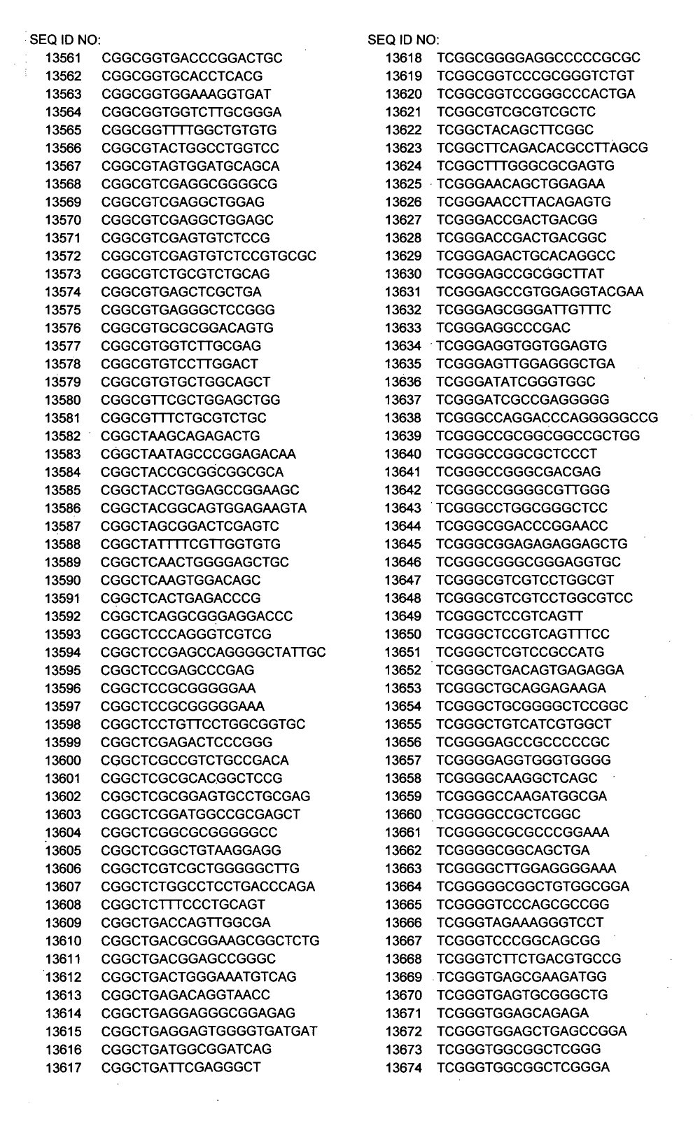

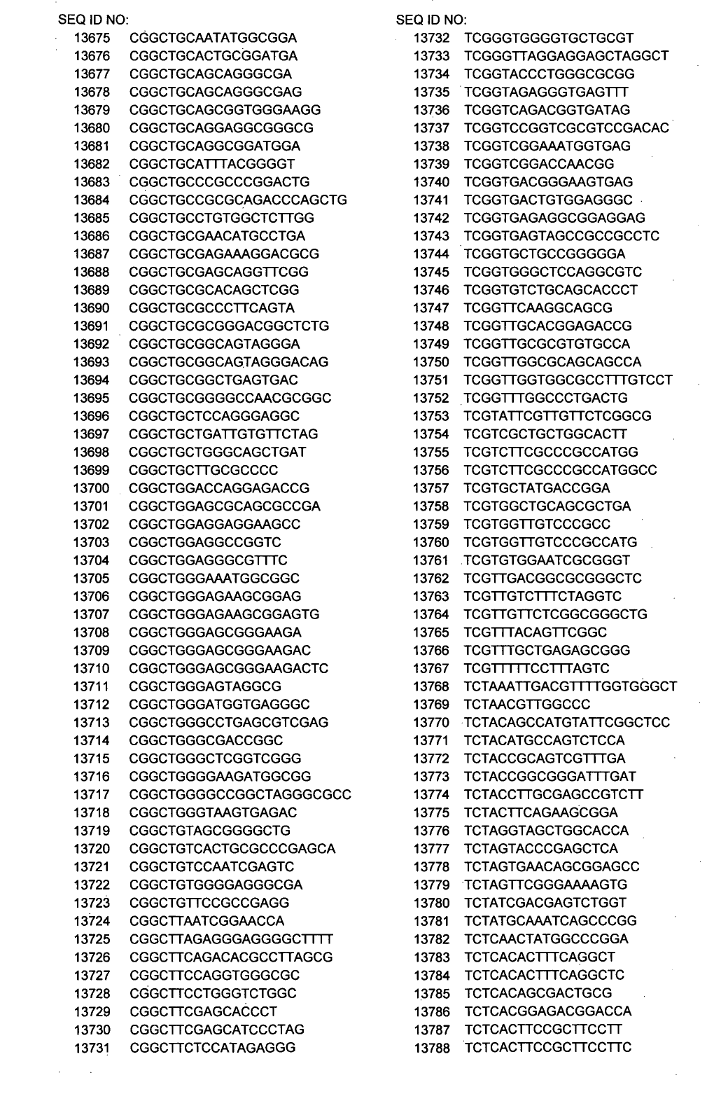

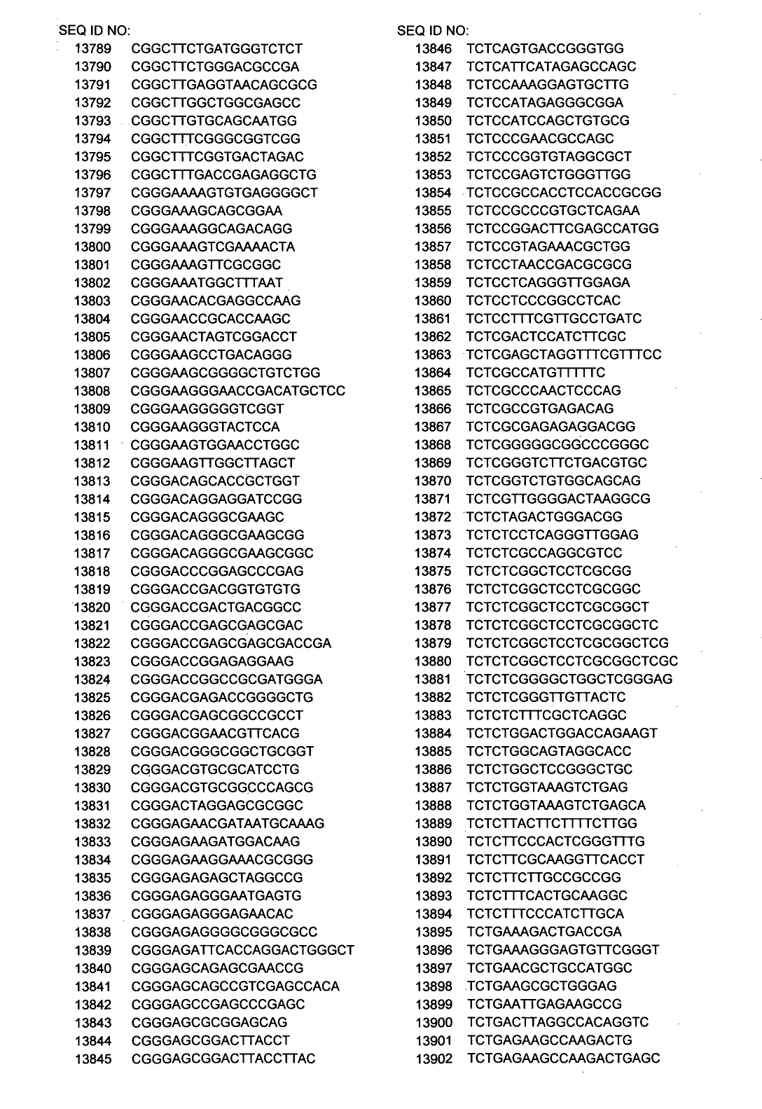

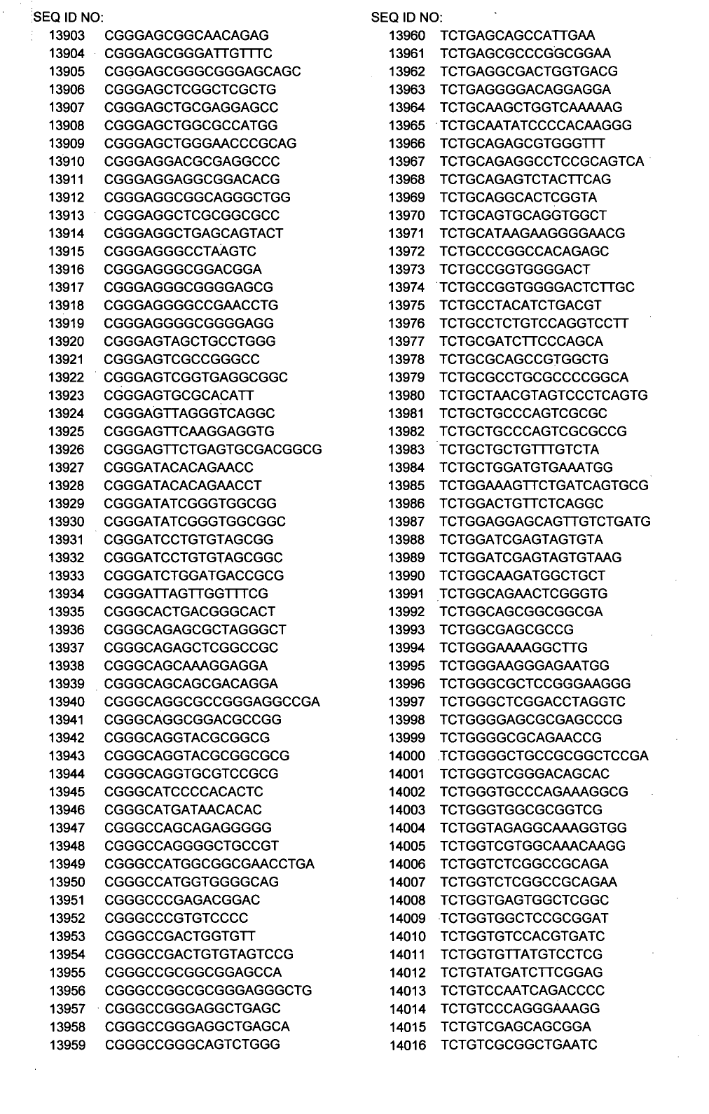

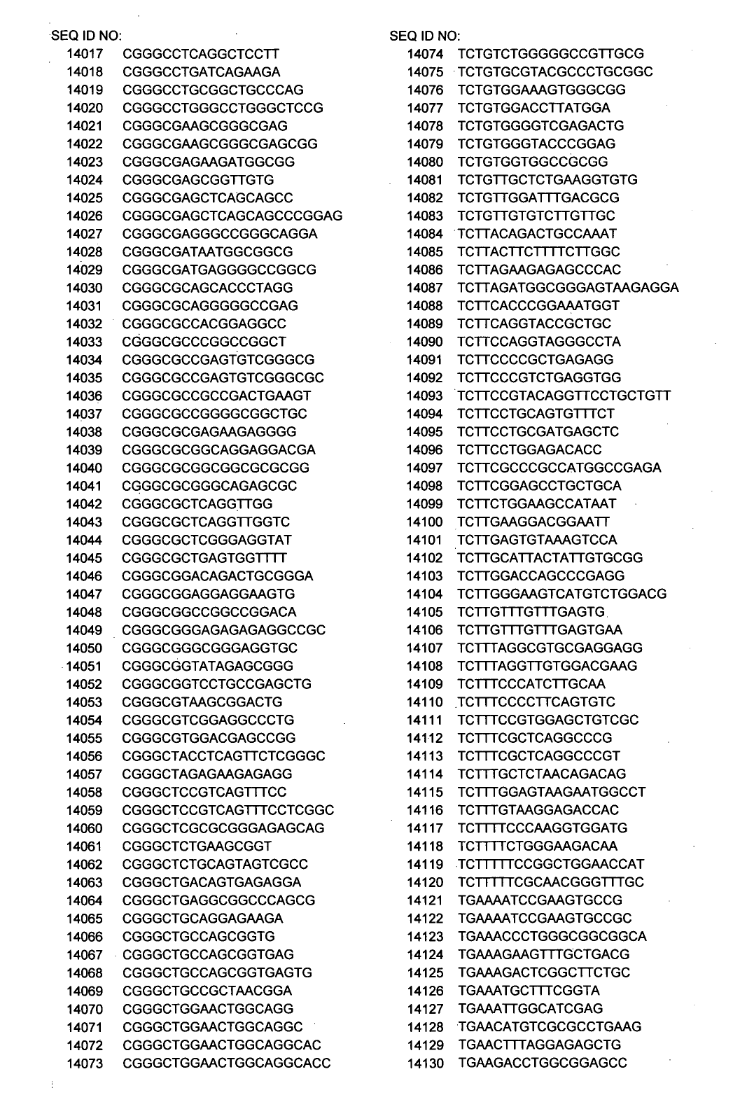

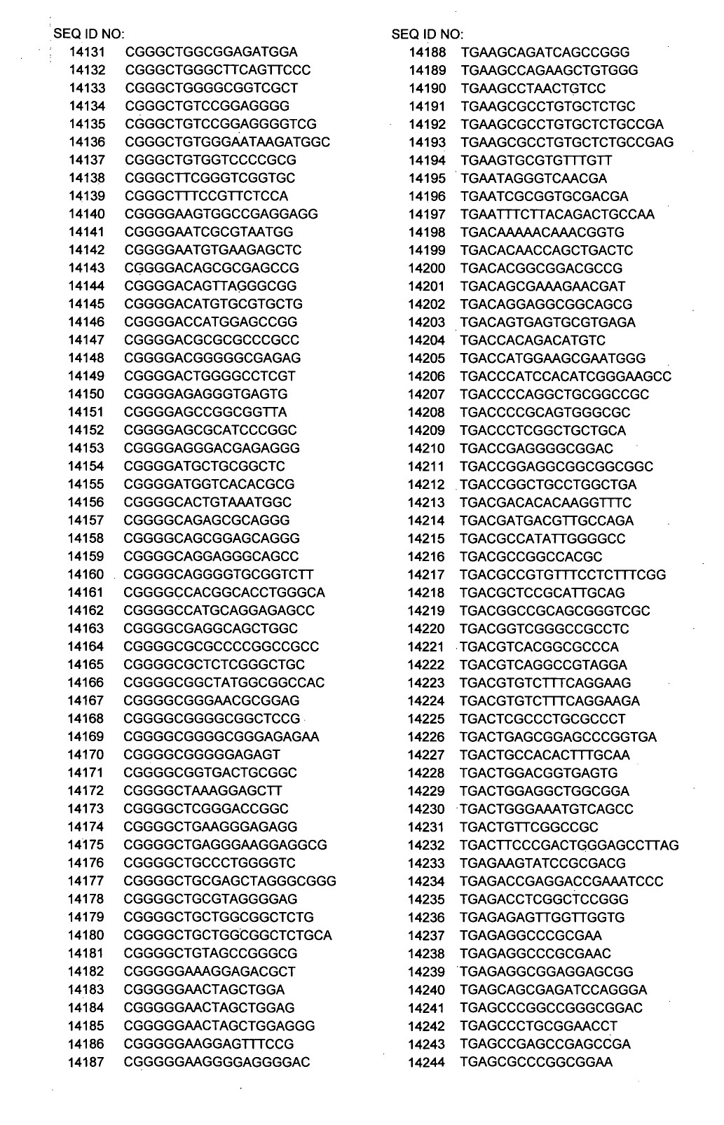

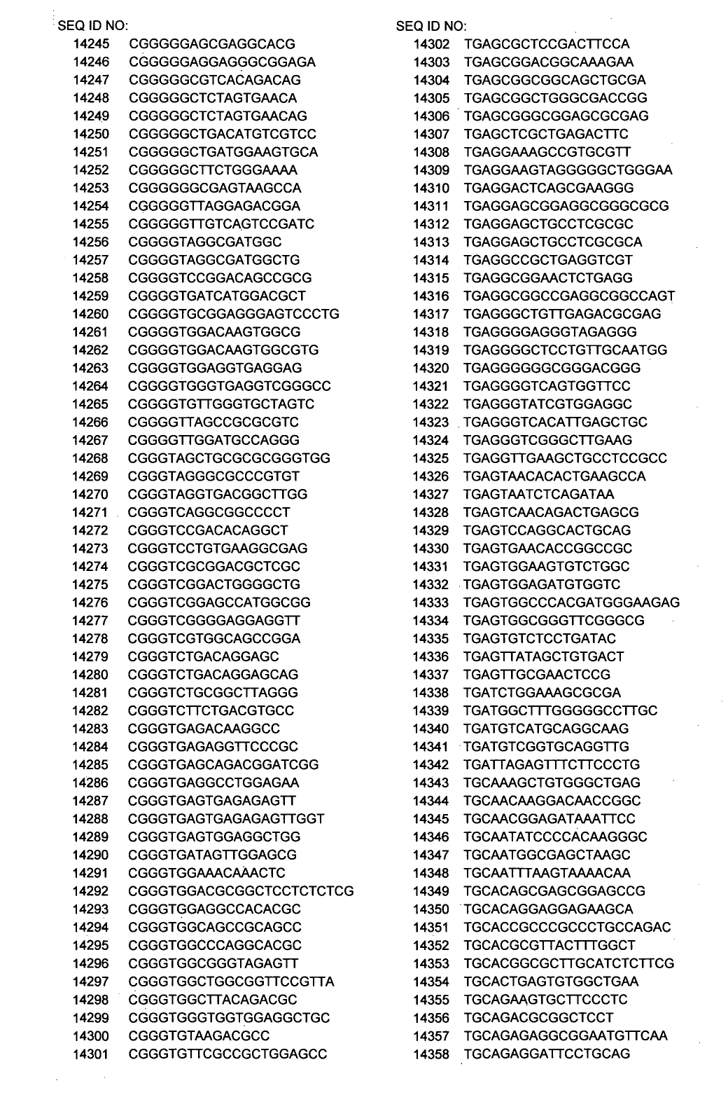

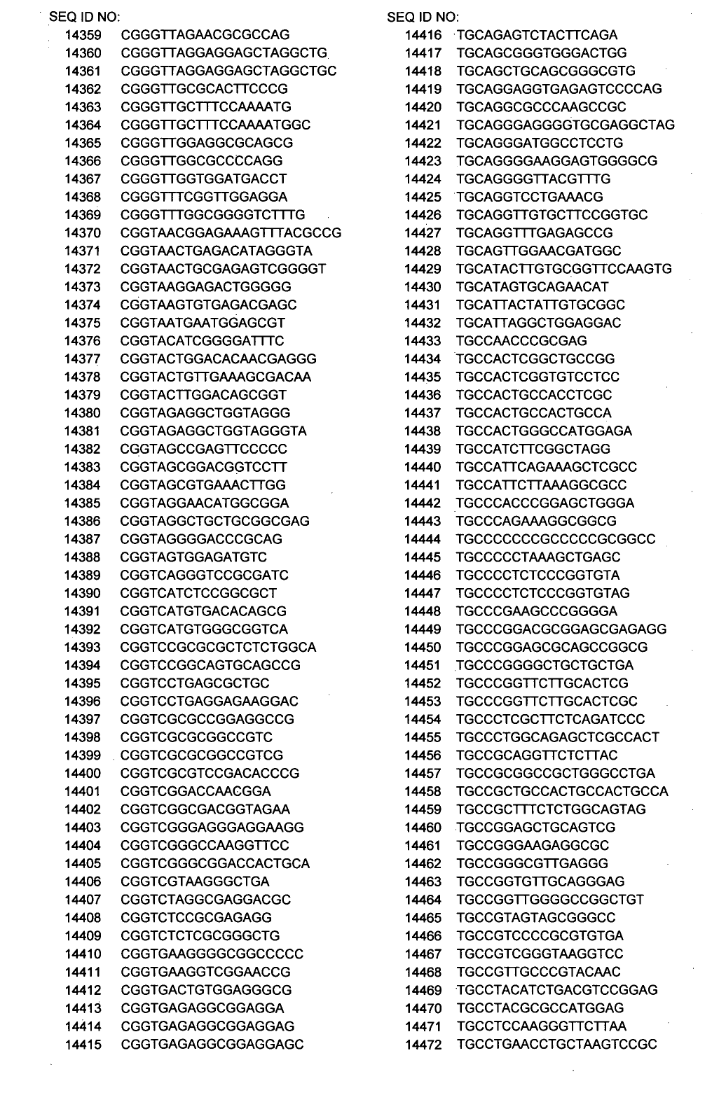

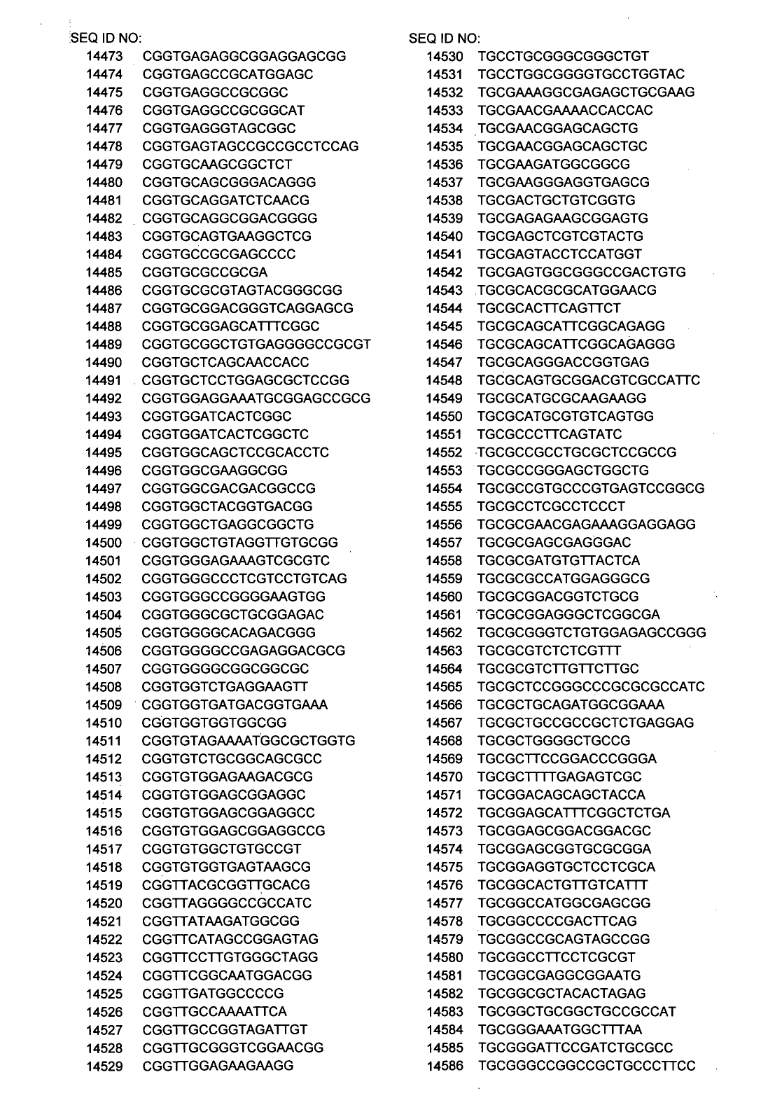

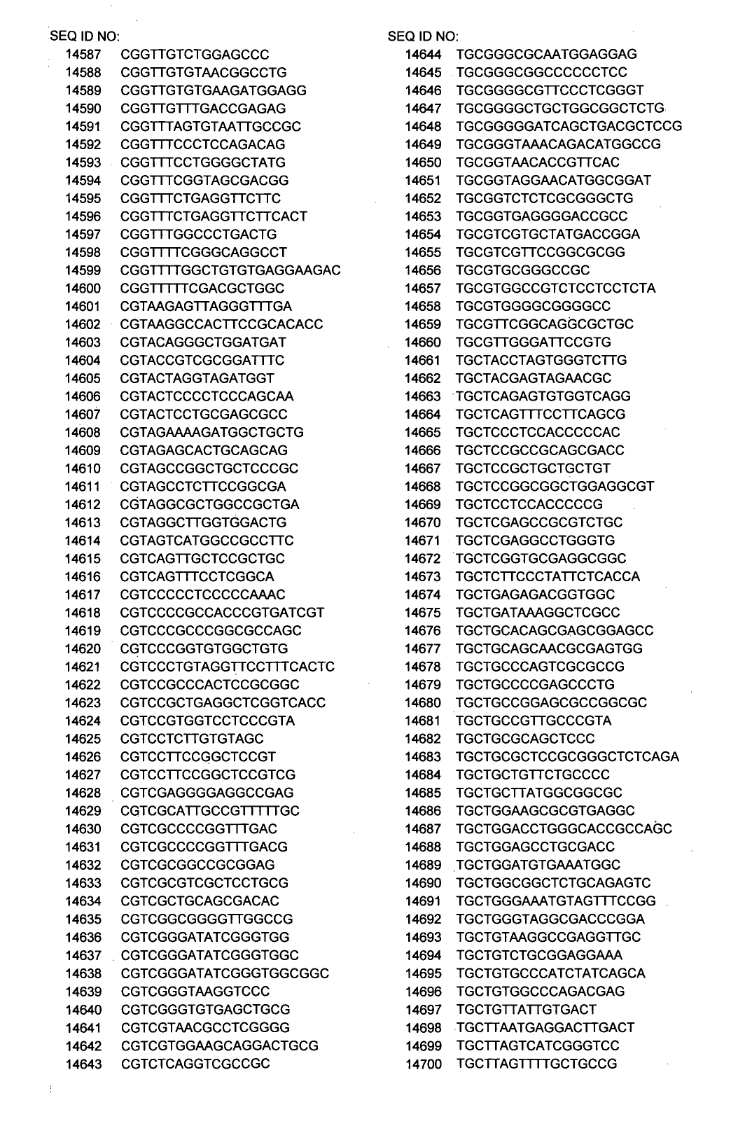

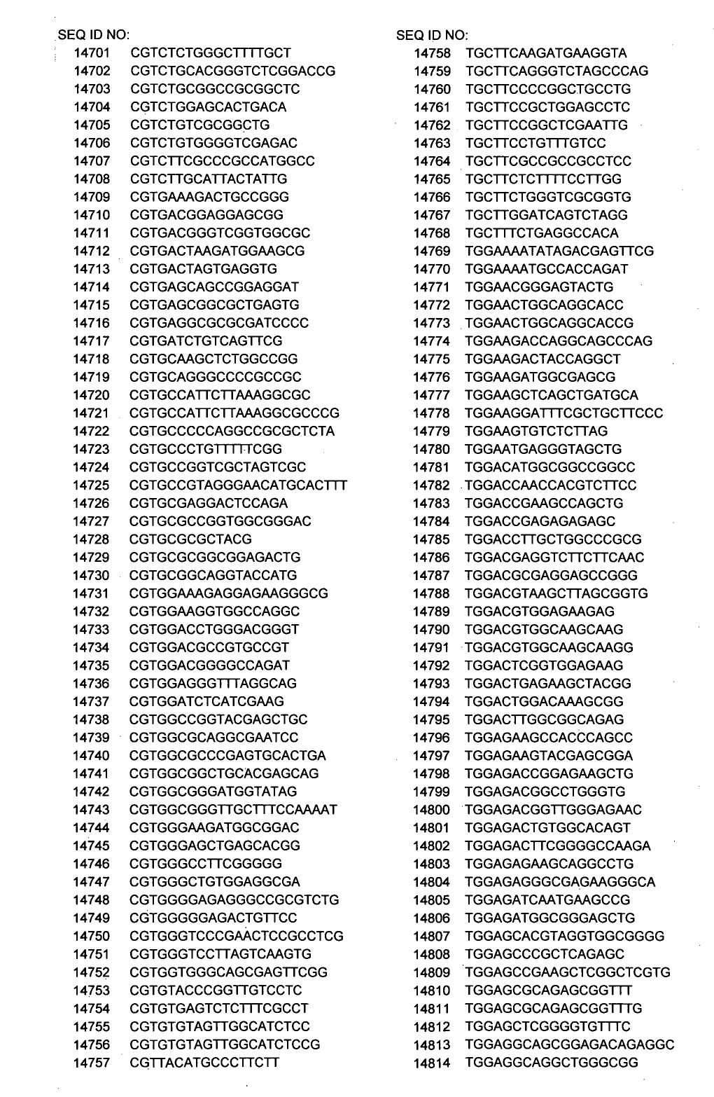

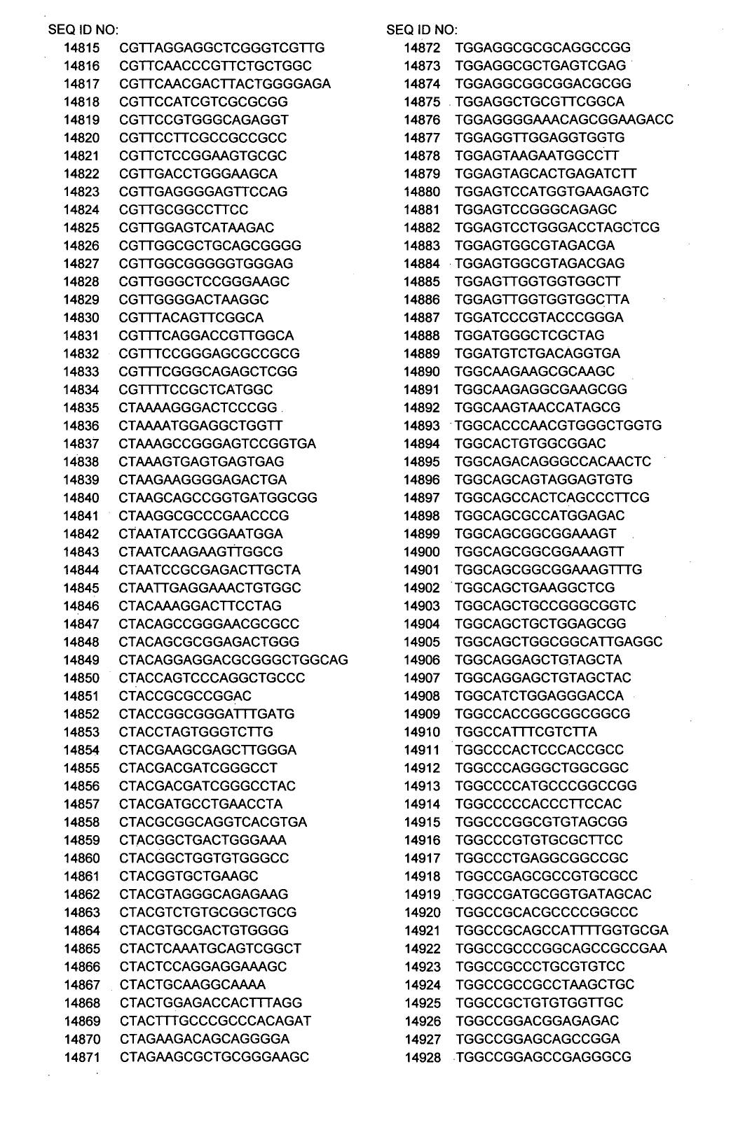

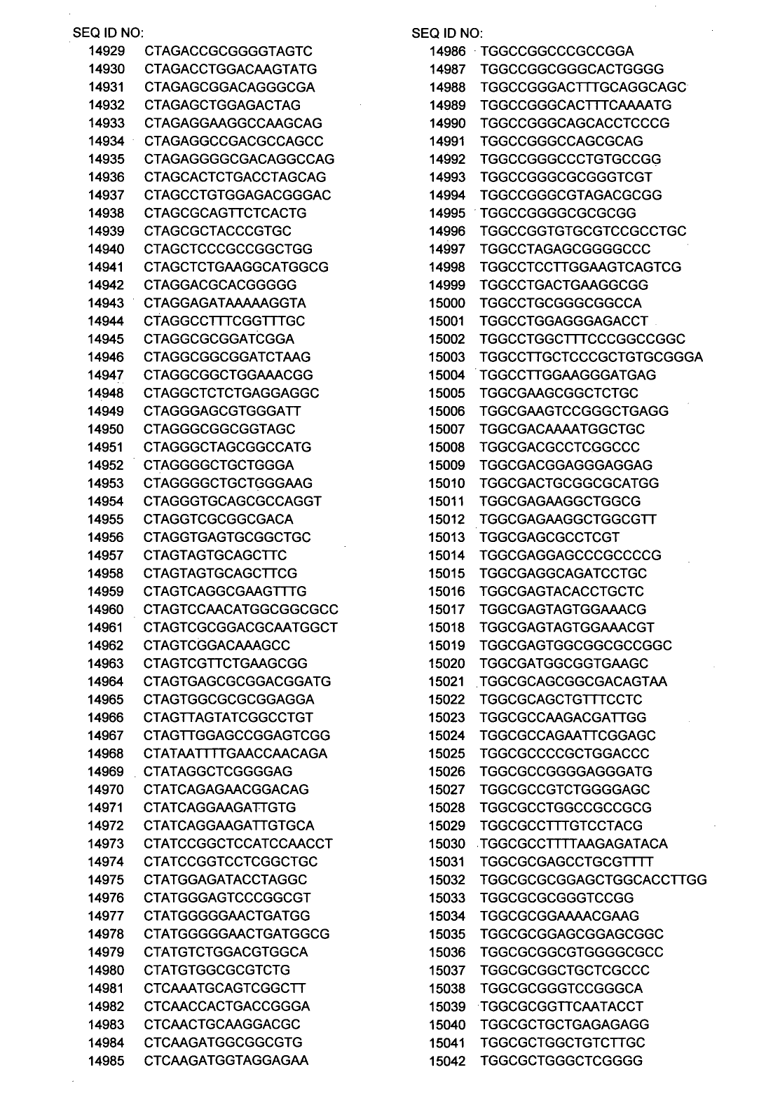

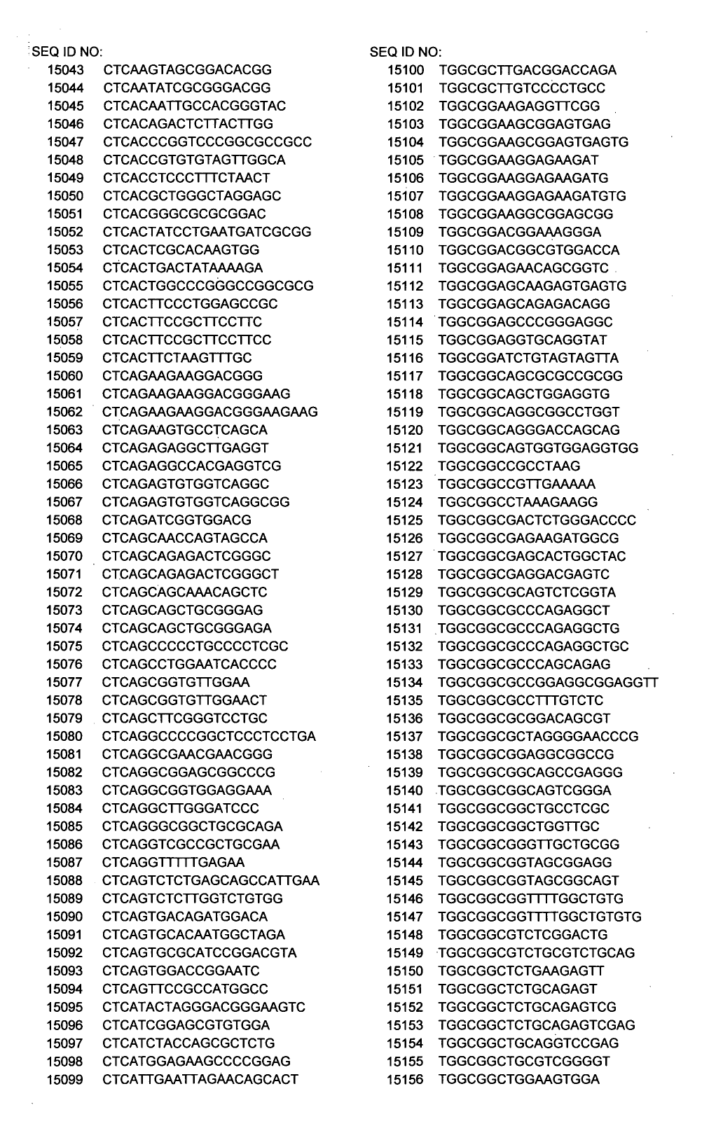

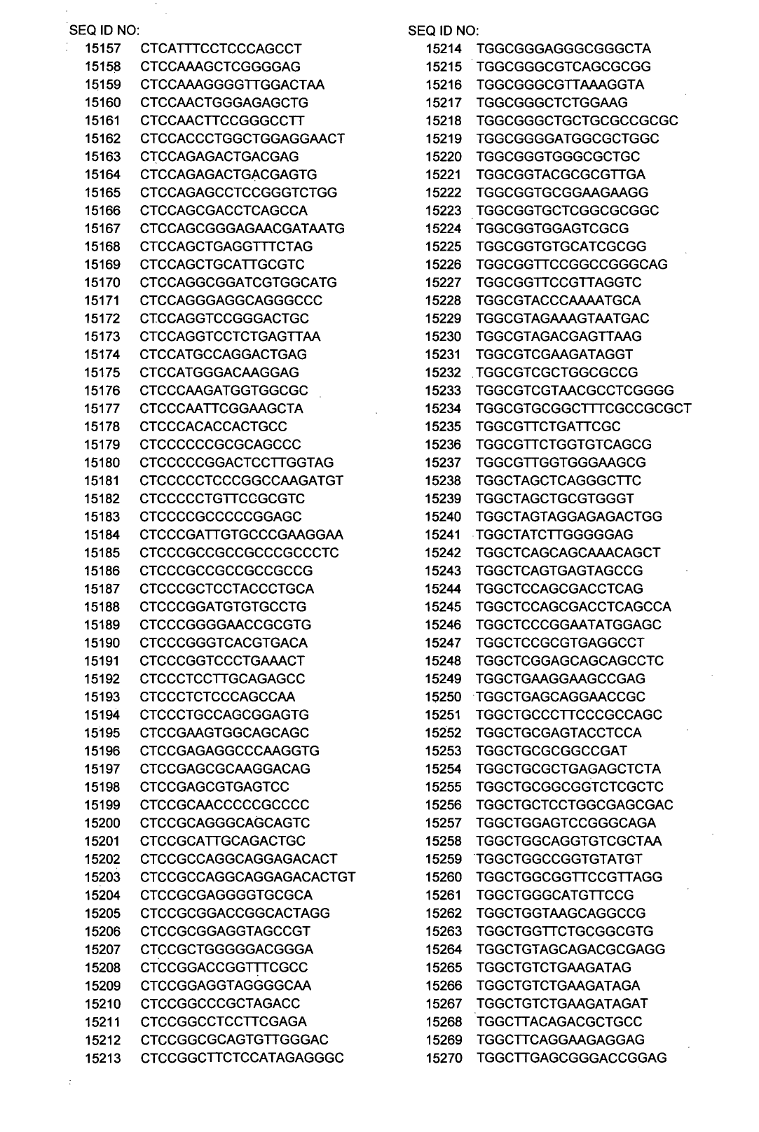

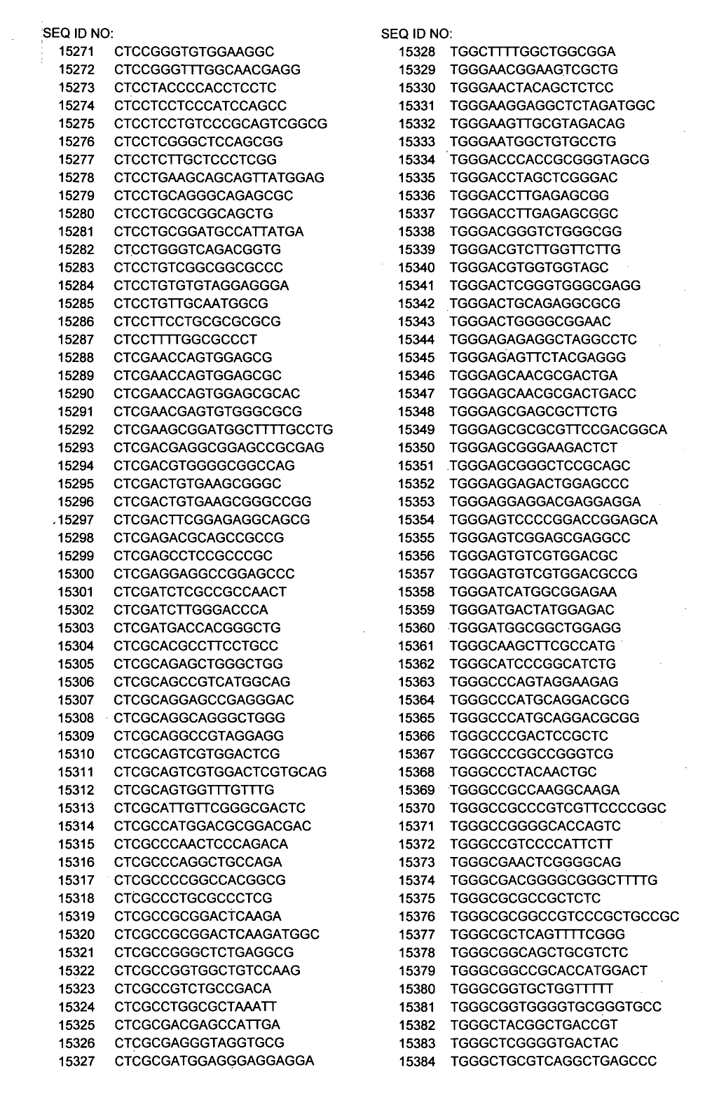

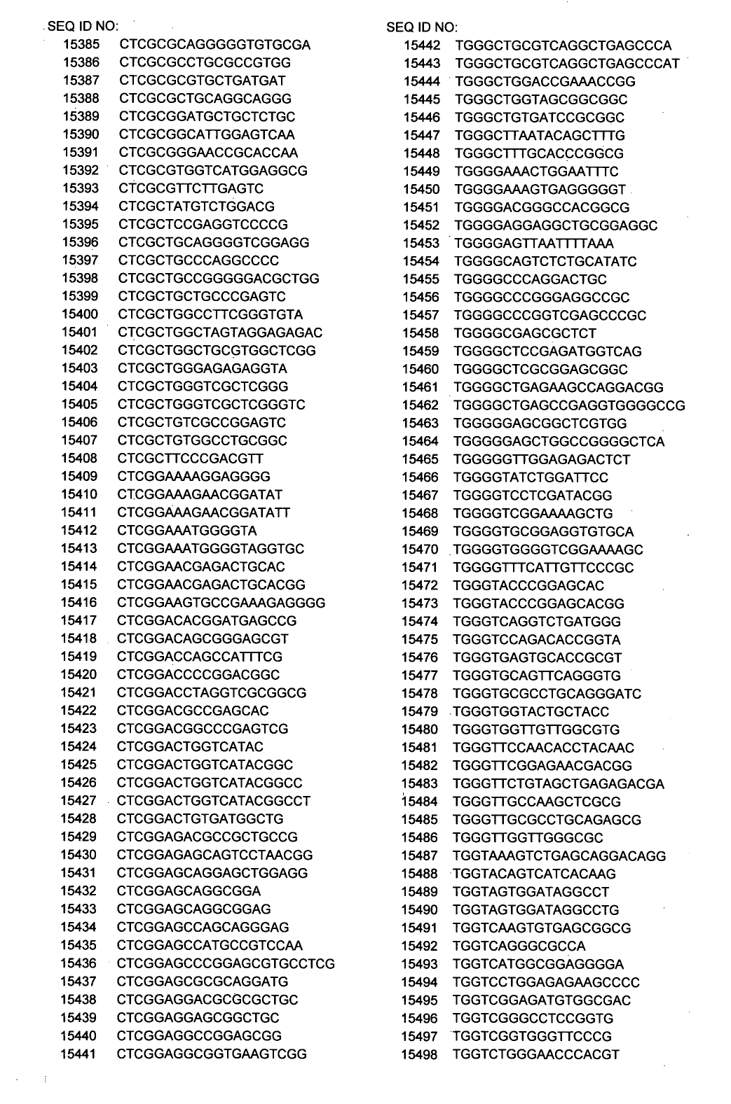

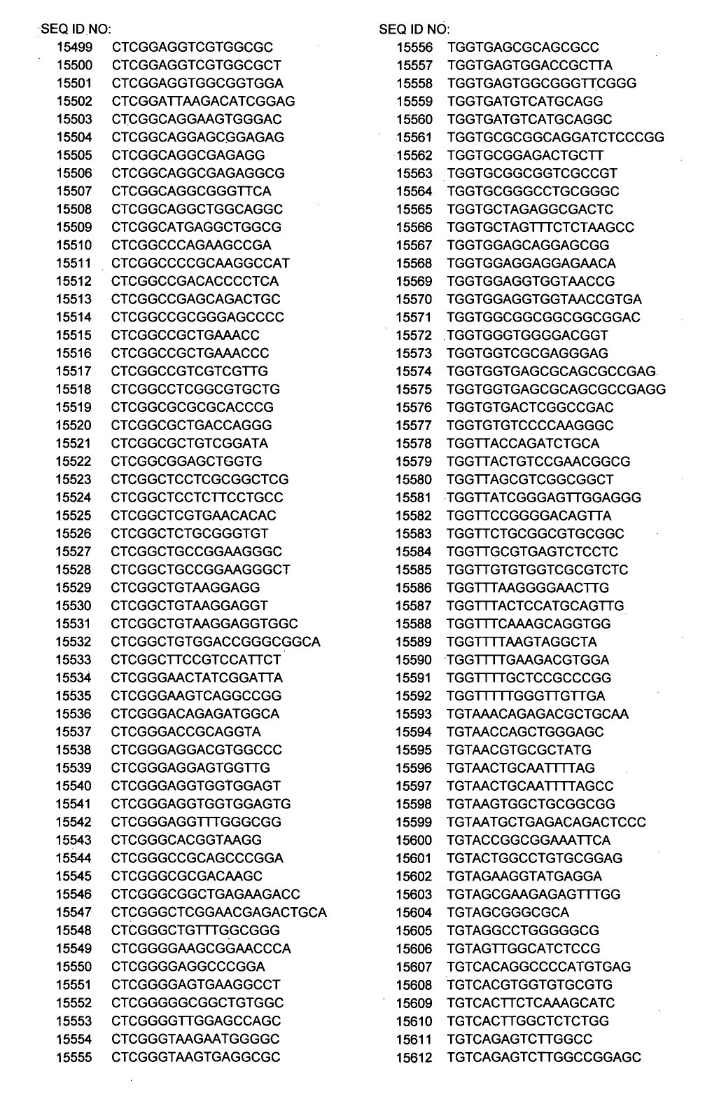

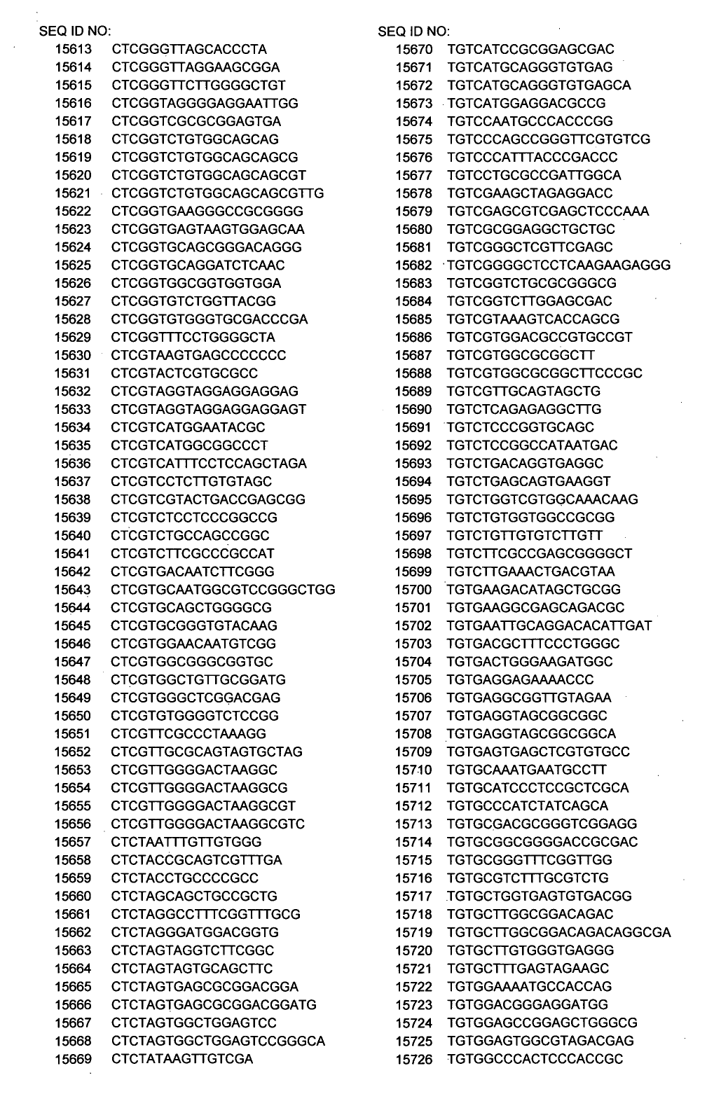

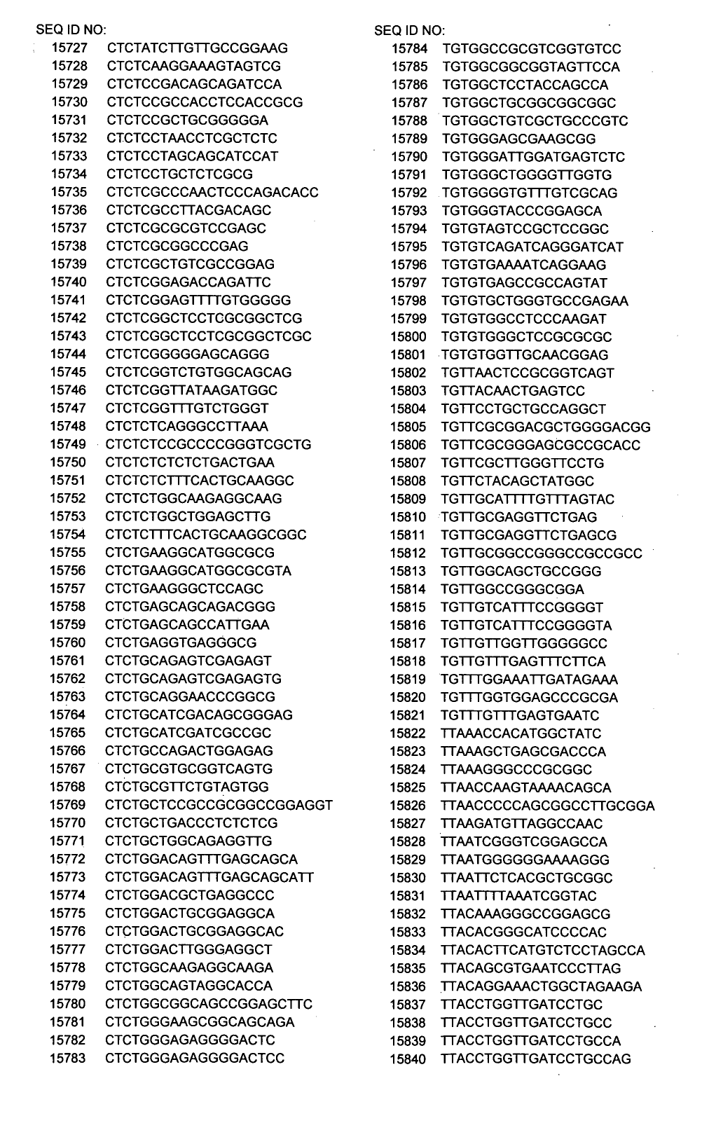

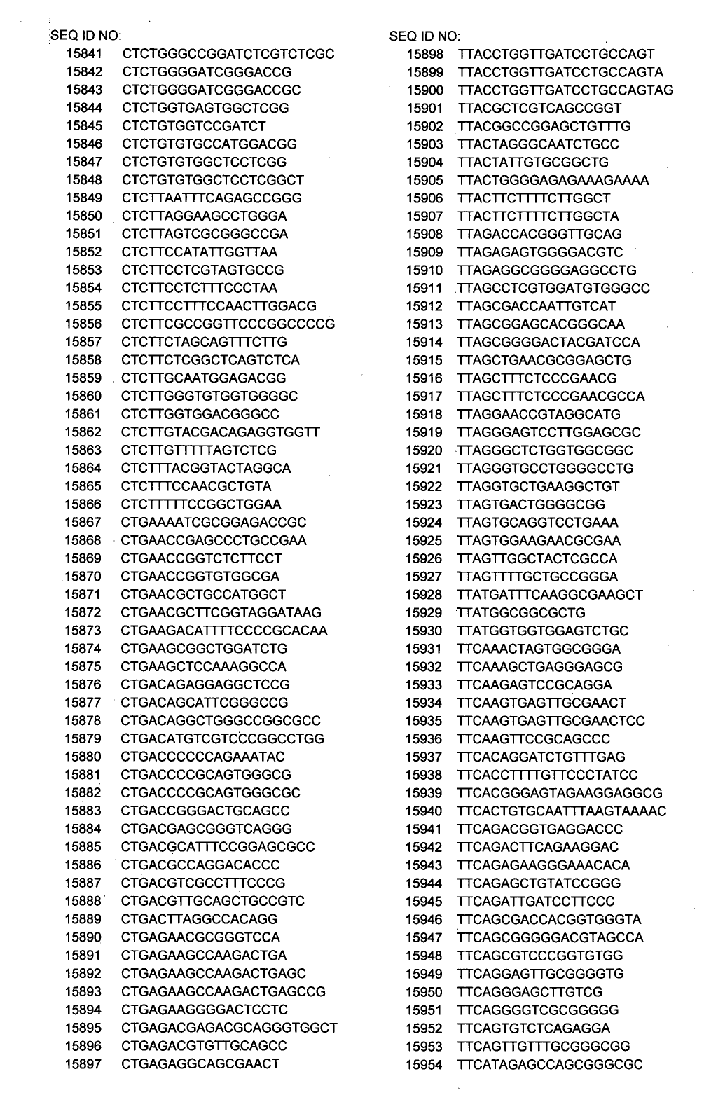

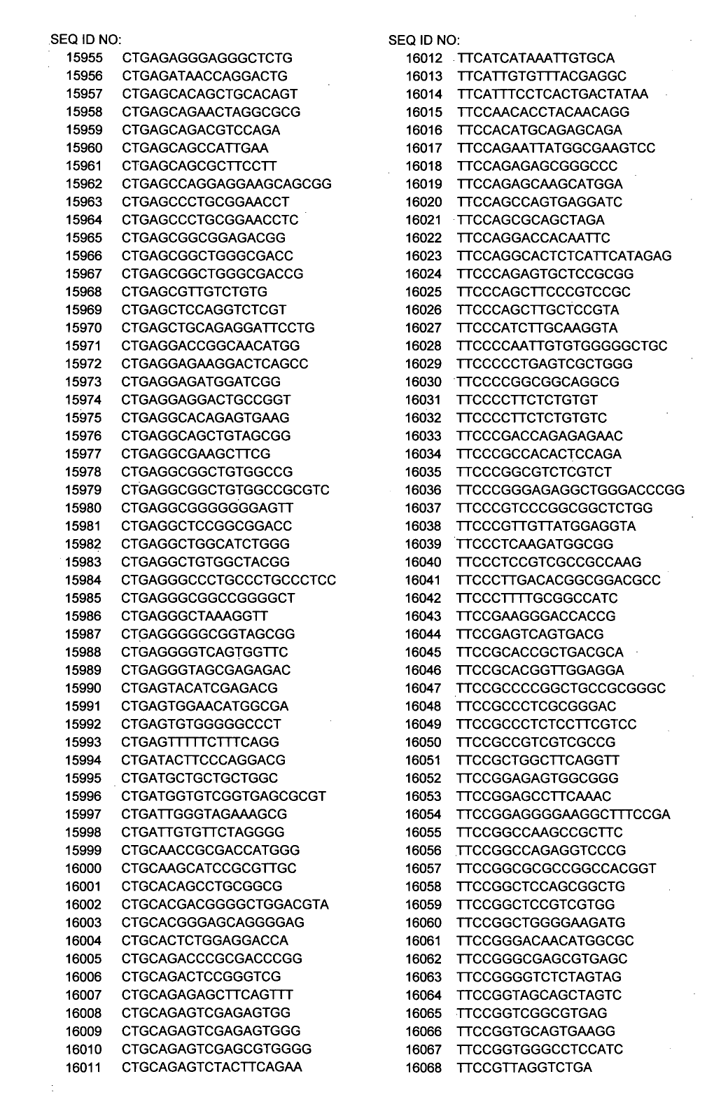

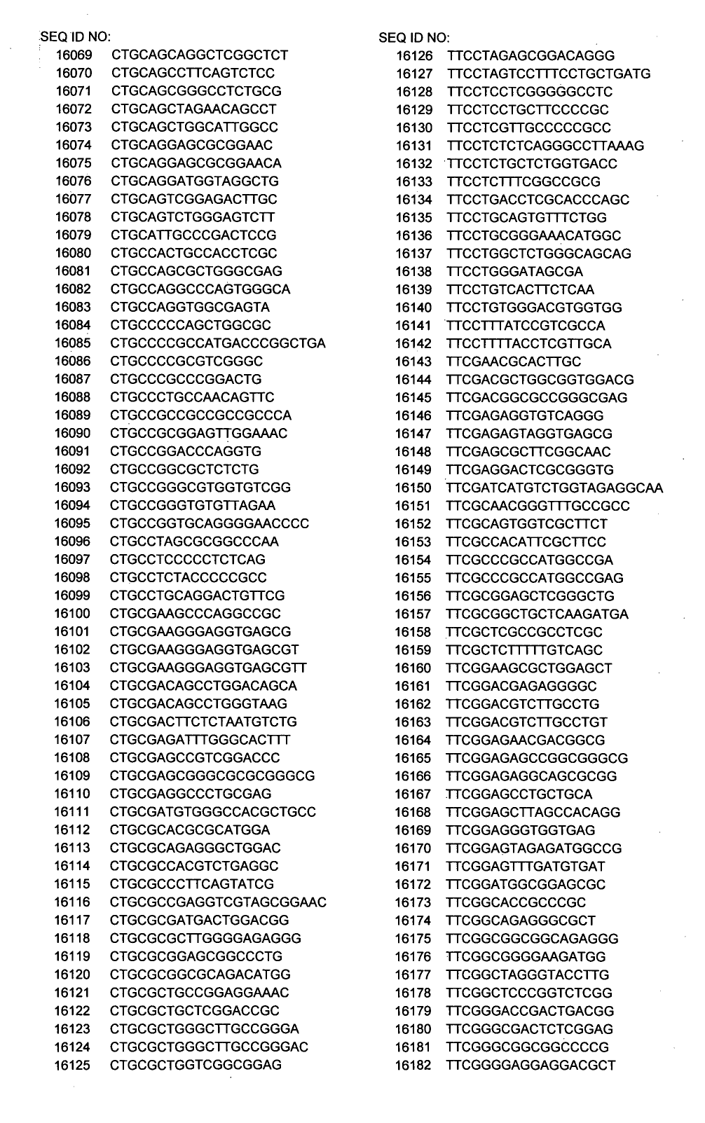

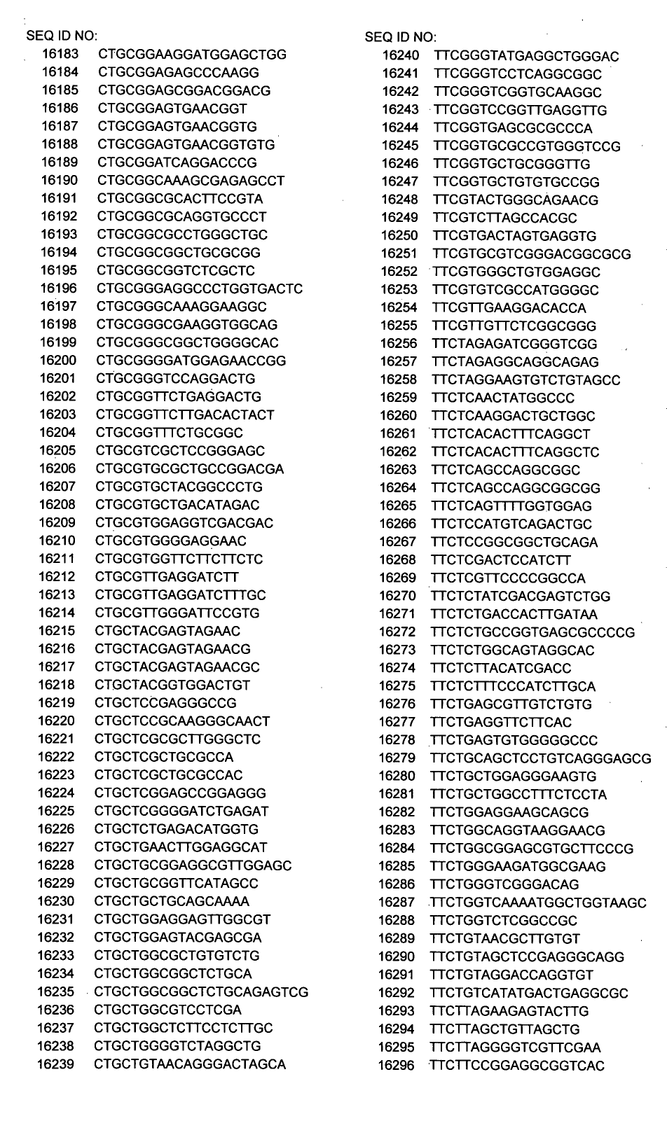

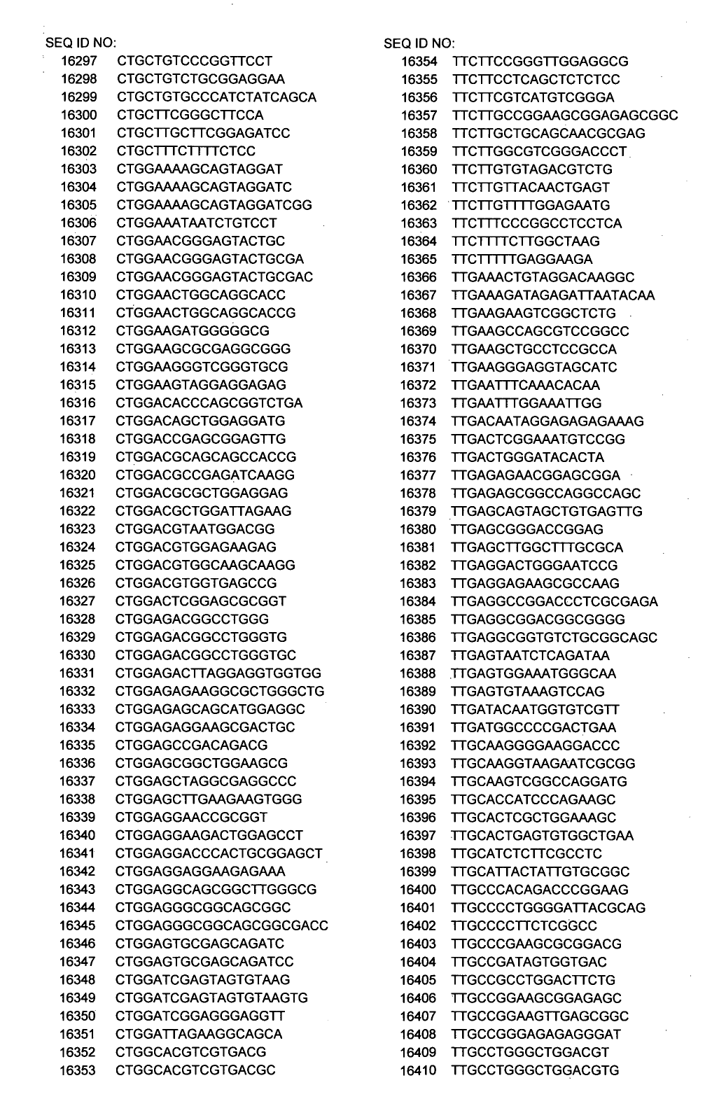

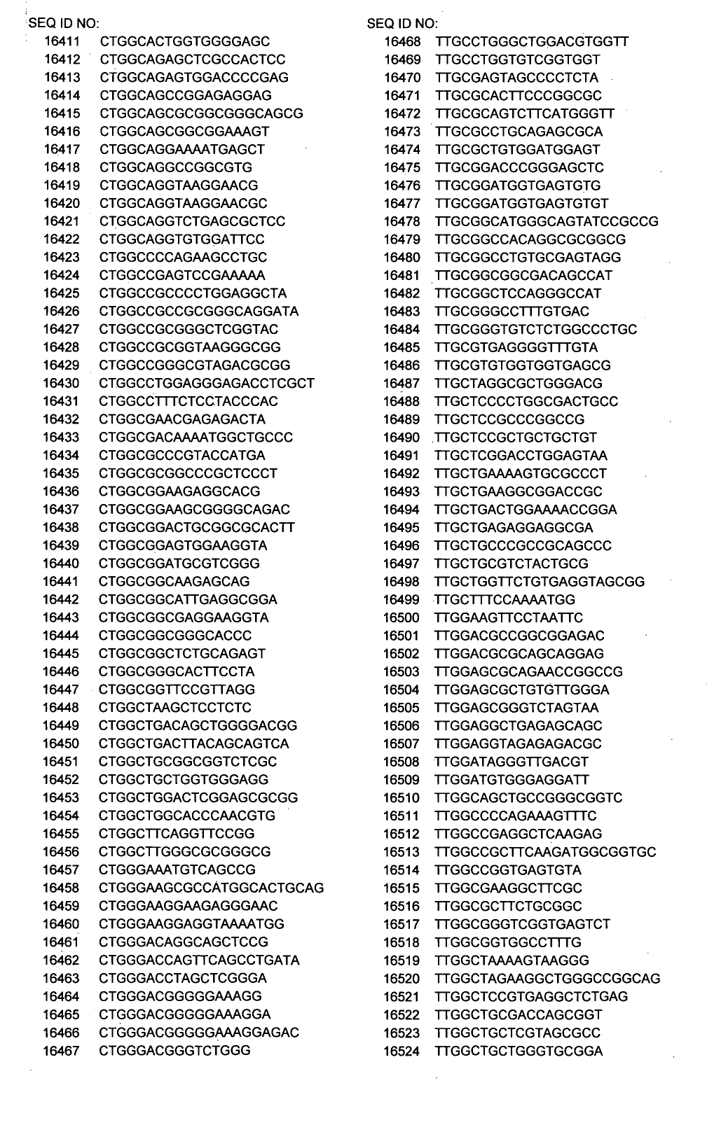

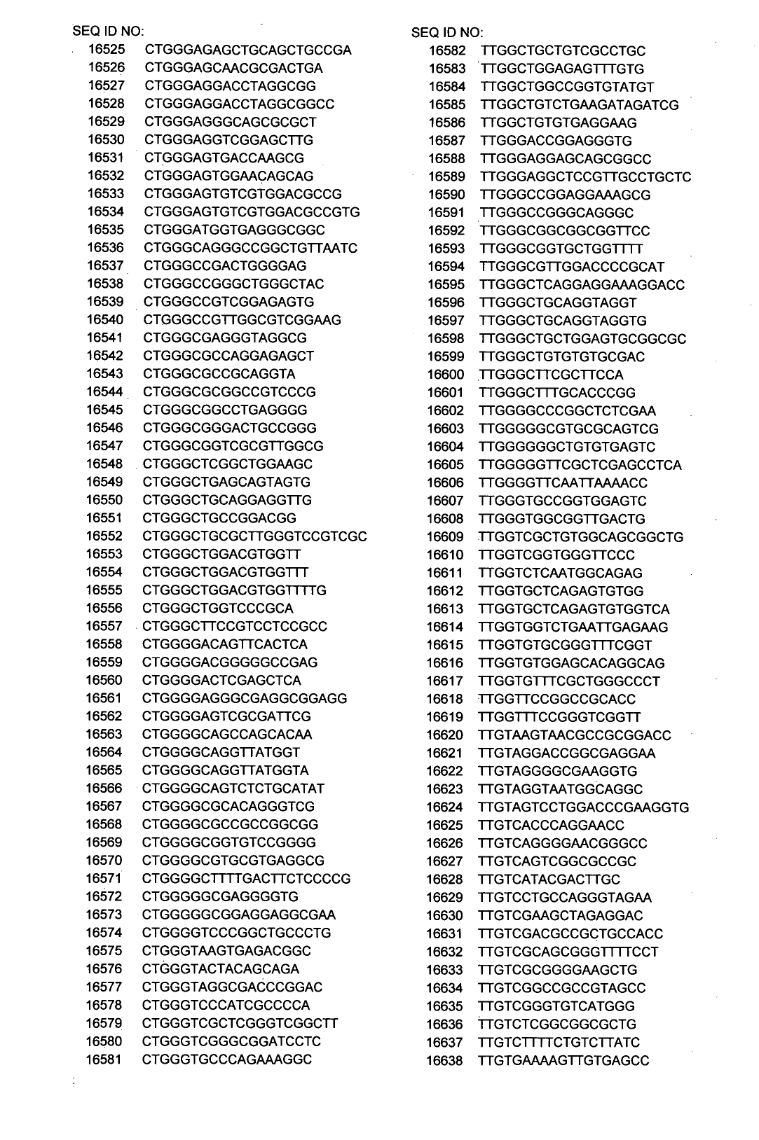

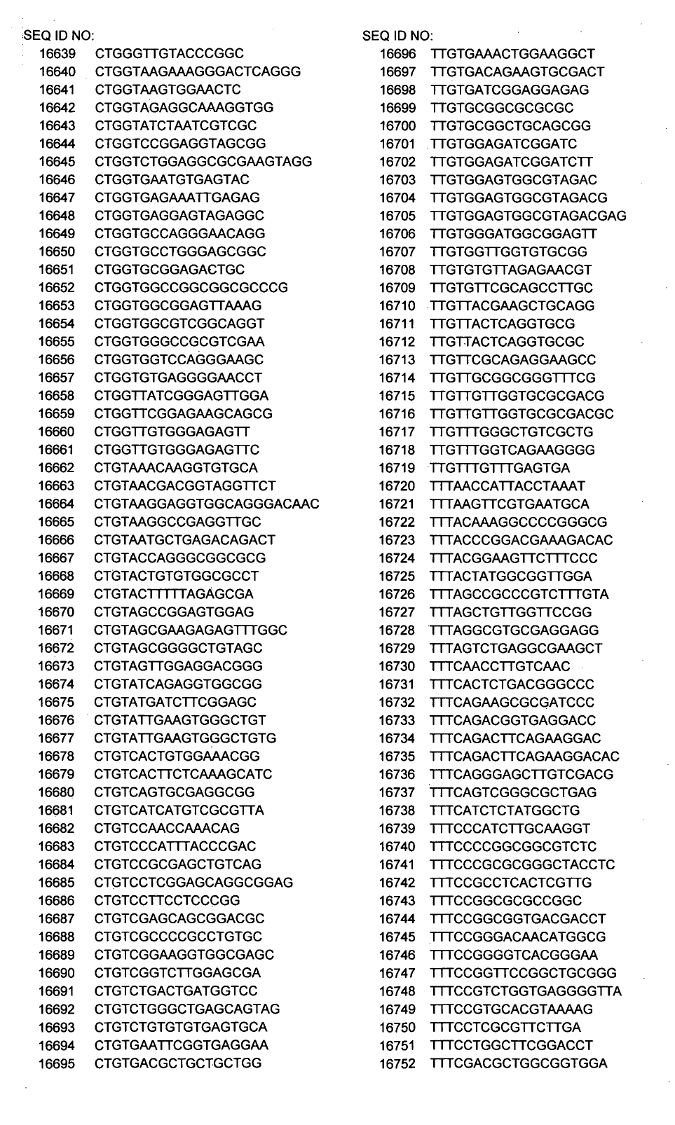

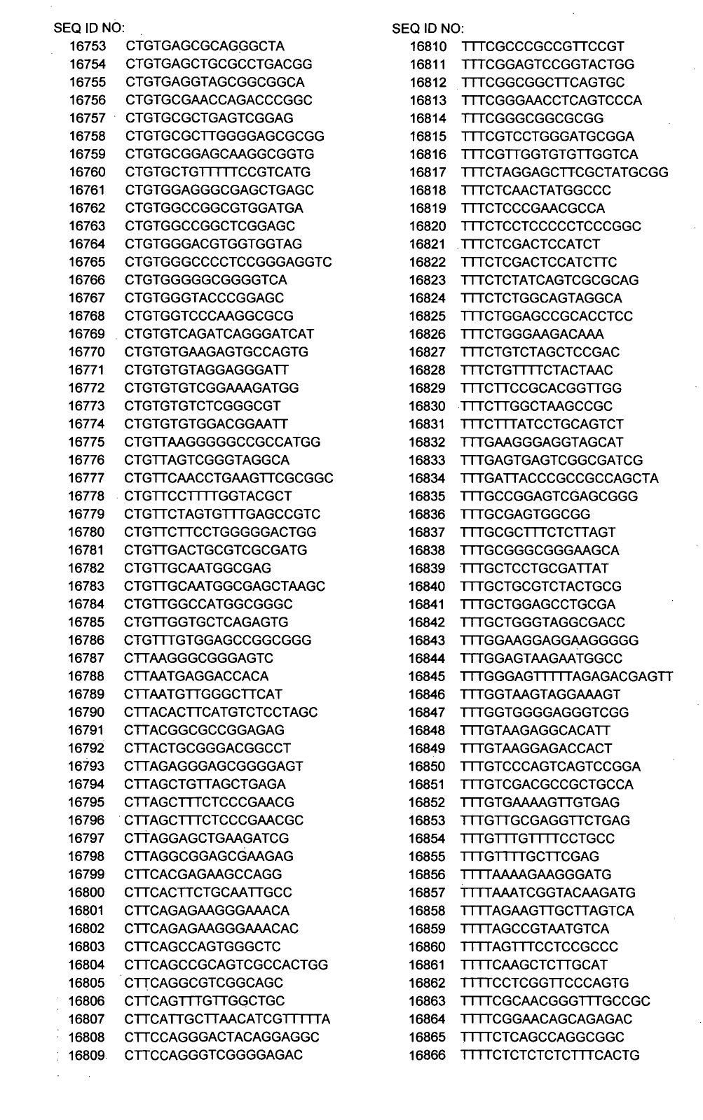

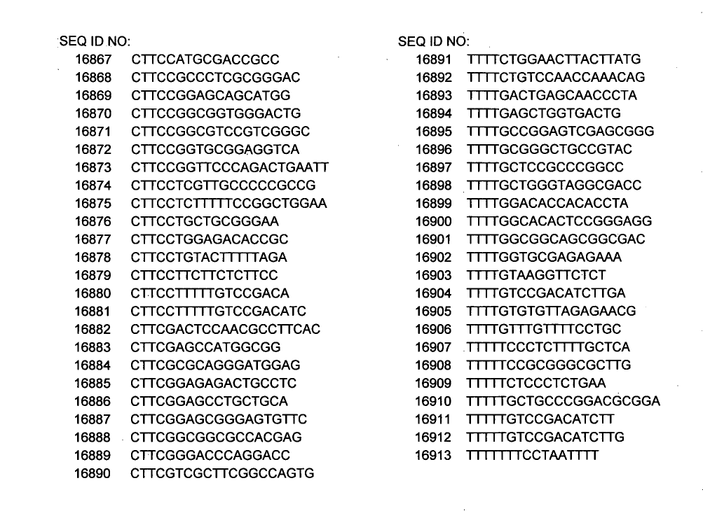

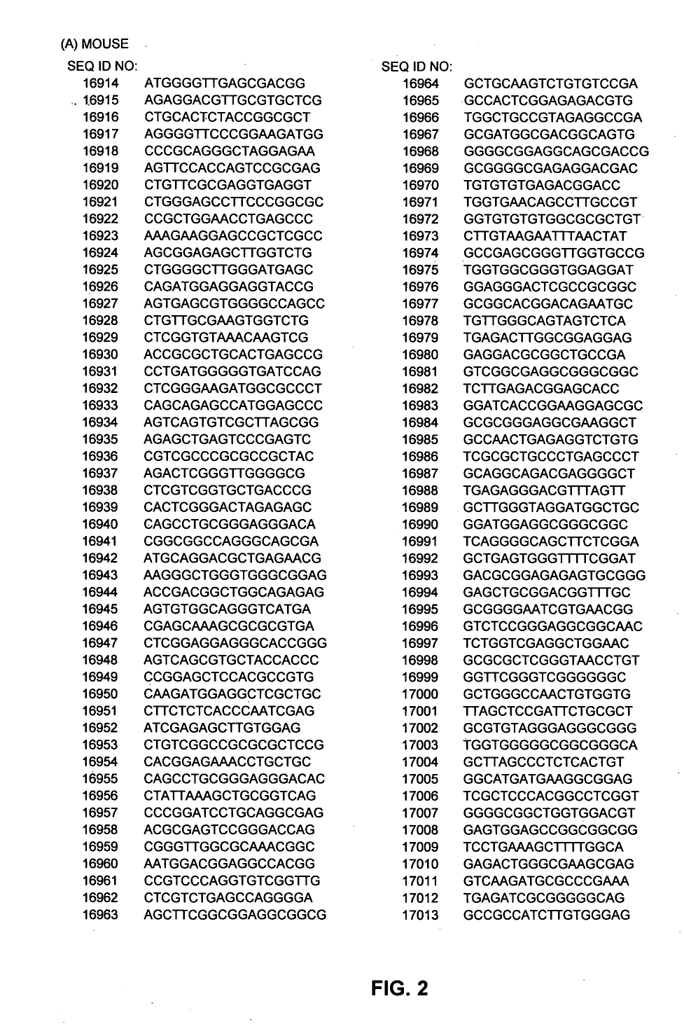

Non-limiting examples of the isolated tiRNA molecules of the invention are set forth in SEQ ID NOS: 1-16913 (FIG. 1 (human)) and SEQ ID NOS: 16914-17213 (FIG. 2 A-C: chicken, mouse and Drosophila)).

Typically, although not exclusively, the isolated tiRNA molecule is located in, or obtainable from, a cell nucleus.

It will also be appreciated that the invention contemplates nucleic acid molecules (e.g. RNA or DNA) complementary to or at least partly complementary to the tiRNA molecules of the invention. Complementary or at least partly complementary nucleic acid molecules may be in DNA or RNA form.

By “at least partly complementary” is meant having at least 60%, at least 70%, at least 75%, at least 80%, at least 90%, or at least 91%, 92%, 93%, 94%, 95%, 96%, 97%, 98% or 99% sequence identity with a nucleotide sequence of a tiRNA molecule.

The invention also provides a modified tiRNA molecule.

A modified tiRNA may be altered by, complexed, labeled or otherwise covalently or non-covalently coupled to one or more other chemical entities. In some embodiments, the chemical entity may be bonded, linked or otherwise attached directly to the tiRNA, or it may be bonded, linked or otherwise attached to the tiRNA via a linking group.

Examples of such chemical entities include, but are not limited to, incorporation of modified bases (e.g inosine, methylinosine, pseudouridine and morpholino), sugars and other carbohydrates such as 2′-O-methyl and locked nucleic acids (LNA), amino groups and peptides (e.g peptide nucleic acids (PNA)), biotin, cholesterol, fluorophores (e.g FITC, Fluoroscein, Rhodamine, HEX, FAM, TET and Oregon Green) radionuclides and metals, although without limitation thereto (Fabani and Gait, 2008; You et al., 2006; Summerton and Weller, 1997). A more complete list of possible chemical modifications can be found at http://www.oligos.com/ModificationsList.htm.

In one particular embodiment, the modified tiRNA is useful as an “antisense inhibitor”. By “antisense inhibitor” is meant a nucleic acid sequence that is either complementary to or at least partly complementary to the tiRNA molecule (Dias and Stein, 2002; Kurreck, 2003; Sahu et al., 2007). The antisense inhibitor pairs with the tiRNA and interferes with tiRNA-mRNA interactions. Experiments showing sequence-specific inhibition of small RNA function have previously been demonstrated both in vitro (Meister et al., 2004; Hutvagner et al., 2004) and in vivo (Krützfeldt et al., 2005).

In another particular embodiment, the modified tiRNA is a “point mutant”. By “point mutant” is meant a tiRNA molecule where 1 or 2 nucleotides have been removed, substituted or otherwise altered. Point mutants of tiRNAs or their targets can be employed to study the function of tiRNAs in disease or to increase the affinity of tiRNAs to variant targets. Small RNA molecules involved in disease processes, including miRNAs, may have “seed-sequences”. By “seed-sequences” is meant nucleic acid sequences that comprise 2-7 nucleotides and are involved in target recognition (Lewis et al., 2003; Lewis et al., 2005). Increasing the mismatch in these sequences is predicted to significantly decrease the gene regulation function of tiRNAs. This approach may be applicable for partial inhibition of tiRNA targets.

In yet another particular embodiment, the modified tiRNA is a “tiRNA mimic”. A “tiRNA mimic” is a single-stranded RNA oligonucleotide that is complementary to or at least partly complementary to the tiRNA. The tiRNA mimic may inactivate pathological tiRNAs through complementary base-pairing. It will also be appreciated that chemical modification to LNA, PNA or morpholino and conjugation to cholesterol may stabilize the tiRNA mimic molecule and facilitate delivery of single-stranded RNA molecules to targets following intraveneous administration (Rooij and Olson, 2007).

The invention also provides a fragment of a tiRNA of the invention. By “fragment” is meant a portion, domain, region or sub-sequence of a tiRNA molecule which comprises one or more structural and/or functional characteristics of a tiRNA molecule. By way of example only, a fragment may comprise at least 5, at least 6, at least 7, at least 8, at least 9, at least 10, at least 11, at least 12, at least 13, at least 14, at least 15, at least 16 or at least 17 nucleotides of a tiRNA molecule.

It will be appreciated that the tiRNA molecules can be chemically modified to facilitate penetration into cells. Examples of such modifications include, but are not limited to, conjugation to cholesterol, Morpholino, 2′O-methyl, PNA or LNA (Partridge et al., 1996; Corey and Abrams, 2001; Kos et al., 2003).

Modified tiRNA molecules also include “variants” of the tiRNA molecules of the invention. Variants include RNA or DNA molecules comprising a nucleotide sequence at least 70%, 75%, 80%, 85%, 90%, 95%, 96%, 97%, 98% or 99% identical to a nucleotide sequence of a tiRNA molecule such as described in FIG. 1 and FIG. 2. Such variants may include one or more point mutations, nucleotide substitutions, deletions or additions.

According to another aspect, there is provided a genetic construct comprising or encoding one or a plurality of the same or different tiRNA molecules, modified tiRNA molecules, at least partly complementary DNA or RNA molecules, or fragments thereof.

It will be appreciated that said tiRNA molecules may be oriented in tandem repeats or with multiple copies of each tiRNA sequence.

As used herein, a “genetic construct” is any artificially constructed nucleic acid molecule comprising heterologous nucleotide sequences.

A genetic construct is typically in DNA form, such as a phage, plasmid, cosmid, artificial chromosome (e.g. a YAC or BAC), although without limitation thereto. The genetic construct suitably comprises one or more additional nucleotide sequences, such as for assisting propagation and/or selection of bacterial or other cells transformed or transfected with the genetic construct.

In one particular embodiment, the genetic construct is a DNA expression construct that comprises one or more regulatory sequences that facilitate transcription of one or more tiRNA molecules, modified tiRNA molecules or fragments thereof.

Such regulatory sequences may include promoters, enhancers, polyadenylation sequences, splice donor/acceptor sites, although without limitation thereto.

Suitable promoters may be selected according to the cell or organism in which the tiRNA molecule(s) is/are to be expressed. Promoters may be selected to facilitate constitutive, conditional, tissue-specific, inducible or repressible expression as is well understood in the art.

It will be appreciated that the tiRNA molecule(s) may be provided as an encoding DNA sequence in an expression construct that, when transcribed, produces the tiRNA molecule as a transcript.

It will also be appreciated that tiRNA molecules appear to be a hitherto unknown form of small, single stranded RNA molecules that occur throughout evolution. Accordingly, tiRNA molecules may be isolated, identified, purified or otherwise obtained from any organism.

Preferably, the organism is a eukaryote.

More preferably, the organism is a metazoan inclusive of all multi-celled animals ranging from jellyfish to insects and vertebrates.

Even more preferably, the organism is a vertebrate, inclusive of mammals, avians such as chickens and ducks and aquaculture species such as fish, although without limitation thereto.

Even more preferably, the organism is a mammal.

Mammals include humans, livestock such as horses, pigs, cows and sheep, domestic animals such as cats and dogs, although without limitation thereto.

In further aspects, the invention therefore provides methods of identifying, purifying or otherwise obtaining a tiRNA molecule.

Broadly, such methods may include analysis of nucleic acid samples obtained from an organism, and/or bioinformatic analysis of genome sequence information.

Preferably, the nucleic acid samples are derived from the genome of a eukaryote.

More preferably, the nucleic acid samples are derived from the genome of a metazoan inclusive of jellyfish, insects and vertebrates.

Even more preferably, the nucleic acid samples are derived from the genome of a vertebrate, inclusive of mammals, avians such as chickens and ducks and aquaculture species such as fish, although without limitation thereto. Even more preferably, the nucleic acid samples are derived from the genome of a mammal.

Mammals include humans, livestock such as horses, pigs, cows and sheep, domestic animals such as cats and dogs, although without limitation thereto.

Preferably, a method for analyzing a nucleic acid sample to identify a tiRNA includes “deep sequencing”. One particularly useful but non-limiting method for identifying transcription start sites, followed by identification of small RNA species, including tiRNAs, in a nucleic acid sample is systematic deep sequencing of CAGE (5′ cap-trapped analysis of gene expression). Examples of specific deep sequencing technologies employed for the identification of TSSs and tiRNAs include, but are not limited to, 454™-, Solexa- and SOLiD-sequencing.

In particular embodiments relating to bioinformatic analyses of genome sequence information, the invention provides a computer-readable storage medium or device encoded with structural information of one or more tiRNA molecules.

The structural information may be nucleotide sequence, sequence length, GC content and/or proximity to a TSS, although without limitation thereto.

A computer-readable storage medium may have computer readable program code components stored thereon for programming a computer (e.g. any device comprising a processor) to perform a method as described herein. Examples of such computer-readable storage media include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one having ordinary skill in the art, notwithstanding possibly significant effort and many design choices motivated by, for example, available time, current technology, and economic considerations, when guided by the concepts and principles disclosed herein will be readily capable of implementing the invention by generating necessary software instructions, programs and/or integrated circuits (ICs) with minimal experimentation.

Typically, the computer-readable storage medium or device is part of a computer or computer network capable of interrogating, searching or querying a genome sequence database.

In one example, a bioinformatic method may utilize a high performance computing station which houses a local mirror of the UCSC Genome Browser.

One further aspect of the invention provides antibodies which bind, recognize and/or have been raised against a tiRNA of the invention, inclusive of fragments and modified tiRNA molecules.

Antibodies may be monoclonal or polyclonal. Antibodies also include antibody fragments such as Fc fragments, Fab and Fab′2 fragments, diabodies and ScFv fragments. Antibodies may be made in a suitable production animal such as a mouse, rat, rabbit, sheep, chicken or goat.

The invention also contemplates recombinant methods of producing antibodies and antibody fragments. For example, antibodies to RNA molecules have been produced by a method utilizing a synthetic phage display library approach to select RNA-binding antibody fragments (Ye et al., 2008).

As is well understood in the art, antibodies may be conjugated with labels selected from a group including an enzyme, a fluorophore, a chemiluminescent molecule, biotin, radioisotope or other label.

Examples of suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution or with a suitable chromogenic or chemiluminescent substrate.

Examples of chromogens include diaminobanzidine (DAB), permanent red, 3-ethylbenzthiazoline sulfonic acid (ABTS), 5-bromo-4-chloro-3-indolyl phosphate (BCIP), nitro blue tetrazolium (NBT), 3,3′,5,5′-tetramethyl benzidine (TNB) and 4-chloro-1-naphthol (4-CN), although without limitation thereto.

A non-limiting example of a chemiluminescent substrate is Luminol™, which is oxidized in the presence of horseradish peroxidase and hydrogen peroxide to form an excited state product (3-aminophthalate).

Fluorophores may be fluorescein isothiocyanate (FITC), tetramethylrhodamine isothiocyanate (TRITC), allophycocyanin (APC), Texas Red (TR), Cy5 or R-Phycoerythrin (RPE), although without limitation thereto.

Radioisotope labels may include ¹²⁵I, ¹³¹I, ⁵¹Cr and ⁹⁹Tc, although without limitation thereto.

Other antibody labels that may be useful include colloidal gold particles and digoxigenin.

In other aspects, the invention provides a method of identifying a tiRNA expression profile as a quantitative or qualitative indicator or measure of gene regulation. These methods may be particularly, although not exclusively, relevant to diagnosis of diseases and conditions associated with differential gene regulation.

In one particular embodiment, said tiRNA expression profile is an indicator and/or measure of gene transcriptional activity.

In one embodiment, the method uses a “nucleic acid array” (tiRNA array).

By “nucleic acid array” is a meant a plurality of nucleic acids, preferably ranging in size from 10, 15, 20 or 50 by to 250, 500, 700 or 900 kb, immobilized, affixed or otherwise mounted to a substrate or solid support. Typically, each of the plurality of nucleic acids has been placed at a defined location, either by spotting or direct synthesis. In array analysis, a nucleic acid-containing sample is labeled and allowed to hybridize with the plurality of nucleic acids on the array. Nucleic acids attached to arrays are referred to as “targets” whereas the labelled nucleic acids comprising the sample are called “probes”. Based on the amount of probe hybridized to each target spot, information is gained about the specific nucleic acid composition of the sample. The major advantage of gene arrays is that they can provide information on thousands of targets in a single experiment and are most often used to monitor gene expression levels and “differential expression”.

“Differential expression” indicates whether the level of a particular tiRNA in a sample is higher or lower than the level of that particular tiRNA in a normal or reference sample.

The physical area occupied by each sample on a nucleic acid array is usually 50-200 μm in diameter thus nucleic acid samples representing entire genomes, ranging from 3,000-32,000 genes, may be packaged onto one solid support. Depending on the type of array, the arrayed nucleic acids may be composed of oligonucleotides, PCR products or cDNA vectors or purified inserts. The sequences may represent entire genomes and may include both known and unknown sequences or may be collections of sequences such as miRNAs. Using array analysis, the expression profiles of normal and diseased tissues, treated and untreated cell cultures, developmental stages of an organism or tissue, and different tissues can be compared.

In one embodiment, gene profiling, such as but not limited to using a tiRNA array, is used to identify mRNAs whose expression shows a positive or inverse correlation with the expression of a specific tiRNA.

It will be appreciated that an absence of tiRNA expression could correlate with a presence of mRNA expression, or vice versa. Alternatively, a presence of tiRNA expression could correlate with a presence of mRNA expression or an absence of tiRNA expression could correlate with an absence of mRNA expression. Furthermore, a level of tiRNA expression could correlate with a level of mRNA expression, whether directly or inversely. It will be appreciated that a level of expression may be measured as a quantitative or a relative expression level.

In another embodiment, gene profiling allows the identification of regulators of disease processes and potential therapeutic targets.

Examples of diseases and conditions that show differential gene regulation include but are not limited to Crohn's disease, Alzheimer's disease, Parkinson's disease, rheumatoid arthritis, myocardial infarction, diabetes, congenital developmental disorders, coronary heart disease and cancer such as breast cancer, lymphoma, leukemia, colorectal cancer, gastric cancer, ovarian cancer, aggressive metastatic brain cancer and pituitary tumors (McKaig et al., 2003; Grünblatt et al., 2007; Liang et al., 2008; Lübke et al., 2008; Ridker, 2007; Zecchini et al., 2008).

It will be appreciated that said gene regulation may refer to aberrant gene transcription.

Further, tiRNAs may be associated with aberrant regulatory activity of oncogenes or tumor suppressors (Zhang et al., 2006) and may therefore become useful biomarkers for cancer diagnostics.

It will be appreciated that said aberrant regulatory activity may in some embodiments refer to aberrant transcriptional activity.

In one particular embodiment, the tiRNAs may be associated with oncogenes such as CITED4, p53, HoxA11, HoxA9, myc and ETS1.

In another particular embodiment, the tiRNAs may be linked to aberrant regulation and/or transcription of genes associated with leukemia such as AF10, ALOX, 12, ARHGEF12, ARNT, AXL, BAX, BCL3, BCL6, BTG1, CAV1, CBFB, CDC23, CDH17, CDX2, CEBPA, CLC, CR1, CREBBP, DEK, DLEU1, DLEU2, EGFR, ETS1, EVI2A, EVI2B, FOXO3A, FUS, GLI2, GMPS, IRF1, KIT, LAF4, LCP1, LDB1, LMO1, LMO2, LYL1, MADH5, MLL3, MLLT2, MLLT3, MOV10L1, MTCP1, NFKB2, NOTCH!, NOTCH3, NPM1, NUP214, NUP98, PBX1, PBX2, PBX3, PBXP1, PITX2, PML, RAB7, RGS2, RUNX1, SET, SP140, TAL1, TAL2, TCL1B, TCL6, THRA, TRA and ZNFN1A1.

In yet another particular embodiment, the tiRNAs may be linked to aberrant regulation and/or transcription of genes associated with Alzheimer's disease such as APP and APOE.

It will also be appreciated that in some particular embodiments, the tiRNAs may be associated with aberrant regulation and/or transcription of genes such as BRCA1 and BRCA2 in breast cancer; HER2, ras, src, hTERT, and Bcl-2 in aggressive metastatic brain cancers; PON1 in coronary heart disease; and homeobox genes (e.g. HoxA10 and SOX2) in congenital developmental disorders.

Other methods of the invention, including but not limited to the herein mentioned tiRNA array, relate to diagnostic applications of the claimed nucleic acid molecules. For example, tiRNAs may be detected in biological samples in order to determine and classify certain cell types or tissue types or tiRNA-associated pathogenic disorders which are characterized by differential expression of tiRNA molecules or tiRNA molecule patterns. Further, the developmental stage of cells, organs and/or tissues may be classified by determining spatial and/or temporal expression patterns of tiRNA molecules.

In another aspect, the invention provides a method of treating a disease or condition in an animal, said method including the step of administering to the animal a therapeutic agent selected from the group consisting of:
(i) an isolated tiRNA molecule;
(ii) a fragment of the isolated tiRNA molecule;
(iii) a modified tiRNA molecule;
(iv) an at least partly complementary RNA or DNA molecule; and/or
(v) an antibody that binds any one of (i)-(iv);
   to thereby treat said disease or condition.

Accordingly, the aforementioned therapeutic agents may be suitable for prophylaxis and/or therapy of animals, including mammals such as humans. For example, the therapeutic agents may be used to treat diseases, conditions, developmental processes and/or disorders associated with developmental dysfunctions including, but not limited to, cancer. Certain tiRNAs may function as tumour-suppressors and thus expression or delivery of these tiRNAs or “tiRNA mimics” to tumor cells may provide therapeutic efficacy.

In one embodiment, the use of chemically modified tiRNAs to target either a specific tiRNA or to disrupt the binding of a tiRNA and its specific mRNA target in vivo may provide a potentially effective means of inactivating pathological tiRNAs.

Alternatively, tiRNAs may be administered to potentiate the effects of natural tiRNAs by promoting the expression of beneficial gene products such as tumour suppressor proteins (Rooij and Olson, 2007).

Therapeutic agents may be delivered to an animal in the form of a pharmaceutical composition comprising a pharmaceutically acceptable carrier diluent or excipient.

Accordingly, the invention provides a pharmaceutical composition comprising a therapeutic agent selected from the group consisting of:
(i) an isolated tiRNA molecule;
(ii) a fragment of the isolated tiRNA molecule;
(iii) a modified tiRNA molecule;
(iv) an at least partly complementary RNA or DNA molecule and/or
(v) an antibody that binds any one of (i)-(iv);
   and a pharmaceutically acceptable carrier, diluent or excipient.

By “pharmaceutically-acceptable carrier, diluent or excipient” is meant a solid or liquid filler, diluent or encapsulating substance that may be safely used in systemic administration. This includes carriers, diluents or excipients suitable for veterinary use.

Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991).

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines. In the case of gene therapy, which contemplates the use of electroporation or liposomal transfection into tissues, the drug may be transfected into cells together with the DNA.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be achieved by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to achieve a beneficial response in a patient over an appropriate period of time. The quantity of agent(s) to be administered may depend on the subject to be treated inclusive of the age, sex, weight and general health condition thereof, factors that will depend on the judgement of the practitioner.

Methods and compositions may be used for treating diseases or conditions in any animal. Animals include and encompass fish, avians (e.g. chickens and other poultry) and mammals inclusive of humans, livestock, domestic pets and performance animals (e.g. racehorses), although without limitation thereto.

So that the invention may be readily understood and put into practical effect, reference is made to the following non-limiting examples.

### EXAMPLES

### Example 1

### Identification of Transcription Start Sites (TSSs) and Small RNAs by Systematic Deep Sequencing

Transcription start sites (TSSs) in THP-1 cells, a human-derived acute monocytic leukemia cell line (Tsuchiya et al., 1982), were identified by systematic deep sequencing of CAGE (5′ cap-trapped analysis of gene expression) tags (Shiraki et al., 2003; Suzuki, 2008) (hereafter referred to as deepCAGE). DeepCAGE was performed on undifferentiated THP-1 cells and at five time points (1, 4, 12, 24, and 96 hours) during macrophage differentiation in response to phorbol 12-myristate 13-acetate (PMA) stimulation. DeepCAGE tags were mapped to the human genome, pooled across time points, and clustered to yield ˜18,000 high confidence active promoters (Suzuki, submitted 2008). These promoters contain ˜20% (˜250,000) of all mapped deepCAGE tags. Promoters that mapped to repeat masker annotations, random chromosomes, assembly gaps, the mitochondrial genome, or annotated small RNAs were removed from the analysis. Less than 0.07% of promoters overlap any annotated small RNA loci (including miRNAs and snoRNAs), showing that the CAGE libraries are not contaminated with small RNAs. The remaining 14,818 promoters were used for all subsequent analysis. On average, promoters spanned 33 nt and were composed of 16 tags, with a mean tag abundance of 2 counts per million (cpm) sequenced tags.

### Bioinformatic Analysis of THP-1 Promoters.

All bioinformatic analysis was done on a high performance computing station which houses a local mirror of the UCSC Genome Browser (Karolchik et al., 2008). Repeat masker annotations, miRNA and snoRNA loci, and assembly gaps were obtained through the local mirror. Intersections required a minimum of 1 base of overlap, and were accomplished using a modified version of UCSC's tool, bedIntersect. Promoter architecture was assessed using a python script incorporating previously published criteria (Carninci et al., 2006). Promoters with less than 10 total tags were excluded from promoter architecture analysis. Using previously reported promoter architecture definitions we found that the promoters used in all tiRNA analyses were predominantly broad with peak (PB, 46.1%), followed by generally broad (BR, 34.4%), single peak (SP, 14.4%), and multimodal (MU, 5.1%) (Carninci et al., 2006).

### THP-1 Small RNA Deep Sequencing

### Cell Culture and RNA Extraction

THP-1 cells were cultured in RPMI, 10% FBS, Penicillin/Streptomycin, 10 mM HEPES, 1 mM Sodium Pyruvate, 50 μM 2-Mercaptoethanol, and treated with 30 ng/ml PMA (Sigma) to differentiate them into macrophage-like cells. In addition to 5 unmixed short RNA libraries from undifferentiated THP-1 cells, mixed short RNA libraries were generated from THP-1 cells over a time-course of PMA differentiation (0, 2, 4, 12, 24, 96 h).

Total RNA was extracted using the standard AGPC (Acid-Guanidinium-Phenol-Chloroform) method, and all precipitations were done with ethanol, instead of Isopropyl alcohol, in order to ensure the recovery of short oligonucleotides. CTAB selective precipitation of long RNA was performed to separate long and short RNAs. Short RNAs (<75 bp) were isolated from the CTAB precipitation supernatant by precipitation with 2 volumes of ethanol. The RNA pellet was resuspended in 7M GuCl and re-ethanol precipitated.

### Mixed Short RNA Library Construction

Short RNAs derived from each time point were tagged with a 4 nt tissue ID tag during the adaptor ligation step. RNA-DNA hybrid oligonucleotide adaptor ligation was carried out using 10 μg total short RNA, 100 μM of a 5′ adaptor, containing an EcoRI recognition site (5′ adaptor sequence: 5′-acgctcacagaattcAAA-3′, upper-case is RNA oligo, lower-case is DNA oligo) and 100 μM of a specific 3′ adaptor containing an EcoRI recognition site and a 4 nt Tissue ID tag (3′ adaptor sequence: 5′-phosphate-UXXxxgaattctcacgaggccagcgt-biotin-3′, upper-case is RNA oligo, lower-case is DNA oligo, XXxx is Tissue ID tag), with T4 RNA Ligase (TaKaRa) for 16 hrs at 15° C. The sample:adaptor mixture ratio was short RNA 1 μg:100 μM 5′adaptor 0.7 μl:100 μM 3′adaptor 0.7 μl. At the end of reaction, samples for each mixed library were pooled, treated with 20 mg/ml Proteinase K (15 mins, 37° C.) and purified by phenol/chloroform extraction and ethanol precipitated to generate purified short RNAs.

Purified short RNAs were separated from adaptor dimers ((100-200 bp) 100 bp) on an 8% denaturing PAGE gel. 100-200 bp short RNAs, running above adaptor dimers, were excised and eluted from the gel in TEN elution buffer (10 mM Tris.HCl pH7.5, 1 mM EDTA pH 7.5, 250 mM NaCl) for 16 hrs˜ at 4° C. The extracted short RNA tags were filtered through MicroSpin Empty Columns (Amersham Biosciences) in TEN buffer three times to remove polyacrylamide contaminant. The filtered sample was purified by ethanol precipitation.

cDNA synthesis was carried out from purified short RNAs using 3′RT-PCR primer (sequence: 5′-biotin-gcacgctggcctcgtgagaattc-3′) with M-MLV Reverse Transcriptase RNase H Minus, Point Mutant (Promega). RT products were calibrated to determine the ratio of products derived from individual samples in the mixed library.

The cDNA fragment derived from short RNA tags were amplified by PCR using adaptor-specific primers: Primer 1 (454shortRNA3′RT-PCRprimer): 5′-biotin-gcacgctggcctcgtgagaattc-3; Primer 2 (454shortRNA5′PCRprimer): 5′-biotin-cagccgacgctcacagaattcaaa-3′. PCR was performed from 5 μl of template RT mixture, 1× buffer, 3 μl of DMSO, 12 μl of 2.5 mM dNTPs, 1.5 μl of 100 uM Primer 1, 1.5 μl of 100 uM Primer 2, 0.5 μl of EX taq polymerase (5 units/μl, TaKaRa) in a total volume of 50 ul. After incubating at 94° C. for 1 min, 12˜14 cycles were performed for 30 sec at 94° C., 30 sec at 57° C., 1 min at 70° C.; followed by 5 mins incubation at 70° C. PCR products were pooled, purified, ethanol precipitated and resuspended in 40 μl of TE buffer. The PCR products were purified on a 12% polyacrylamide gel. The appropriate 60˜80 by fraction was cut out of the gel, eluted in 500 μl of SAGE elution buffer (2.5 mM Tris.HCl pH7.5/1.25 mM ammonium acetate/0.17 mM EDTA pH 7.5) for 16 hrs at room temperature. The extracted short RNA tags were filtered twice through with MicroSpin Empty Columns (Amersham Biosciences) by centrifugation at 3000 rpm for 2 min in SAGE buffer. The resulting extract was purified by ethanol precipitation, resuspended in 25 μl of 0.1×TE buffer and quantified with Picogreen.

PCR-amplified, gel-purified short RNA tags were re-amplified in a total volume of 100 μl containing 2 ng of short RNA tags, 6 μl of DMSO, 12 μl of 2.5 mM dNTPs, 2 μl of 100 uM Primer 1, 2 μl of 100 uM Primer 2, 0.8 μl of EX taq polymerase (5 units/μl, TaKaRa). All PCR products were used in subsequent steps. After incubating at 94° C. for 1 min, 8˜9 cycles were performed at 30 sec at 94° C., 30 sec at 57° C., 1 min at 70° C. followed by 5 mins at 70° C. The PCR products were pooled, purified, ethanol-precipitated and redissolved in 50 μl of TE buffer.

PCR products were further purified with G-50 micro-columns (GE Healthcare), ethanol precipitated and resuspended in 100 μl of TE buffer. The concentration was measured with Picogreen. PCR products were digested with EcoRI (Fermentas) in several reactions (3 μg/reaction), followed by Proteinase K treatment (20 mg/ml, 45 C, 15 minutes).

The desired 25˜40-bp DNA tags derived from short RNAs were separated from the free DNA ends derived from the ligated adaptors (cut off during restriction) by incubation with streptavidin-coated magnetic beads, which capture the biotin-labeled DNA ends. The cleaved tags were mixed with the beads (700 μl) and incubated at room temperature for 15 mins with mild agitation. Then the supernatant was collected after removal of the magnetic beads. The beads were rinsed with 50 μl of 1×BW buffer (Beads wash buffer: 1M NaCl, 0.5 mM EDTA, 5 mM Tris-HCl (pH7.5)), and pooled 25˜42-nt tags from both supernatant were extracted by phenol/chloroform followed by ethanol precipitation and resuspension in 40 μl of TE buffer, or purified through Microcon YM10 columns with buffer exchange into 0.1×TE. The short RNA tags were further purified on a 12% polyacrylamide gel. The desired 25˜42-nt fraction was cut out of the gel, crushed, and eluted in SAGE elution buffer (2.5 mM Tris.HCl pH7.5, 1.25 mM ammonium acetate, 0.17 mM EDTA pH 7.5) for 16 hrs at room temperature, followed by purification, concentration with YM10 columns, and ethanol precipitation. The DNA was finally resuspended in 6 μl of 0.1×TE buffer and quantified with Picogreen.

The short RNA tags (total yield) and 454 A, B adaptors (1/20 quantity of short RNA tags) were concatenated in a 10 μl reaction with T4 DNA ligase (NEB) for 16 hrs at 15° C. Proteinase K digestion was carried out by adding 70 μl of TE buffer and 20 mg/ml Proteinase K and digesting at 45 C for 15 minutes. Concatenated tags were purified with GFX columns (Amersham) to eliminate short concatamers (<100 bp). The eluted sample (50 ul) was transferred to Roche for 454 sequencing.

### Unmixed Short RNA Library Construction

An additional 5 unmixed short RNA libraries, each containing a specific range of short RNA lengths, were constructed from undifferentiated THP-1 (referred to as control 0 h small RNAs within the main text). Unmixed Short RNA libraries were constructed using the mixed library protocol (above).

### Short RNA Library Sequencing and Tag Extraction

Concatamerized tags derived from short RNAs were sequenced using the GS FLX 454 sequencer (Roche) (Margulies et al., 2005). We used in-house developed algorithms for linker masking and the extraction of short RNA tags. Short RNA tags were extracted with the following parameters: EcoRI ligated doublet linker (12-16 bp) masking: maximum mismatch, 2 by allowed; short RNA tag length, no limits.

### Example 2

### Mapping of Small RNAs to the Human Genome

Small RNAs were isolated from unstimulated THP-1 cells, and at 2, 4, 12, 24, and 96 hours after PMA stimulation and sequenced using the Roche FLX Genome Sequencer (see above). From over 10 million sequence reads we obtained a total of 1.9 million distinct small RNA tags. Small RNA tags were mapped to the human genome (not allowing mismatches) using an in-house software package (see below), and were pooled across time points as was done with promoters identified by deepCAGE. We obtained a total of 57,198 tags that mapped uniquely to the genome, which were furthered screened to remove tags that mapped to repeat masker annotations, random chromosomes, the mitochondrial genome, known miRNA and snoRNA loci, and unannotated sequences with high homology to tRNAs or rRNAs.

Relative expression can be assessed by the number of times a small RNA is detected among all sequences obtained. In contrast to known miRNAs, which are highly expressed (average of 200 cpm per uniquely mapped tags), the remaining 22,976 small RNAs are weakly expressed, occurring on average twice per million uniquely mapped tags.

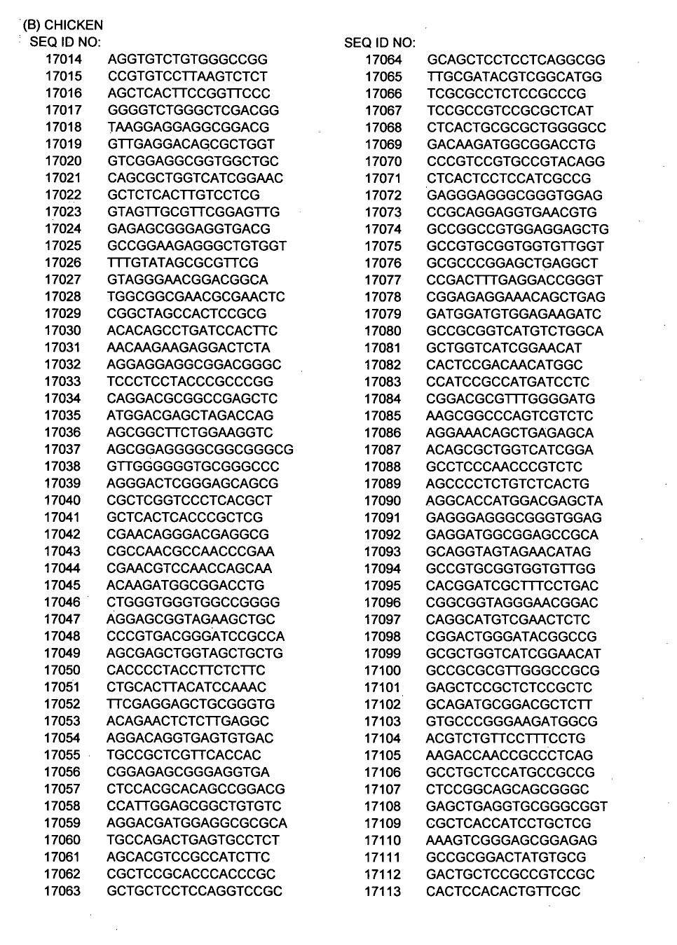

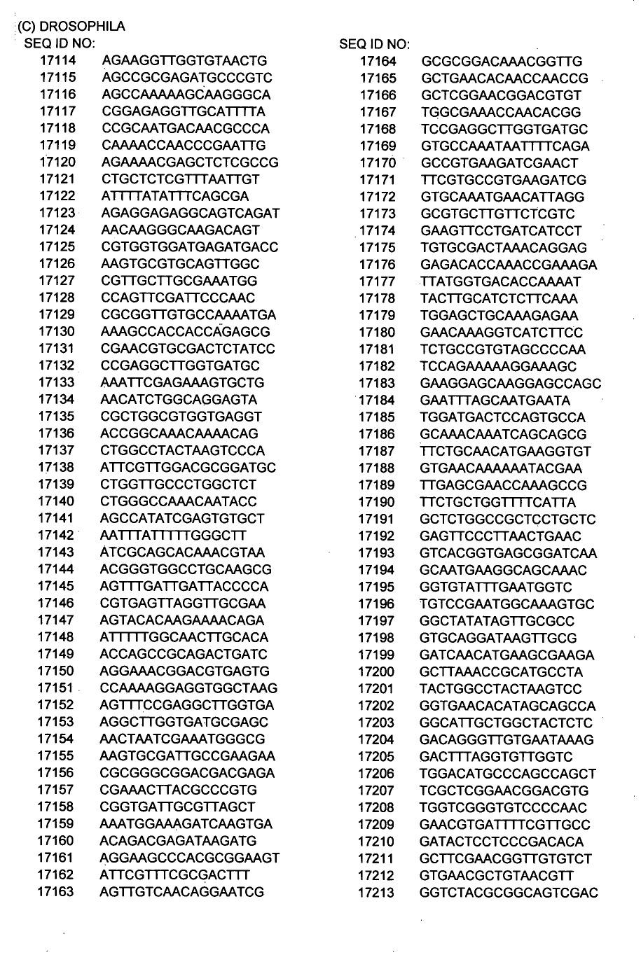

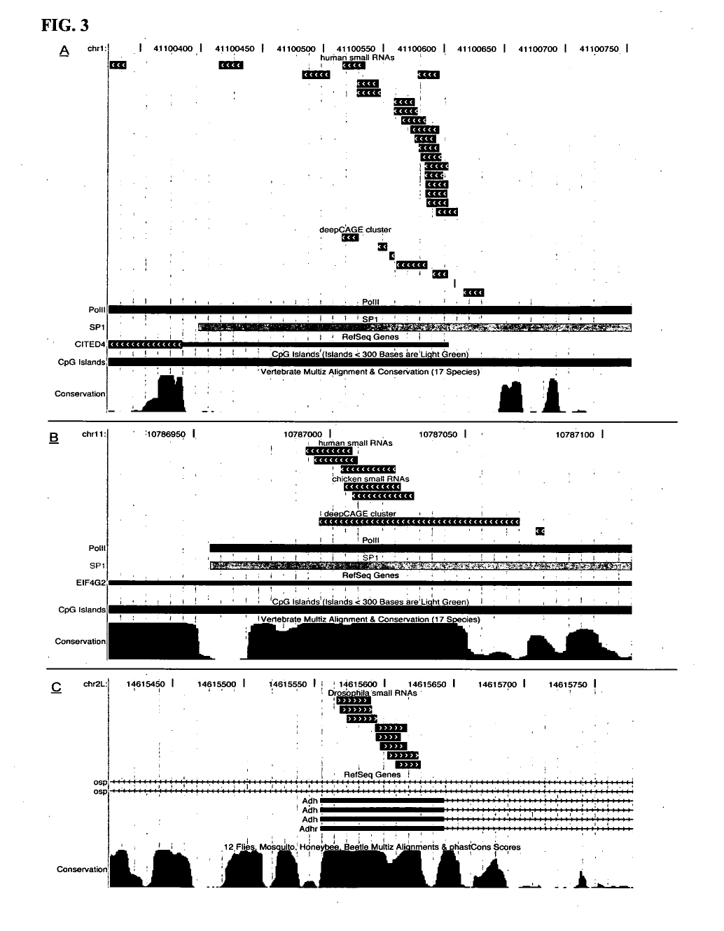

Previous deep sequencing studies have disregarded low abundance non-miRNA tags as spurious, inconsequential, or degradation products. We reasoned, however, that small RNAs in these libraries were only cloned and sequenced if they possessed a terminal 5′ phosphate, thus selecting against degradation products, and that a non-random genomic distribution would suggest that these tags are biologically meaningful. Comparison of promoters with the small RNA dataset revealed many regions of active transcription where small RNAs are abundant (FIG. 3). Indeed, we found that small RNAs in our filtered set are greater than 190 fold enriched at active promoters.

### THP-1 Small RNA Mapping

Small RNAs were mapped using ‘lochash’, an in-house application written in C++ designed to quickly locate large numbers of short (as small as 8 nucleotides) sequence element as specified in multifasta file, against a target genome. An exhaustive search of probes against the target genome (NCBI Build 36.1 of the human genome) was performed using a comprehensive hash table of all Nmers, which facilitates quick elimination of query sequences which do not have exact matches. Small RNAs were queried against both strands of the target genome, and filtered to remove any small RNA tags that mapped more than once. Intersections with genomic features (e.g. known small RNA loci, repeats) were performed as described for promoters (above).

### Example 3

### Distribution and Size Characteristics of tiRNAs from a Human Cell Line, THP-1

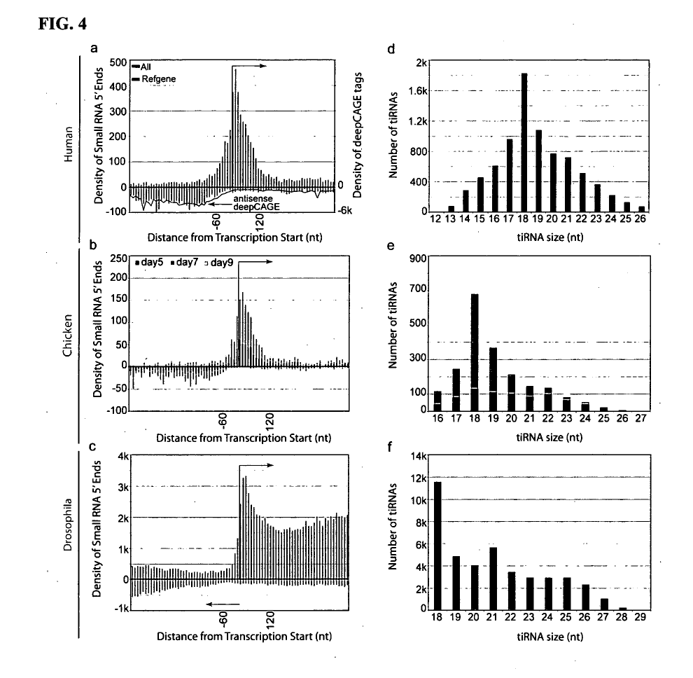

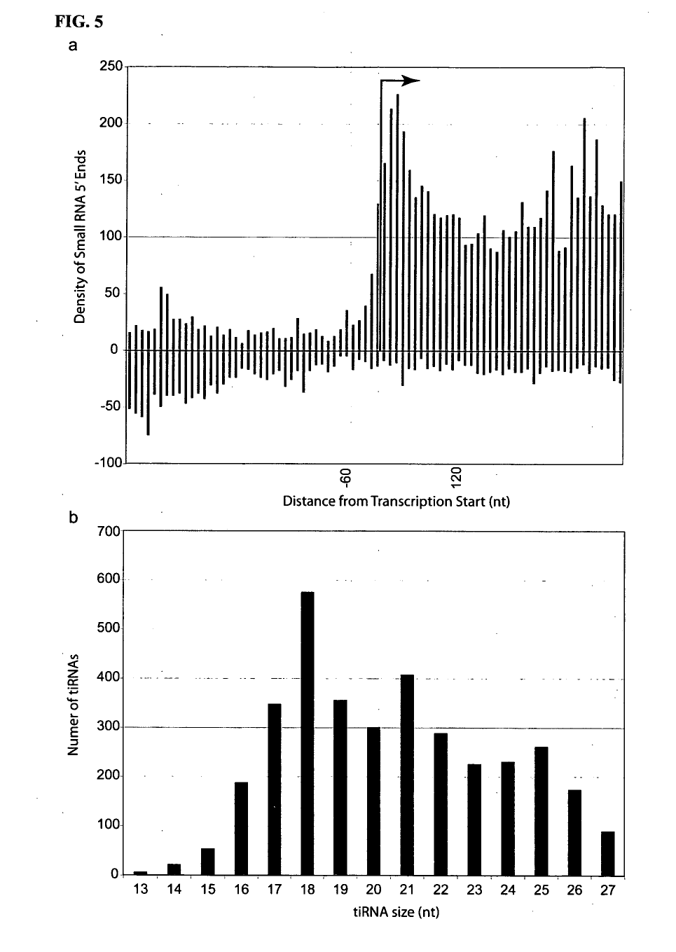

To examine the distribution of THP-1 small RNAs with respect to TSSs identified by deepCAGE we plotted small RNA density with respect to the most highly expressed deepCAGE tag from each promoter. Indeed, we found that small RNAs in our filtered set are greater than 190 fold enriched at active promoters. Within a 400 nt window in 10 nt bins either side of the TSS small RNAs were found to occur mainly just downstream of the TSS, with a dominant peak at +10-+20 nt (FIG. 4A). In total, regions −60 to +120 nt from the TSS encompassed 2312 small RNAs (>10% of the entire unannotated small RNA dataset) and 2824 promoters, due to the fact that many promoters are found close to one another. We termed these small RNAs “transcription initiation RNAs” (tiRNAs).

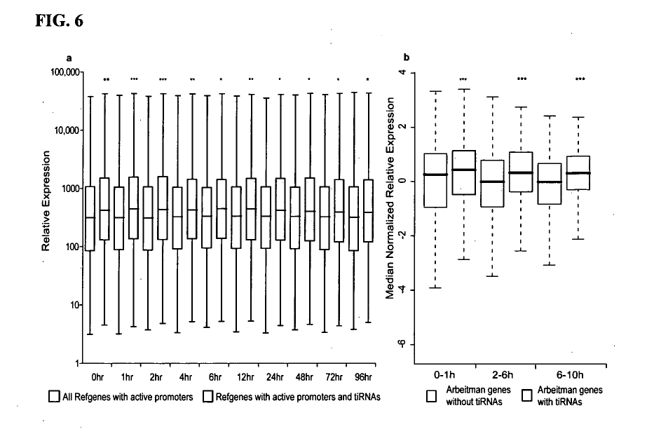

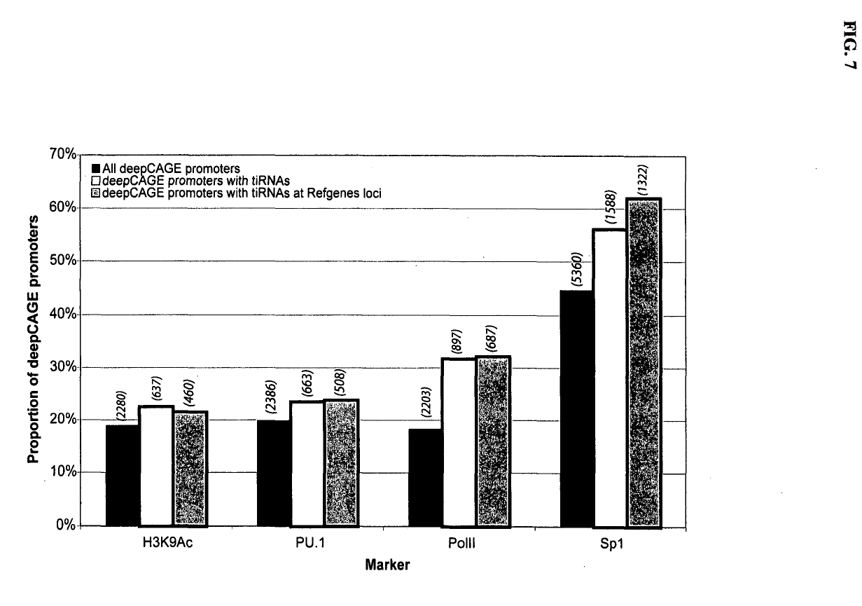

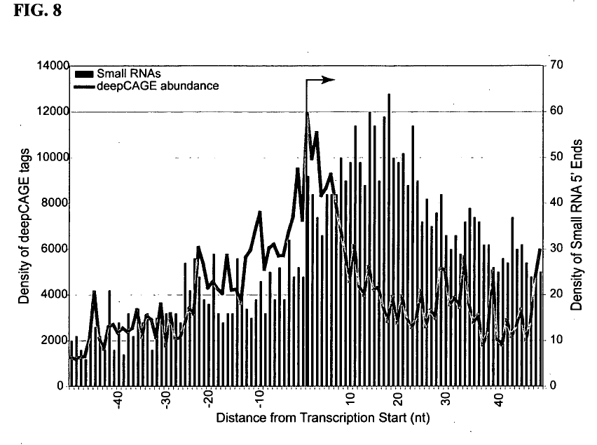

Plotting tiRNA density at higher resolution revealed that although the 5′ end of some tiRNAs coincides with the most highly expressed deepCAGE tag in a promoter, tiRNAs are predominantly 10-30 nt downstream (FIG. 4A, FIG. 8). This suggests that tiRNAs are not merely truncated or degraded 5′ ends of highly expressed transcripts. This distribution does not correlate with the abundance of deepCAGE tags downstream of the dominant transcription start site (FIG. 8), and was conserved in the subset of promoters with robust single-peak transcription starts sites (FIG. 9), many of which are associated with TATA-boxes (Carninci et al., 2006).

Further strengthening our results, and demonstrating that tiRNAs are not related to aberrant transcription, we found that the majority (74%) of tiRNAs and the promoters they are associated with (75%) map to Refgene promoter regions, and display the same density distributions observed for the dataset as a whole (FIG. 4A). When the analysis was extended to deepCAGE tags not incorporated within active promoters (see above, an additional ˜1.2 million deepCAGE tags) a further 6192 tiRNAs were identified, yielding a total of 8505 tiRNAs, or 38% of the total unannotated small RNA dataset. These tiRNAs intersect with an additional 776 Refgene promoters.

THP-1 tiRNA Analysis

Small RNA distributions with respect to the TSS were calculated by tabulating the number of small RNA 5′ ends in each bin—e.g. the number of small RNA 5′ ends that map to bases 0 to +10 relative to the transcription start. Because some TSSs map close to one another, a small RNA can be counted in more than one bin. However, we found this occurred for less than 15% of small RNAs, and thus did not substantially affect the results.

To ensure that sequence composition biases at promoters were not affecting small RNA mapping we examined all promoter regions (−60 to +120 nts relative to the most highly expressed CAGE tag) with evidence of tiRNAs and created an index of all Nmers (14-23 nts) that are unique in the human genome. We found that unique 18 mer Nmers are not overrepresented at these regions, and are found as often as expected in a random model. We then analyzed the number of unique small RNA mappings at these regions and compared them with the expected number of mappings, based on the unique Nmer index. We found fewer small RNAs of every size class (except 14 mers, which are the most weakly represented), with respect to 18 mers, than we would expect by chance.

### Bootstrap Analysis

A perl script executing a bootstrap analysis was used to estimate the likelihood of small RNAs overlapping promoters (for THP-1 small RNAs) or a Refgene TSSs (for Gallus gallus and Drosophila small RNAs, see below). For these analyses small RNAs and promoters were collapsed down to individual loci using UCSC's featureBits tool, eliminating the possibility that multiple small RNAs and promoters mapping to the same region could artificially enhance the results. Small RNAs were randomly assigned new chromosomal locations, and the number intersecting with promoters or Refgene TSSs was tabulated. This process was repeated for 10⁵ iterations. Fold enrichment was determined by dividing the number of observed overlaps by the average number of overlaps in all iterations.

### Example 4

### Regulation and Function of tiRNAs

To assess the regulation and function of tiRNAs, we analyzed the transcriptional activity of promoters associated with tiRNAs. Using the most highly expressed deepCAGE tag per promoter as a proxy for promoter activity revealed that promoters with tiRNAs were more highly expressed than promoters without tiRNAs (average 53 cpm vs 30 cpm; P<10⁻⁸), and that Refgene promoters associated with tiRNAs are even more highly expressed (average 60 cpm; P<10⁻¹⁰). Additionally, using previously reported promoter architecture definitions (Carninci et al., 2006) we found that promoters with tiRNAs are predominantly broad and broad with peak (48% and 31%, respectively), consistent with the dataset as a whole.

THP-1 response to PMA was examined in detail using Illumina bead-based arrays (Suzuki, submitted 2008). Refgenes with evidence of tiRNAs at their promoters are highly expressed at all time points (FIG. 6). Interestingly, Refgenes with tiRNAs at their promoters exhibit no Gene Ontology term enrichment.

### THP-1 Promoters at Refgene TSSs

Refgene annotations were obtained from the local mirror of the UCSC Genome Browser. A promoter mapping within −300 to +100 nt relative to an annotated Refgene TSSs was defined as mapping with a Refgene promoter. Correspondingly, these genes were identified as “present” by deepCAGE. The most highly expressed deepCAGE tags from promoters mapping within Refgene promoter regions are tightly associated with annotated TSSs. Nearly one third map to the first nucleotide of an annotated Refgene TSS, and nearly two thirds map within 50 nt of the annotated Refgene TSS. A two-tailed T-test was used to test if deepCAGE expression levels were different between populations.

### THP-1 Refgene Expression and Gene Ontology Analysis

Refgenes associated with tiRNA promoters were identified, and refSeq mRNA accession numbers were retrieved and mapped to the Human illumina V2 probe centric “genome” in Genespring v7.3.1. RIKEN quantile normalized data generated from PMA treated THP-1 biological replicates was used to examine expression levels (Suzuki, submitted 2008). A chi-squared test was used to determine statistical significance. Gene Ontology enrichment was assessed using the web-based FatiGO+ platform (Al-Shahrour et al., 2007).

### Example 5

### Enrichment for Sp1 and RNA Polymerase II at Promoters with tiRNAs

To assess if promoters with tiRNAs showed enrichment for other genomic features indicative of active transcription we examined these loci for evidence of H3K9-acetylation or binding of RNA Polymerase II and the transcription factors Sp1 and PU.1 in THP-1 cells (Suzuki, submitted 2008). Active promoters with tiRNAs exhibit pronounced enrichment for binding of RNA Polymerase II and Sp1 but, unexpectedly, show no significant correlation with H3K9-acetylation or Pu.1 binding (FIG. 7). Although tiRNAs were on average more weakly expressed (0.75 cpm per uniquely mapped tags) than unannotated small RNAs as a whole, they show specific size and sequence composition characteristics. The vast majority are less than 22 nucleotides, and almost one quarter are 18 nt (FIG. 4D). This pattern was not due to a bias towards unique 18 mers in promoter regions, or against unique n-mers of shorter length.

To ascertain if the tiRNA size distribution is unique to small RNAs proximal to TSSs we binned all unannotated small RNAs by position within annotated Refgenes. Parsing Refgene annotations into deciles to normalize for gene size we found that the most 5′ and most 3′ deciles of Refgenes contained the greatest number of small RNAs. However, we found nearly four times as many small RNAs at the 5′ ends of Refgenes as in 3′ ends, and noted that over one third of 3′ end small RNAs can be classified as tiRNAs due to their proximity to a deepCAGE tag in the 3′ end of the Refgene, leaving only ˜700 3′ end tags that were not associated with a deepCAGE tag. The size distribution of these remaining 3′end small RNAs is significantly different from tiRNAs and does not show a dominance of 18 nt small RNAs (FIG. 10).

The tiRNAs do not exhibit characteristics common to other small structural and regulatory RNAs. Less than 0.5% of tiRNAs intersect with an Evofold prediction (Pedersen et al., 2006), and only a third overlap with a phastCons element (Siepel et al., 2005). Additionally, unlike miRNAs, which are typically ˜50% GC (Griffiths-Jones et al., 2008), tiRNAs average 72% GC. Indeed, congruent with their location at TSSs with broad promoters, 88% of tiRNAs overlap an annotated CpG island (Gardiner-Garden and Frommer, 1987; Karolchik et al., 2008), and 92% contain a CpG dinucleotide, which correlates with their association with Sp1 binding sites (Kaczynski et al., 2003).

### THP-1 Promoter ChIP-Chip Analysis

Loci showing H3K9-acetylation or Pu.1, Sp1, or Pol II binding were obtained as described previously (Suzuki, submitted 2008). ChIp-chip data were analysed such that a base must be bound to the protein or marker of interest in both replicates at time 0 or 96 h to be included. 0 h and 96 h ChIP-chip data were pooled and clustered such that any “present” base must have at least one other “present” base within 35 nt.

THP-1 tiRNA Characteristic Analysis

Evofold, phastCons, and CpG island loci were obtained from the local mirror of the UCSC Genome Browser. Intersections between tiRNAs and these genomic features were performed using a modified version of UCSC's bedIntersect. Sequence analysis was performed using python scrips and basic Unix tools. A one-tailed T-test was used to test if size distributions were different between tiRNAs and 3′ end small RNAs.

### THP-1 0 h Timepoint Analysis

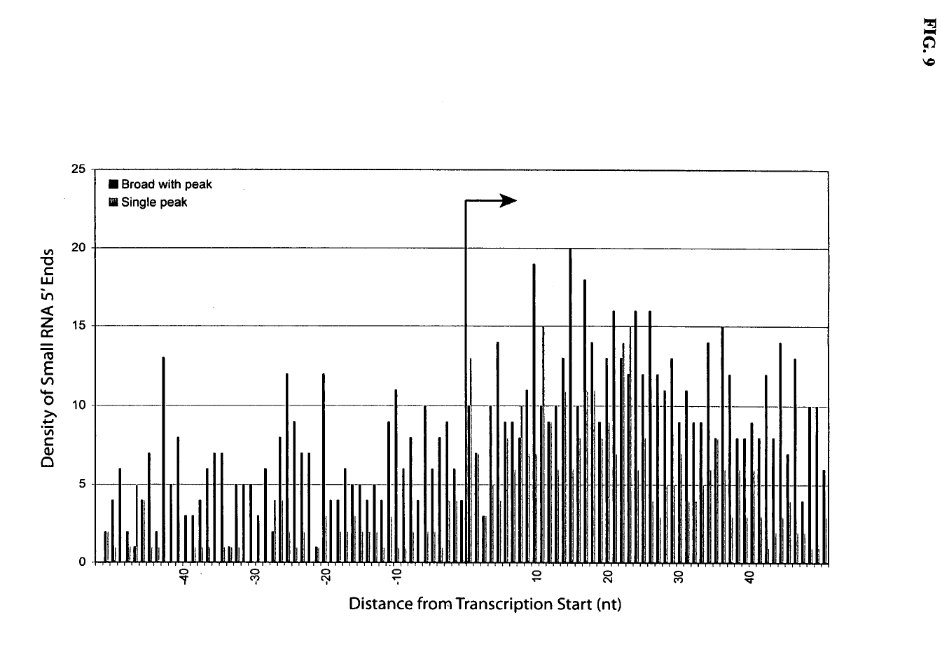

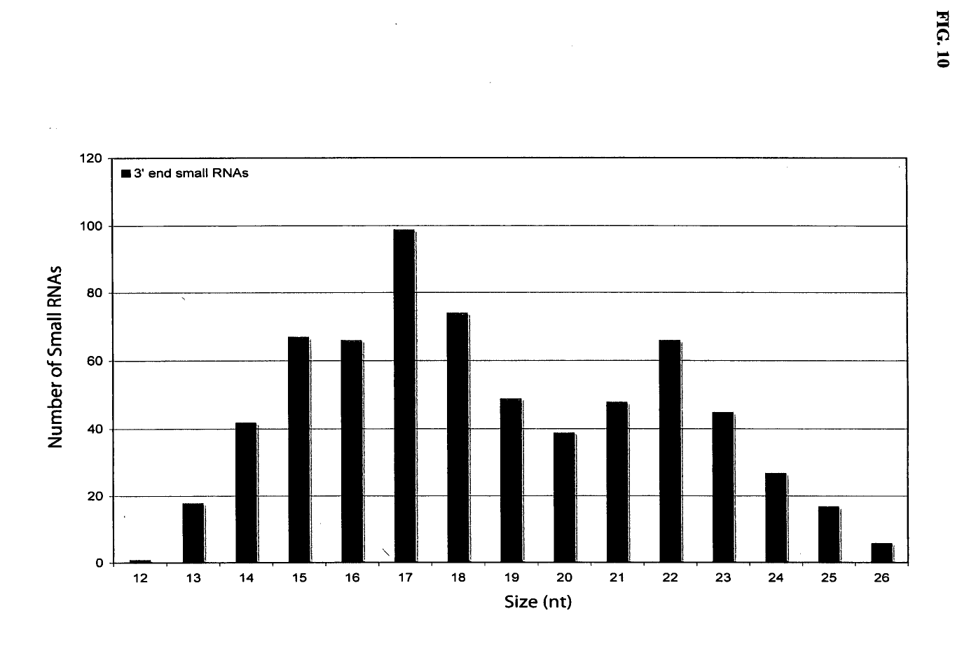

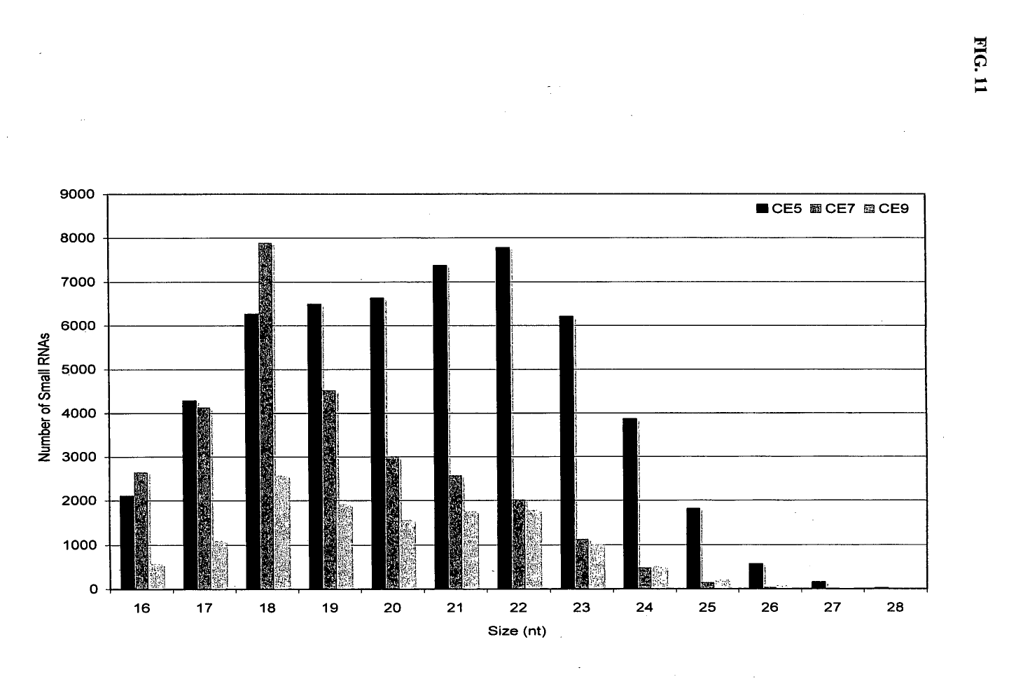

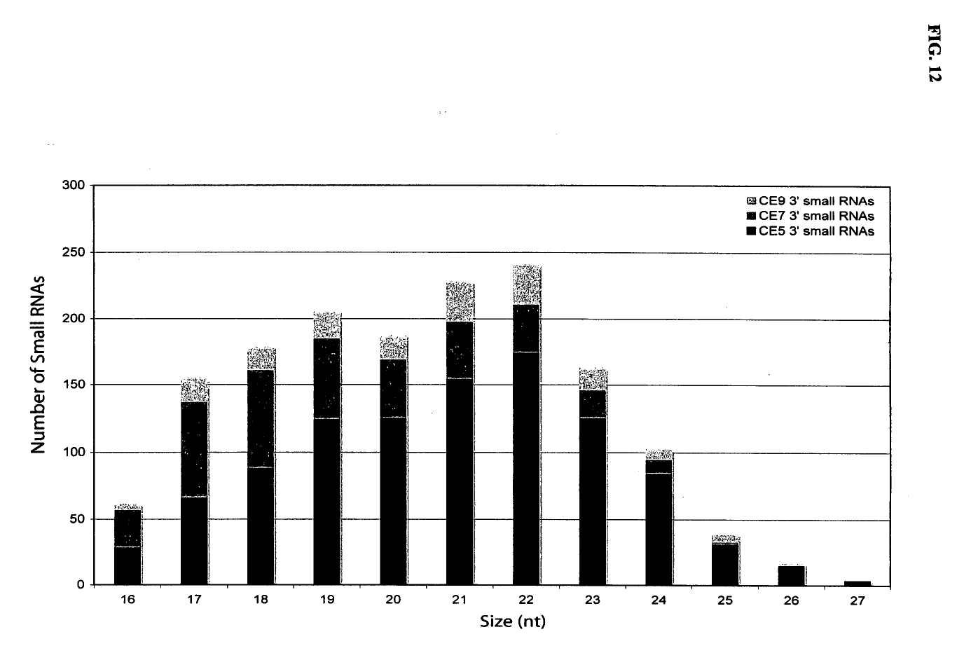

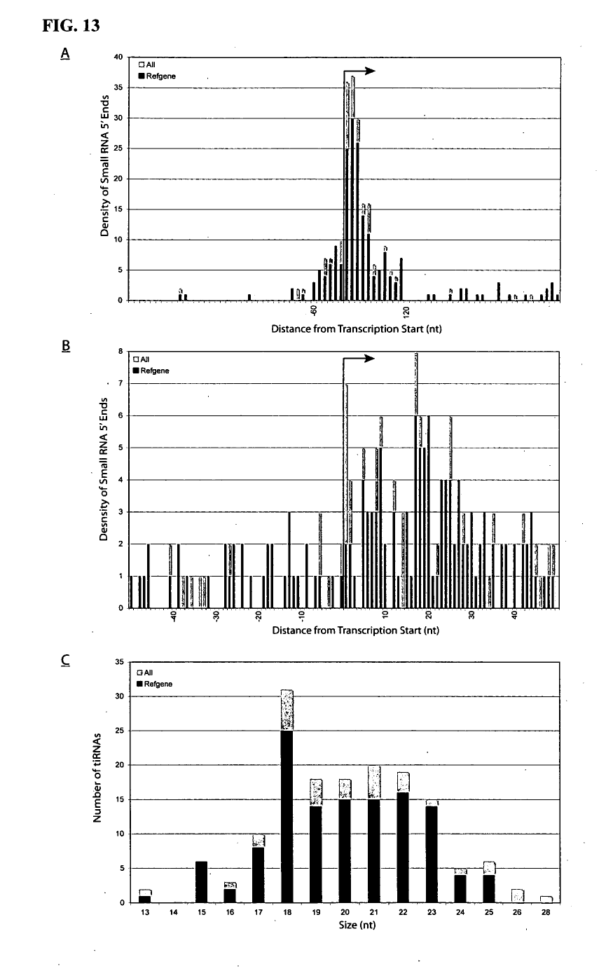

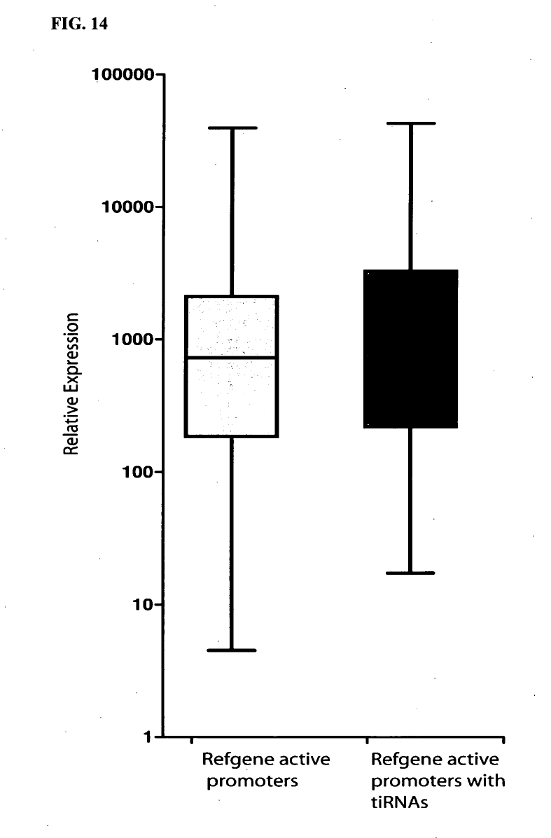

To ensure that pooling the deepCAGE and small RNA deep sequencing data across time points after THP-1 stimulation with PMA was not distorting the results, we restricted our analysis to the control time point at 0 h. Using deepCAGE tags detected in at least two replicates at 0 h, we found that all trends observed for the pooled dataset are recapitulated at 0 h, although overall less robustly. We found 156 small RNAs >200 fold enriched at 240 active promoters present at 0 h, which map to regions −60 to +120 nt relative to the TSS, with the highest density of tags 10 nt or further downstream (FIG. 13 A,B). The vast majority of these tiRNAs and their associated promoters map to Refgene TSSs (79% and 83% respectively), which are highly expressed (FIG. 14) and are enriched for Sp1 and RNA PolII binding (FIG. 15). 0 h tiRNAs are dominantly 18 nt (FIG. 13C), and have no intersection with Evofold predictions. Only one third intersect with a phastCons element. Consistent with tiRNAs from the pooled dataset we found that 0 h tiRNAs were ˜72% GC.

### Example 6

### tiRNAs in Chicken (Gallus gallus)

To determine if tiRNAs are present in other vertebrate species we then analysed small RNA libraries that were prepared from chicken embryos collected at day 5, day 7 and day 9 of incubation (hereafter referred to as CE5, CE7 and CE9 respectively) (Glazov et al., 2008). These represent the chicken embryonic developmental stages 25-27, 30-31 and 35, which cover major morphological changes (Hamburger and Hamilton, 1992). Interestingly, we found that the size distribution of uniquely mapping small RNAs at each time point varies considerably (Glazov et al., Submitted 2008) with later time points exhibiting proportionally more RNAs less than 20 nt (FIG. 11). Consistent with the human datasets, we found that small RNAs (less than 22 nt) were also over-represented at Refgene TSSs in chicken. Moreover, their fold enrichment at TSSs was directly related to the proportion of small RNAs in the dataset (FIG. 11). CE5 displayed the weakest enrichment at Refgene TSSs at 16×, while both CE7 and CE9 showed ˜60× enrichment at TSSs. CE5, 7 and 9 intersected 320, 507, and 231 Refgene TSSs, respectively. As in human cells, the small RNAs from the chicken libraries are tightly clustered −60 to +120 nt of Refgene TSSs, and show a density of small RNAs downstream of +10 nt (FIG. 4B). In total we found a total of 1886 tiRNAs which are dominantly 18 nt (FIG. 4E), in contrast to variable size distributions in 3′ end associated small RNAs, which show enrichment for sizes more frequently associated with miRNAs (FIG. 11). Chicken tiRNAs from all three libraries show expression levels (on average <0.85 cpm mapped tags), conservation levels (35% overlap with a phastCons element), and GC profiles (˜65% GC, >87% intersect a CpG island) consistent with human tiRNAs. We mapped chicken tiRNAs from CE5, CE7, and CE9 to the human genome. We found that >40% of chicken tiRNAs mapped to regions −60 to +120 nt relative to the most abundant human deepCAGE tag in a promoter, and >80% of chicken tiRNAs from each library map to regions −60 to +120 to any deepCAGE tag, suggesting that tiRNAs are positionally conserved.

Gallus gallus Small RNA Analysis

Solexa deep sequenced chicken small RNA tags were obtained from Glasov et al (Glazov et al., Submitted 2008). Tags were mapped to UCSC genome build galGal3 (v2.1 draft assembly, Genome Sequencing Center, Washington University School of Medicine) using Vmatch (http://www.vmatch.de/). Tags were included in subsequent analyses only if they mapped uniquely and without mismatches. Repeat masker annotations, genome assembly gaps, and Refgene, phastCons, and CpG island coordinates were obtained directly through the UCSC Genome Browser mirror. Known small RNA loci were compiled from miRBase (v 10.0) (Griffiths-Jones et al., 2008), and sequence homology searches with known mammalian snoRNAs. Small RNAs intersecting with any repeats, known small RNAs, assembly gaps, or the mitochondrial genome were removed from all analyses. Refgene TSSs coordinates were extracted from the UCSC Genome Browser. Bootstrap enrichment was preformed as described above. Small RNA distributions with respect to the TSS were calculated by tabulating the number of small RNA 5′ ends in each bin, as described above. Due to the paucity of Refgene annotations in the Gallus gallus genome, and therefore the limited number of TSSs used in this analysis, small RNAs mapping to multiple bins was observed less than 2% of cases. A one-tailed T-test was used to test if size distributions were different between tiRNAs and 3′ end small RNAs.

### Example 7

### tiRNAs in Drosophila

To investigate if tiRNAs are present in organisms outside the vertebrate lineage we queried publicly available Drosophila deep sequencing libraries (Ruby et al., 2007; Yin and Lin, 2007). Consistent with the human and chicken results, Drosophila small RNAs are enriched (>3 fold) in regions −60 to +120 nt relative to annotated Refgene start sites (FIG. 4C), are found 10 nt or more downstream of the TSS, are GC rich (>53%), and are dominantly 18 nt (FIG. 4F). In total we identified 1972 Drosophila tiRNAs, less than 1% of which overlap an Evofold prediction. The breadth of the Drosophila libraries allowed us to investigate if tiRNAs are disproportionately represented in specific areas of the body. More than 6% of tags derived from Drosophila heads are tiRNAs—nearly twice the proportion observed for any other library (Table 1). We also investigated whether tiRNAs are associated with genes that are regulated at the postinitiation stage of transcription (Mellor et al., 2008). This would be consistent with the observation that at noninduced but poised promoters, RNA Pol II pauses soon after promoter escape in the region around +20 to +40, with a peak of binding at +50 (ref. 26), positions which correlate well with peak tiRNA incidence. We intersected Drosophila tiRNAs from the Ruby et al. and Chung et al. datasets with stalled loci from 2-4 h embryos (Zeitlinger et al., 2008). At most one-third of the tiRNAs in any tissue or developmental-time-point library associate with a maximum of one quarter of stalled loci (Table 1). TiRNAs mapping to stalled loci are most abundant (˜threefold enriched) in embryonic and cultured S2 and K2 cell libraries (which may show an undifferentiated cell-type transcriptional state), consistent with the origin of the stalled gene dataset. This indicates that tiRNA expression may be influenced by RNA Pol II stalling, but tiRNAs are not exclusively associated with stalled transcripts.

### Drosophila Small RNA Analysis

Drosophila melanogaster deep sequencing libraries were obtained through NCBI GEO. Libraries GSE7448 (Ruby et al., 2007) and GSE11624 (Chung et al. 2008) were mapped to genome using Vmatch (http://www.vmatch.de/). Acquisition of genomic features and removal of small tags that mapped to small RNAs, repeats, etc. was accomplished as described above (Gallus gallus small RNA analysis). Bootstrap enrichment was preformed as described above. Small RNA distributions with respect to the TSS were calculated by tabulating the number of small RNA 5′ ends in each bin, as described above. Small RNAs mapping to multiple bins was observed in less than 10% of cases.

### Example 8

### tiRNAs and Disease Associated Genes

We have identified tiRNAs at a suite of oncogenes, including CITED4, p53, HoxA11, HoxA9, and myc in human THP-1 cells, a monocytic leukemia cell line. Importantly, we have also identified THP-1 tiRNAs at ETS1, which is known to be associated with monocytic leukemia progression and prognosis (FIG. 16), consistent with the origin of the model cell line.

We predict that tiRNAs are involved in gene expression by interacting directly with RNA Polymerase II, transcription factors, or other DNA binding proteins, or indirectly via chromatin modification (more below), and are dis-regulated in disease states. For example, we expect that the following genes will show aberrant tiRNA expression in leukemias: AF10, ALOX, 12, ARHGEF12, ARNT, AXL, BAX, BCL3, BCL6, BTG1, CAV1, CBFB, CDC23, CDH17, CDX2, CEBPA, CLC, CR1, CREBBP, DEK, DLEU1, DLEU2, EGFR, ETS1, EVI2A, EVI2B, FOXO3A, FUS, GLI2, GMPS, IRF1, KIT, LAF4, LCP1, LDB1, LMO1, LMO2, LYL1, MADH5, MLL3, MLLT2, MLLT3, MOV10L1, MTCP1, NFKB2, NOTCH1, NOTCH3, NPM1, NUP214, NUP98, PBX1, PBX2, PBX3, PBXP1, PITX2, PML, RAB7, RGS2, RUNX1, SET, SP140, TAL1, TAL2, TCL1B, TCL6, THRA, TRA, ZNFN1A1 (Leukemia associated genes were obtained from http://www.bioinformatics.org/legend/leuk_db.htm#g3)

Likewise, we predict that genes associated with other disease states will also show altered tiRNA expression. For example tiRNA expression will be altered at APP and APOE in Alzheimer's disease; BRCA1 and BRCA2 in breast cancer; HER2, ras, src, hTERT, and Bcl-2 in aggressive metastatic brain cancers; PON1 in coronary heart disease; and homeobox genes (e.g. HoxA10 and SOX2) in congenital developmental disorders.

To systematically examine tiRNA dis-regulation in these systems we will perform high throughput next generation deep sequencing (using an appropriate small RNA sequencing device, e.g. the Illumina Solexa Genome Analyzer II) on matched disease and normal tissues. Experiments will include biological and technical replicates and synthetic RNA spike-ins to facilitate normalization across libraries. A gene's tiRNA expression will be defined as the number of deep sequencing reads that map within −60-120 nt of the transcription start site. Disease gene tiRNA expression will be assessed, and those showing aberrant tiRNA levels will be functionally characterized using synthetic tiRNA-mimics and siRNAs against the tiRNAs. We predict that inhibition of tiRNA expression will selectively decrease gene expression, and that introduction of tiRNA mimics will increase gene expression.

### Example 9

### Human tiRNAs are Nuclear Localized

High throughput next generation deep sequencing was performed to determine tiRNA subcellular localization. Cultured THP-1 cells were grown to high density, and nuclear and cytosolic RNA fractions were isolated. RNA fraction quality was assessed on the Agilent Bioanalyzer. We employed Northern blots and qRT-PCR to detect nuclear specific (snoRNA and snRNA) and cytosolic specific (tRNA) small RNAs to ensure sample purity. Synthetic small RNA spike-ins were added to each sample to facilitate cross-library comparison. THP-1 nuclear and cytosolic 15-35 nt small RNA libraries were sequenced on the Illumina Solexa Genome Analyzer II.

tiRNAs are found almost exclusively in the nuclear fraction of THP-1 cells (Table 2 and FIG. 17). Small RNAs from the nuclear fraction are highly enriched at regions −60-120 nt relative to Refgene TSSs, are dominantly 18 nt, and intersect with more than a third of human Refgene annotations. In contrast, the cytosolic fraction contains very few promoter-proximal small RNAs, and hardly any are 18 nt. This data conclusively shows that tiRNAs are nuclear phenomenon.

### Example 10

### Genes with a High Abundance of tiRNAs are Enriched for 23 Specific Chromatin Marks

Human Refgenes with THP-1 derived tiRNAs were assessed for enrichment of 38 chromatin marks, RNA Polymerase II (PolII) and CTCF binding, and H2AZ, a rare histone (Barski et al. Cell (2007) vol. 129 (4) pp. 823-37 & Wang et al. Nature Genetics (2008) vol. 40 (7) pp. 897-903). Using the nuclear small RNA deep sequencing set, genes with tiRNAs were parsed into two groups: those having a high tiRNA abundance (total tag count >8,677 genes) or low tiRNA abundance (1 tiRNA, 2929 genes). The average chromatin mark or protein binding intensity was assessed at 1 nt resolution 200 nt up and downstream of the TSS.

Genes with a high density of tiRNAs show enrichment for 23 chromatin marks (H2AK5ac, H2AK9ac, H2AZ, H2BK120ac, H2BK12ac, H2BK20ac, H2BK5ac, H3K18ac, H3K23ac, H3K27ac, H3K36ac, H3K36me1, H3K4ac, H3K4me3, H3K79me2, H3K79me3, H3K9ac, H4K12ac, H4K16ac, H4K20me1, H4K5ac, H4K8ac, H4K91ac), PolII binding and H2AZ histones. These data suggest that tiRNAs are directly involved in the regulation of chromatin modification and gene expression.

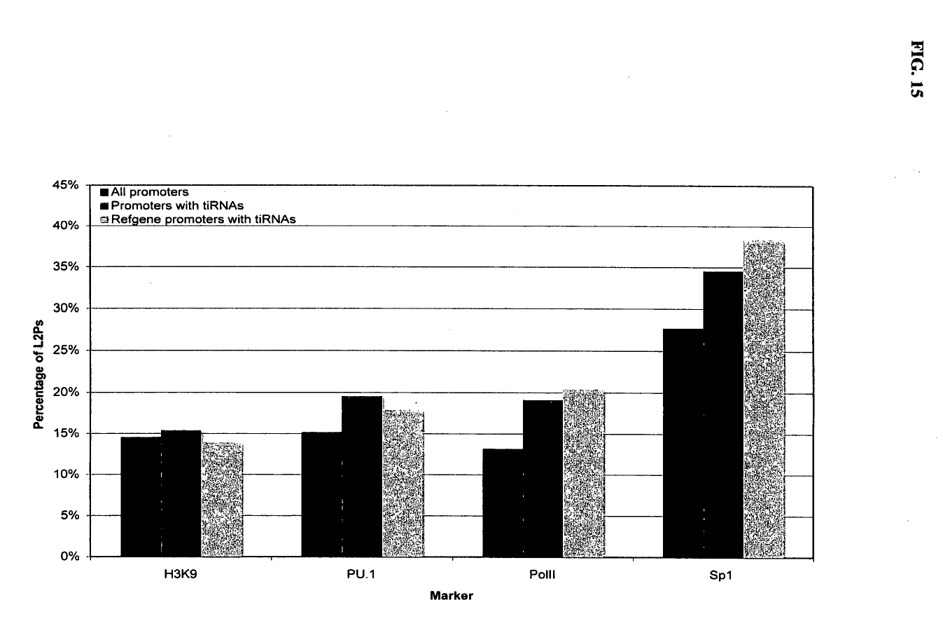

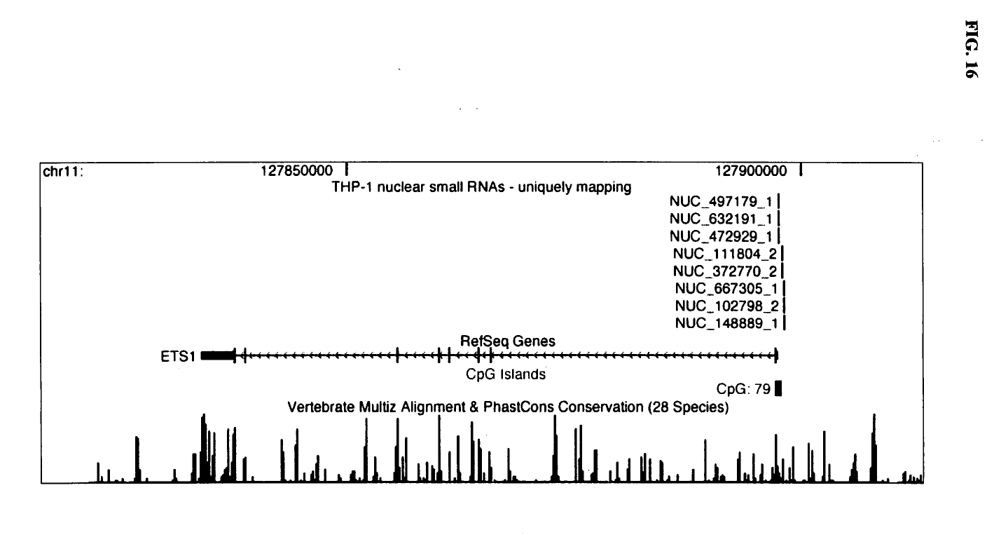

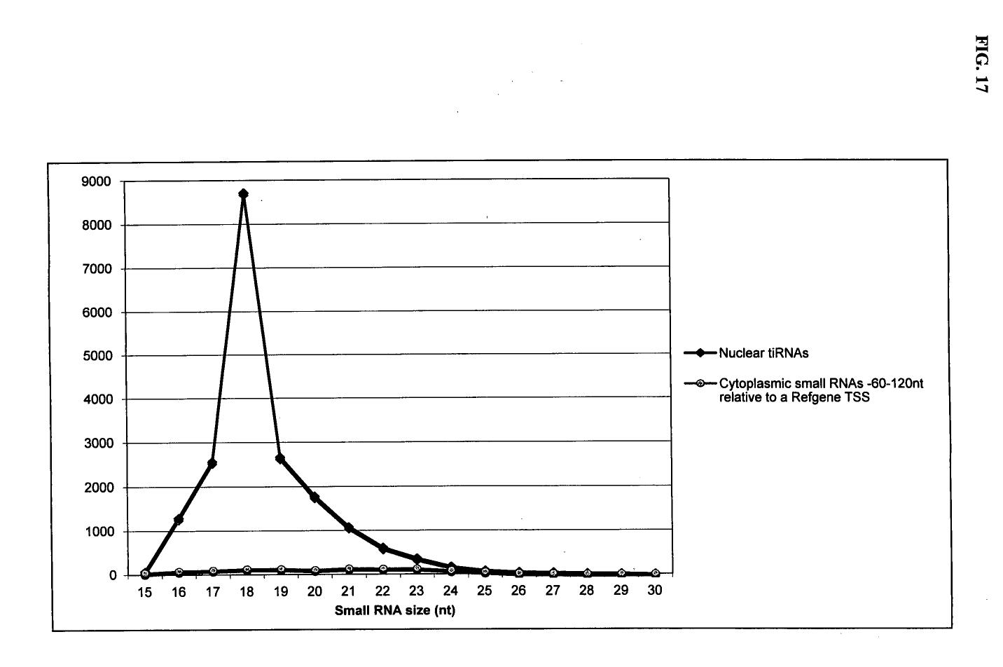

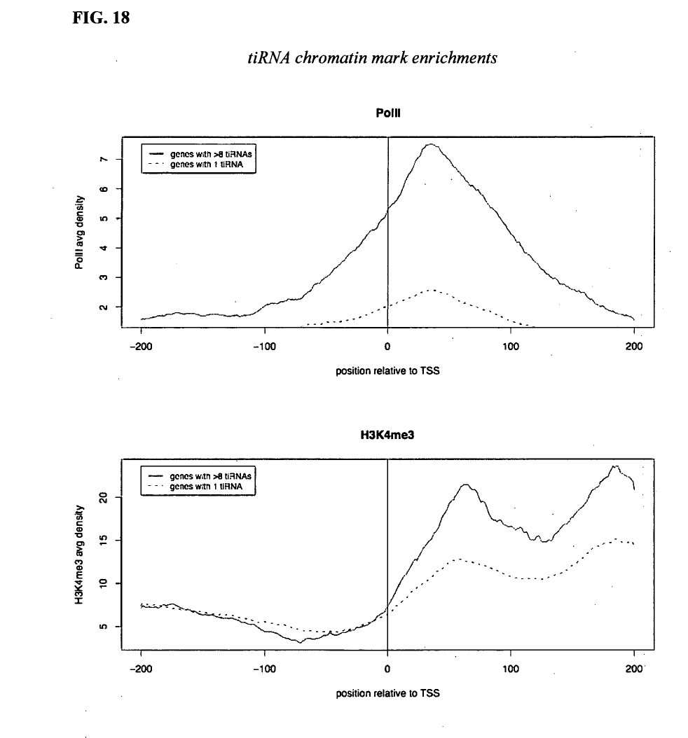

In each of the following graphs (FIG. 18) solid lines depicts the chromatin or protein binding density of genes with a high number of tiRNAs (solid red) or few tiRNAs (dashed blue). The TSS is denoted as a solid black vertical line. Gray bars at +10 and +30 indicate the region of tiRNA biogenesis.

### Example 11

### Unannotated 18 nt Nuclear Small RNAs are Enriched at Specific Chromatin Marks

The nuclear THP-1 small RNA data has a large abundance (˜80,000 sequences) of small RNAs that are dominantly 18 nt but do not map to canonical Refgene or UCSC KnownGene promoter regions. To assess if these 18 nt regions are tiRNA-like and are also enriched for specific chromatin marks we performed a bootstrap enrichment analysis, excluding canonical promoters and regions proximal to THP-1 deepCAGE clusters. To ensure that the analysis was not biased by known genomic features, THP-1 nuclear small RNA data were parsed to remove any sequences that mapped to repeats, small RNAs (e.g. tRNAs, snRNAs, snoRNAs, and miRNAs), assembly gaps, “random” chromosomes, or proximal to TSSs. We also analyzed a subset of this data, which was further parsed to remove any small RNAs that mapped within a UCSC KnownGene annotation.

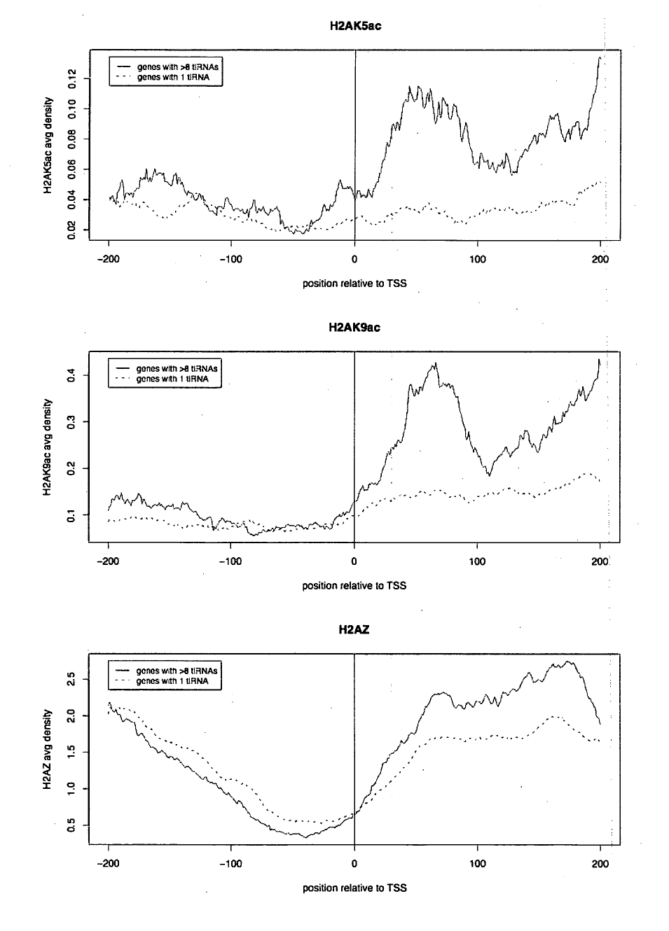

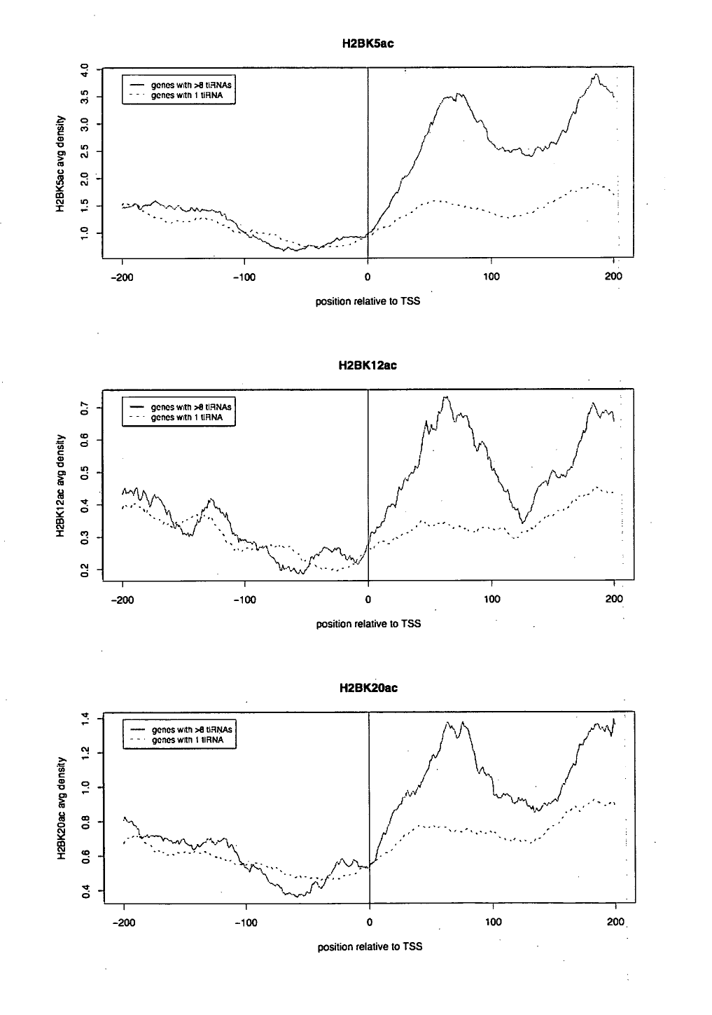

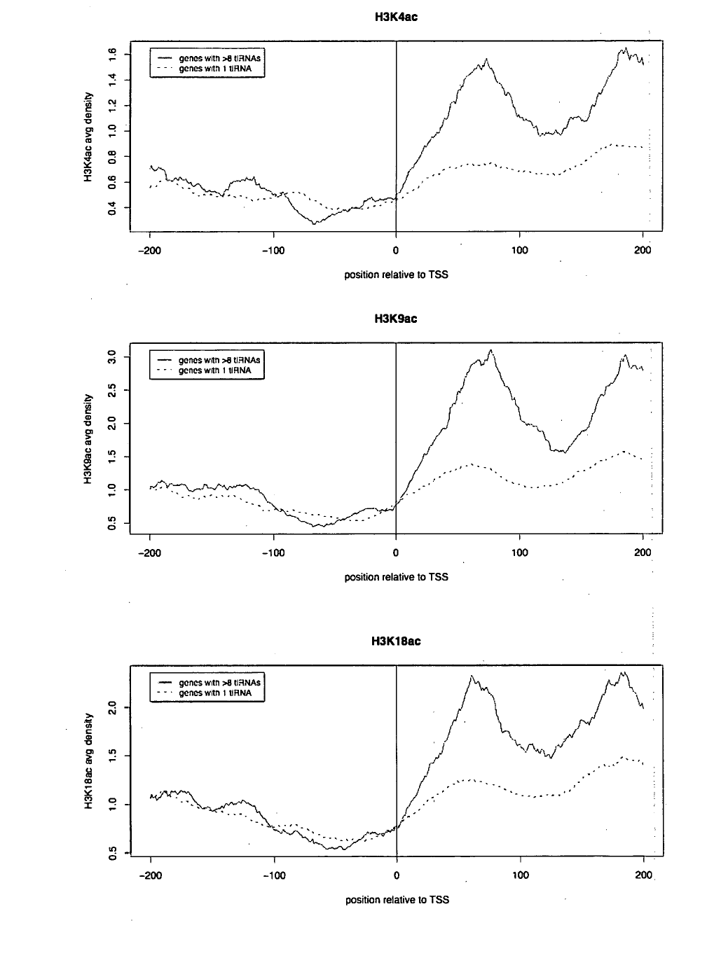

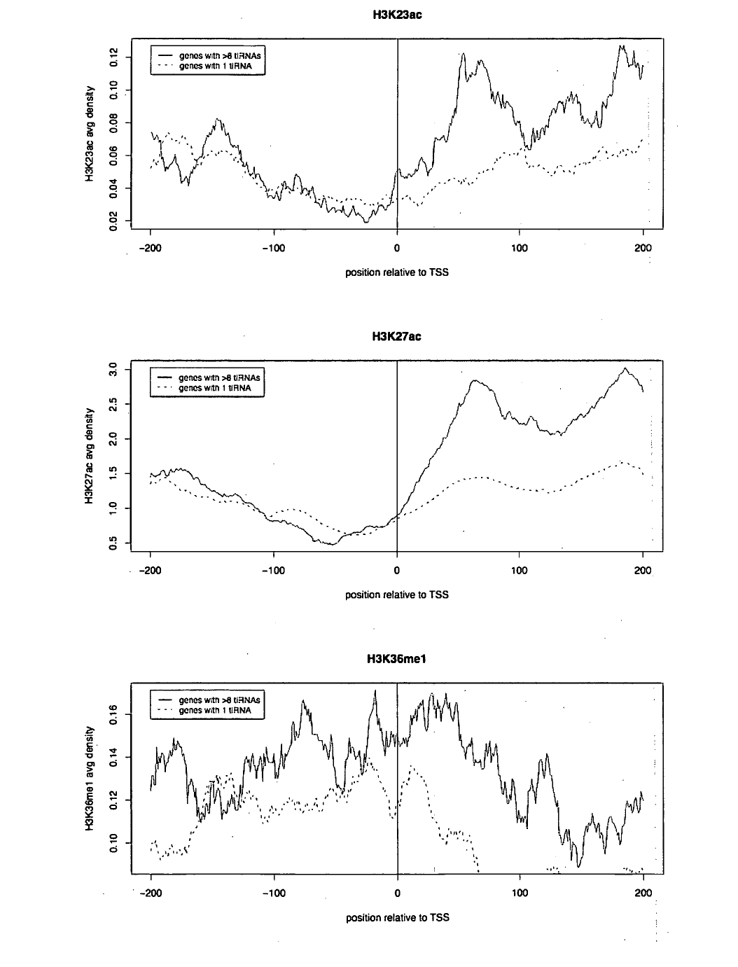

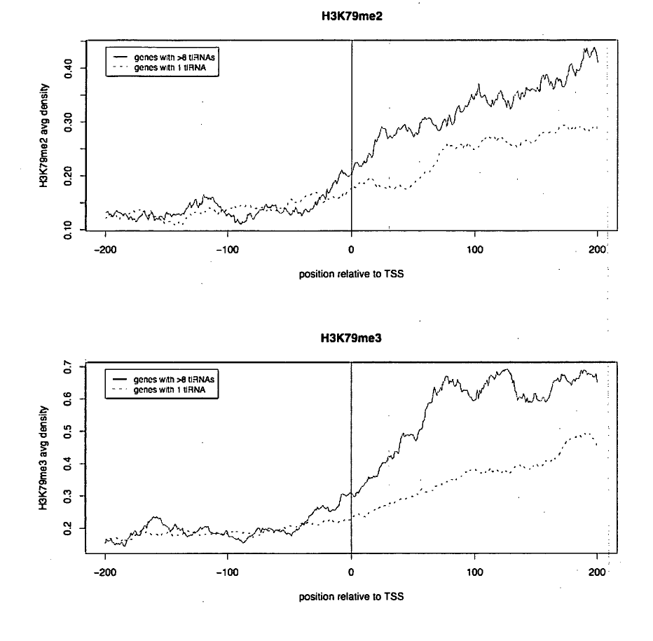

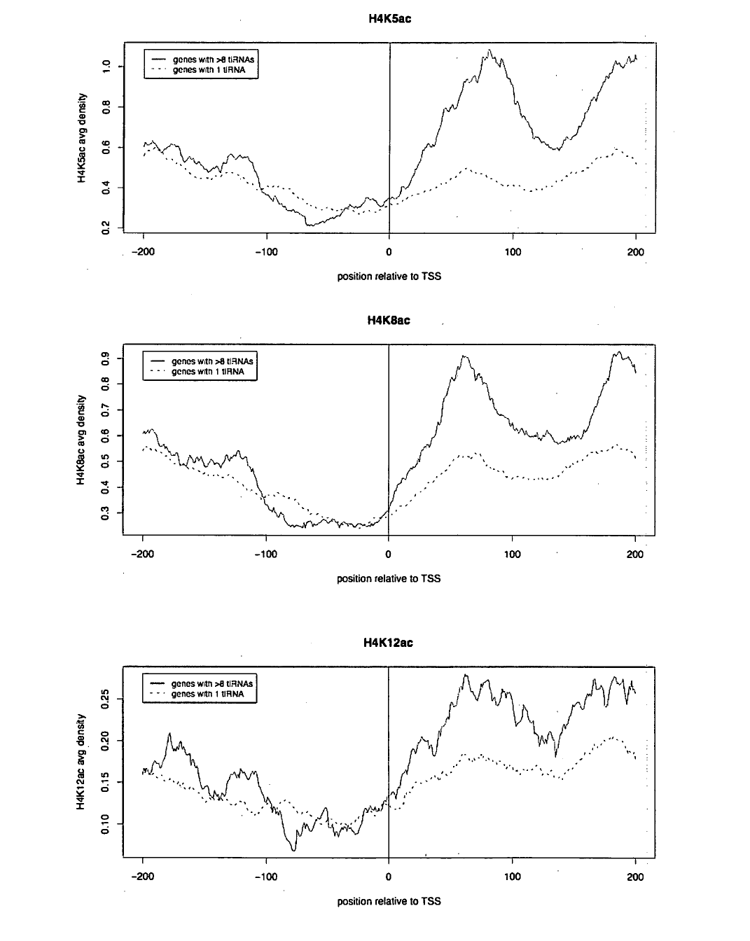

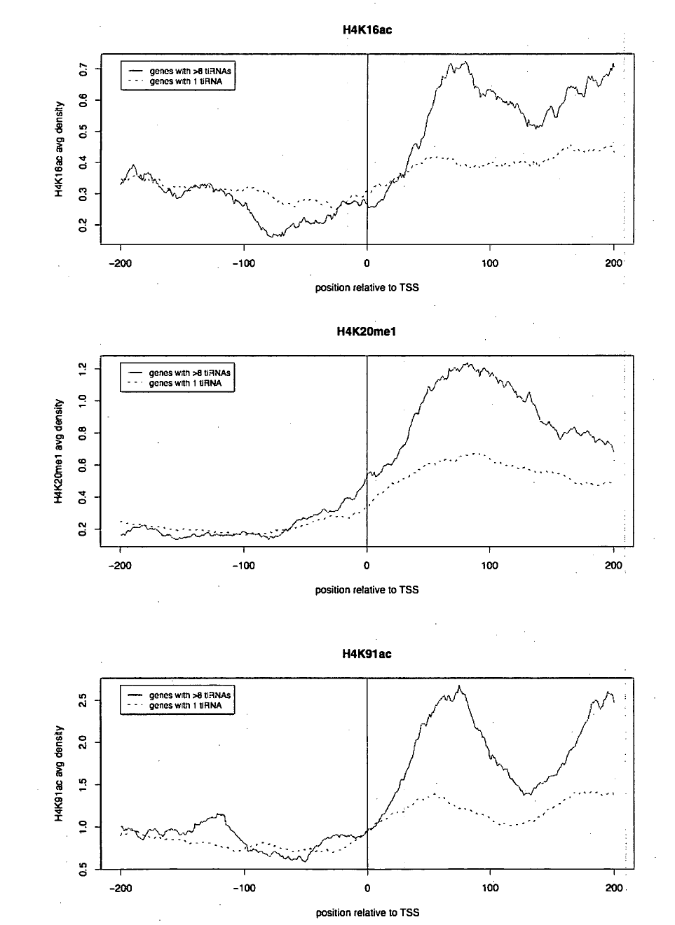

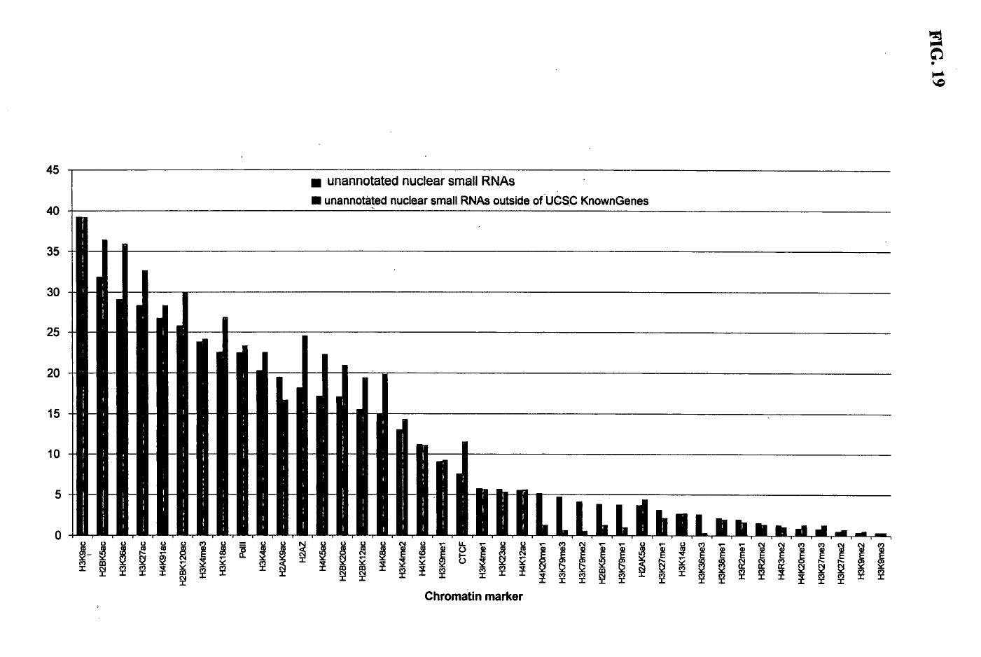

Nuclear-specific 18 nt small RNAs are highly enriched at regions with “activating” chromatin marks (e.g. H3K9ac, H3K4me3, and H3K120ac) and are under enriched at regions with “silencing” chromatin marks (FIG. 19). This enrichment is independent known tiRNA associations with these chromatin markers (since TSS proximal regions were completely excluded from the analysis), and suggests that 18 nt nuclear small RNAs, of which tiRNAs are a dominant subset, are generally associated with active chromatin and are involved in gene regulation by facilitating changes to chromatin structure.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

TABLE 1
Drosophila tiRNAs

| | | | | | | |
|---|---|---|---|---|---|---|
| | | Unannotated small | tiRNA | tiRNA abundance at | Number of genes | Number of stalled genes |
| Sample ID | Description | RNA abundance | abundance (%)^{a} | stalled genes (%)^{b} | with tiRNAs (%)^{c} | with tiRNAs (%)^{d} |

GSE7448

| | | | | | | | | | | |
|---|---|---|---|---|---|---|---|---|---|---|
| GSM180328 | Adult heads | 14,555 | 1,020 | (7) | 32 | (3) | 159 | (1) | 35 | (22) |
| GSM180329 | Adult bodies | 15,961 | 573 | (4) | 33 | (6) | 223 | (1) | 24 | (11) |
| GSM180330 | Early embryo | 8,569 | 82 | (1) | 22 | (27) | 106 | (1) | 26 | (25) |
| GSM180331 | Early embryo | 11,509 | 129 | (1) | 41 | (32) | 162 | (1) | 38 | (23) |
| GSM180332 | Mid embryo | 5,329 | 86 | (2) | 28 | (33) | 116 | (1) | 25 | (22) |
| GSM180333 | Late embryo | 14,547 | 314 | (2) | 57 | (18) | 332 | (2) | 56 | (17) |
| GSM180334 | 1st 3rd instars | 9,990 | 225 | (2) | 25 | (11) | 214 | (1) | 26 | (12) |
| GSM180335 | Imaginal discs | 16,162 | 283 | (2) | 21 | (7) | 222 | (1) | 26 | (12) |
| GSM180336 | Pupae (0-4 d) | 5,673 | 122 | (2) | 15 | (12) | 116 | (1) | 17 | (15) |
| GSM180337 | S2 cells | 19,252 | 171 | (1) | 54 | (32) | 219 | (1) | 51 | (23) |

GSE11624

| | | | | | | | | | | |
|---|---|---|---|---|---|---|---|---|---|---|
| GSM240749 | Female heads | 46,966 | 3,139 | (7) | 154 | (5) | 764 | (4) | 149 | (20) |
| GSM272651 | S2 and KC cells | 70,062 | 1,757 | (3) | 574 | (33) | 1,657 | (8) | 389 | (23) |
| GSM272652 | S2 cells | 327,046 | 5,799 | (2) | 1,665 | (29) | 3,699 | (18) | 678 | (18) |
| GSM272653 | KC cells | 108,486 | 4,031 | (4) | 1,473 | (37) | 2,787 | (14) | 591 | (21) |
| GSM275691 | Imaginal disc | 99,916 | 3,235 | (3) | 339 | (10) | 2,067 | (10) | 286 | (14) |
| GSM286601 | Male heads | 23,324 | 2,099 | (9) | 94 | (4) | 464 | (2) | 94 | (20) |
| GSM286602 | Male body | 56,524 | 3,633 | (6) | 251 | (7) | 1,072 | (5) | 146 | (14) |
| GSM286603 | Female body | 90,494 | 4,513 | (5) | 368 | (8) | 1,506 | (7) | 200 | (13) |
| GSM286604 | Embryo (0-1 h) | 241,146 | 11,207 | (5) | 1,026 | (9) | 2,134 | (10) | 327 | (15) |
| GSM286613 | Embryo* (0-1 h) | 126,413 | 1,972 | (2) | 370 | (19) | 1,725 | (8) | 286 | (17) |
| GSM286605 | Embryo (2-6 h) | 213,042 | 4,273 | (2) | 838 | (20) | 2,284 | (11) | 430 | (19) |
| GSM286606 | Embryo* (2-6 h) | 47,944 | 1,050 | (2) | 209 | (20) | 510 | (2) | 97 | (19) |
| GSM286607 | Embryo (6-10 h) | 102,773 | 2,875 | (3) | 943 | (33) | 1,241 | (6) | 315 | (25) |
| GSM286611 | Embryo* (6-10 h) | 90,311 | 3,358 | (4) | 1,143 | (34) | 1,966 | (10) | 454 | (23) |

^{a}Percentage of unannotated small RNA abundance,
^{b}tiRNA abundance,
^{c}all Refgenes, or
^{d}of stalled genes.
*Biological replicate libraries.

TABLE 2
tiRNAs are nuclear enriched

| | | Cytoplasmic |
|---|---|---|
| | Nuclear small | small RNA |
| | RNA fraction | fraction |
| Number of small RNAs within | 15,012 | 927 |
| −60-120 nt of a human Refgene TSS | | |
| Dominant small RNA size | 18 nt | 21 nt |
| Total abundance of small RNAs within | 19,481 | 1143 |
| −60-120 nt of a Refgene TSS | | |
| Number of genes with small RNAs within | 7014 | 914 |
| −60-120 nt of a Refgene TSS | | |
| Total tiRNA enrichment | ~12 fold | — |

### REFERENCES

F. Al-Shahrour et al., Nucl Acids Res 35: W91 (2007).

A. Barski et al., Cell 129 (4): 823 (2007).

P. Carninci et al., Nat Genet 38: 626 (2006).

W J. Chung et al., Curr. Biol 18: 795 (2008).

D R. Corey and J M. Abrams, Genome Biol 2: 1015.1 (2001).

N. Dias and C A. Stein, Mol Cancer Ther 1: 347 (2002).

C Y. Chu and T M. Rana, J Cell Physiol 213: 412 (2007).

G. Dieci et al., Trends Genet 23: 614 (2007).

M M. Fabani and M J. Gait, RNA 14: 336 (2008).

C R. Faehnle et al., Curr Opin Chem Biol 11: 569 (2007).

M. Gardiner-Garden and M. Frommer, J Mol Biol 196: 261 (1987).

E A. Glazov et al., Genome Research, 18:957 (2008).

S. Griffiths-Jones et al., Nucleic Acids Res 36: D154 (2008).

E. Grünblatt et al., J Alzheimers Dis, 12: 291 (2007).

V. Hamburger et al., Dev Dyn 195: 231 (1992).

http://www.oligos.com/ModificationsList.htm

G. Hutvagner et al., PLoS Biology, 2: 465 (2004).

J. Kaczynski et al., Genome Biol 4: 206 (2003).

P. Kapranov et al., Science 316: 1484 (2007).

D. Karolchik et al., Nucl Acids Res 36: D773 (2008).

R. Kos et al., Dev Dyn 226: 470 (2003).

J. Krützfeldt et al., Nature, 438: 685 (2005).

J. Kurreck, Eur J Biochem, 270: 1628 (2003).

B P. Lewis et al., Cell, 115: 787 (2003).

B P. Lewis et al., Cell, 120: 15 (2005).

W S. Liang et al., Physiol Genomics (2008).

A K. Lübke et al., Arthritis Res Ther, 18: R9 (2008).

M. Margulies et al., Nature 437, 376 (2005).

J S. Mattick and I V. Makunin, Hum Mol Genet 14: R121 (2005).

B C. Mc Kaig et al., Am J Pathol 162: 1355 (2003).

G. Meister et al., RNA, 10: 544 (2004).

J. Mellor et al., Curr. Opin. Genet. Dev. 18:116(2008)

M. Partridge et al., Antisense Nucleic Acid Drug Dev 6: 169 (1996).

J S. Pedersen et al., PLoS Comput Biol 2: e33 (2006).

R S. Pillai et al., Trends Cell Biol 17: 118 (2007).

P M. Ridker, Nutr Rev, 65: S253 (2007).

E. van Rooij and E N. Olson, J Clin Invest 117: 2369 (2007).

J G. Ruby et al., Genome Res 17: 1850 (2007).

N K. Sahu et al., Curr Pharm Biotechnol 8: 291 (2007).

T. Shiraki et al., Proc Natl Acad Sci USA 100: 15776 (2003).

A. Siepel et al., Genome Res 15: 1034 (2005).

J. Summerton and D. Weller, Antisense Nucleic Acid Drug Dev 7: 187 (1997).

H. Suzuki, Submitted (2008).

O. Tam et al., Nature 453:534 (2008).

B. Tews et al., Oncogene 26: 5010 (2007).

S. Tsuchiya et al., Cancer Res 42: 1530 (1982).

S. Vasudevan et al., Science 318: 1931 (2007).

Z. Wang et al., Nature Genetics 40 (7): 897 (2008).

J D. Ye et al., Proc Natl Acad Sci USA. 105: 82 (2008).

H. Yin and H. Lin, Nature 450: 304 (2007).

Y. You et al., Nucl Acids Res 34: e60 (2006).

S. Zecchini et al., Cancer Res 68: 1110 (2008).

J. Zeitlinger et al., Nat. Genetics 39:512 (2008).

B. Zhang et al., Dev Biol 302: 1 (2007).

1-49. (canceled)

50. A substantially single-stranded isolated RNA molecule, wherein said isolated RNA molecule comprises a nucleotide sequence:
(i) consisting of no more than 25 contiguous nucleotides;
(ii) corresponding to a non-protein-coding genomic DNA sequence located between −200 and +300 nucleotides from a transcription start site (TSS) in a genome of an organism; and
(iii) having an average GC content that is greater than 60%.

51. The isolated RNA molecule of claim 50, wherein said nucleotide sequence consists of 14-22 contiguous nucleotides.

52. The isolated RNA molecule of claim 50, wherein said nucleotide sequence consists of 18 or 19 contiguous nucleotides.

53. The isolated RNA molecule of claim 50, wherein said genomic DNA sequence is located between −60 and +120 nucleotides from said TSS in said genome.

54. The isolated RNA molecule of claim 50, wherein said nucleotide sequence is located within at least one CpG island.

55. The isolated RNA molecule of claim 50, wherein said nucleotide sequence comprises at least one CpG dinucleotide.

56. The isolated RNA molecule of claim 50 having a 5′ end that corresponds to a genomic DNA sequence located between −50 and +70 nucleotides from a TSS in a genome.

57. The isolated RNA molecule of claim 50, wherein said isolated RNA molecule is located at or near a TSS and wherein said TSS is associated with an RNA polymerase II promoter and/or an Sp1 transcription factor binding site.

58. The isolated RNA molecule of claim 50, wherein said genome is of a human.

59. The isolated RNA molecule of claim 50, comprising a nucleotide sequence selected from any one of the nucleotide sequences set forth in SEQ ID NOs: 1 to 17213, or a nucleotide sequence at least partly complementary thereto.

60. A modified RNA molecule comprising the isolated RNA molecule of claim 50, or a nucleotide sequence at least 70% identical thereto.

61. A fragment of the isolated RNA molecule of claim 50, wherein said fragment comprises at least 5 nucleotides of said isolated RNA molecule.

62. A genetic construct comprising or encoding one or more of the isolated RNA molecules of claim 50.

63. A host cell containing the genetic construct of claim 62.

64. A method of identifying the isolated RNA molecule of claim 50, said method including the step of isolating one or more of said isolated RNA molecules from a nucleic acid sample.

65. The method of claim 64, wherein said nucleic acid sample is from a human.

66. A method of identifying a genomic DNA sequence, said method including the step of identifying a DNA sequence in a genome of an organism which is complementary to the nucleotide sequence of the isolated RNA molecule of claim 50.

67. A method of identifying a regulatory region of a genome, said method including the step of identifying the isolated RNA molecule of claim 50.

68. The method of claim 67, wherein said regulatory region is a transcriptionally active region.

69. The method of claim 67, wherein said genome is of a human.

70. A method of determining whether a mammal has, or is predisposed to, a disease or condition associated with one or more regulatory regions of a genome, said method including the step of determining whether said mammal comprises one or more of the isolated RNA molecules according to claim 50, wherein the or each nucleotide sequence of said one or more isolated RNA molecules corresponds to a genomic DNA sequence associated with said disease or condition.

71. The method of claim 70, wherein said one or more regulatory regions is a transcriptionally active location and/or region.

72. The method of claim 70, wherein said mammal is a human.

73. A nucleic acid array comprising a plurality of the isolated RNA molecules of claim 50, or one or more isolated nucleic acids respectively complementary thereto, immobilized, affixed or otherwise mounted to a substrate.

74. A kit comprising one or more of the isolated RNA molecules of claim 50, or one more isolated nucleic acids respectively complementary thereto, and one or more detection reagents.

75. A method of treating a disease or condition in a mammal, said method including the step of administering to said mammal a therapeutic agent comprising the isolated RNA molecule of claim 50, to thereby treat said disease or condition.

76. The method of claim 75, wherein said disease or condition is associated with aberrant regulation of one or more genes.

77. The method of claim 75, wherein said disease or condition is associated with aberrant transcriptional activity of one or more genes.

78. The method of claim 75, wherein said disease or condition is selected from the group consisting of Crohn's disease, Alzheimer's disease, Parkinson's disease, rheumatoid arthritis, myocardial infarction, diabetes, congenital developmental disorders, coronary heart disease and cancer such as breast cancer, lymphoma, leukemia, aggressive metastatic brain cancers, colorectal cancer, gastric cancer, ovarian cancer and pituitary tumors.

79. The method of claim 75, wherein said mammal is a human.

80. A pharmaceutical composition comprising a therapeutic agent comprising the isolated RNA molecule of claim 50, and a pharmaceutically acceptable carrier, diluent or excipient.

81. A pharmaceutical composition comprising a therapeutic agent comprising the isolated RNA molecule of claim 50, and a pharmaceutically acceptable carrier, diluent or excipient, for use in treating a disease or condition in a mammal.

82. The pharmaceutical composition of claim 80, wherein said disease or condition is associated with aberrant regulation of one or more genes.

83. The pharmaceutical composition of claim 80, wherein said disease or condition is associated with aberrant transcriptional activity of one or more genes.

84. The pharmaceutical composition of claim 80, wherein said disease or condition is selected from the group consisting of Crohn's disease, Alzheimer's disease, Parkinson's disease, rheumatoid arthritis, myocardial infarction, diabetes, congenital developmental disorders, coronary heart disease and cancer such as breast cancer, lymphoma, leukemia, aggressive metastatic brain cancers, colorectal cancer, gastric cancer, ovarian cancer and pituitary tumors.

85. The pharmaceutical composition of claim 80, wherein said mammal is a human.
